(19) United States
(12) Patent Application Publication
(10) Pub. No.: US 2025/0289827 A1
(43) Pub. Date: Sep. 18, 2025

(54) MORPHIC FORMS OF A MUTANT BRAF DEGRADER AND METHODS OF MANUFACTURE THEREOF

(71) Applicant: C4 THERAPEUTICS, INC., Watertown, MA (US)

(72) Inventors: Robert T. Yu, Arlington, MA (US); Minsheng He, Andover, MA (US); Matthew J. Schnaderbeck, Methuen, MA (US); Bridget Kreger, Acton, MA (US); Roy Macfarlane Pollock, Medford, MA (US); Siyi Jiang, Shanghai (CN); Meiqi Li, Shanghai (CN); Bolu Chen, Shanghai (CN); Jiannan Lu, Cambridge, MA (US)

(73) Assignee: C4 THERAPEUTICS, INC., Watertown, MA (US)

(21) Appl. No.: 19/224,081

(22) Filed: May 30, 2025

Related U.S. Application Data
(63) Continuation of application No. PCT/US2023/082137, filed on Dec. 1, 2023.
(60) Provisional application No. 63/438,422, filed on Jan. 11, 2023.
(60) Provisional application No. 63/599,450, filed on Nov. 15, 2023.

(30) Foreign Application Priority Data
Dec. 2, 2022 (CN) 202211627337.6

(51) Int. Cl. C07D 491/107; A61K 31/517; A61P 35/00

(52) U.S. Cl. CPC C07D 491/107; A61K 31/517; A61P 35/00

(57) ABSTRACT

Advantageous isolated morphic forms of (3R)-3-[6-[2-cyano-3-[[ethyl(methyl)sulfamoyl]amino]-6-fluorophenoxy]-4-oxoquinazolin-3-yl]-8-[2-[1-[3-(2,4-dioxo-1,3-diazinan-1-yl)-5-fluoro-1-methylindazol-6-yl]-4-hydroxypiperidin-4-yl]acetyl]-1-oxa-8-azaspiro[4.5]decane (Compound 1), which is a mutant BRAF degrader, and methods to prepare Compound 1 morphic forms for therapeutic applications are provided in the invention. The invention also provides improved methods for the synthesis of Compound 1, new pharmaceutical compositions comprising Compound 1, and new uses of Compound 1.

### CROSS-REFERENCE TO RELATED APPLICATIONS

This application is a continuation of International Patent Application No. PCT/US2023/082137, filed in the U.S. Receiving Office on Dec. 1, 2023, which claims the benefit of Chinese Patent Application No. 202211627337.6 filed Dec. 2, 2022, U.S. Provisional Application No. 63/438,422 filed Jan. 11, 2023, and U.S. Provisional Application No. 63/599,450 filed Nov. 15, 2023. The entirety of each of these applications is hereby incorporated by reference herein for all purposes.

### FIELD OF THE INVENTION

The present invention provides advantageous isolated morphic forms of (3R)-3-[6-[2-cyano-3-[[ethyl(methyl)sulfamoyl]amino]-6-fluorophenoxy]-4-oxoquinazolin-3-yl]-8-[2-[1-[3-(2,4-dioxo-1,3-diazinan-1-yl)-5-fluoro-1-methylindazol-6-yl]-4-hydroxypiperidin-4-yl]acetyl]-1-oxa-8-azaspiro[4.5]decane (Compound 1), which is a mutant BRAF degrader, and methods to prepare Compound 1 morphic forms for therapeutic applications as described further herein. The invention also provides improved methods for the synthesis of Compound 1 and isotopic, for example deuterium, derivatives thereof, new pharmaceutical compositions comprising Compound 1, and new uses of Compound 1.

### BACKGROUND

BRAF is a serine/threonine protein kinase that is a member of the signal transduction protein kinases. BRAF plays a critical role in the mitogen activated protein kinase (MAPK) signaling pathway and is mutated in approximately 8% of all human cancers including melanoma (˜60/0), colorectal (˜10%), and lung adenocarcinoma (˜5%). BRAF mutations have also been identified in thyroid cancer, and others. The most common mutation in BRAF is V600E (Class I), which occurs in half of malignant melanomas. This mutation hyperactivates ERK and works as a RAF inhibitor-sensitive monomer. Other common activating mutations include Class II mutations such as G469A and Class III mutations such as G466V. Class II and III mutations activate ERK by promoting RAF homo- or hetero-dimerization.

The BRAF protein presents a mechanism for signaling propagation that requires protein homo-dimerization (BRAF-BRAF) or hetero-dimerization with other RAF proteins (BRAF-RAF1 or BRAF-ARAF). When BRAF is mutated, as observed in oncology indications with BRAF V600E/K substitutions, BRAF signaling becomes independent of homodimers and/or heterodimers. The kinase activity becomes hyperactivated as a monomeric protein and drives cellular proliferative signals.

Several BRAF inhibitors have been described that can inhibit monomeric BRAF but not dimeric BRAF including vemurafenib, dabrafenib, and encorafenib. However, resistance usually emerges within a year, including RAS mutation, BRAFV600E amplification, and BRAFV600E intragenic deletion or splice variants. These inhibitors are also ineffective against non-V600 BRAF mutants (Class II & III) that activate ERK by promoting RAF homo- or hetero-dimerization.

Despite the therapeutic benefits of available BRAF inhibitors, the duration of the antitumor response to these drugs can be limited by the emergence of drug resistance.

Examples of BRAF inhibitors are described in patent applications filed by Hoffman La Roche AG including WO2021/116055, WO2021/116050, WO2022/129259, WO2022/129260, WO2022/258612, and WO2022/258600.

C4 Therapeutics Inc. filed patent application WO2022/261250 which describes BRAF degrading compounds. Additional, non-limiting examples of BRAF degrading compounds include those described in WO2018/119448, WO2019/199816, WO2020/051564, WO2021/255212, and WO2022/047145.

Despite these efforts there remains a need for new therapeutic drugs and approaches to treat BRAF mediated cancers, and in particular drugs that treat mutant BRAF mediated cancers.

### SUMMARY OF THE INVENTION

Stable and crystalline morphic forms of Compound 1 have been discovered including Compound 1 Form B and Compound 1 sodium salt Form F.

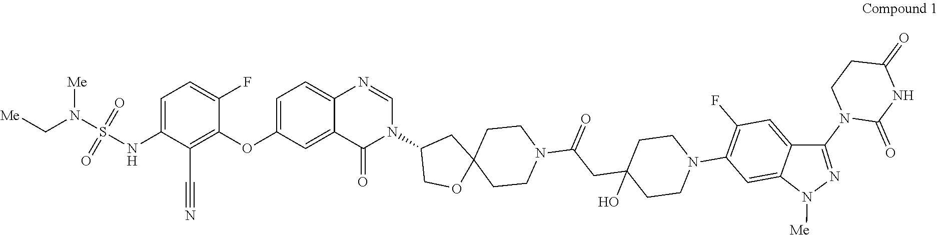 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

Morphic Form B is superior to other morphic forms of Compound 1 due to its high stability, scalability, and reproducibility. For example, when Compound 1 Form B is tested for stability over a one-week period at 25° C./92% relative humidity in an open container, 40° C./75% RH in an open container, and at 60° C. in a tight container, Compound 1 Form B demonstrates excellent chemical and physical stability without any changes in purity, crystal form and crystallinity (See Example 15, bulk stability). When Compound 1 Form B is tested in a water sorption and desorption experiment at 25° C. (See Example 16), Compound 1 Form B demonstrates excellent stability and no changes in crystallinity with only slight hygroscopicity (water uptake of 1.6% at 95% RH). Compound 1 Form B also shows excellent morphic form stability under compression (2 MPa and 10 MPa, Example 17), dry grinding and wet granulation conditions (Examples 18-19). Wet granulation experiments in the presence of water or ethanol also indicate no changes in crystallinity (Example 19). These advantageous and beneficial stability characteristics of Compound 1 Form B are useful properties that increase the shelf life of Compound 1 Form B and pharmaceutical compositions thereof and allow for increased purity and reproducibility in manufacturing scale preparations. High stability of Compound 1 Form B under high humidity conditions is useful and beneficial for development and manufacture of pharmaceutical compositions. These properties can also be used to modulate the delivery rate of Compound 1 for improved therapeutic effect.

Sodium salt Form F also shows good physicochemical characteristics including good crystallinity, stoichiometry, high dehydration temperature and good counter ion safety. When Compound 1 sodium salt Form F is tested for stability over a one-week period at 25° C./92% relative humidity in an open container, 40° C./75% RH in an open container, and at 60° C. in a tight container, Compound 1 sodium salt Form F demonstrates excellent morphic form stability (no form change) (See Example 25). Form F shows only a slight decrease in chemical purity (1.2%) after one-week stress at 25° C./92% RH and slight crystallinity decrease after stress at 60° C. in a tight container over one week. In a water sorption and desorption experiment at 25° C. (Example 27), Compound 1 sodium salt Form F demonstrates excellent stability and no changes in crystallinity. These advantageous and beneficial stability characteristics of Compound 1 sodium salt Form F are useful properties that increase shelf life of Compound 1 sodium salt Form F and pharmaceutical compositions thereof and allow for increased purity and reproducibility in manufacturing scale preparations. These properties can also be used for manufacture of pharmaceutical compositions containing Compound 1 sodium salt Form F and to modulate the delivery rate of Compound 1 for improved therapeutic effect.

Compound 1 is a small-molecule mutant BRAF degrader that degrades mutant BRAF, for example a Class I, Class II, and/or Class III mutant BRAF, via the ubiquitin proteasome pathway. Compound 1 binds to the ubiquitously expressed E3 ligase protein cereblon (CRBN) and alters the substrate specificity of the CRBN E3 ubiquitin ligase complex, resulting in the recruitment and ubiquitination of mutant BRAF, such as, for example, BRAF V600E. Compound 1 effectively degrades Class I mutant BRAF such as V600E, Class II mutant BRAF such as G469A, Class III mutant BRAF such as G466V mutations, and splice variants such as p61-BRAF^{V600E}. For example, in A375 cells, Compound 1 potently degrades BRAF^{V600E} (Eₘₐₓ=26% (i.e., 74% of BRAF protein degraded); DC₅₀=14 nM at 24 hr) and inhibits ERK phosphorylation (IC₅₀=11 nM at 24 hr) and cell growth (GI₅₀=94 nM at 96 hr).

In other aspects a new advantageous pharmaceutical composition comprising Compound 1 or a Compound 1 morphic form according to the present invention that is suitable for administration to humans is provided. This advantageous pharmaceutical composition comprises Compound 1 or a pharmaceutically acceptable salt thereof and one or more pharmaceutically acceptable excipients wherein the pharmaceutical composition is a tablet comprising both intragranular and extragranular particles and wherein the intragranular particles comprise Compound 1. In certain embodiments, Compound 1 or a pharmaceutically acceptable salt thereof and one or more pharmaceutically acceptable excipients are spray dried to form a spray dried dispersion that is then processed into the intragranular particles. In certain embodiments, the intragranular excipients added before the spray drying step include a solubility enhancer, such as hypromellose acetate succinate; and a permeation enhancer, such as d-α-tocopherol polyethylene glycol succinate. In certain embodiments, one or more additional intragranular excipients are added to the spray dried dispersion. In certain embodiments, the intragranular excipients added to the spray dried dispersion include, but are not limited to, a filler, such as mannitol; a mucoadhesive/disintegrant, such as croscarmellose sodium; a flow aid, such as colloidal silicon dioxide; and a binder/glidant, such as microcrystalline cellulose. In certain embodiments, the spray dried dispersion and the intragranular excipients added after the spray drying step are blended, milled, and optionally roller-compacted together into ribbons, and optionally with addition of a lubricant, such as magnesium stearate, added as a further intragranular excipient. In certain embodiments, the material or ribbons are then milled and blended with extragranular excipients.

Non-limiting examples of extragranular excipients include, but are not limited to, a binder/glidant, such as microcrystalline cellulose; a mucoadhesive/disintegrant, such as croscarmellose sodium; and a lubricant, such as magnesium stearate. In certain embodiments, an addition of a disintegrant as an extragranular excipient will contribute to effective disintegration of the tablet into small fragments. In certain embodiments, the presence of a disintegrant as an extragranular and intragranular excipient will ensure more efficient disintegration of the tablet.

This pharmaceutical formulation comprising intragranular and extragranular excipients has improved pharmaceutical properties, for example improved tablet disintegration, drug release, dissolution characteristics, and/or mechanical strength.

In certain embodiments, the pharmaceutical composition comprising Compound 1 is produced from a morphic form described herein, for example Compound 1 Form B. In certain embodiments, Compound 1 Form B can be dissolved and then spray dried to form a solid spray dry dispersion with one or more pharmaceutically acceptable excipients. In some aspects, the pharmaceutical composition comprises Compound 1, a solubility enhancer, a permeation enhancer, a filler, one or more binders and or glidants, and one or more flow aids. Non-limiting examples of pharmaceutically acceptable excipients include hypromellose (for example hypromellose acetate succinate), vitamin E (for example, d-alpha-tocopherol polyethylene glycol succinate), mannitol, cellulose (for example microcrystalline cellulose), croscarmellose sodium, silicon dioxide (for example untreated fumed colloidal), and magnesium stearate. In certain embodiments, a pharmaceutical composition according to the present invention is formulated into a dosage unit form, such as an oral dosage unit form. In certain embodiments, a pharmaceutical composition according to the present invention is formulated into a tablet dosage form.

In certain non-limiting embodiments, the pharmaceutical composition comprising Compound 1 comprises the following excipients.

| | | Percent |
|---|---|---|
| Class | Representative Components | (w/w) |
| Active | Compound 1, for example | about 5% to about 20% |
| Ingredient | Compound 1 prepared from | for example, about 10% |
| | Compound 1 Form B | |
| Solubility | Hypromellose, for example | about 15% to about 55% |
| enhancer | hypromellose acetate | for example, about 35% |
| | succinate | |
| Permeation | Vitamin E, for example d-α- | about 1% to about 10% |
| enhancer | tocopherol polyethylene | for example, about 5% |
| | glycol succinate | |
| Filler | Mannitol | about 20% to about 40% |
| | | for example, about 28% |
| Binder/Glidant | Cellulose, for example | about 10% to about 30% |
| | microcrystalline cellulose | for example, about 20% |
| Mucoadhesive/ | Croscarmellose, for example | about 0.5% to about 5% |
| Disintegrant | croscarmellose sodium | for example, about 2% |

A process for manufacturing a pharmaceutical composition comprising Compound 1 or a pharmaceutically acceptable salt thereof or a Compound 1 morphic form according to the present invention is also provided. In certain non-limiting aspects, a process for manufacturing a pharmaceutical composition comprising Compound 1 includes: (i) a spray drying step to provide a spray-dried intermediate (SDI) containing Compound 1 and pharmaceutically acceptable excipients; (ii) a granulation step to provide a granulate containing Compound 1 and one or more pharmaceutically acceptable excipients with a desired bulk density between about 0.4 to 0.6 g/mL for example about 0.48 to 0.54 g/mL; and (iii) a tableting step to provide a pharmaceutical composition comprising Compound 1 and pharmaceutically acceptable excipients in an oral dosage unit form.

A pharmaceutical composition comprising Compound 1 or a Compound 1 morphic form of the present invention can be used to treat a mutant BRAF mediated cancer, for example but not limited to melanoma, lung cancer including for example non-small cell lung cancer, colorectal cancer including for example microsatellite stable colorectal cancer, thyroid cancer including for example anaplastic thyroid cancer, or ovarian cancer. In certain non-limiting embodiments, a pharmaceutical composition comprising Compound 1 or a Compound 1 morphic form of the present invention is used to treat a solid tumor that is mediated by a V600X mutant BRAF.

Additional non-limiting examples of disorders that can be treated include solid tumor malignancies that have a mutant BRAF driver.

In certain embodiments, a pharmaceutical composition comprising Compound 1 or a pharmaceutically acceptable salt thereof or a Compound 1 morphic form of the present invention can be administered to treat a cancer that has developed resistance to a BRAF inhibitor.

The present invention also provides an improved method of producing Compound 1 or a pharmaceutically acceptable salt thereof or isotopic derivative thereof such as a deuterated derivative at manufacturing scale.

In certain aspects, the manufacturing scale production of Compound 1 or a pharmaceutically acceptable salt thereof includes the reaction of Compound A with Compound B according to the following reaction Scheme 1 resulting in an amide bond formation In a non-limiting embodiment, a deuterium is placed on an atom that is at, alpha, beta, or gamma to a site of metabolism.

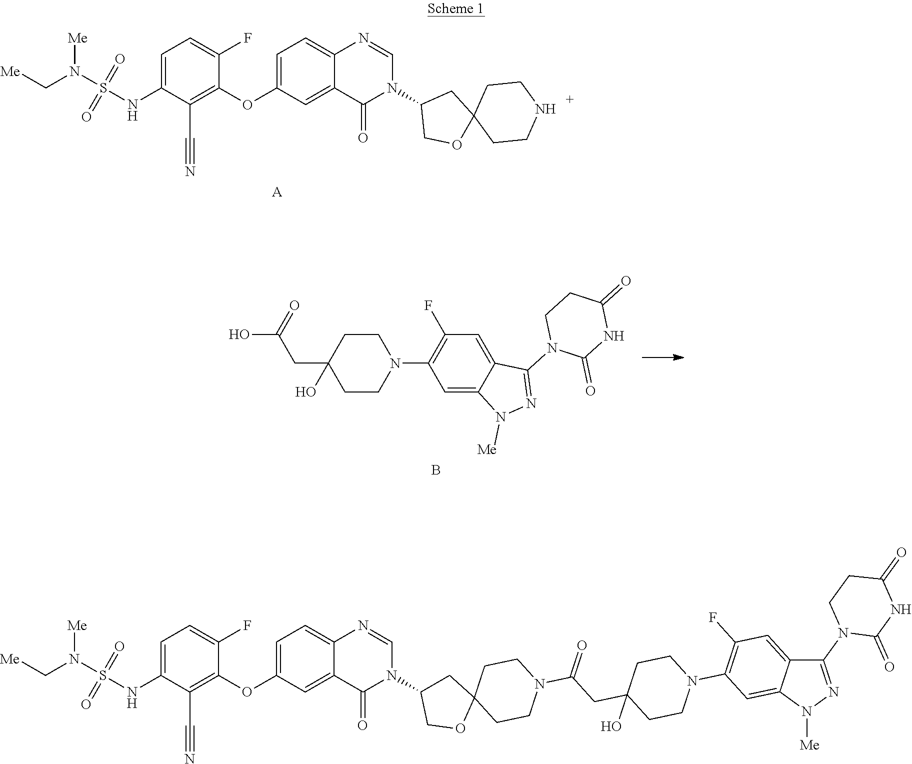

In certain embodiments, to facilitate amide bond formation between Compound A and Compound B, the carboxylic acid group in Compound B is activated by transforming the carboxylic acid hydroxyl into a more reactive group. In certain embodiments, activation of carboxylic acid group in Compound B is performed by reacting Compound B with a coupling agent which activates a carboxylic acid. In certain embodiments, the coupling agent is TSTU (2-succinimido-1,1,3,3-tetramethyluronium tetrafluoroborate). TSTU acts as an efficient coupling agent through rapid activation of carboxylic acid to form the N-succinimidyl active ester, which reacts with a primary amine to form a carboxamide. This reaction has been amenable to scaleup and is effective in the presence of water and selective towards amines in the presence of the alcohol group.

In certain embodiments, reaction between Compounds A and B leading to Compound 1 can be performed using separate activating reagents resulting in an amide bond formation. In certain embodiments, the reaction between Compounds A and B is performed using a combination of N-hydroxysuccinimide (NHS) and EDCl (1-ethyl-3-(3-dimethylaminopropyl)carbodiimide hydrochloride) or a combination of NHS and DCC (dicyclohexyl carbodiimide).

In certain embodiments, Compound A and/or Compound B have at least one desired isotopic substitution of an atom, at an amount above the natural abundance of the isotope, i.e., enriched. In certain embodiments, isotopic derivatives of Compound A and/or Compound B are used in the synthesis described in Scheme 1. In certain non-limiting embodiments, Compound A and/or Compound B include a deuterium atom or multiple deuterium atoms. In certain embodiments, Compound A has formula A-d₃ and Compound B has formula B-d₄:

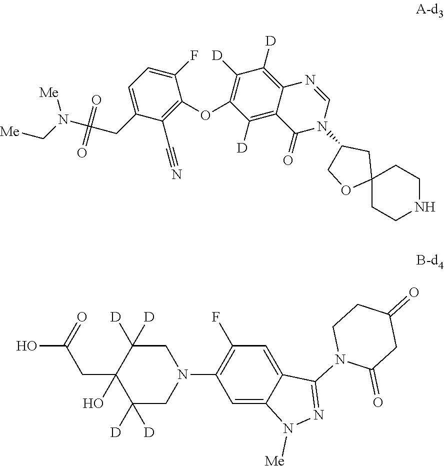

In certain embodiments, Compound A or an isotopic derivative thereof for example a deuterium derivative is prepared from Compound C of the formula:

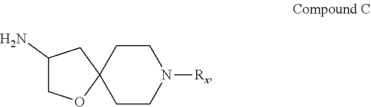

wherein R_{X} is hydrogen or an amine protecting group. In certain embodiments, non-limiting examples of amine protecting group R_{X} include, but are not limited to, carbobenzyloxy (Cbz), p-methoxybenzyl carbonyl (Moz or MeOZ), tert-butyloxycarbonyl (BOC), 9-fluorenylmethyloxycarbonyl (Fmoc), benzoyl (Bz), benzyl (Bn), carbamate, p-methoxybenzyl (PMB), 3,4-dimethoxybenzyl (DMPM), p-methoxyphenyl (PMP) and tosyl (Ts). In certain embodiments, the amine protecting group R_{X} is tert-butyloxycarbonyl (BOC).

The improved synthetic procedures described herein include methods to prepare chirally pure Compound C. Compound C is used in the synthesis of Compound A as a mixture of (R)- and (S)-stereoisomers or as an individual (R)-stereoisomer:

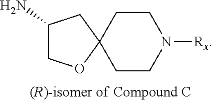

In certain embodiments, the (R)-stereoisomer of Compound C is prepared using a transaminase-catalyzed reductive amination of the corresponding ketone precursor:

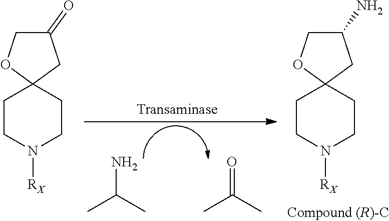

In this asymmetric synthesis reaction, isopropylamine for example can server as a sacrificial amine-donating source.

In other embodiments, the (R)-stereoisomer of Compound C is prepared from a racemic Compound C through diastereomeric recrystallisation of a salt formed between the racemic Compound C and a chiral resolving acid followed by release of the desired enantiomer with a base. In certain embodiments, the chiral resolving acid is pyroglutamic acid. In certain embodiments, the chiral resolving acid is D-mandelic acid, and the base used to recover the separated individual (R)- and (S)-enantiomers of Compound C from their diastereomeric salts with D-mandelic acid is an appropriate inorganic base such as sodium hydroxide as illustrated in Scheme 2. In other aspects the base used to recover the separated enantiomers is an organic base.

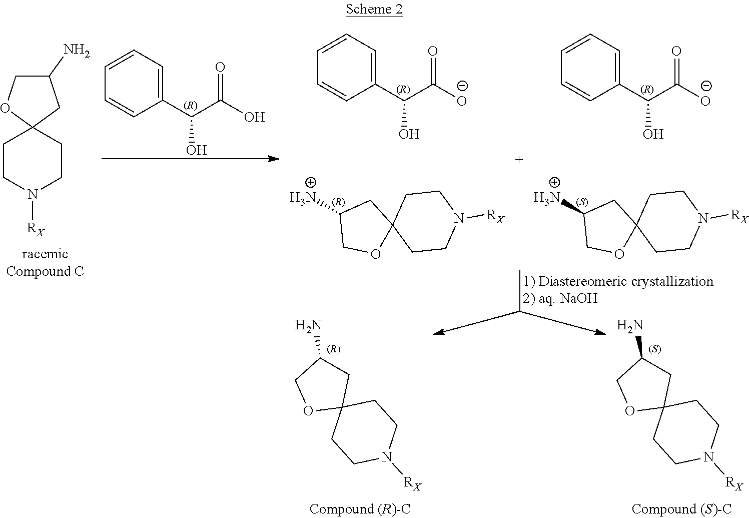

In certain aspects, Compound (R)-C is used to prepare Compound A or an isotopic derivative thereof as depicted in Scheme 3.

These two processes allow chirally pure material to be prepared without having to use expensive chiral chromatography. The improved process also avoids losing large quantities of advanced synthetic material that have the undesired chirality. Thus, the throughput and scalability of the synthetic sequence has been significantly improved.

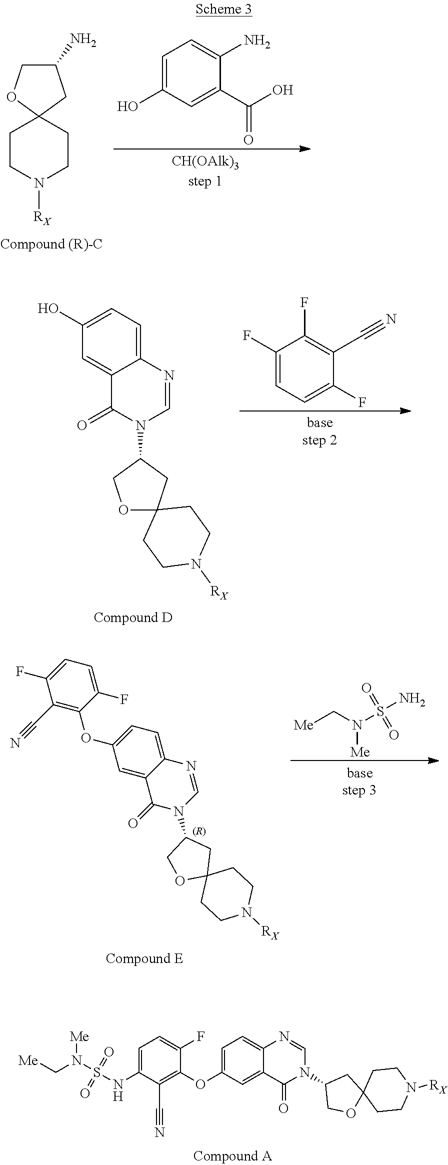

In certain embodiments, step 1 in Scheme 3 includes reacting 5-hydroxyanthranilic acid (2-amino-5-hydroxybenzoic acid) or an isotopic derivative thereof such as a deuterated derivative with Compound (R)-C and trialkyl orthoformate CH(OAlk)₃, such as triethyl orthoformate CH(OEt)₃, to generate Compound D or an isotopic derivative thereof containing a quinazolin-4-one ring system. In certain embodiments, an isotopic derivative of 2-amino-5-hydroxybenzoic acid is 2-amino-3,4,6-trideuterio-5-hydroxybenzoic acid. In certain embodiments, step 2 in Scheme 3 includes a nucleophilic aromatic substitution reaction (S_{N}Ar) between 2,3,6-trifluorobenzonitrile and hydroxyl of the quinazolinone fragment in Compound D in the presence of a base, such as an organic or inorganic base, for example potassium carbonate, to give Compound E or an isotopic derivative thereof as a single enantiomer. In certain embodiments, in step 3 in Scheme 3, Compound E or an isotopic derivative thereof reacts with [ethyl(methyl)sulfamoyl]amine in the presence of a base, such as an organic or inorganic base, for example cesium carbonate, under nucleophilic aromatic substitution (S_{N}Ar) conditions, to afford Compound A or an isotopic derivative thereof without loss of its enantiomeric purity (for example >99% e.e.). In certain embodiments, in an optional step, when the nitrogen atom in Compound A or an isotopic derivative thereof such as a deuterated derivative is protected with a nitrogen protecting group R_{X}, the protecting group, such as carbobenzyloxy (Cbz), p-methoxybenzyl carbonyl (Moz or MeOZ), tert-butyloxycarbonyl (BOC), 9-fluorenylmethyloxycarbonyl (Fmoc), benzoyl (Bz), benzyl (Bn), carbamate, p-methoxybenzyl (PMB), 3,4-dimethoxybenzyl (DMPM), p-methoxyphenyl (PMP) or tosyl (Ts), may be removed to release the free amino group in Compound A. In certain embodiments, when the amino protecting group R_{X} is BOC, the deprotection reaction is performed under acidic conditions, such as, but not limited to, hydrochloric acid in aqueous media and/or organic solvent, for example hydrochloric acid in acetone or ethyl acetate.

This synthetic sequence can be conducted at manufacturing scale to produce chirally pure isomers (for example greater than 90%, 95% or 99% enantiomeric purity).

In certain embodiments, Compound B or an isotopically enriched derivative thereof such as a deuterated derivative is prepared according to the reaction sequence depicted in Scheme 4.

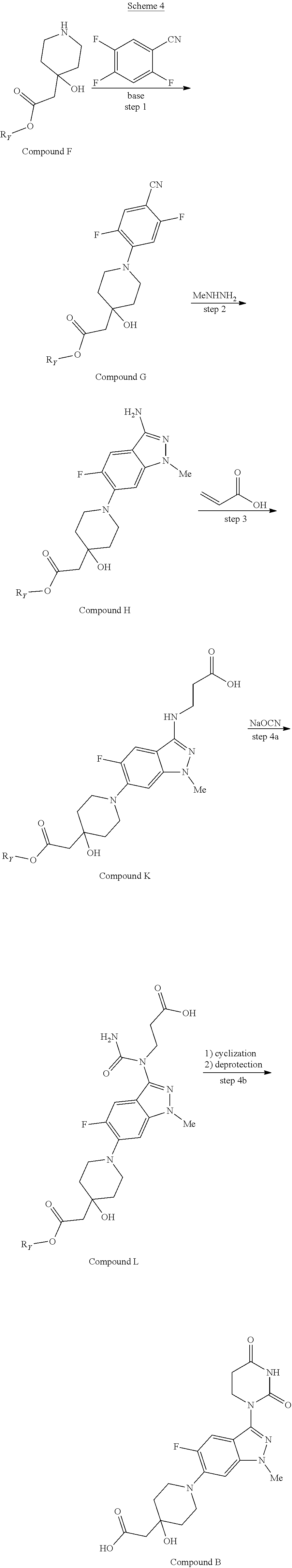

In certain embodiments, the synthetic route for Compound B or an isotopic derivative or a salt thereof according to the present invention as shown in Scheme 4 includes four steps. In certain embodiments, the first step includes reaction of Compound F or an isotopic derivative thereof, having a carboxylic group optionally protected by a protecting group R_{Y}, with 2,4,5-trifluorobenzonitrile under the nucleophilic aromatic substitution reaction (S_{N}Ar) conditions to afford Compound G. In certain embodiments, the isotopic derivative of Compound F is tert-butyl 2-(3,3,5,5-tetradeuterio-4-hydroxy-4-piperidyl)acetate. Examples of the carboxylic acid protecting group R_{Y} include, but are not limited to, tert-butyl (^{t}Bu), trityl (Trt), 2,4-dimethoxybenzyl (Dmb), 9-fluorenylmethyl (Fm), and benzyl (Bn). In certain embodiments, the carboxylic acid protecting group R_{Y} is tert-butyl (^{t}Bu). In certain embodiments, the nucleophilic aromatic substitution reaction of step 1 is carried out in the presence of base. In certain embodiments, the base is an organic or inorganic base and includes but is not limited to, sodium carbonate, potassium carbonate, cesium carbonate, or N,N-diisopropylethylamine (DIEA, Hunig's base).

The resulting intermediate Compound G is converted to Compound H containing the amino indazole ring system by heating with methylhydrazine in a suitable solvent, such as an organic or inorganic solvent, including water and NMP (N-methyl-2-pyrrolidone). In certain embodiments, in step 3 of Scheme 4, Compound K is prepared from Compound H in the reaction of Compound H with acrylic acid (or its derivative, such as an ester, for example methyl or ethyl ester) under the Michael addition conditions. In certain embodiments, the Michael addition conditions include basic or acidic conditions. In certain embodiments, the Michael addition conditions include use of diluted aqueous hydrochloric acid as a medium for the Michael addition reaction. In certain embodiments, Compound K is converted into Compound B in a condensation reaction with a cyanate salt via an intermediate formation of urea Compound L, optionally followed by removal (deprotection) of the protecting group R_{Y} to afford Compound B (steps 4a-b in Scheme 4) according to the present invention. In certain embodiments, the cyanate salt used in step 4a of Scheme 4 is sodium or potassium cyanate used in the presence of acetic acid. In certain embodiments, cyclisation and deprotection step 4b in Scheme 4 is performed under acidic condition, which include, but not limited to, use of diluted hydrochloric acid.

The synthetic method according to the present invention as illustrated and described with reference to the Schemes 1-4 has several advantageous and beneficial aspects. The advantageous features of the improved method of producing Compound 1 and/or isotopic such as deuterium derivatives thereof according to the present invention include high yield and/or high purity of Compound 1. Isotopic such as deuterium derivatives and synthetic intermediates thereof, including chemical and chiral purity are also provided. The advantageous features of the improved method of producing Compound 1 according to the present invention also include high reliability, reproducibility, scalability, and/or atom efficiency.

Another advantageous and beneficial aspect of the improved synthetic method according to the present invention is that it is a palladium-free synthesis of synthetic intermediates and Compound 1 and/or isotopic such as deuterium derivatives thereof. Using palladium-free conditions in the synthesis of intermediate compounds and precursors for the preparation of Compound 1 or its isotopic such as deuterium derivatives provided in the present invention allows one to avoid contamination of the final drug substance with traces of toxic and undesirable palladium which may be difficult to remove due to palladium complexing with intermediate compounds and precursors which carry over to downstream and/or final steps of the synthesis and, therefore, can eliminate additional purification steps. Palladium is a known toxic heavy metal that may damage bone marrow, kidneys or liver and should be avoided, if possible, in pharmaceutical compositions and drug substances. By avoiding the use of a palladium catalyst, the need for expensive and time-consuming palladium scavenging is avoided.

Therefore, the palladium-free synthesis of Compound 1 and its isotopic such as deuterium derivatives according to the present invention is advantageous for subsequent use of Compound 1, morphic forms and derivatives thereof as drug substances and in pharmaceutical compositions.

In other aspects a method of treating a mutant BRAF mediated cancer that has metastasized to the brain or central nervous system (CNS) is provided comprising administering an effective amount of Compound 1, or a pharmaceutically acceptable salt or morphic form thereof, to a patient in need thereof. In certain embodiments the cancer that has metastasized to the brain or CNS is colorectal cancer, melanoma, or non-small cell lung cancer. In certain embodiments of this aspect Compound 1 or a pharmaceutically acceptable salt thereof is administered to the patient in need thereof. In other embodiments of this aspect a morphic form of Compound 1 or a pharmaceutically acceptable salt thereof or a pharmaceutical composition prepared from a morphic form of Compound 1 is administered to the patient in need thereof.

Compound 1 has a high level of blood brain barrier penetration (see FIG. 52A and Example 34). When tested in an intracranial melanoma model Compound 1 dramatically reduced the bioluminescence signal relative to vehicle control or encorafenib treated mice, representing a decrease in tumor burden (see FIG. 40). This pronounced reduction in bioluminescence signal, as an indicator of tumor burden growth, resulted in a significant increase in survival time for the mice with intracranial melanoma tumors compared to encorafenib (see FIG. 41).

In certain aspects, Compound 1 or a pharmaceutically acceptable salt thereof is used to treat a mutant BRAF mediated cancer that has metastasized to the brain or CNS, wherein the BRAF has mutated from the wild type. There are a number of possibilities for BRAF mutations. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, V600M and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N. In certain embodiments the BRAF mutation is a V600 mutation, for example a V600E BRAF that mediates a cancer that has metastasized to the brain or CNS.

In certain embodiments, Compound 1 or a pharmaceutically acceptable salt thereof, may treat a mutant BRAF mediated cancer that has metastasized to the brain or CNS wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments, Compound 1 or a pharmaceutically acceptable salt thereof, may treat a mutant BRAF mediated cancer that has metastasized to the brain or CNS wherein the mutation is a splice variant, for example p61-BRAF^{V600E}.

In certain embodiments, Compound 1 or a pharmaceutically acceptable salt thereof, is used to treat a mutant BRAF mediated cancer that has metastasized to the brain or CNS, wherein the cancer is mediated by two or more mutant proteins, for example a cancer mediated by a BRAF^{V600E}/NRAS^{Q61K} or BRAF^{V600E}/NRAS^{Q61R} double mutant. Non-limiting examples of double mutant cancers include colorectal cancer which is mediated by a BRAF mutation, for example BRAF^{V600E}, and a mutation of NRAS, MEK1, or phosphatidylinositol-4,5-bisphosphate 3-kinase (PI3K), for example, BRAF^{V600E}/MAP2K1^{P124S}, BRAF^{V600E}/PIK3CA^{H1047R} or BRAF^{V600E}/PIK3CA^{P449T}.

In certain embodiments, Compound 1 or a pharmaceutically acceptable salt thereof, is used to treat a mutant BRAF mediated cancer that has metastasized to the brain or CNS, wherein the cancer is resistant to at least one BRAF inhibitor, for example a cancer that is resistant to or has acquired resistance to a BRAF inhibitor selected from dabrafenib, vemurafenib, and encorafenib. In certain aspects the cancer that is resistant to treatment with a BRAF inhibitor has a RAF protein homo-dimerization or hetero-dimerization promoting mutation. For example, in certain embodiments Compound 1 or a pharmaceutically acceptable salt thereof, is used to treat a mutant BRAF mediated cancer that has metastasized to the brain or CNS wherein the cancer has one or more mutations that promote RAF protein dimerization. In certain embodiments the cancer with one or more RAF protein dimerization promoting mutations is resistant to treatment with a BRAF inhibitor for example dabrafenib, vemurafenib, or encorafenib. In certain embodiments the RAF protein dimer is a homo-dimer of BRAF-BRAF. In other embodiments the RAF protein dimer is a hetero-dimer with other RAF proteins (BRAF-RAF1 or BRAF-ARAF) In certain embodiments, Compound 1 or a pharmaceutically acceptable salt thereof, is used to treat a cancer that has metastasized to the brain or CNS, wherein the cancer may have developed an escape mutation such as BRAF V600E/NRAS^{Q61K} or BRAF V600F/NRAS^{Q61R} double mutant cancer.

In certain embodiments, Compound 1 or a pharmaceutically acceptable salt thereof, is used to treat melanoma that has metastasized to the brain or CNS. In other embodiments, Compound 1 or a pharmaceutically acceptable salt thereof, is used to treat colorectal or lung cancer that has metastasized to the brain or CNS.

In alternative aspects the S-enantiomer or a mixture of enantiomers or a pharmaceutically acceptable salt thereof are used instead of Compound 1 in a treatment or pharmaceutical composition descried herein.

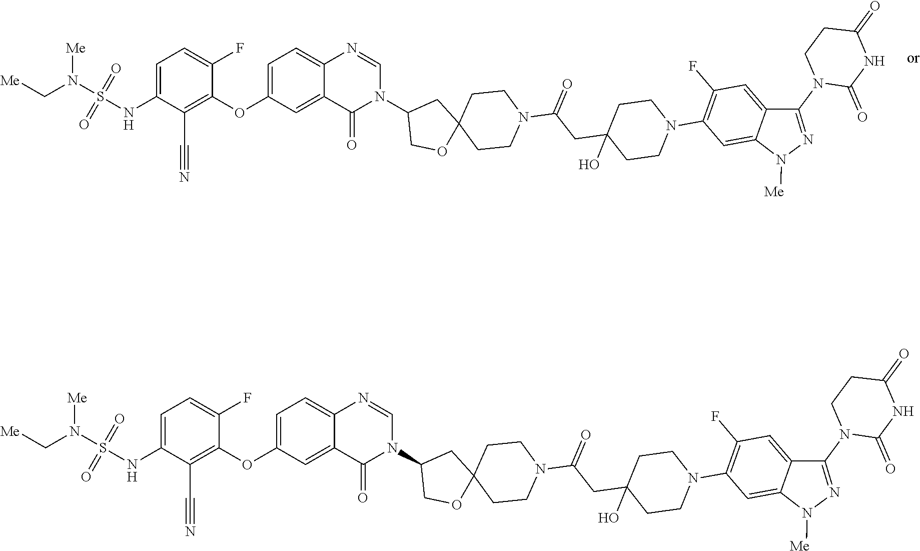 [SMILES: CCN(C)S(=O)(=O)Nc1ccc(F)c(Oc2ccc3ncn(C4COC5(CCN(C(=O)CC6(O)CCN(c7cc8c(cc7F)c(N7CCC(=O)NC7=O)nn8C)CC6)CC5)C4)c(=O)c3c2)c1C#N ; CCN(C)S(=O)(=O)Nc1ccc(F)c(Oc2ccc3ncn([C@@H]4COC5(CCN(C(=O)CC6(O)CCN(c7cc8c(cc7F)c(N7CCC(=O)NC7=O)nn8C)CC6)CC5)C4)c(=O)c3c2)c1C#N]

Other features and advantages of the present application are apparent from the following detailed description.

Thus, the present invention includes at least the following features:
(a) a morphic form of Compound 1 as described herein;
(b) Compound 1 Form B;
(c) Compound 1 Sodium salt Form F;
(d) A pharmaceutical composition comprising a Compound 1 morphic form of any one of embodiments (a)-(c) and one or more pharmaceutically acceptable excipients;
(e) a pharmaceutical composition prepared from a Compound 1 morphic form of any one of embodiments (a)-(c), for example Morphic Form B;
(f) the pharmaceutical composition of (e) wherein the pharmaceutical composition is prepared from a spray dried dispersion of Compound 1 and one or more pharmaceutically acceptable excipients;
(g) a pharmaceutical composition comprising Compound 1 or a pharmaceutically acceptable salt thereof, a solubility enhancer, a permeation enhancer, a filler, a binder/glidant, and/or a mucoadhesive/disintegrant.
(h) a method for the treatment of a disorder that is mediated by BRAF comprising administering an effective amount of a Compound 1 morphic form or pharmaceutical composition of any one of embodiments (a)-(g);
(i) use of a Compound 1 morphic form or pharmaceutical composition of any one of embodiments (a)-(g) in the treatment of a disorder that is mediated by BRAF;
(j) use of a compound or pharmaceutical composition of any one of embodiments (a)-(g) in the manufacture of a medicament for the treatment of a disorder that is mediated by BRAF;
(k) a method for the treatment of a cancer comprising administering an effective amount of a Compound 1 morphic form or pharmaceutical composition of any one of embodiments (a)-(g);
(l) a Compound 1 morphic form or pharmaceutical composition of any one of embodiments (a)-(g) for use in the treatment of a cancer;
(m) use of a Compound 1 morphic form or pharmaceutical composition of any one of embodiments (a)-(g) for the treatment of a cancer;
(n) use of a Compound 1 morphic form or pharmaceutical composition of any one of embodiments (a)-(g) in the manufacture of a medicament for the treatment of a cancer;
(o) a method to prepare Compound 1 Form B as described herein;
(p) a method to prepare Compound 1 sodium salt Form F as described herein;
(q) a method to treat a mutant BRAF mediated cancer that has metastasized to the brain or CNS comprising administering an effective amount of Compound 1 or a pharmaceutically acceptable salt thereof;
(r) the method of (q), wherein the mutant BRAF mediated cancer is melanoma;
(s) use of Compound 1 or a pharmaceutically acceptable salt thereof in the treatment of a mutant BRAF mediated cancer that has metastasized to the brain or CNS;
(t) use of Compound 1 or a pharmaceutically acceptable salt thereof in the manufacture of a medicament for the treatment of a mutant BRAF mediated cancer that has metastasized to the brain or CNS; and
(u) the use of (s) or (t), wherein the mutant BRAF mediated cancer is melanoma.

### BRIEF DESCRIPTION OF THE FIGURES

FIG. 1 depicts the XRPD pattern of Compound 1 Form B. The experiment was conducted as described in Example 8 and Example 28.

FIG. 2 depicts the DSC thermogram of Compound 1 Form B. The experiment was conducted as described in Example 28.

FIG. 3 depicts the TGA thermogram of Compound 1 Form B. The experiment was conducted as described in Example 28.

FIG. 4 depicts the DVS isotherm plot of Compound 1 Form B at 25° C. The experiment was conducted as described in Example 28.

FIG. 5 depicts the DVS change in mass plot of Compound 1 Form B at 25° C. The experiment was conducted as described in Example 28.

FIG. 6 depicts the XRPD overlay of Compound 1 Form B before and after DVS test. The experiment was conducted as described in Example 28.

FIG. 7 depicts the XRPD pattern of Compound 1 Form A. The experiment was conducted as described in Example 8 and Example 28.

FIG. 8 depicts the DSC thermogram of Compound 1 Form A. The experiment was conducted as described in Example 28.

FIG. 9 depicts the TGA thermogram of Compound 1 Form A. The experiment was conducted as described in Example 28.

FIG. 10 depicts the XRPD pattern of Compound 1 Form C. The experiment was conducted as described in Example 8 and Example 28.

FIG. 11 depicts the DSC thermogram of Compound 1 Form C. The experiment was conducted as described in Example 28.

FIG. 12 depicts the TGA thermogram of Compound 1 Form C. The experiment was conducted as described in Example 28.

FIG. 13 depicts an overlay of the XRPD patterns of Compound 1 Form B samples before compression, after compression under 2 MPa and after compression under 10 MPa. The experiment was conducted as described in Example 28.

FIG. 14 depicts an overlay of the XRPD patterns of Compound 1 Form B samples before dry grinding, and after dry grinding for 1, 2, and 5 minutes. The experiment was conducted as described in Example 28.

FIG. 15 depicts an overlay of the XRPD patterns of Compound 1 Form B samples before wet granulation and after wet granulation in the presence of water or ethanol. The experiment was conducted as described in Example 28.

FIG. 16 depicts a comparison of the XRPD pattern of Compound 1 Form C, the XRPD pattern obtained after Compound 1 Form C was subjected to about 8 equilibration cycles in 2-methyltetrahydrofuran, and the XRPD pattern of Compound 1 Form B. As a result of equilibration, Form C converted to more stable Form B. The experiment was conducted as described in Example 28.

FIG. 17 depicts the XRPD pattern of Compound 1 sodium salt Form F. The experiment was conducted as described in Example 28.

FIG. 18 depicts the DSC thermogram of Compound 1 sodium salt Form F. The experiment was conducted as described in Example 28.

FIG. 19 depicts the TGA thermogram of Compound 1 sodium salt Form F. The experiment was conducted as described in Example 28.

FIG. 20 depicts the DVS isotherm plot of Compound 1 sodium salt Form F at 25° C. The experiment was conducted as described in Example 28.

FIG. 21 depicts the DVS change in mass plot of Compound 1 sodium salt Form F at 25° C. The experiment was conducted as described in Example 28.

FIG. 22 depicts the XRPD overlay of Compound 1 sodium salt Form F before and after DVS test. The experiment was conducted as described in Example 28.

FIG. 23 depicts the XRPD pattern of Compound 1 sodium salt Form D. The experiment was conducted as described in Example 28.

FIG. 24 depicts the DSC thermogram of Compound 1 sodium salt Form D. The experiment was conducted as described in Example 28.

FIG. 25 depicts the TGA thermogram of Compound 1 sodium salt Form D. The experiment was conducted as described in Example 28.

FIG. 26 depicts the XRPD pattern of Compound 1 sodium salt Form E. The experiment was conducted as described in Example 28.

FIG. 27 depicts the DSC thermogram of Compound 1 sodium salt Form E. The experiment was conducted as described in Example 28.

FIG. 28 depicts the TGA thermogram of Compound 1 sodium salt Form E. The experiment was conducted as described in Example 28.

FIG. 29 depicts the XRPD pattern of Compound 1 potassium salt Form G. The experiment was conducted as described in Example 28.

FIG. 30 depicts the DSC thermogram of Compound 1 potassium salt Form G. The experiment was conducted as described in Example 28.

FIG. 31 depicts the TGA thermogram of Compound 1 potassium salt Form G. The experiment was conducted as described in Example 28.

FIG. 32 depicts the XRPD pattern of Compound 1 potassium salt Form H. The experiment was conducted as described in Example 28.

FIG. 33 depicts the DSC thermogram of Compound 1 potassium salt Form H. The experiment was conducted as described in Example 28.

FIG. 34 depicts the TGA thermogram of Compound 1 potassium salt Form H. The experiment was conducted as described in Example 28.

FIG. 35 depicts a comparison of the XRPD pattern of Compound 1 sodium salt Form D obtained in slurry equilibration of Compound 1 Form A with 1 equivalent of sodium hydroxide in methanol, amorphic XRPD patterns obtained in slurry equilibration of Compound 1 Form A with 1 equivalent sodium hydroxide in acetone or acetonitrile, and the XRPD pattern of Compound 1 Form A. The experiment was conducted as described in Example 21.

FIG. 36 depicts a comparison of the XRPD patterns of Compound 1 sodium salt Form E (prepared in methanol) and Form F (prepared in acetone or acetonitrile) obtained in slurry equilibration of Compound 1 Form A with 1 equivalent of sodium bicarbonate NaHCO₃, with the XRPD patterns of Form A and Form B. The experiment was conducted as described in Example 21.

FIG. 37 depicts a comparison of the XRPD patterns of Compound 1 Form I obtained by slurry equilibration of Form A in the presence of 1 eq. of ammonia in methanol and acetonitrile with the XRPD pattern of Form A. The experiment was conducted as described in Example 21.

FIG. 38 depicts a single crystal structure of (R)-8-(tert-butoxycarbonyl)-1-oxa-8-azaspiro[4.5]decan-3-aminium (R)-2-hydroxy-2-phenylacetate. The experiment was conducted as described in Example 32.

FIG. 39 is a line graph depicting the BRAF V600E mutant human colorectal adenocarcinoma HT29 cell viability following 72 hours post treatment with Compound 1. The X-axis is the concentration of Compound 1 in nM, and the Y-axis is % cell viability. The experiment was conducted as described in Example 33.

FIG. 40 is a line graph depicting the change in luciferase bioluminescence signal over time in the BRAF V600E mutant A375-luciferase intracranial model after administration of the test compounds (vehicle, encorafenib administered at 35 mg/kg, compound 1 administered at 10 mg/kg and 30 mg/kg), representing tumor burden. The X-axis is days after the start of the treatment, and the Y-axis is bioluminescence (photons/second) corresponding to the luciferase signal. Error bars represent standard error of the mean (SEM). The experiment was conducted as described in Example 34.

FIG. 41 is a Kaplan-Meier survival curve depicting the survival rate in the BRAF V600E A375-luciferase intracranial tumor model, after treatment with the test compounds (vehicle, encorafenib administered at 35 mg/kg, compound 1 administered at 10 mg/kg and 30 mg/kg). The X-axis is days after the start of the treatment, and the Y-axis is the probability of survival. The experiment was conducted as described in Example 34.

FIG. 42 is a line graph depicting the plasma concentrations of test compounds (encorafenib administered at 35 mg/kg, compound 1 administered at 10 mg/kg and 30 mg/kg) over time in the BRAF V600E mutant A375-luciferase intracranial model. The X-axis is time after the treatment, and the Y-axis is compound concentration in plasma in ng/mL. Error bars represent standard error of the mean (SEM). The experiment was conducted as described in Example 34.

FIG. 43 is a line graph depicting the brain concentrations of test compounds (encorafenib administered at 35 mg/kg, compound 1 administered at 10 mg/kg and 30 mg/kg) over time in the BRAF V600E mutant A375-luciferase intracranial model. The X-axis is time after the treatment, and the Y-axis is compound concentration in plasma in ng/g. Error bars represent standard error of the mean (SEM). The experiment was conducted as described in Example 34.

FIG. 44 is a line graph depicting BRAF V600E protein degradation in the A375 CNS tumor after administration of the test compounds (encorafenib administered at 35 mg/kg, compound 1 administered at 10 mg/kg and 30 mg/kg) over time. The X-axis is time after the treatment, and the Y-axis is mutant BRAF levels (normalized). Error bars represent standard error of the mean (SEM). The experiment was conducted as described in Example 34.

FIG. 45 is a line graph depicting the change in human colorectal adenocarcinoma (HT-29 CDX model of CRC) tumor size over time after administration of the test compounds (10 μL/kg of vehicle, 11 mg/kg of cetuximab, 10 mg/kg of compound 1, 10 mg/kg of compound 1+11 mg/kg of cetuximab, 35 mg/kg of encorafenib, and 35 mg/kg encorafenib+11 mg/kg cetuximab) over time. The X-axis is days after the treatment, and the Y-axis is tumor size in mm³. Error bars represent standard error of the mean (SEM). The experiment was conducted as described in Example 35.

FIG. 46 is a line graph depicting the change in tumor size in BRAF V600E mutant NSCLC xenograft model (PDX model of NSCLC) over time after administration of the test compounds (vehicle control, 0.1 mg/kg of trametinib, 100 mg/kg of dabrafenib, 100 mg/kg of dabrafenib+0.1 mg/kg of trametinib, 10 mg/kg of compound 1, and 10 mg/kg of compound 1+0.1 mg/kg of trametinib) over time. At day 28 the mice treated with compound 1 with or without trametinib were taken off treatment and monitored for tumor outgrowth until day 45. The X-axis is days after the initiation of the treatment, and the Y-axis is tumor size in mm³ Error bars represent standard error of the mean (SEM). The experiment was conducted as described in Example 36.

FIG. 47 is a line graph depicting the change in tumor size in melanoma PDX model of BRAF inhibitor resistant melanoma with a BRAF kinase domain duplication acquired during treatment with the BRAF inhibitor dabrafenib in combination with the MEK inhibitor trametinib. over time after administration of the test compounds (vehicle control, 0.1 mg/kg of trametinib, 100 mg/kg of dabrafenib, 100 mg/kg of dabrafenib+0.1 mg/kg of trametinib, 10 mg/kg of compound 1, and 10 mg/kg of compound 1+0.1 mg/kg of trametinib) over time. At day 21 the mice treated with compound 1+trametinib were taken off treatment and monitored for tumor outgrowth until day 38. The X-axis is days after the initiation of treatment, and the Y-axis is tumor volume/size in mm³. The experiment was conducted as described in Example 37.

FIG. 48 is a line graph depicting the change in tumor size in A2058 human melanoma cancer cell derived xenografts CDX model of BRAF inhibitor resistant melanoma with a MEK1 P124S mutation in addition to BRAF V600E over time after administration of the test compounds (10 μL/g of vehicle control, 100 mg/kg of dabrafenib, 35 mg/kg of encorafenib and 10 mg/kg of compound 1) over time. The X-axis is days after the treatment, and the Y-axis is tumor volume/size in mm³. Error bars represent standard error of the mean (SEM). The experiment was conducted as described in Example 38.

FIG. 49 is a line graph depicting body weight changes after the administration of test compounds in Female BALB/c nude Mice bearing A2058 melanoma xenografts carrying the oncogenic mutations BRAF V600E and MEK1 P124S. The X-axis is days after the treatment, and the Y-axis is group mean body weight in grams. Data points represent group mean body weight.

Error bars represent standard error of the mean (SEM). The experiment was conducted as described in Example 39.

FIG. 50 is a line graph depicting percent body weight (BW) change over time in the mice implanted with the A2058 melanoma CDX. BW change was calculated based on animal weight on the first day of grouping. The X-axis is days after the treatment, and the Y-axis is percent body weight change. Data points represent percent group mean change in BW. Error bars represent standard error of the mean (SEM). The experiment was conducted as described in Example 39.

FIG. 51 is a line graph depicting the tumor volume in cubic millimeters after administration of test compounds in female BALB/c nude mice bearing A2058 xenografts. The X-axis is days after the treatment, and the Y-axis is tumor size in millimeters. Data points represent group mean tumor volume. Error bars represent standard error of the mean (SEM). The experiment was conducted as described in Example 39.

FIG. 52A is a bar graph depicting the concentration of compound 1 in plasma and tumor collected from mice implanted with the A2058 melanoma CDX over time, at 1 h, 6 h, 12 h, 24 h, and 48 h time points after administration, the Y-axis is concentration of compound 1 in ng/g. Error bars represent standard error of the mean (SEM). The experiment was conducted as described in Example 39.

FIG. 52B is a bar graph depicting the concentration of encorafenib in plasma and tumor collected from mice implanted with the A2058 melanoma CDX over time, at 1 h, 6 h, 12 h, 24 h, and 48 h time points after compound administration. The Y-axis is concentration of encorafenib 1 in ng/g. Error bars represent standard error of the mean (SEM). The experiment was conducted as described in Example 39.

FIG. 52C is a bar graph depicting the concentration of dabrafenib in plasma and tumor collected from mice implanted with the A2058 melanoma CDX over time, at level at 6 hour, 12 hour, 24 hour, and 48 hour time points after administration. The Y-axis is concentration of dabrafenib in ng/g. Error Bars Represent Standard Error of the Mean (SEM). The experiment was conducted as described in Example 39.

FIG. 53A is a bar graph depicting the Western Blot analysis of p-ERK and ERK expression level at 6 hour, 12 hour, 24 hour, and 48 hour time points in A2058 tumor after single dose administration of 10 mg/kg of compound 1. Expression level with vehicle at 6 h after administration was used as control. The experiment was conducted as described in Example 39.

FIG. 53B is a bar graph depicting the Western Blot analysis of p-ERK and ERK expression level at 6 hour, 12 hour, 24 hour, and 48 hour time points in A2058 tumor after administration of 35 mg/kg of encorafenib. Expression level with vehicle at 6 h after administration was used as control. The experiment was conducted as described in Example 39.

FIG. 53C is a bar graph depicting the Western Blot analysis of p-ERK and ERK expression level at 6 hour, 12 hour, 24 hour, and 48 hour time points in A2058 tumor after administration of 100 mg/kg of dabrafenib. Expression level with vehicle at 6 h after administration was used as control. The experiment was conducted as described in Example 39.

FIG. 54A is a bar graph depicting the Western Blot analysis of BRAF (V600E) expression level at 6 hour, 12 hour, 24 hour, and 48 hour time points in A2058 tumor after administration of 10 mg/kg of compound 1. Expression level with vehicle at 6 h after administration was used as control. The experiment was conducted as described in Example 39.

FIG. 54B is a bar graph depicting the Western Blot analysis of BRAF (V600E) expression level at 6 hour, 12 hour, 24 hour, and 48 hour time points in A2058 tumor after administration of 35 mg/kg of encorafenib. Expression level with vehicle at 6 h after administration was used as control. The experiment was conducted as described in Example 39.

FIG. 54C is a bar graph depicting the Western Blot analysis of BRAF (V600E) expression level at 6 hour, 12 hour, 24 hour, and 48 hour time points in A2058 tumor after administration of 100 mg/kg of dabrafenib. Expression level with vehicle at 6 hours after administration was used as a control. The experiment was conducted as described in Example 39.

### DETAILED DESCRIPTION OF THE INVENTION

### Definitions

Unless otherwise defined, all technical and scientific terms used herein have the same meaning as commonly understood by one of ordinary skill in the art to which this application belongs. In the specification, singular forms also include the plural unless the context clearly dictates otherwise. Although methods and materials similar or equivalent to those described herein can be used in the practice and testing of the present application, suitable methods and materials are described below. All publications, patent applications, patents, and other references mentioned herein are incorporated by reference. The references cited herein are not admitted to be prior art to the claimed application. In the case of conflict, the present specification, including definitions, will control. In addition, the materials, methods, and examples are illustrative only and are not intended to be limiting.

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The term “alkyl”, alone or in combination, signifies a straight-chain, branched-chain, or cyclic alkyl group with 1 to 8 carbon atoms, particularly a straight, branched-chain, or cyclic alkyl group with 1 to 6 carbon atoms and more particularly a straight or branched-chain alkyl group with 1 to 4 carbon atoms. Examples of straight-chain, branched-chain, or cyclic C₁-C₆ alkyl are methyl, ethyl, propyl, cyclopropyl, isopropyl, butyl, cyclobutyl, isobutyl, tert-butyl, pentyl, cyclopentyl, isopentyl, hexyl, isohexyl, and cyclohexyl. In certain embodiments “alkyl” is methyl. In certain embodiments “alkyl” is ethyl.

The present invention includes compounds described herein with at least one desired isotopic substitution of an atom, at an amount above the natural abundance of the isotope, i.e., enriched. Isotopes are atoms having the same atomic number but different mass numbers, i.e., the same number of protons but a different number of neutrons. If isotopic substitutions are used, the common replacement is at least one deuterium for hydrogen.

More generally, examples of isotopes that can be incorporated into compounds of the invention include isotopes of hydrogen, carbon, nitrogen, oxygen, and fluorine such as ²H, ³H, ¹¹C, ¹³C, ¹⁴C, ¹⁵N, ¹⁷O, ¹⁸O, ¹⁸F, and ³⁵S, respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Additionally, any hydrogen atom present in the compound of the invention may be substituted with an ¹⁸F atom, a substitution that may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in any compound described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃, etc.). In certain other embodiments, when two substituents are combined to form a cycle, the unsubstituted carbons may be deuterated. This substitution can improve the properties of Compound 1, for example, deuterium substitution at a metabolic site can reduce the rate of metabolism (e.g., the kinetic isotope effect). Similarly, deuterium substitution near a metabolic site can also reduce the rate of metabolism. In certain embodiments deuterium is substituted at a metabolic site or at the alpha, beta, or gamma position.

The compounds of the present invention may form a solvate with a solvent (including water). Therefore, in one non-limiting embodiment, the invention includes a solvated form of the compounds described herein. The term “solvate” refers to a molecular complex of a compound of the present invention (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g., D₂O, acetone-d₆, DMSO-d₆. A solvate can be in a liquid or solid form.

A “dosage form” means a unit of administration of an active agent. Examples of dosage forms include tablets, capsules, injections, suspensions, liquids, emulsions, particles, spheres, creams, ointments, suppositories, inhalable forms, transdermal forms, buccal, sublingual, topical, gel, mucosal, and the like. A “dosage form” can also include an implant, for example an implant inserted into a tumor or abnormal cell proliferation.

“Parenteral” administration of a compound includes, e.g., subcutaneous (s.c.), intravenous (i.v.), intramuscular (i.m.), or intrasternal injection, or infusion techniques.

The term “a pharmaceutically acceptable salt” refers to a salt that is suitable for use in contact with the tissues of humans and animals. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further may optionally include solvates of the compound or its salt.

Examples of pharmaceutically acceptable salts include, but are not limited to, mineral or organic acid salts of basic residues. For example, conventional non-toxic base salts include those derived from inorganic bases such as sodium hydroxide, sodium bicarbonate, sodium carbonate, potassium hydroxide, potassium bicarbonate, and potassium carbonate. Lists of additional suitable salts may be found, e.g., in Remington's Pharmaceutical Sciences, 17th ed., Mack Publishing Company, Easton, Pa., p. 1418 (1985).

The term “carrier” means a diluent, excipient, or vehicle that an active agent is used or delivered in.

The term “pharmaceutically acceptable excipient” denotes an ingredient used in the manufacture of the dosage form and is suitably non-toxic such as a disintegrator, a binder, a filler, a solvent, a buffer, a tonicity agent, a stabilizer, an antioxidant, a surfactant or a lubricant used in formulating pharmaceutical products.

A “patient” or “host” or “subject” is a human or non-human animal in need of treatment, of any of the disorders as specifically described herein, for example that is modulated by BRAF, and in particular, mutated BRAF. Typically, the host is a human. A “host” may alternatively refer to for example, a mammal, primate (e.g., human), horse, dog, cat, cow, sheep, goat, bird and the like.

“Therapeutically effective amount” means an amount of a compound that, when administered to a host in need thereof, is sufficient to treat the disease state. The “therapeutically effective amount” will vary depending on the disease state being treated, the severity of the disease treated, the age and relative health of the host, the route and form of administration, the judgment of the attending medical or veterinary practitioner, and other factors.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to be a specific disclosure of each subrange such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “inhibitor” denotes a compound which competes with, reduces, or prevents the binding of a particular ligand to a particular receptor, or which reduces or prevents the function of a particular protein.

If one of the starting materials or compounds of the present invention contains one or more functional groups which are not stable or are reactive under the reaction conditions of one or more reaction steps, appropriate protecting groups (as described e.g., in “Protective Groups in Organic Chemistry” by P. G. M. Wuts, 5th Ed., 2007, Wiley, New York) can be introduced before the critical step applying methods well known in the literature. Such protecting groups can be removed at a later stage of the synthesis using standard methods described in the literature. Examples of protecting groups are tert-butyl (tBu), tert-butoxycarbonyl (Boc), 9-fluorenylmethyl carbamate (Fmoc), 2-trimethylsilylethyl carbamate (Teoc), carbobenzyloxy (Cbz), and p-methoxybenzyloxycarbonyl (Moz).

The compound of the present invention if not otherwise described in context can contain an asymmetric center and can be present in the form of an optically pure enantiomer, mixture of enantiomers such as, for example, a racemate, mixture of diastereoisomers, diastereoisomeric racemate or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Whenever a chiral carbon is present in a chemical structure, unless otherwise indicated in context or as drawn, it is intended that all stereoisomers associated with that chiral carbon are encompassed by the structure as pure stereoisomers as well as mixtures thereof.

In the embodiments, where chirally pure isomers (or optically pure enantiomers) are provided, chirally pure means that the compound contains greater than 90% of the desired isomer by mole, particularly greater than 95% of the desired isomer by mole, greater than 98% of the desired isomer by mole, greater than 99% of the desired isomer by mole, said mole percent based upon the total mole of the isomer(s) of the compound. Chirally pure or chirally enriched compounds may be prepared by chirally selective synthesis or by separation of enantiomers.

### Compound 1 Morphic Forms

An investigation of morphic forms of Compound 1 was performed for Compound 1 itself (free form) and for salts of Compound 1 (salt forms). In total, three crystalline Compound 1 free forms were identified as polymorphs, including Form A, which is a hydrate, and two anhydrates, identified as Form B and Form C. Five salts and their polymorphs were identified from the salt investigation, including Compound 1 sodium salt Form D, Compound 1 sodium salt Form E, Compound 1 sodium salt Form F, Compound 1 potassium salt Form G, and Compound 1 potassium salt Form H.

### Morphic Forms of Free Compound 1

The formation of morphic forms of Compound 1 was investigated in a variety of crystallization conditions. Conditions tested included equilibration, slow cooling, slow evaporation, precipitation by addition of anti-solvent, and vapor diffusion.

Three crystalline forms were identified for Compound 1: Form A, the starting material, and two anhydrates, Form B and Form C.

### Compound 1 Form A

Form A is a low crystalline form. It was obtained from IPA, MTBE, EtOH, IPAc and toluene by equilibration at 25° C., from IPA, IPAc, toluene, DMSO/water (v:v=23:77) and DMSO/water (v:v=57:43) by temperature cycle, and from acetone by anti-solvent addition experiments. No improvement in crystallinity was observed after equilibration for 2 weeks. It contains about 1.9% of water by weight according to KF result. DSC shows a broad endothermic peak from about 13° C. It melts at Tₒₙₛₑₜ of 167.9° C. with an enthalpy of 18 J/g. TGA shows about 2.7% weight loss at about 160° C. ¹H NMR shows no detectable residual solvent.

In certain embodiments Form A is characterized by an XRPD pattern with one or more peaks within +/−0.4, 0.3, or 0.2° 2theta of the peaks listed in Peak List #1 (see Example 28 for the XRPD method description).

Peak List #1

| | | Net | Gross | Rel. |
|---|---|---|---|---|
| Angle | d Value | Intensity | Intensity | Intensity |
| 15.332° | 5.77435 Å | 296.820 | 753.411 | 52.9% |
| 18.385° | 4.82177 Å | 560.964 | 1135.83 | 100.0% |
| 24.883° | 3.57537 Å | 139.745 | 722.469 | 24.9% |
| 25.266° | 3.52202 Å | 243.102 | 815.932 | 43.3% |

1. In certain embodiments Compound 1 Form A is characterized by an XRPD pattern which has at least three peaks selected from 15.3, 18.4, 24.9, and 25.3+/−0.4° 2theta. In other embodiments Compound 1 Form A is characterized by an XRPD pattern which has at least three peaks selected from 15.3, 18.4, 24.9, and 25.3+/−0.3° 2theta. Or in other embodiments Compound 1 Form A is characterized by an XRPD pattern which has at least three peaks selected from 15.3, 18.4, 24.9, and 25.3+/−0.2° 2theta.

In certain embodiments, Form A is characterized by a melting onset of about 167.9° C. f 10° C. and/or melting enthalpy of about 18 J/g±10 J/g on DCS thermogram. In certain embodiments, Form A is characterized by a weight loss of about 2.7% at about 160.0° C.±10° C. as measured by TGA.

In certain embodiments, Form A is characterized by a solubility of at least about 250 mg/mL in DMSO when solubilized at about 25° C.±5° C. as described in Example 7.

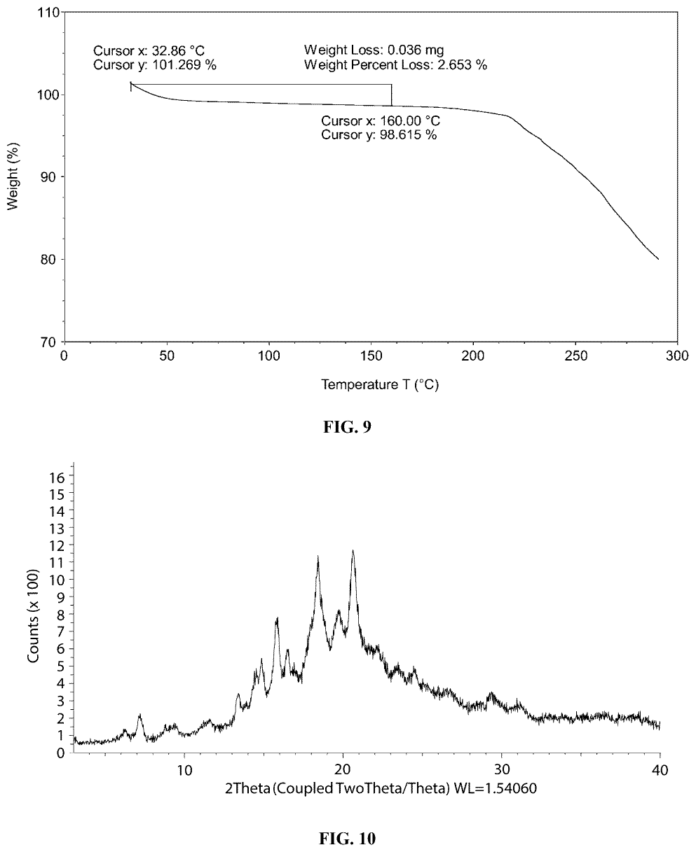

FIG. 7 depicts the XRPD pattern of Compound 1 Form A. FIG. 8 depicts the DSC thermogram of Compound 1 Form A. FIG. 9 depicts the TGA thermogram of Compound 1 Form A.

### Compound 1 Form B

Form B is an anhydrate. It was obtained from tested solvent systems by equilibration. Form B is of medium crystallinity. DSC shows a melting peak at Tₒₙₛₑₜ of 194.4° C. Form B decomposes upon melting. TGA shows about 1.1% weight loss at about 170° C. ¹H NMR shows no detectable residual solvent. Since Form B can also be isolated from organic solvent for example methanol or acetonitrile and water mixture with water activity above about 0.9, this anhydrate should also be stable in aqueous media.

Morphic Form B is superior to the other morphic forms of Compound 1 because of its high stability, scalability, and reproducibility. For example, when Compound 1 Form B is tested for stability over a one-week period at 25° C./92% relative humidity in an open container, 40° C./75% RH in an open container, and at 60° C. in a tight container, Compound 1 Form B demonstrates excellent chemical and physical stability without any changes in purity, crystal form and/or crystallinity. When Compound 1 Form B is tested in water sorption and desorption experiment at 25° C. in a 40-0-95-0-40% RH cycle, Compound 1 Form B demonstrates excellent stability and no changes in crystallinity with only slight hygroscopicity (water uptake of 1.6% at 95% RH). Compound 1 Form B also shows excellent morphic form stability under compression (2 MPa and MPa), dry grinding and wet granulation conditions. Wet granulation experiments in the presence of water or ethanol also indicate no changes in crystallinity. Compound 1 Form B also demonstrates morphic form stability in aqueous media over a broad pH range spanning from 1.2 to 7.0.

In certain embodiments Form B is characterized by an XRPD pattern with one or more peaks within +/−0.4, 0.3, or 0.2° 2theta of the peaks listed in Peak List #2 (see Example 28 for the XRPD method description).

Peak List #2

| | | Net | Gross | Rel. |
|---|---|---|---|---|
| Angle | d Value | Intensity | Intensity | Intensity |
| 5.011° | 17.62222 Å | 40.8684 | 166.131 | 1.1% |
| 5.246° | 16.83322 Å | 63.4373 | 188.216 | 1.8% |
| 7.243° | 12.19511 Å | 155.932 | 295.897 | 4.4% |
| 7.533° | 11.72682 Å | 636.039 | 779.575 | 17.8% |
| 8.845° | 9.98971 Å | 996.604 | 1159.69 | 27.9% |
| 10.037° | 8.80547 Å | 1262.21 | 1442.94 | 35.4% |
| 10.502° | 8.41688 Å | 726.694 | 908.900 | 20.4% |
| 10.945° | 8.07710 Å | 43.1933 | 223.884 | 1.2% |
| 12.555° | 7.04491 Å | 912.477 | 1110.65 | 25.6% |
| 14.697° | 6.02235 Å | 743.321 | 996.079 | 20.8% |
| 15.356° | 5.76556 Å | 1064.30 | 1336.00 | 29.8% |
| 15.730° | 5.62934 Å | 89.3915 | 369.033 | 2.5% |
| 16.404° | 5.39957 Å | 3566.43 | 3855.24 | 100.0% |
| 16.673° | 5.31303 Å | 1021.37 | 1312.00 | 28.6% |
| 17.334° | 5.11181 Å | 288.181 | 578.786 | 8.1% |
| 17.723° | 5.00052 Å | 384.456 | 672.078 | 10.8% |
| 18.143° | 4.88574 Å | 56.4248 | 338.344 | 1.6% |
| 18.640° | 4.75652 Å | 598.644 | 884.458 | 16.8% |
| 18.982° | 4.67164 Å | 328.193 | 620.273 | 9.2% |
| 19.315° | 4.59167 Å | 406.576 | 703.138 | 11.4% |
| 20.106° | 4.41273 Å | 335.890 | 636.585 | 9.4% |
| 20.375° | 4.35510 Å | 408.920 | 708.945 | 11.5% |
| 21.130° | 4.20120 Å | 151.667 | 444.183 | 4.3% |
| 21.325° | 4.16325 Å | 360.730 | 649.963 | 10.1% |
| 21.916° | 4.05234 Å | 69.1334 | 354.864 | 1.9% |
| 22.276° | 3.98766 Å | 370.710 | 656.735 | 10.4% |
| 22.680° | 3.91748 Å | 702.451 | 986.558 | 19.7% |
| 23.071° | 3.85198 Å | 487.910 | 767.897 | 13.7% |
| 23.760° | 3.74180 Å | 376.488 | 643.796 | 10.6% |
| 24.010° | 3.70336 Å | 174.543 | 435.533 | 4.9% |
| 24.744° | 3.59524 Å | 106.413 | 359.431 | 3.0% |
| 25.110° | 3.54365 Å | 260.101 | 515.679 | 7.3% |
| 25.337° | 3.51245 Å | 721.902 | 978.085 | 20.2% |
| 25.813° | 3.44865 Å | 113.072 | 368.092 | 3.2% |
| 26.395° | 3.37399 Å | 167.819 | 416.931 | 4.7% |
| 26.656° | 3.34152 Å | 219.098 | 463.952 | 6.1% |
| 27.266° | 3.26810 Å | 105.726 | 349.010 | 3.0% |
| 27.846° | 3.20136 Å | 125.997 | 372.339 | 3.5% |
| 28.661° | 3.11217 Å | 93.0172 | 345.339 | 2.6% |
| 28.802° | 3.09724 Å | 104.026 | 357.944 | 2.9% |
| 29.345° | 3.04111 Å | 200.487 | 457.848 | 5.6% |
| 29.685° | 3.00703 Å | 181.283 | 438.608 | 5.1% |
| 30.167° | 2.96015 Å | 39.5660 | 293.963 | 1.1% |
| 31.482° | 2.83945 Å | 155.721 | 386.468 | 4.4% |
| 31.859° | 2.80664 Å | 66.7041 | 286.130 | 1.9% |
| 33.026° | 2.71014 Å | 151.086 | 367.407 | 4.2% |
| 34.111° | 2.62635 Å | 97.2394 | 334.272 | 2.7% |
| 35.028° | 2.55963 Å | 49.5358 | 297.606 | 1.4% |
| 35.760° | 2.50890 Å | 42.0468 | 301.991 | 1.2% |
| 36.114° | 2.48512 Å | 40.7743 | 303.661 | 1.1% |

1. In certain embodiments Compound 1 Form B is characterized by an XRPD pattern which has at least three peaks selected from 7.5, 8.8, 10.0, 10.5, 12.6, 14.7, 15.4, 16.4, 16.7, 18.6, 22.7, and 25.3+/−0.4° 2theta.

Other embodiments include but are not limited to the following:

2. The morphic form of embodiment 1, wherein Compound 1 Form B is characterized by an XRPD pattern which has at least three peaks selected from 7.5, 8.8, 10.0, 10.5, 12.6, 14.7, 15.4, 16.4, 16.7, 18.6, 22.7, and 25.3+/−0.3° 2theta.

3. The morphic form of embodiment 1, wherein Compound 1 Form B is characterized by an XRPD pattern which has at least three peaks selected from 7.5, 8.8, 10.0, 10.5, 12.6, 14.7, 15.4, 16.4, 16.7, 18.6, 22.7, and 25.3+/−0.2° 2theta.

4. The morphic form of any one of embodiments 1-3, that exhibits at least four of the listed peaks.

5. The morphic form of any one of embodiments 1-3, that exhibits at least five of the listed peaks.

6. The morphic form of any one of embodiments 1-3, that exhibits at least six of the listed peaks.

7. The morphic form of any one of embodiments 1-3, that exhibits at least seven of the listed peaks.

8. The morphic form of any one of embodiments 1-3, that exhibits at least eight of the listed peaks.

9. The morphic form of any one of embodiments 1-3, that exhibits at least nine of the listed peaks.

10. The morphic form of any one of embodiments 1-3, that exhibits at least ten of the listed peaks.

11. The morphic form of any one of embodiments 1-10, wherein the XRPD includes a peak at 16.4+/−0.2° 2theta.

12. The morphic form of any one of embodiments 1-11, wherein the XRPD includes a peak at 10.0+/−0.2° 2theta.

13. The morphic form of any one of embodiments 1-12, wherein the XRPD includes a peak at 15.4+/−0.2° 2theta.

14. The morphic form of any one of embodiments 1-13, wherein the XRPD includes a peak at 16.7+/−0.2° 2theta.

15. The morphic form of any one of embodiments 1-14, wherein the XRPD includes a peak at 8.8+/−0.2° 2theta.

16. The morphic form of any one of embodiments 1-15, wherein the XRPD includes a peak at 12.6+/−0.2° 2theta.

17. The morphic form of any one of embodiments 1-16, wherein the XRPD includes a peak at 14.7+/−0.2° 2theta.

18. The morphic form of any one of embodiments 1-17, wherein the XRPD includes a peak at 10.5+/−0.2° 2theta.

19. The morphic form of any one of embodiments 1-18, wherein the XRPD includes a peak at 25.3+/−0.2° 2theta.

20. The morphic form of any one of embodiments 1-19, wherein the XRPD includes a peak at 22.7+/−0.2° 2theta.

In certain embodiments, Compound 1 Form B is characterized by a melting onset of about 194.4° C.±10° C. and/or melting enthalpy of about 71.3 J/g 10 J/g on DCS thermogram. In certain embodiments, Form B is characterized by a weight loss of about 1.1% at about 170.0° C.±10° C. as measured by TGA.

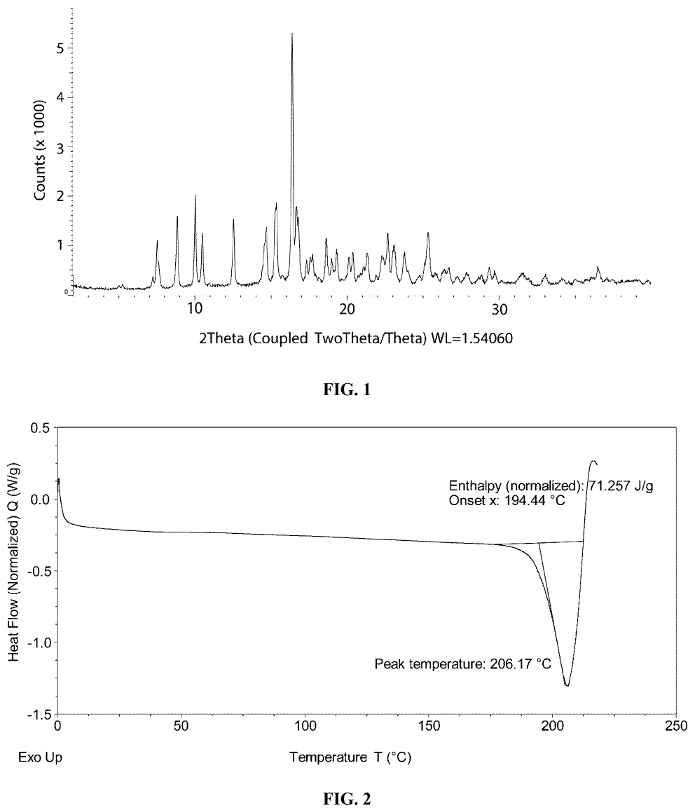

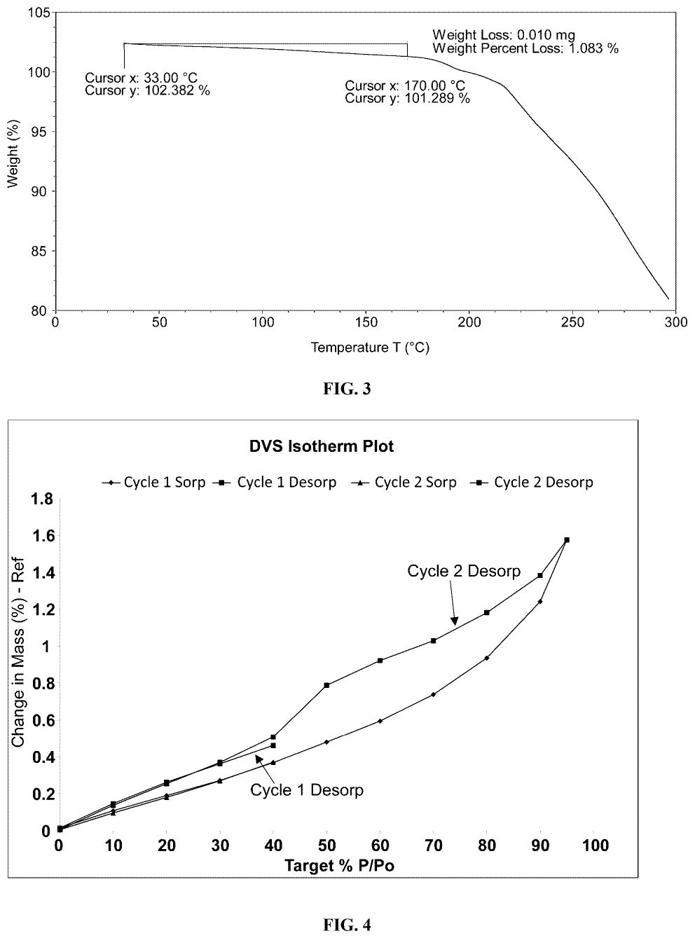

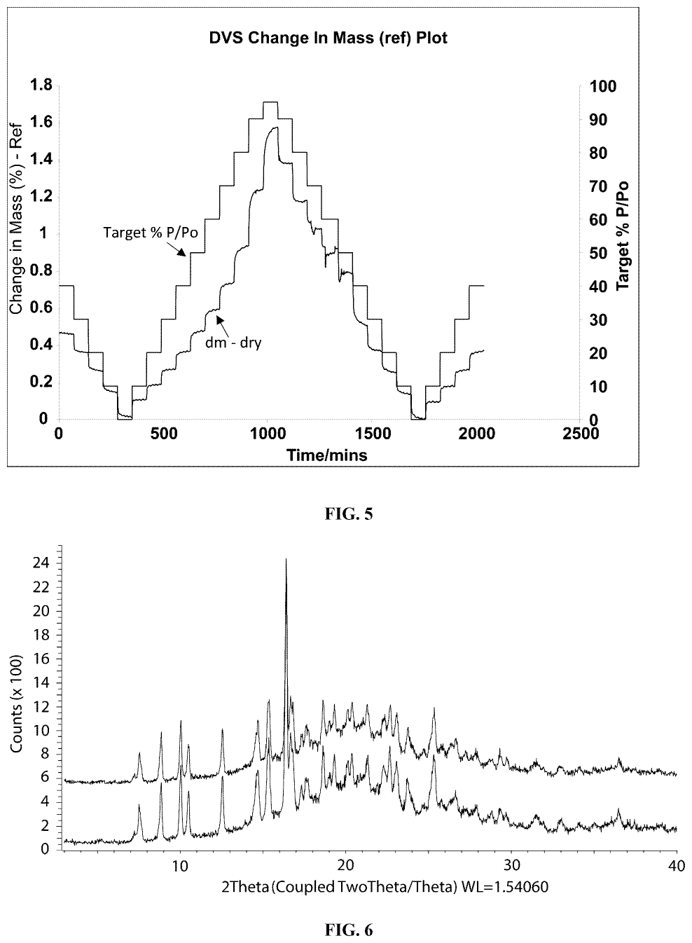

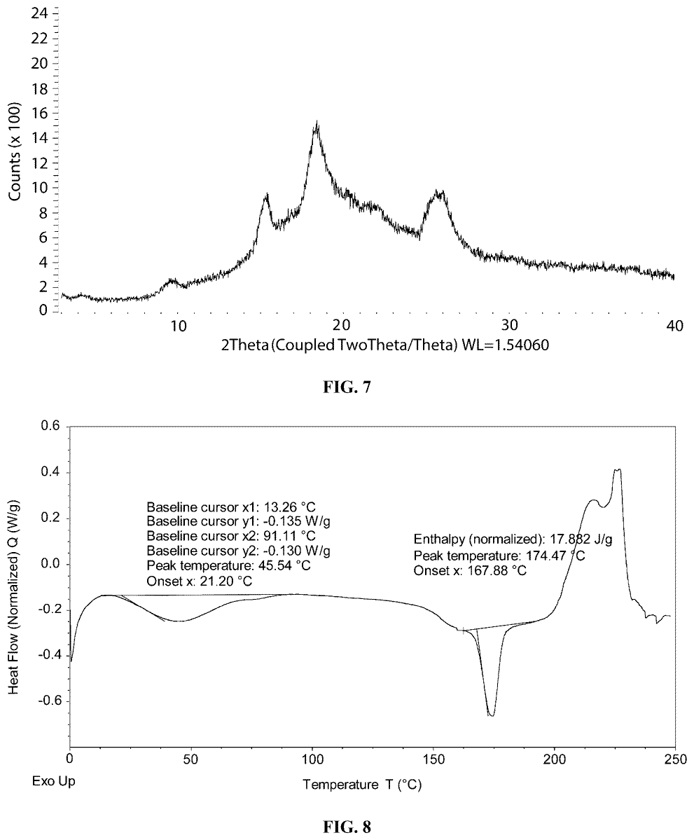

In certain embodiments, Compound 1 Form B is characterized by a solubility of at least about 250 mg/mL in DMSO when solubilized at about 25° C.±5° C. as described in Example 7. FIG. 1 depicts the XRPD pattern of Compound 1 Form B. FIG. 2 depicts the DSC thermogram of Compound 1 Form B. FIG. 3 depicts the TGA thermogram of Compound 1 Form B.

### Compound 1 Form C

Form C is an anhydrate. It was obtained from ethyl acetate and 2-methyltetrahydrofuran (2-MeTHF) by equilibration at 25° C., and from 2-MeTHF by temperature cycling. Form C is of medium crystallinity. DSC shows a broad endothermic peak from 25.9° C., and a melting peak at Tₒₙₛₑₜ of 185.3° C. It decomposes upon melting. TGA shows about 2.2% weight loss at about 180° C. ¹H NMR shows about 2.7% ethyl acetate residue by weight (0.2 equivalent by molar ratio). Form C is a metastable anhydrate. It converted to anhydrate Form B after, for example, about 8 temperature cycles.

In certain embodiments Form C is characterized by an XRPD pattern with one or more peaks within +/−0.4, 0.3, or 0.2° 2theta of the peaks listed in Peak List #3 (see Example 28 for the XRPD method description).

Peak List #3

| | | Net | Gross | Rel. |
|---|---|---|---|---|
| Angle | d Value | Intensity | Intensity | Intensity |
| 6.248° | 14.13390 Å | 22.6856 | 97.3480 | 4.3% |
| 7.195° | 12.27712 Å | 82.0108 | 160.168 | 15.6% |
| 8.831° | 10.00508 Å | 33.5302 | 120.836 | 6.4% |
| 9.392° | 9.40884 Å | 21.9799 | 116.992 | 4.2% |
| 13.396° | 6.60433 Å | 100.559 | 296.884 | 19.1% |
| 14.525° | 6.09340 Å | 160.403 | 412.273 | 30.4% |
| 14.867° | 5.95396 Å | 194.890 | 461.869 | 37.0% |
| 15.823° | 5.59633 Å | 318.014 | 622.973 | 60.3% |
| 16.496° | 5.36962 Å | 155.377 | 483.317 | 29.5% |
| 17.944° | 4.93921 Å | 260.826 | 627.750 | 49.5% |
| 18.416° | 4.81378 Å | 496.333 | 872.852 | 94.1% |
| 19.725° | 4.49716 Å | 277.851 | 673.027 | 52.7% |
| 20.635° | 4.30090 Å | 527.269 | 928.512 | 100.0% |
| 22.161° | 4.00799 Å | 145.955 | 544.662 | 27.7% |
| 23.481° | 3.78567 Å | 71.1339 | 454.809 | 13.5% |
| 24.561° | 3.62161 Å | 72.6506 | 435.165 | 13.8% |
| 29.365° | 3.03906 Å | 85.0661 | 327.154 | 16.1% |

1. In certain embodiments Compound 1 Form C is characterized by an XRPD pattern which has at least three peaks selected from 7.2, 13.4, 14.5, 14.9, 15.8, 16.5, 17.9, 18.4, 19.7, 20.6, 22.2, and 29.4+/−0.4° 2theta.

2. The morphic form of embodiment 1, wherein Compound 1 Form C is characterized by an XRPD pattern which has at least three peaks selected from 7.2, 13.4, 14.5, 14.9, 15.8, 16.5, 17.9, 18.4, 19.7, 20.6, 22.2, and 29.4+/−0.3° 2theta.

3. The morphic form of embodiment 1, wherein Compound 1 Form C is characterized by an XRPD pattern which has at least three peaks selected from 7.2, 13.4, 14.5, 14.9, 15.8, 16.5, 17.9, 18.4, 19.7, 20.6, 22.2, and 29.4+/−0.2° 2theta.

Other embodiments include but are not limited to the following:

4. The morphic form of any one of embodiments 1-3, that exhibits at least four of the listed peaks.

5. The morphic form of any one of embodiments 1-3, that exhibits at least five of the listed peaks.

6. The morphic form of any one of embodiments 1-3, that exhibits at least six of the listed peaks.

7. The morphic form of any one of embodiments 1-3, that exhibits at least seven of the listed peaks.

8. The morphic form of any one of embodiments 1-3, that exhibits at least eight of the listed peaks.

9. The morphic form of any one of embodiments 1-3, that exhibits at least nine of the listed peaks.

10. The morphic form of any one of embodiments 1-3, that exhibits at least ten of the listed peaks.

11. The morphic form of any one of embodiments 1-10, wherein the XRPD includes a peak at 20.6+/−0.2° 2theta.

12. The morphic form of any one of embodiments 1-11, wherein the XRPD includes a peak at 18.4+/−0.2° 2theta.

13. The morphic form of any one of embodiments 1-12, wherein the XRPD includes a peak at 15.8+/−0.2° 2theta.

14. The morphic form of any one of embodiments 1-13, wherein the XRPD includes a peak at 19.7+/−0.2° 2theta.

15. The morphic form of any one of embodiments 1-14, wherein the XRPD includes a peak at 17.9+/−0.2° 2theta.

16. The morphic form of any one of embodiments 1-15, wherein the XRPD includes a peak at 14.9+/−0.2° 2theta.

17. The morphic form of any one of embodiments 1-16, wherein the XRPD includes a peak at 14.5+/−0.2° 2theta.

18. The morphic form of any one of embodiments 1-17, wherein the XRPD includes a peak at 16.5+/−0.2° 2theta.

19. The morphic form of any one of embodiments 1-18, wherein the XRPD includes a peak at 22.2+/−0.2° 2theta.

20. The morphic form of any one of embodiments 1-19, wherein the XRPD includes a peak at 13.4+/−0.2° 2theta.

In certain embodiments, Form C is characterized by a melting onset of about 185.3° C.±10° C. and/or melting enthalpy of about 33.9 J/g±10 J/g on DCS thermogram. In certain embodiments, Form C is characterized by a weight loss of about 2.2% at about 180.0° C.±10° C. as measured by TGA.

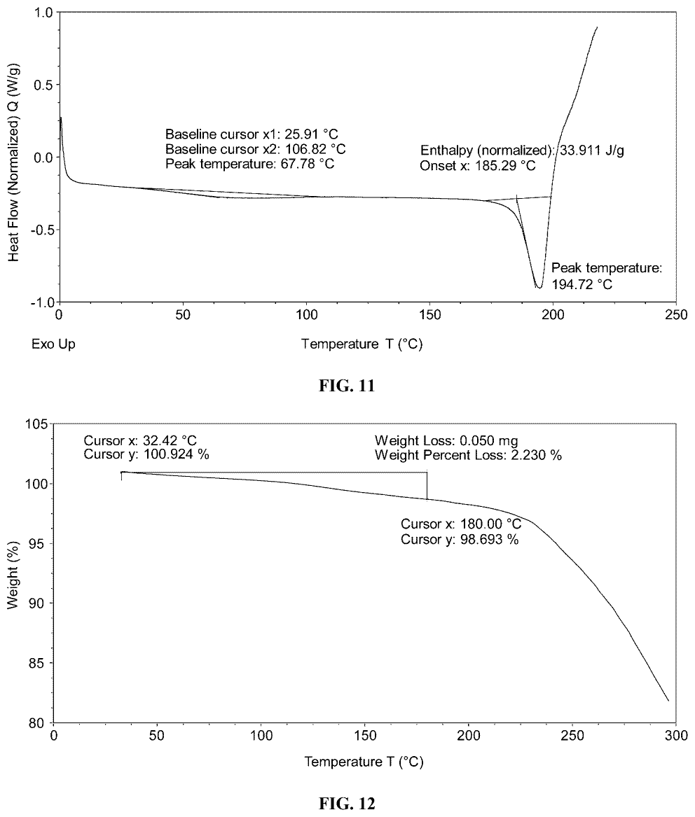

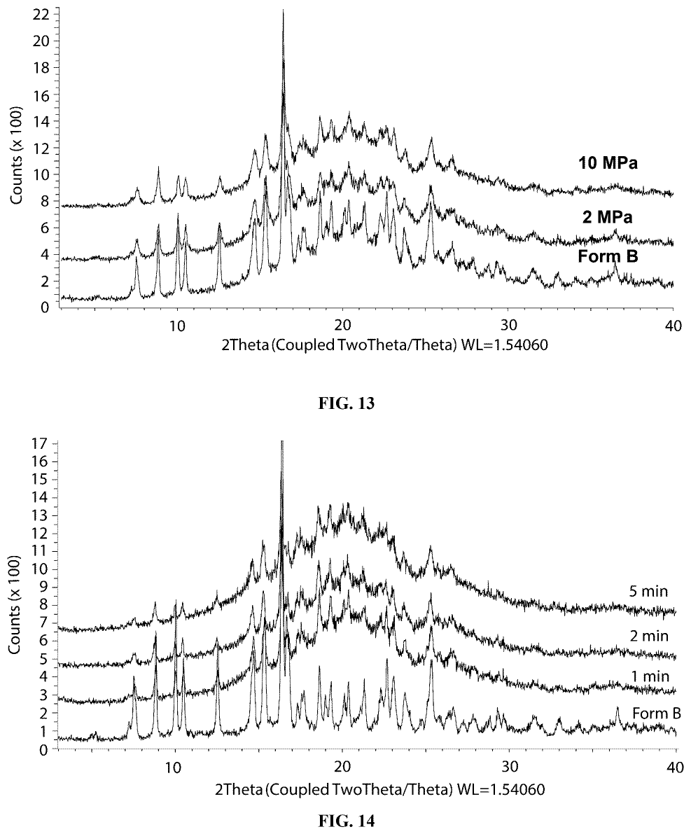

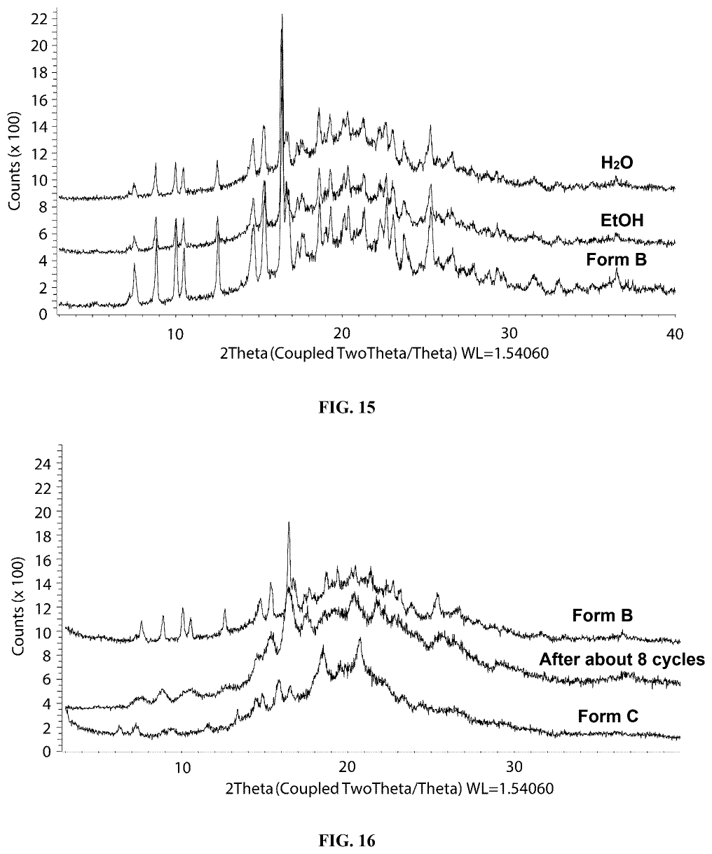

FIG. 10 depicts the XRPD pattern of Compound 1 Form C. FIG. 11 depicts the DSC thermogram of Compound 1 Form C. FIG. 12 depicts the TGA thermogram of Compound 1 Form C.

### Compound 1 Salt Morphic Forms

Formation of Compound 1 salt morphic forms was investigated in a variety of crystallization conditions. Conditions tested included slurry equilibration, precipitation by addition of anti-solvent, and re-slurry.

Five crystalline forms were identified among Compound 1 salt morphic forms: sodium salt Form D, sodium salt Form E, sodium salt Form F, potassium salt Form G, and potassium salt Form H. Among these salt Forms D to H, the sodium salt Form F shows good physicochemical characteristics including good crystallinity, reasonable stoichiometry, high dehydration onset and good counter ion safety.

### Compound 1 Sodium Salt Form F

Compound 1 sodium salt Form F is a hydrate. Sodium salt Form F has medium crystallinity. DSC shows a dehydration peak from 13.7° C., 64.7° C., 116.5° C. and a melting point at Tₒₙₛₑₜ of 194.7° C. TGA shows about 4.8% weight loss at about 180° C. HPLC shows 98.9% chemical purity. IC and HPLC shows that the stoichiometric of form and sodium salt is 1:1.2. ¹H NMR shows no detectable residual solvent. KF (Karl Fischer analysis) shows it contains about 4.6% water by weight, equivalent to 2.5 water molecules. This hydrate form is stable under vacuum drying at about 30° C.

Sodium salt Form F shows about 1% chemical purity decrease after photostability evaluation. Sodium salt Form F shows slight chemical purity decrease (about 1.2%) after stress at 25° C./92% RH. Sodium salt Form F also shows a slight crystallinity decrease after stress at 60° C. in a closed container over 1 week, which may be due to partial dehydration.

Hygroscopicity of the sodium salt Form F was evaluated by a dynamic vapor sorption (DVS) test at 25° C. The sodium salt Form F is slightly hygroscopic below 80% RH. It then becomes hygroscopic and shows 12.6% water uptake from 80% RH to 95% RH at 25° C. After the DVS test, the sodium salt Form F shows no form change and no crystallinity decrease.

In certain embodiments Form F is characterized by an XRPD pattern with one or more peaks within +/−0.4, 0.3, or 0.2° 2theta of the peaks listed in Peak List #4 (see Example 28 for the XRPD method description).

Peak List #4

| | | Net | Gross | Rel. |
|---|---|---|---|---|
| Angle | d Value | Intensity | Intensity | Intensity |
| 6.383° | 13.83683 Å | 34.3259 | 187.502 | 1.3% |
| 6.796° | 12.99598 Å | 269.394 | 427.352 | 10.2% |
| 7.829° | 11.28358 Å | 44.2707 | 204.293 | 1.7% |
| 9.509° | 9.29309 Å | 245.928 | 429.865 | 9.3% |
| 9.779° | 9.03780 Å | 160.529 | 351.049 | 6.1% |
| 10.982° | 8.05010 Å | 155.718 | 374.650 | 5.9% |
| 12.322° | 7.17717 Å | 324.600 | 611.089 | 12.3% |
| 13.619° | 6.49670 Å | 643.363 | 1001.90 | 24.5% |
| 14.336° | 6.17342 Å | 254.083 | 653.253 | 9.7% |
| 14.958° | 5.91794 Å | 878.321 | 1307.26 | 33.4% |
| 15.458° | 5.72756 Å | 504.970 | 954.123 | 19.2% |
| 16.014° | 5.52988 Å | 493.157 | 960.893 | 18.7% |
| 16.594° | 5.33789 Å | 157.114 | 639.878 | 6.0% |
| 17.493° | 5.06577 Å | 524.960 | 1022.21 | 20.0% |
| 18.315° | 4.84021 Å | 611.400 | 1112.56 | 23.2% |
| 19.006° | 4.66559 Å | 222.853 | 720.377 | 8.5% |
| 19.695° | 4.50406 Å | 2630.53 | 3118.15 | 100.0% |
| 20.282° | 4.37502 Å | 964.342 | 1438.58 | 36.7% |
| 21.096° | 4.20788 Å | 226.850 | 674.942 | 8.6% |
| 21.687° | 4.09457 Å | 857.549 | 1281.19 | 32.6% |
| 23.269° | 3.81960 Å | 105.409 | 484.861 | 4.0% |
| 24.023° | 3.70151 Å | 627.478 | 1022.51 | 23.9% |
| 24.459° | 3.63651 Å | 560.546 | 961.168 | 21.3% |
| 24.776° | 3.59062 Å | 304.551 | 707.665 | 11.6% |
| 25.399° | 3.50398 Å | 418.496 | 822.613 | 15.9% |
| 26.457° | 3.36613 Å | 145.855 | 569.779 | 5.5% |
| 26.646° | 3.34278 Å | 171.444 | 602.531 | 6.5% |
| 28.023° | 3.18154 Å | 226.853 | 696.110 | 8.6% |
| 28.930° | 3.08383 Å | 171.911 | 652.603 | 6.5% |
| 29.194° | 3.05648 Å | 175.184 | 657.162 | 6.7% |
| 29.974° | 2.97871 Å | 324.273 | 804.653 | 12.3% |
| 30.273° | 2.94999 Å | 537.578 | 1015.22 | 20.4% |
| 30.431° | 2.93502 Å | 701.084 | 1176.79 | 26.7% |
| 31.077° | 2.87548 Å | 193.565 | 657.960 | 7.4% |
| 32.485° | 2.75401 Å | 57.9739 | 478.585 | 2.2% |
| 33.467° | 2.67542 Å | 127.030 | 529.125 | 4.8% |
| 34.525° | 2.59581 Å | 551.568 | 950.992 | 21.0% |
| 37.125° | 2.41976 Å | 120.467 | 532.398 | 4.6% |
| 37.342° | 2.40617 Å | 101.064 | 512.077 | 3.8% |
| 37.423° | 2.40118 Å | 109.110 | 519.630 | 4.1% |
| 39.126° | 2.30049 Å | 167.162 | 589.969 | 6.4% |

1. In certain embodiments Compound 1 sodium salt Form F is characterized by an XRPD pattern which has at least three peaks selected from 13.6, 15.0, 15.5, 16.0, 17.5, 18.3, 19.7, 20.3, 21.7, 24.0, 24.5, 30.3, 30.4, and 34.5+/−0.4° 2theta.

2. The morphic form of embodiment 1, wherein Compound 1 sodium salt Form F is characterized by an XRPD pattern which has at least three peaks selected from 13.6, 15.0, 15.5, 16.0, 17.5, 18.3, 19.7, 20.3, 21.7, 24.0, 24.5, 30.3, 30.4, and 34.5+/−0.3° 2theta.

3. The morphic form of embodiment 1, wherein Compound 1 sodium salt Form F is characterized by an XRPD pattern which has at least three peaks selected from 13.6, 15.0, 15.5, 16.0, 17.5, 18.3, 19.7, 20.3, 21.7, 24.0, 24.5, 30.3, 30.4, and 34.5+/−0.2° 2theta.

Other embodiments include but are not limited to the following:

4. The morphic form of any one of embodiments 1-3, that exhibits at least four of the listed peaks.

5. The morphic form of any one of embodiments 1-3, that exhibits at least five of the listed peaks.

6. The morphic form of any one of embodiments 1-3, that exhibits at least six of the listed peaks.

7. The morphic form of any one of embodiments 1-3, that exhibits at least seven of the listed peaks.

8. The morphic form of any one of embodiments 1-3, that exhibits at least eight of the listed peaks.

9. The morphic form of any one of embodiments 1-3, that exhibits at least nine of the listed peaks.

10. The morphic form of any one of embodiments 1-3, that exhibits at least ten of the listed peaks.

11. The morphic form of any one of embodiments 1-10, wherein the XRPD includes a peak at 19.7+/−0.2° 2theta.

12. The morphic form of any one of embodiments 1-11, wherein the XRPD includes a peak at 20.3+/−0.2° 2theta.

13. The morphic form of any one of embodiments 1-12, wherein the XRPD includes a peak at 15.0+/−0.2° 2theta.

14. The morphic form of any one of embodiments 1-13, wherein the XRPD includes a peak at 21.7+/−0.2° 2theta.

15. The morphic form of any one of embodiments 1-14, wherein the XRPD includes a peak at 30.4+/−0.2° 2theta.

16. The morphic form of any one of embodiments 1-15, wherein the XRPD includes a peak at 13.6+/−0.2° 2theta.

17. The morphic form of any one of embodiments 1-16, wherein the XRPD includes a peak at 24.0+/−0.2° 2theta.

18. The morphic form of any one of embodiments 1-17, wherein the XRPD includes a peak at 18.3+/−0.2° 2theta.

19. The morphic form of any one of embodiments 1-18, wherein the XRPD includes a peak at 24.5+/−0.2° 2theta.

20. The morphic form of any one of embodiments 1-19, wherein the XRPD includes a peak at 34.5+/−0.2° 2theta.

In certain embodiments, Form F is characterized by any one of the following peaks: 59.8° C.±10° C., 132.5±10° C., and 197.7±10° C. and/or melting enthalpy of about 26.4 J/g±10 J/g and 41.8 J/g 10 J/g as measured by DSC. In certain embodiments, Form F is characterized by a weight loss of about 2.7% at about 100.0° C.±10° C. and about 3.2% in the temperature range of about 100.0° C.-170.0° C.±10° C. as measured by TGA.

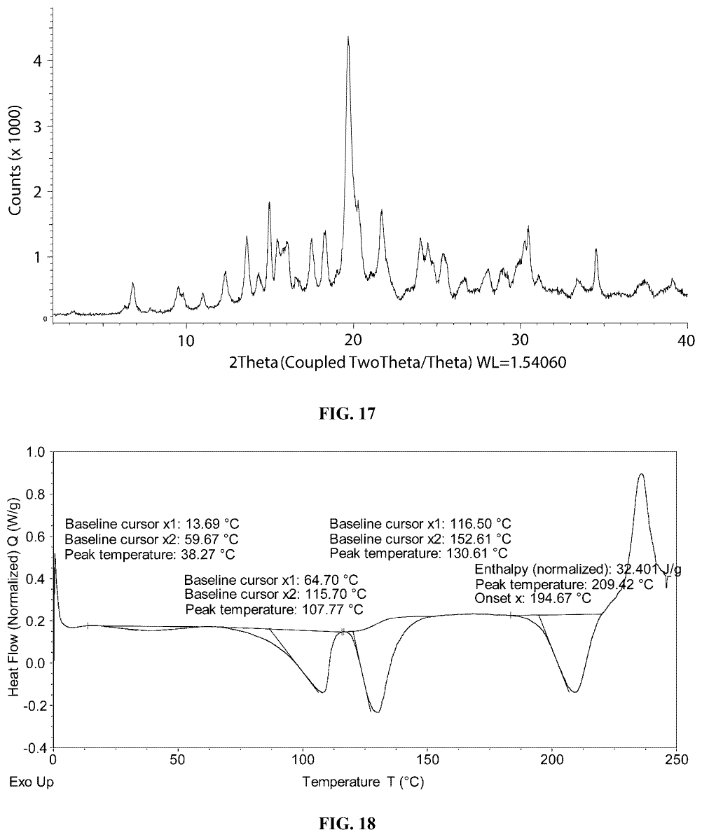

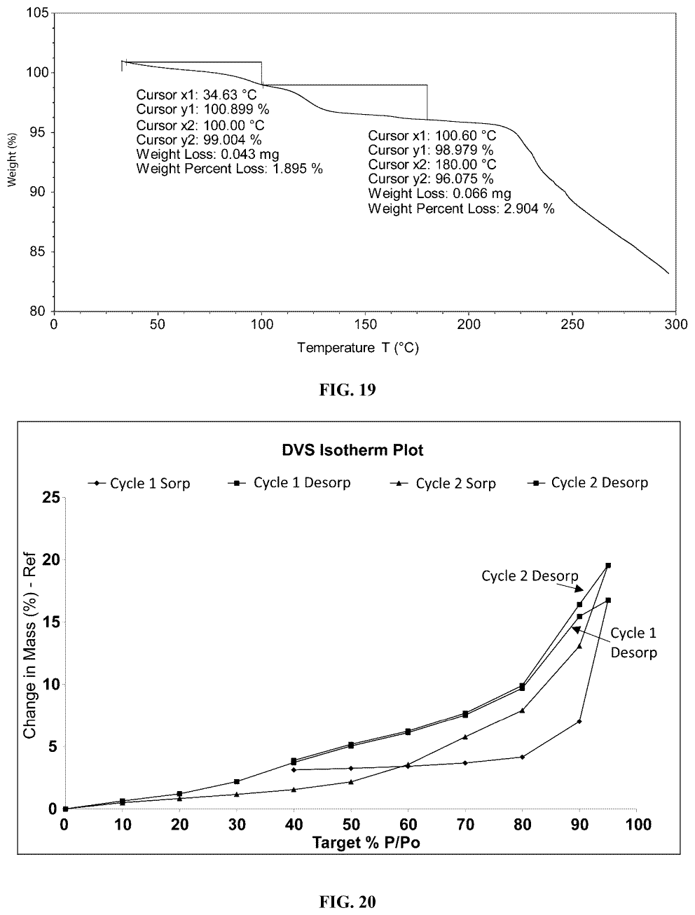

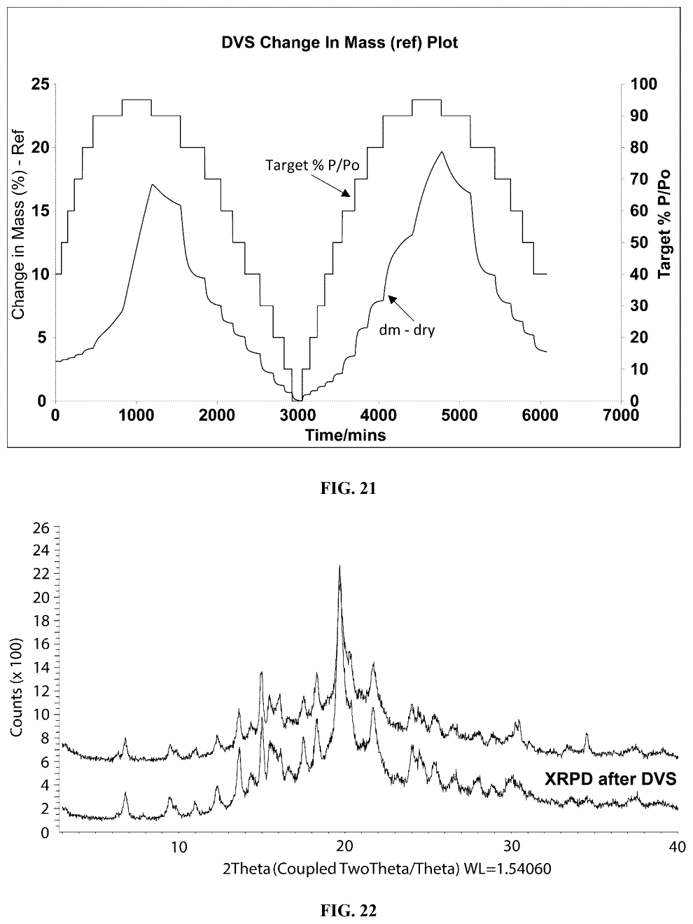

FIG. 17 depicts the XRPD pattern of Compound 1 sodium salt Form F. FIG. 18 depicts the DSC thermogram of Compound 1 sodium salt Form F. FIG. 19 depicts the TGA thermogram of Compound 1 sodium salt Form F.

### Compound 1 Sodium Salt Form D

Compound 1 sodium salt Form D is a hydrate with medium crystallinity. DSC shows a dehydration peak from 4.5° C., peak at 67.5° C., endothermic peak from 117.9° C. and a melting point at Tₒₙₛₑₜ of 229.5° C. TGA shows about 6.9% weight loss at about 150° C. HPLC shows 97.4% chemical purity. IC and HPLC shows that the stoichiometric salt ratio of the compound to Na⁺ is 1:1. ¹H NMR shows no detectable residual solvent.

In certain embodiments Form D is characterized by an XRPD pattern with one or more peaks within +/−0.4, 0.3, or 0.2° 2theta of the peaks listed in Peak List #5 (see Example 28 for the XRPD method description).

Peak List #5

| | | Net | Gross | Rel. |
|---|---|---|---|---|
| Angle | d Value | Intensity | Intensity | Intensity |
| 6.159° | 14.33968 Å | 158.411 | 195.502 | 28.6% |
| 7.159° | 12.33714 Å | 38.6965 | 83.0252 | 7.0% |
| 7.664° | 11.52592 Å | 144.802 | 190.350 | 26.1% |
| 9.257° | 9.54592 Å | 12.9387 | 61.5612 | 2.3% |
| 9.901° | 8.92606 Å | 57.0232 | 105.851 | 10.3% |
| 11.696° | 7.55993 Å | 68.1315 | 136.958 | 12.3% |
| 12.466° | 7.09463 Å | 62.3276 | 148.390 | 11.2% |
| 13.556° | 6.52687 Å | 206.011 | 312.720 | 37.2% |
| 15.342° | 5.77064 Å | 238.693 | 383.929 | 43.1% |
| 15.684° | 5.64551 Å | 124.137 | 279.173 | 22.4% |
| 17.458° | 5.07581 Å | 554.295 | 748.125 | 100.0% |
| 19.736° | 4.49475 Å | 51.7382 | 265.883 | 9.3% |
| 20.832° | 4.26059 Å | 111.566 | 323.655 | 20.1% |
| 24.024° | 3.70131 Å | 57.1907 | 231.517 | 10.3% |
| 25.171° | 3.53519 Å | 48.1641 | 209.618 | 8.7% |
| 27.516° | 3.23895 Å | 67.1110 | 194.787 | 12.1% |
| 29.286° | 3.04710 Å | 35.1525 | 141.336 | 6.3% |

1. In certain embodiments Compound 1 sodium salt Form D is characterized by an XRPD pattern which has at least three peaks selected from 6.2, 7.7, 11.7, 12.5, 13.6, 15.3, 15.7, 17.5, 20.8, and 27.5+/−0.4° 2theta.

2. The morphic form of embodiment 1, wherein Compound 1 sodium salt Form D is characterized by an XRPD pattern which has at least three peaks selected from 6.2, 7.7, 11.7, 12.5, 13.6, 15.3, 15.7, 17.5, 20.8, and 27.5+/−0.3° 2theta.

3. The morphic form of embodiment 1, wherein Compound 1 sodium salt Form D is characterized by an XRPD pattern which has at least three peaks selected from 6.2, 7.7, 11.7, 12.5, 13.6, 15.3, 15.7, 17.5, 20.8, and 27.5+/−0.2° 2theta.

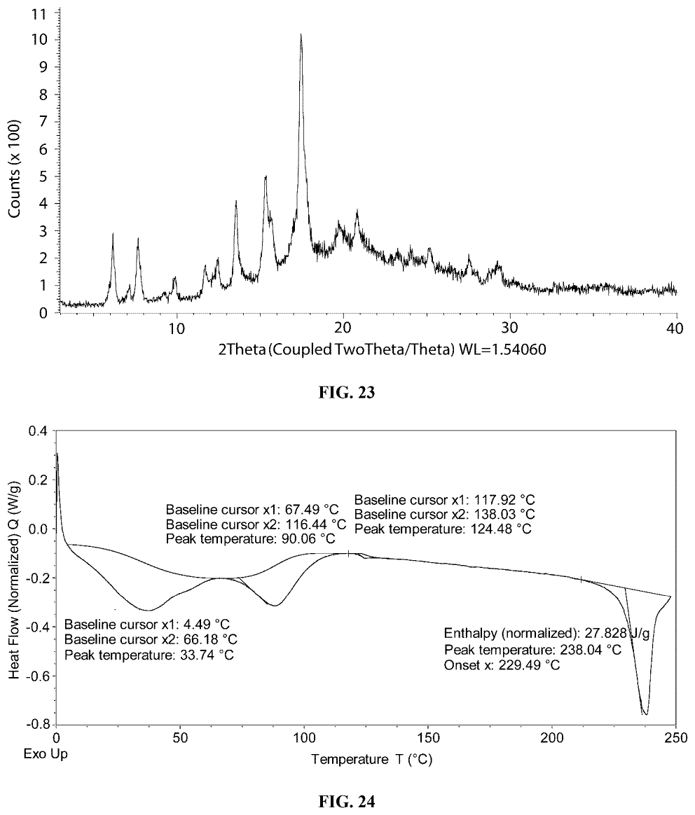

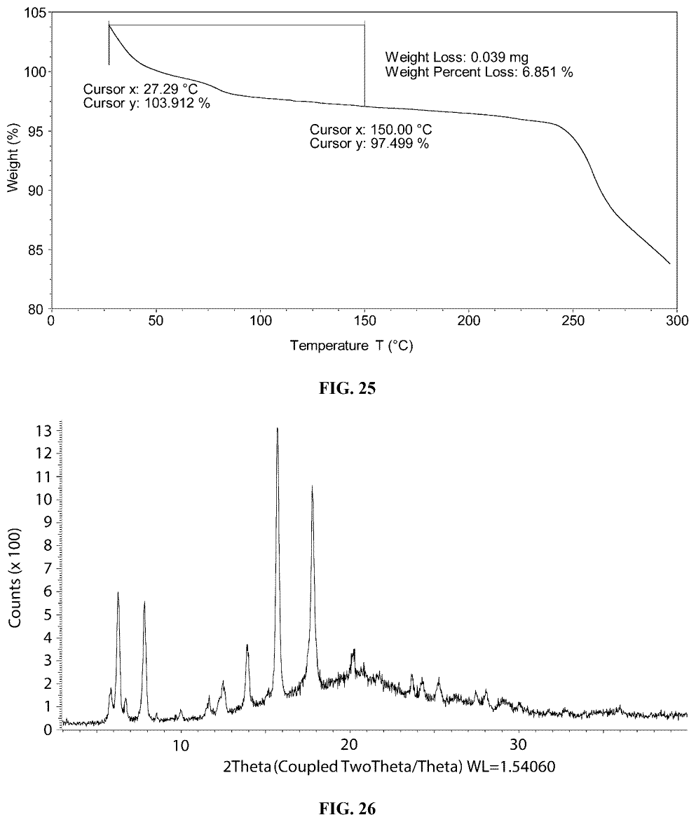

FIG. 23 depicts the XRPD pattern of Compound 1 sodium salt Form D. FIG. 24 depicts the DSC thermogram of Compound 1 sodium salt Form D. FIG. 25 depicts the TGA thermogram of Compound 1 sodium salt Form D.

### Compound 1 Sodium Salt Form E

Compound 1 sodium salt Form E is a hydrate. Sodium salt Form E has medium crystallinity. DSC shows a dehydration peak from 5.6° C., peak at 61.1° C., and a melting point at Tₒₙₛₑₜ of 228.7° C. TGA shows about 10.8% weight loss at about 180° C. HPLC shows about 98% chemical purity. IC and HPLC shows that the stoichiometric salt ratio of the compound to Na⁺ is 1:1. ¹H NMR shows no detectable residual solvent. Water content according to KF analysis is 10.2% by weight.

In certain embodiments Form E is characterized by an XRPD pattern with one or more peaks within +/−0.4, 0.3, or 0.2° 2theta of the peaks listed in Peak List #6 (see Example 28 for the XRPD method description).

Peak List #6

| | | Net | Gross | Rel. |
|---|---|---|---|---|
| Angle | d Value | Intensity | Intensity | Intensity |
| 5.836° | 15.13111 Å | 91.6615 | 129.974 | 12.0% |
| 6.276° | 14.07064 Å | 342.001 | 384.717 | 44.7% |
| 6.717° | 13.14834 Å | 60.1951 | 105.918 | 7.9% |
| 7.832° | 11.27988 Å | 333.725 | 380.209 | 43.6% |
| 8.537° | 10.34977 Å | 22.4216 | 64.6097 | 2.9% |
| 9.996° | 8.84169 Å | 25.0816 | 67.5016 | 3.3% |
| 11.664° | 7.58107 Å | 63.8628 | 125.649 | 8.3% |
| 12.465° | 7.09514 Å | 86.0772 | 159.594 | 11.2% |
| 13.898° | 6.36680 Å | 181.243 | 281.054 | 23.7% |
| 15.707° | 5.63740 Å | 765.226 | 907.872 | 100.0% |
| 17.779° | 4.98489 Å | 574.266 | 752.763 | 75.0% |
| 20.184° | 4.39587 Å | 91.9218 | 288.153 | 12.0% |
| 22.900° | 3.88038 Å | 36.7650 | 204.169 | 4.8% |
| 23.660° | 3.75746 Å | 62.8726 | 216.749 | 8.2% |
| 24.271° | 3.66414 Å | 56.4524 | 200.204 | 7.4% |
| 25.269° | 3.52174 Å | 69.6166 | 196.814 | 9.1% |
| 27.460° | 3.24540 Å | 36.9208 | 142.275 | 4.8% |
| 28.070° | 3.17632 Å | 50.3707 | 145.285 | 6.6% |

1. In certain embodiments Compound 1 sodium salt Form E is characterized by an XRPD pattern which has at least three peaks selected from 5.8, 6.3, 7.8, 12.5, 13.9, 15.7, 17.8, 20.2, and 25.3+/−0.4° 2theta.

2. The morphic form of embodiment 1, wherein Compound 1 sodium salt Form E is characterized by an XRPD pattern which has at least three peaks selected from 5.8, 6.3, 7.8, 12.5, 13.9, 15.7, 17.8, 20.2, and 25.3+/−0.3° 2theta.

3. The morphic form of embodiment 1, wherein Compound 1 sodium salt Form E is characterized by an XRPD pattern which has at least three peaks selected from 5.8, 6.3, 7.8, 12.5, 13.9, 15.7, 17.8, 20.2, and 25.3+/−0.2° 2theta.

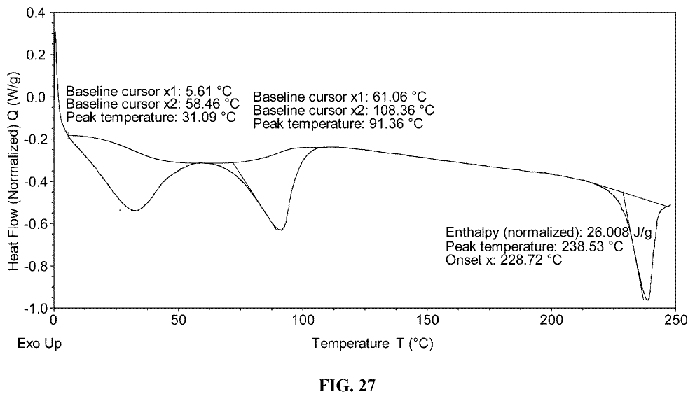

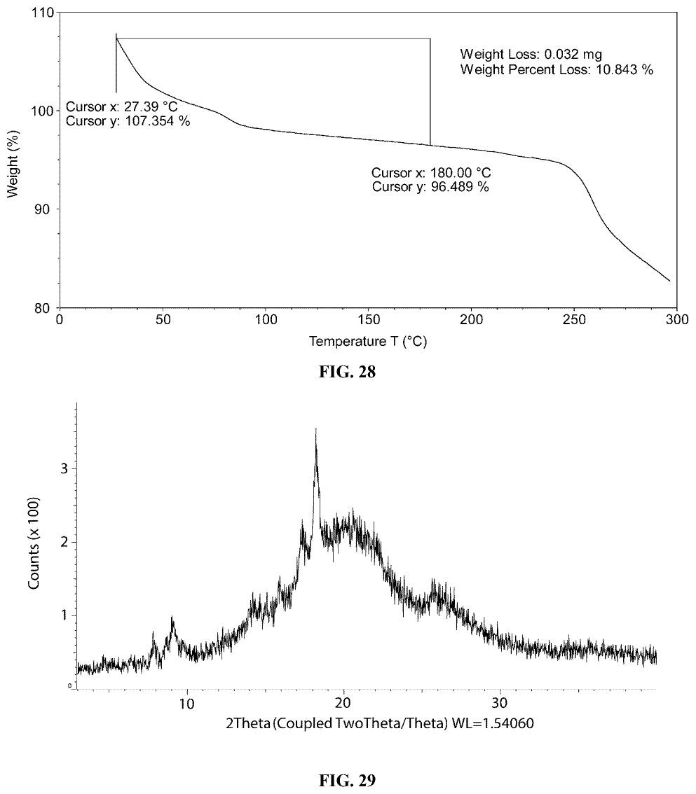

FIG. 26 depicts the XRPD pattern of Compound 1 sodium salt Form E. FIG. 27 depicts the DSC thermogram of Compound 1 sodium salt Form E. FIG. 28 depicts the TGA thermogram of Compound 1 sodium salt Form E.

### Compound 1 Potassium Salt Form G

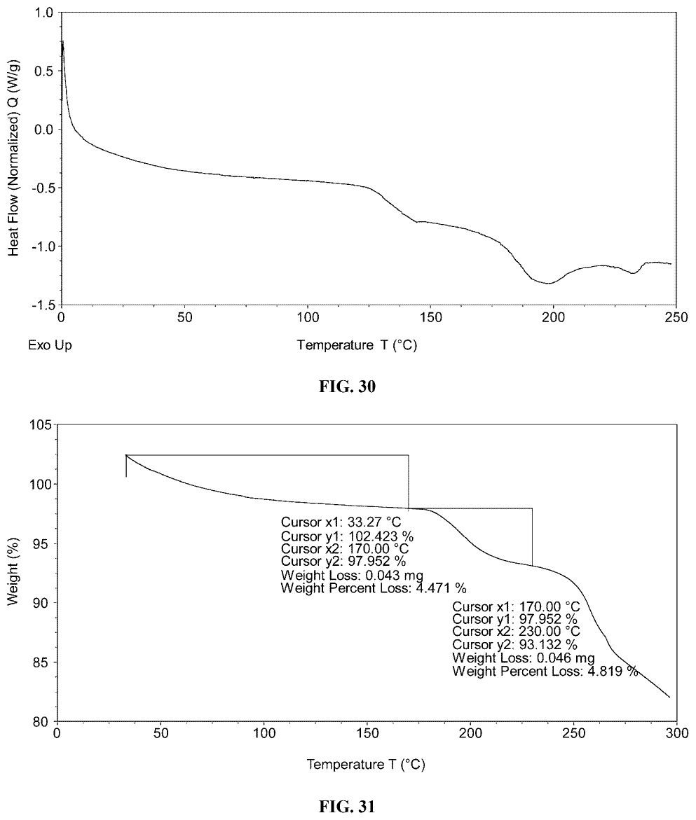

Compound 1 potassium salt Form G is a solvate. Potassium salt Form G has low crystallinity. DSC shows multiple thermal events (FIG. 30). TGA shows about 4.5% weight loss at about 170° C. and about 4.8% weight loss in the temperature range of about 170° C.-230° C. HPLC shows about 99% chemical purity. IC and HPLC shows that the stoichiometric salt ratio of the compound to K⁺ is 1:1. ¹H NMR shows 2.5% acetone residual solvent by weight (0.42 equivalent by molar ratio).

FIG. 29 depicts the XRPD pattern of Compound 1 potassium salt Form G. FIG. 30 depicts the DSC thermogram of Compound 1 potassium salt Form G. FIG. 31 depicts the TGA thermogram of Compound 1 potassium salt Form G.

### Compound 1 Potassium Salt Form H

Compound 1 potassium salt Form H is a hydrate. Potassium salt Form H has medium crystallinity. DSC shows a dehydration peak from 4.2° C., peak at 87.2° C., endothermic peak from 123.0° C., and a melting point at Tₒₙₛₑₜ of 221.7° C. TGA shows about 6.6% weight loss at about 170° C. HPLC shows about 99% chemical purity. IC and HPLC shows that the stoichiometric salt ratio of the compound to K⁺ is 1:1.2. ¹H NMR shows no detectable residual solvent.

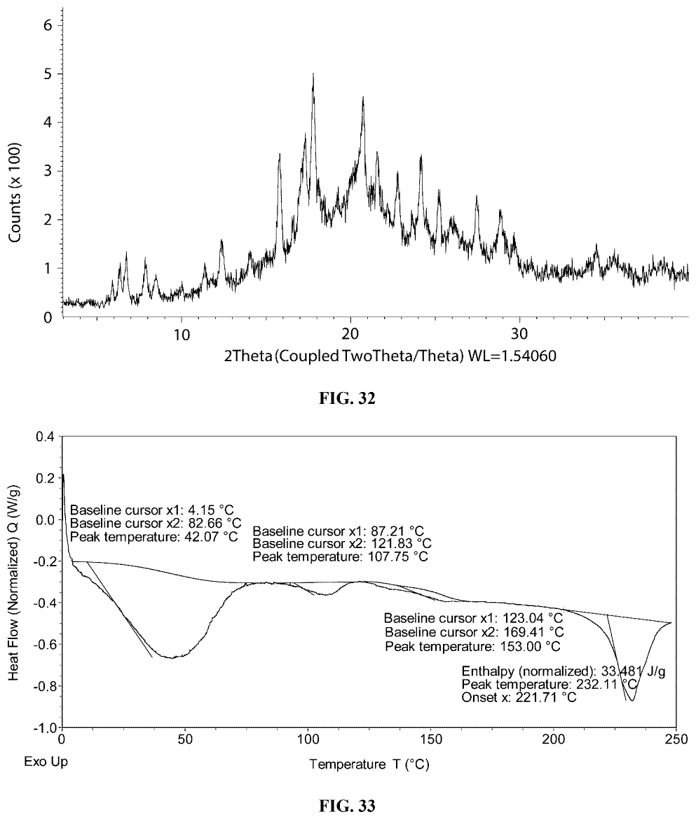

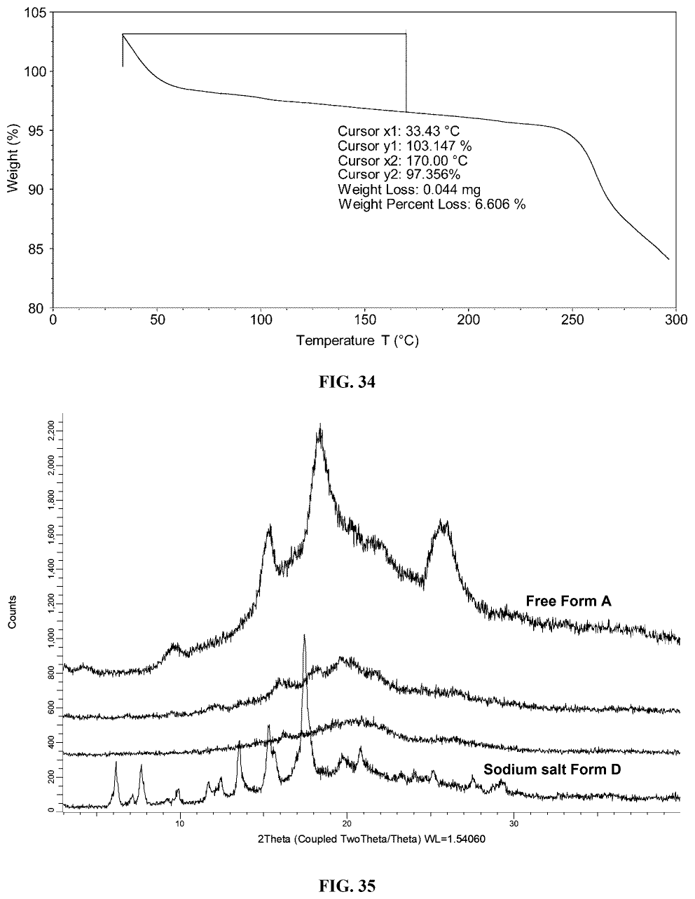

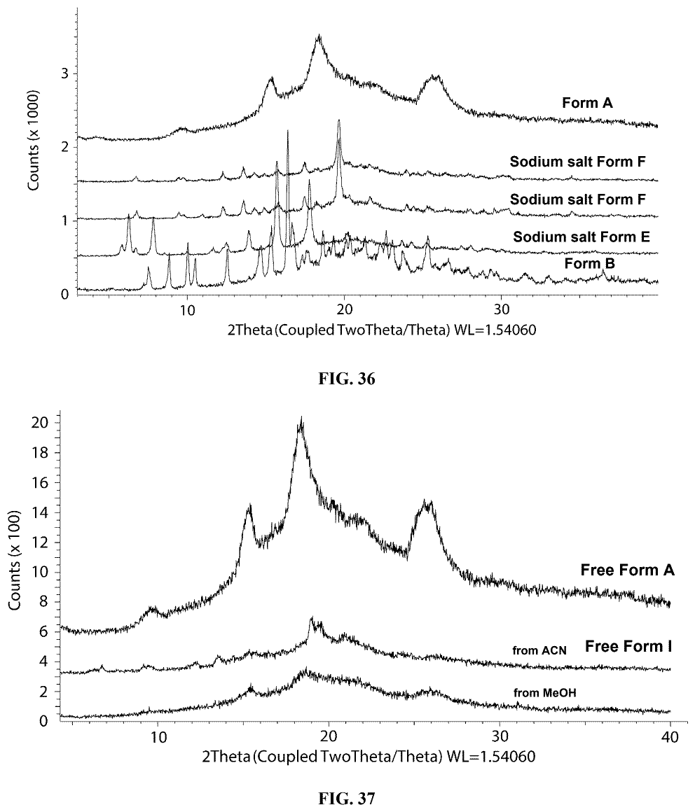

FIG. 32 depicts the XRPD pattern of Compound 1 potassium salt Form H. FIG. 33 depicts the DSC thermogram of Compound 1 potassium salt Form H. FIG. 34 depicts the TGA thermogram of Compound 1 potassium salt Form H.

### Additional Embodiments

1. An isolated crystalline Form B of the compound of structure:

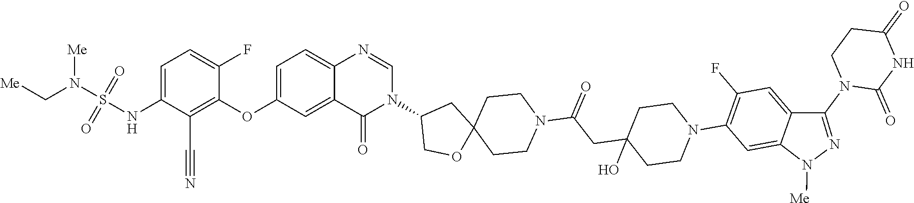 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

characterized by an X-ray powder diffraction (XRPD) pattern comprising at least five 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

2. The isolated crystalline Form B of embodiment 1, wherein the XRPD pattern comprises at least six 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

3. The isolated crystalline Form B of embodiment 1, wherein the XRPD pattern comprises at least seven 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

4. The isolated crystalline Form B of embodiment 1, wherein the XRPD pattern comprises at least eight 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

5. The isolated crystalline Form B of embodiment 1, wherein the XRPD pattern comprises at least nine 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

6. The isolated crystalline Form B of embodiment 1, wherein the XRPD pattern comprises at least ten 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

7. The isolated crystalline Form B of embodiment 1, wherein the XRPD pattern comprises at least eleven 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

8. The isolated crystalline Form B of any one of embodiments 1-7, wherein the XRPD pattern comprises at least three 2theta values selected from 7.5±0.1°, 8.8±0.1°, 10.0±0.1°, 10.5±0.1°, 12.6±0.1°, 14.7±0.1°, 15.4±0.1°, 16.4±0.1°, 16.7±0.1°, 18.6±0.1°, 22.7±0.1°, and 25.3±0.1°.

9. The isolated crystalline Form B of any one of embodiments 1-8, wherein the XRPD pattern comprises at least the 2theta value of 16.4±0.2°.

10. The isolated crystalline Form B of any one of embodiments 1-9, wherein the XRPD pattern comprises at least the 2theta value of 10.0±0.2°.

11. The isolated crystalline Form B of any one of embodiments 1-10, wherein the XRPD pattern comprises at least the 2theta value of 15.4±0.2°.

12. The isolated crystalline Form B of any one of embodiments 1-11, wherein the XRPD pattern comprises at least the 2theta value of 16.7±0.2°.

13. The isolated crystalline Form B of any one of embodiments 1-12, wherein the XRPD pattern comprises at least the 2theta value of 8.8±0.2°.

14. The isolated crystalline Form B of any one of embodiments 1-13, wherein the XRPD pattern comprises at least the 2theta value of 12.6±0.2°.

15. The isolated crystalline Form B of any one of embodiments 1-14, wherein the XRPD pattern comprises at least the 2theta value of 14.7±0.2°.

16. The isolated crystalline Form B of any one of embodiments 1-15, wherein the XRPD pattern comprises at least the 2theta value of 10.5±0.2°.

17. The isolated crystalline Form B of any one of embodiments 1-16, wherein the XRPD pattern comprises at least the 2theta value of 25.3±0.2°.

18. The isolated crystalline Form B of any one of embodiments 1-17, wherein the XRPD pattern comprises at least the 2theta value of 22.7±0.2°.

19. The isolated crystalline Form B of any one of embodiments 1-18, characterized by an XRPD pattern having the characteristic 2theta values of FIG. 1.

20. The isolated crystalline Form B of any one of embodiments 1-19 that has differential scanning calorimetry (DSC) onset endotherm of about 194±20° C.

21. The isolated crystalline Form B of any one of embodiments 1-20 that has differential scanning calorimetry (DSC) onset endotherm of about 194±10° C.

22. A pharmaceutical composition comprising the isolated crystalline Form B of any one of embodiments 1-21 in a pharmaceutically acceptable carrier.

23. A pharmaceutical composition comprising Compound 1, a solubility enhancer, a permeation enhancer, a filler, a binder/glidant, and a mucoadhesive/disintegrant, wherein Compound 1 is of structure:

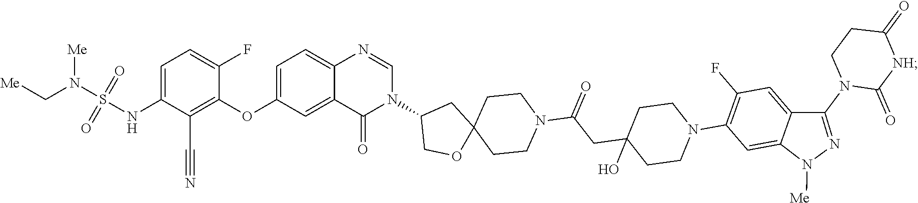 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

or a pharmaceutically acceptable salt thereof.

24. The pharmaceutical composition of embodiment 23, wherein:
a. the solubility enhancer is hypromellose acetate succinate;
b. the permeation enhancer is vitamin E
c. the filler is mannitol;
d. the binder/glidant is cellulose; and
e. the mucoadhesive agent is croscarmellose sodium.

25. A method of treating a mutant BRAF mediated disorder comprising administering an effective amount of the isolated crystalline Form B of any one of embodiments 1-21 or a pharmaceutical composition of any of embodiments 22-24 to a patient in need thereof.

26. The method of embodiment 25, wherein the patient is a human.

27. The method of embodiment 25 or 26, wherein the mutant BRAF mediated disorder is a cancer.

28. The method of embodiment 27, wherein the mutant BRAF mediated cancer is melanoma.

29. The method of embodiment 27, wherein the mutant BRAF mediated cancer is lung cancer.

30. The method of embodiment 27, wherein the mutant BRAF mediated cancer is non-small cell lung cancer.

31. The method of embodiment 27, wherein the mutant BRAF mediated cancer is colorectal cancer.

32. The method of embodiment 27, wherein the mutant BRAF mediated cancer is microsatellite stable colorectal cancer.

33. The method of embodiment 27, wherein the mutant BRAF mediated cancer is thyroid cancer.

34. The method of embodiment 27, wherein the mutant BRAF mediated cancer is ovarian cancer.

35. The method of embodiment 25, wherein the mutant BRAF mediated disorder is cholangiocarcinoma, erdeheim-chester disease, langerhans histiocytosis, ganglioglioma, glioma, glioblastoma, hairy cell leukemia, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

36. The method of any one of embodiments 25-35, wherein the patient also receives an additional active agent.

37. The method of embodiment 36, wherein the additional active agent is a MEK inhibitor.

38. The method of embodiment 37, wherein the MEK inhibitor is trametinib.

39. The method of embodiment 36, wherein the additional active agent is an immune checkpoint inhibitor.

40. The method of embodiment 39, wherein the immune checkpoint inhibitor is selected from nivolumab, pembrolizumab, cemiplimab, ipilimumab, relatlimab, atezolizumab, avelumab, and durvalumab.

41. The method of embodiment 36, wherein the additional active agent is cetuximab or panitumumab.

42. The isolated crystalline Form B according to any one of embodiments 1-21 or a pharmaceutical composition of any of embodiments 22-24 for the therapeutic treatment of a mutant BRAF mediated disorder.

43. The isolated crystalline Form B of embodiment 42, wherein the mutant BRAF mediated disorder is a cancer.

44. The isolated crystalline Form B of embodiment 43, wherein the mutant BRAF mediated cancer is melanoma.

45. The isolated crystalline Form B of embodiment 43, wherein the mutant BRAF mediated cancer is lung cancer.

46. The isolated crystalline Form B of embodiment 43, wherein the mutant BRAF mediated cancer is non-small cell lung cancer.

47. The isolated crystalline Form B of embodiment 43, wherein the mutant BRAF mediated cancer is colorectal cancer.

48. The isolated crystalline Form B of embodiment 43, wherein the mutant BRAF mediated cancer is microsatellite stable colorectal cancer.

49. The isolated crystalline Form B of embodiment 43, wherein the mutant BRAF mediated cancer is thyroid cancer.

50. The isolated crystalline Form B of embodiment 43, wherein the mutant BRAF mediated cancer is ovarian cancer.

51. The isolated crystalline Form B of embodiment 42, wherein the mutant BRAF mediated disorder is cholangiocarcinoma, erdeheim-chester disease, langerhans histiocytosis, ganglioglioma, glioma, glioblastoma, hairy cell leukemia, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

52. The isolated crystalline Form B according to any one of embodiments 1-21 or a pharmaceutical composition of any of embodiments 22-24 for use in the treatment of a mutant BRAF mediated disorder

53. The isolated crystalline Form B of embodiment 52, wherein the mutant BRAF mediated disorder is a cancer.

54. The isolated crystalline Form B of embodiment 53, wherein the mutant BRAF mediated cancer is melanoma.

55. The isolated crystalline Form B of embodiment 53, wherein the mutant BRAF mediated cancer is lung cancer.

56. The isolated crystalline Form B of embodiment 53, wherein the mutant BRAF mediated cancer is non-small cell lung cancer.

57. The isolated crystalline Form B of embodiment 53, wherein the mutant BRAF mediated cancer is colorectal cancer.

58. The isolated crystalline Form B of embodiment 53, wherein the mutant BRAF mediated cancer is microsatellite stable colorectal cancer.

59. The isolated crystalline Form B of embodiment 53, wherein the mutant BRAF mediated cancer is thyroid cancer.

60. The isolated crystalline Form B of embodiment 53, wherein the mutant BRAF mediated cancer is ovarian cancer.

61. The isolated crystalline Form B of embodiment 52, wherein the mutant BRAF mediated disorder is cholangiocarcinoma, erdeheim-chester disease, langerhans histiocytosis, ganglioglioma, glioma, glioblastoma, hairy cell leukemia, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

62. Use of the isolated crystalline Form B according to any one of embodiments 1-21 or a pharmaceutical composition of any of embodiments 22-24 in the manufacture of a medicament for the treatment of a mutant BRAF mediated disorder.

63. The use of embodiment 62, wherein the mutant BRAF mediated disorder is a cancer.

64. The use of embodiment 63, wherein the mutant BRAF mediated cancer is melanoma.

65. The use of embodiment 63, wherein the mutant BRAF mediated cancer is lung cancer.

66. The use of embodiment 63, wherein the mutant BRAF mediated cancer is non-small cell lung cancer.

67. The use of embodiment 63, wherein the mutant BRAF mediated cancer is colorectal cancer.

68. The use of embodiment 63, wherein the mutant BRAF mediated cancer is microsatellite stable colorectal cancer.

69. The use of embodiment 63, wherein the mutant BRAF mediated cancer is thyroid cancer.

70. The use of embodiment 63, wherein the mutant BRAF mediated cancer is ovarian cancer.

71. The use of embodiment 62, wherein the mutant BRAF mediated disorder is cholangiocarcinoma, erdeheim-chester disease, langerhans histiocytosis, ganglioglioma, glioma, glioblastoma, hairy cell leukemia, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

72. The isolated crystalline Form B according to any one of embodiments 1-21 for use as a therapeutically active substance.

73. A process to prepare a compound of formula:

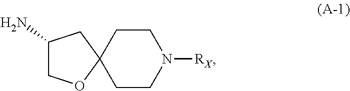

comprising a transaminase catalyzed reductive amination of a compound of formula:

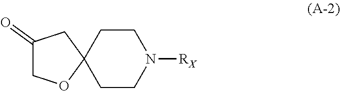

in the presence of isopropylamine or an isopropylamine salt and a first base to give the compound of formula A-1;
wherein R_{X} is selected from the group consisting of hydrogen and an amino-protecting group.

74. The process according to embodiment 73, wherein:
the amino-protecting group R_{X} is tert-butyloxycarbonyl (BOC);
the first base is sodium hydroxide or potassium hydroxide; and the isopropylamine salt is isopropylamine hydrochloride.

75. A process to prepare a compound of formula:

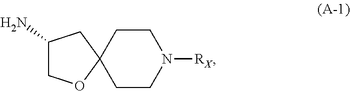

wherein the process comprises the steps of:
   (i) reacting a compound of formula:

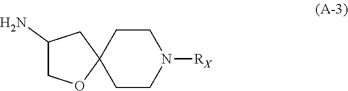

with a chiral acid to give a mixture of diastereomeric salts formed between the compound of formula A-3 and the chiral acid;
(ii) crystallizing the mixture of diastereomeric salts prepared in the step (i) from a first solvent to give an individually isolated diastereomeric salt formed between an anion of the chiral acid and a cation of N-protected (R)-1-oxa-8-azaspiro[4.5]decan-3-aminium of formula:

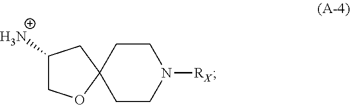

and
(iii) treating the individually isolated diastereomeric salt containing the cation of formula A-4 isolated in the step (ii) with a second base to release the compound of formula A-1;
wherein R_{X} is selected from the group consisting of hydrogen and an amino-protecting group.

76. The process according to embodiment 75, wherein:
the amino-protecting group R_{X} is tert-butyloxycarbonyl (BOC);
the chiral acid is (R)-mandelic acid;
the first solvent is acetonitrile; and
the second base is sodium hydroxide or potassium hydroxide.

77. A process to prepare a compound of formula:

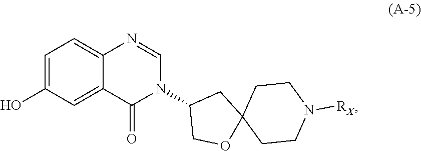

wherein the process comprises reacting 2-amino-5-hydroxybenzoic acid with a compound of formula

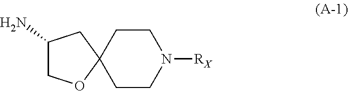

in the presence of a trialkyl orthoformate in a second solvent;
wherein the compound of formula A-1 is prepared in a process according to any one of embodiments 73-76; and
R_{X} is selected from the group consisting of hydrogen and an amino-protecting group.

78. The process according to embodiment 77, wherein:
the amino-protecting group R_{X} is tert-butyloxycarbonyl (BOC);
trialkyl orthoformate is triethyl orthoformate; and
the second solvent is n-butanol.

79. A process to prepare a compound of formula:

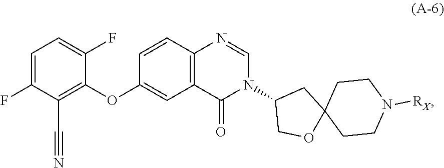

comprising reacting 2,3,6-trifluorobenzonitrile with a compound of formula:

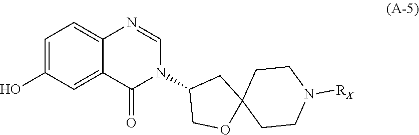

in the presence of a third base in a third solvent;
wherein
R_{X} is selected from the group consisting of hydrogen and an amino-protecting group; and the compound of formula A-5 is prepared in the process according to any one of embodiments 77-78.

80. The process according to embodiment 79, wherein:
the third base is potassium carbonate and the third solvent is acetonitrile; and
the amino-protecting group R_{X} is tert-butyloxycarbonyl (BOC).

81. A process to prepare a compound of formula:

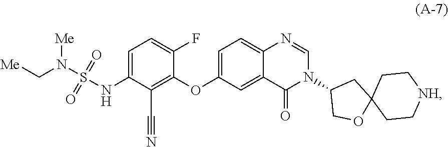 [SMILES: CCN(C)S(=O)(=O)Nc1ccc(F)c(Oc2ccc3ncn([C@H]4COC5(CCNCC5)C4)c(=O)c3c2)c1C#N]

comprising the steps of
(i) reacting [methyl(sulfamoyl)amino]ethane NH₂SO₂N(Me)Et with a compound of formula:

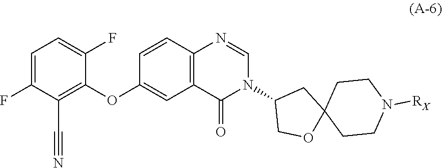

in the presence of a fourth base in a fourth solvent to give a compound of formula A-8:

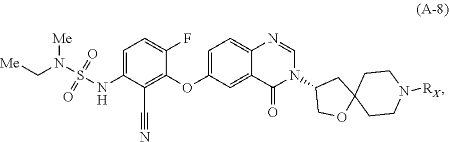

wherein:
the compound of formula A-6 is prepared in the process according to any one of embodiments 79-80;
R_{X} is selected from the group consisting of hydrogen and an amino-protecting group; and
(ii) when R_{X} is the amino-protecting group, then removing the amino-protecting group attached to piperidine nitrogen atom of the compound of formula A-8 in the presence of a first acid.

82. The process according to embodiment 81, wherein:
the fourth base is cesium carbonate and the fourth solvent is N,N-dimethylacetamide;
the amino-protecting group R_{X} is tert-butyloxycarbonyl (BOC); and
the first acid is hydrochloric acid.

83. A process to prepare a compound of formula:

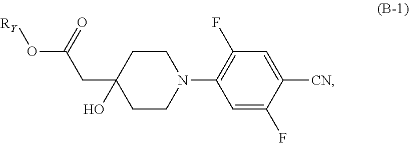

comprising reacting 2,4,5-trifluorobenzonitrile with a compound of formula:

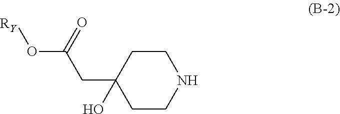

in the presence of a fifth base in a fifth solvent;
wherein R_{Y} is hydrogen or a carboxylic acid protecting group.

84. The process according to embodiment 83, wherein:
the fifth base is N,N-diisopropylethylamine and the fifth solvent is N,N-dimethylformamide; and
R_{Y} is tert-butyl (Bu).

85. A process to prepare a compound of formula:

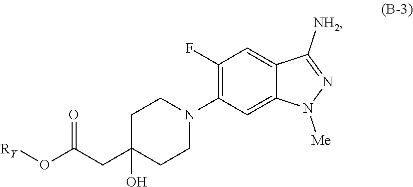

comprising reacting methylhydrazine with a compound of formula:

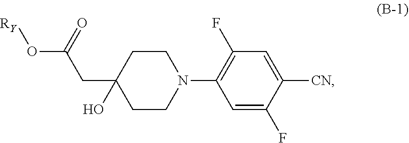

in the presence of a sixth solvent,
wherein the compound of formula B-1 is prepared in the process according to any one of embodiments 83-84;
wherein R_{Y} is hydrogen or a carboxylic acid protecting group.

86. The process according to embodiment 85, wherein:
the sixth solvent is N-methyl-2-pyrrolidone; and
R_{Y} is tert-butyl.

87. A process to prepare a compound of formula:

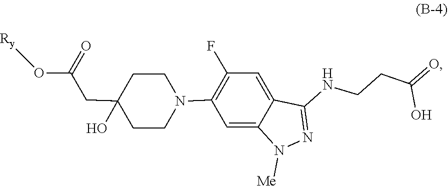

comprising reacting acrylic acid with a compound of formula:

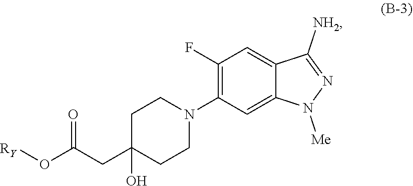

in a seventh solvent;
wherein:
the compound of formula B-3 is prepared in the process according to any one of embodiments 85-86; and
R_{Y} is hydrogen or a carboxylic acid protecting group.

88. The process according to embodiment 87, wherein:
the seventh solvent is an aqueous solution of hydrochloric acid; and
R_{Y} is tert-butyl.

89. A process to prepare a compound of formula:

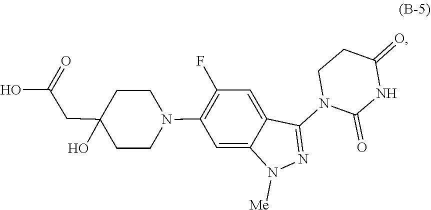 [SMILES: Cn1nc(N2CCC(=O)NC2=O)c2cc(F)c(N3CCC(O)(CC(=O)O)CC3)cc21]

comprising the steps of:
(i) first reacting a metal cyanate salt with a compound of formula:

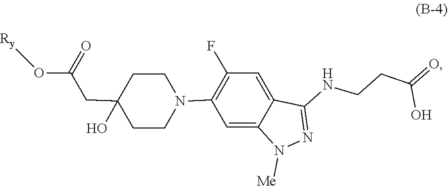

in the presence of a second acid to give a reaction mixture containing a first intermediate compound of formula:

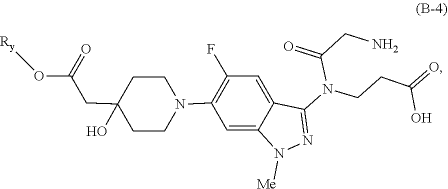

wherein:
the compound of formula B-4 is prepared in the process according to any one of embodiments 87-88; and
R_{Y} is hydrogen or a carboxylic acid protecting group;
(ii) then cyclizing the first intermediate compound of formula B-6 from step (i) optionally in the presence of a third acid to give a second intermediate compound of formula

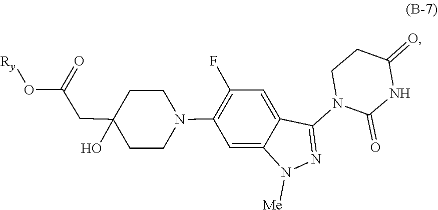

and
(iii) then, when R_{Y} is the carboxylic acid protecting group, removing the carboxylic acid protecting group R_{Y} from the second intermediate compound of formula B-7 from step (ii) in the presence of a fourth acid to give the compound of formula B-5.

90. The process according to embodiment 89, wherein
R_{Y} is tert-butyl;
a metal cyanate salt is sodium cyanate or potassium cyanate;
the second acid is acetic acid;
the third acid is hydrochloric acid or acetic acid; and
the fourth acid is hydrochloric acid.

91. A process to prepare a compound of formula:

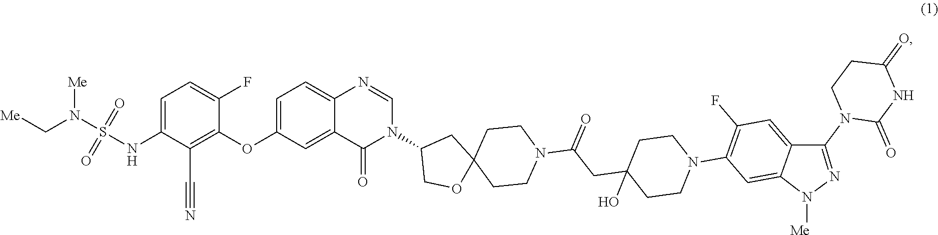 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

wherein the process comprises the steps of:
(i) first reacting a compound of formula:

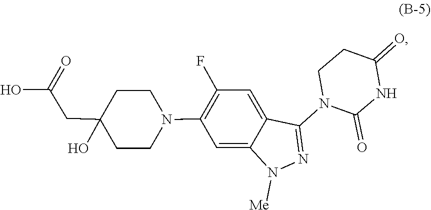 [SMILES: Cn1nc(N2CCC(=O)NC2=O)c2cc(F)c(N3CCC(O)(CC(=O)O)CC3)cc21]

with a carboxylic acid hydroxyl activating reagent in the presence of a sixth base and an eighth solvent to give a third intermediate compound of formula:

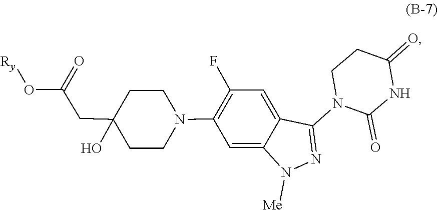

in which the carboxylic acid hydroxyl is converted into a more reactive group —OR_{Z}; wherein the compound of formula B-5 is prepared in the process according to any of embodiments 89-90; and
(ii) then reacting (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane of formula

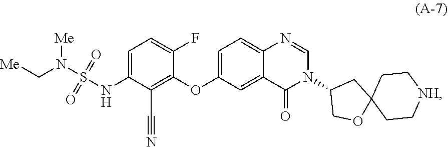 [SMILES: CCN(C)S(=O)(=O)Nc1ccc(F)c(Oc2ccc3ncn([C@H]4COC5(CCNCC5)C4)c(=O)c3c2)c1C#N]

with the third intermediate compound of formula B-8 prepared in the step (ii) in the presence of a seventh base and a ninth solvent to give the compound of formula 1,
wherein the compound of formula A-7 is prepared in the process according to any of embodiments 81-82.

92. The process according to embodiment 91, wherein
(i) the sixth base is N,N-diisopropylethylamine;
   the eighth solvent is acetonitrile;
   the carboxylic acid hydroxyl activating reagent is 2-succinimido-1,1,3,3-tetramethyluronium tetrafluoroborate and the Rz group is 2,5-dioxopyrrolidin-1-yl; and
(ii) the seventh base is triethylamine and the ninth solvent is N,N-dimethylformamide.

93. A method of treating a brain or central nervous system (CNS) metastasis of a mutant BRAF mediated cancer comprising administering an effective amount of a compound of structure

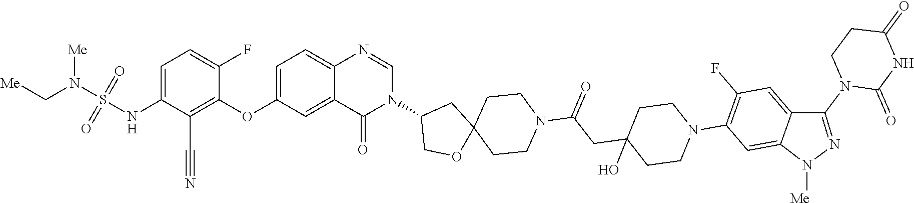 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

or a pharmaceutically acceptable salt thereof to a human patient in need thereof.

94. The method of embodiment 93, wherein the cancer has metastasized to the brain.

95. The method of embodiment 93 or 94, wherein the cancer has metastasized to the CNS.

96. The method of any one of embodiments 93-95, wherein the mutant BRAF mediated cancer is melanoma.

97. The method of any one of embodiments 93-95, wherein the mutant BRAF mediated cancer is lung cancer.

98. The method of any one of embodiments 93-95, wherein the mutant BRAF mediated cancer is non-small cell lung cancer.

99. The method of any one of embodiments 93-95, wherein the mutant BRAF mediated cancer is colorectal cancer.

100. The method of any one of embodiments 93-95, wherein the mutant BRAF mediated cancer is microsatellite stable colorectal cancer.

101. The method of any one of embodiments 93-95, wherein the mutant BRAF mediated cancer is thyroid cancer.

102. The method of any one of embodiments 93-95, wherein the mutant BRAF mediated cancer is ovarian cancer.

103. The method of any one of embodiments 93-95, wherein the mutant BRAF mediated cancer is cholangiocarcinoma, erdeheim-chester disease, langerhans histiocytosis, ganglioglioma, glioma, glioblastoma, hairy cell leukemia, metanephric adenoma, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

104. The method of any one of embodiments 93-103, wherein the patient also receives an additional active agent.

105. The method of embodiment 104, wherein the additional active agent is a MEK inhibitor.

106. The method of embodiment 105, wherein the MEK inhibitor is trametinib.

107. The method of embodiment 104, wherein the additional active agent is an immune checkpoint inhibitor.

108. The method of embodiment 107, wherein the immune checkpoint inhibitor is selected from nivolumab, pembrolizumab, cemiplimab, ipilimumab, relatlimab, atezolizumab, avelumab, and durvalumab.

109. The method of embodiment 104, wherein the additional active agent is cetuximab or panitumumab.

110. The method of any one of embodiments 93-109, wherein the mutant BRAF has a V600 mutation.

111. The method of embodiment 110, wherein the V600 mutation is V600E, V600K, V600R, V600D, V600M, or V600N.

112. The method of embodiment 110, wherein the V600 mutation is V600E.

113. The method of any one of embodiments 93-112, wherein the mutant BRAF has a mutation selected from G469A, G469V, G469L, G469R, L597Q, and K601E.

114. The method of any one of embodiments 93-113, wherein the mutant BRAF has a mutation selected from G466A, G466E, G466R, G466V, S467L, G469E, N581I, D594E, D594G, and D594N.

115. The method of any one of embodiments 93-114, wherein the mutant BRAF has a mutation selected from G464I, G464R, N581T, L584F, E586K, G593D, G596C, L597R, L597S, S605I, S607F, N684T, E26A, V130M, L745L, and D284E.

116. The method of any one of embodiments 93-115, wherein the mutant BRAF has splice mutation.

117. The method of embodiment 116, wherein the splice mutation is a p61-BRAF V600 splice mutation.

118. The method of any one of embodiments 93-117, wherein the mutant BRAF mediated cancer has a NRAS, MEK1, or PI3K mutation.

119. The method of embodiment 118, wherein the cancer has a NRAS mutation.

120. The method of embodiment 119, wherein the NRAS mutation is Q61K.

121. The method of embodiment 119, wherein the NRAS mutation is Q61R.

122. The method of embodiment 118, wherein the cancer has a MEK1 mutation.

123. The method of embodiment 118, wherein the cancer has a PI3K mutation.

124. The method of embodiment 123, wherein PI3K mutation is PIK3CA^{H1047R} or PIK3CA^{P449T}.

125. The method of any one of embodiments 93-123, wherein the mutant BRAF mediated cancer has one or more mutations that promote RAF protein homo-dimerization or hetero-dimerization.

126. The method of embodiment 125, wherein the one or more mutations promote RAF protein homo-dimerization.

127. The method of embodiment 125, wherein the one or more mutations promote RAF protein hetero-dimerization.

### Treatment of Disorders Mediated by BRAF

Compound 1 is a small-molecule that degrades mutant BRAF, for example a Class I, Class IL, and/or Class III mutant BRAF, via the ubiquitin proteasome pathway. Compound 1 binds to the ubiquitously expressed E3 ligase protein cereblon (CRBN) and alters the substrate specificity of the CRBN E3 ubiquitin ligase complex, resulting in the recruitment and ubiquitination of mutant BRAF, such as, for example, BRAF V600E. Compound 1 effectively degrades Class I mutant BRAF such as V600E, Class II mutant BRAF such as G469A, Class III mutant BRAF such as G466V mutations.

A pharmaceutical composition comprising an effective amount of Compound 1 or a pharmaceutically acceptable salt thereof or a Compound 1 morphic form of the present invention can thus be used to treat a mutant BRAF mediated cancer, for example melanoma, lung cancer including for example non-small cell lung cancer, colorectal cancer including for example microsatellite stable colorectal cancer, thyroid cancer including for example anaplastic thyroid cancer, or ovarian cancer. In certain embodiments, a pharmaceutical composition comprising an effective amount of Compound 1 or a pharmaceutically acceptable salt thereof or a morphic form thereof is used to treat a solid tumor that is mediated by a V600X mutant BRAF. Non-limiting examples of disorders that can be treated with a pharmaceutical composition comprising an effective amount of Compound 1 or a pharmaceutically acceptable salt thereof or a morphic form thereof include melanoma, non-small cell lung carcinoma, thyroid cancer, colorectal cancer, and other solid tumor malignancies that have a mutant BRAF driver.

In certain embodiments a pharmaceutical composition comprising an effective amount of Compound 1 or a pharmaceutically acceptable salt thereof or a morphic form thereof can treat a cancer that has developed resistance to a BRAF inhibitor. For example, a Compound 1 is effective in the treatment of a G466V mutant BRAF lung tumor cell line. In certain embodiments a pharmaceutical composition comprising an effective amount of Compound 1 or a morphic form thereof is orally bioavailable.

In certain aspects, a pharmaceutical composition comprising an effective amount of Compound 1 or a pharmaceutically acceptable salt thereof or a morphic form thereof is used to treat a BRAF mediated cancer, wherein the BRAF has mutated from the wild type. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, V600M and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N.

In certain embodiments a pharmaceutical composition comprising an effective amount of Compound 1 or a morphic form thereof treats a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments a pharmaceutical composition comprising an effective amount of Compound 1 or a morphic form thereof treats a BRAF mutant mediated disorder wherein the mutation is a splice variant, for example p61-BRAF^{V}600E

Compound 1, a pharmaceutical composition comprising an effective amount of Compound 1 or a morphic form thereof or pharmaceutical composition thereof, or a pharmaceutically acceptable salt of Compound 1 or pharmaceutical composition thereof, can be used to treat a patient with any disorder mediated by a mutant BRAF.

BRAF is a serine/threonine protein kinase that is a member of the signal transduction protein kinases. BRAF V600X mutations, in particular BRAF V600E/K mutations are often observed in a variety of human tumors including melanoma, thyroid cancer, colorectal cancer, lung cancer and others. Non-limiting examples of V600X mutations include V600E, V600K, V600R, V600D, V600M, and V600N. Despite the therapeutic benefits exerted by available BRAF inhibitors in the clinic in many of these indications, the duration of the antitumor response to these drugs is limited by the acquisition of drug resistance.

The BRAF protein presents a mechanism for signaling propagation that requires protein homo-dimerization (BRAF-BRAF) or hetero-dimerization with other RAF proteins (BRAF-RAF1 or BRAF-ARAF). When BRAF is mutated, as observed in oncological indications with BRAF V600X substitution, BRAF signaling becomes independent from the generation of homodimers and/or heterodimers. In this context, the kinase becomes hyperactivated as a monomeric protein and drives cellular proliferative signals.

Because currently available inhibitors only block BRAF activity in its monomeric form and are ineffective on BRAF homodimers or heterodimers, it is not surprising that many BRAF-resistance inducing mechanisms act by restoring RAF homodimerization and heterodimerization mediated signaling.

Targeted protein degradation induces target ubiquitination by recruiting an E3 ligase thus promoting proteasome-mediated disruption of the engaged target. The degradation of BRAF through targeted degradation offers an advantage over conventional inhibition since it eliminates scaffolding activities of BRAF V600E/K and particularly, induces BRAF protein elimination. This activity prevents the dimerization-mediated mechanisms of resistance.

BRAF protein abrogation can represent a strategy to delay the onset of resistance as well as target tumors that acquire resistance to available inhibitors. This offers novel therapeutic opportunities in the treatment of BRAF V600X mutated tumors like melanoma, colorectal cancer, and lung cancer.

Another aspect of the present invention provides a Compound 1 morphic form as described herein or isotopic derivative of Compound 1, pharmaceutically acceptable salt, hydrate, or solvate thereof, or a pharmaceutical composition thereof, for use in the manufacture of a medicament for treating or preventing cancer in a patient in need thereof, wherein there is a need of BRAF inhibition for the treatment or prevention of cancer.

In certain aspects, a pharmaceutical composition comprising an effective amount of Compound 1 or a morphic form thereof is used to treat a BRAF mediated cancer, wherein the BRAF has mutated from the wild type. There are a number of possibilities for BRAF mutations. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, V600M and V600N. Non-limiting examples of Class II mutations include G469A, G469V, G469L, G469R, L597Q, and K601E. Non-limiting examples of Class III mutations include G466A, G466E, G466R, G466V, S467L, G469E, N581L, D594E, D594G, and D594N.

In certain embodiments a pharmaceutical composition comprising an effective amount of Compound 1 or a morphic form thereof treats a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments the BRAF mutation is an exon 11 mutation.

In certain embodiments the BRAF mutation is an exon 15 mutation.

In certain embodiments the BRAF mutation is a G464 mutation.

In certain embodiments the BRAF mutation is a G466 mutation.

In certain embodiments the BRAF mutation is a G466R mutation.

In certain embodiments the BRAF mutation is a G466E mutation.

In certain embodiments the BRAF mutation is a G469 mutation.

In certain embodiments the BRAF mutation is a G469E mutation.

In certain embodiments the BRAF mutation is a D594 mutation.

In certain embodiments the BRAF mutation is a D594A mutation.

In certain embodiments the BRAF mutation is a L597 mutation.

In certain embodiments the BRAF mutation is a L597R mutation.

In certain embodiments the BRAF mutation is a L597S mutation.

In certain embodiments the BRAF mutation is a L597Q mutation.

In certain embodiments the BRAF mutation is a V600 mutation.

In certain embodiments the BRAF mutation is a V600E mutation.

In certain embodiments the BRAF mutation is a V600K mutation.

In certain embodiments the BRAF mutation is a V600R mutation.

In certain embodiments the BRAF mutation is a V600D mutation.

In certain embodiments the BRAF mutation is a V600M mutation.

In certain embodiments the BRAF mutation is a V600N mutation.

In certain embodiments the BRAF mutation is a K601 mutation.

In certain embodiments the BRAF mutation is a K601E mutation.

In certain embodiments the BRAF mutation is a K601N mutation.

In certain embodiments a pharmaceutical composition comprising an effective amount of Compound 1 or a morphic form thereof treats a BRAF mutant mediated disorder wherein the mutation is a splice variant, for example p61-BRAF^{V600E} or BRAF kinase domain duplication or BRAF amplification.

In certain embodiments a pharmaceutical composition comprising an effective amount of Compound 1 or a morphic form thereof is used to treat a disorder that is mediated by two or more mutant proteins, for example a cancer mediated by a BRAF^{V600E}/NRAS^{Q61K} or BRAF^{V600E}/NRAS^{Q61R} double mutant.

In certain embodiments, a pharmaceutical composition comprising an effective amount of Compound 1 or a morphic form thereof is used to treat a cancer that is resistant to at least one BRAF inhibitor, for example a cancer that is resistant to or has acquired resistance to a BRAF inhibitor selected from dabrafenib, vemurafenib and encorafenib.

In certain embodiments a pharmaceutical composition comprising an effective amount of Compound 1 or a morphic form thereof is used to treat a cancer that has developed an escape mutation such as BRAF V600E NRAS^{Q61K} double mutant cancer.

In certain embodiments a pharmaceutical composition comprising an effective amount of Compound 1 or a morphic form thereof is used to treat melanoma.

Non-limiting examples of melanoma include nonacral cutaneous melanoma, acral melanoma, mucosal melanoma, uveal melanoma, and leptomeningeal melanoma, each of which can be primary or metastatic.

In certain embodiments a pharmaceutical composition comprising an effective amount of Compound 1 or a morphic form thereof is used to treat triple negative breast cancer, for example triple negative breast cancer with a G464V BRAF mutant.

In certain embodiments a pharmaceutical composition comprising an effective amount of Compound 1 or a morphic form thereof is used to treat lung cancer, for example lung adenocarcinoma with a G466V BRAF mutant.

In certain embodiments a pharmaceutical composition comprising an effective amount of Compound 1 or a morphic form thereof is used to treat melanoma with a V600 BRAF mutant.

In certain embodiments a pharmaceutical composition comprising an effective amount of Compound 1 or a pharmaceutically acceptable salt thereof or a morphic form thereof treats a BRAF mutant mediated disorder wherein the mutation is a splice variant, for example p61-BRAF^{V600E}.

In certain embodiments a pharmaceutical composition comprising an effective amount of Compound 1 or a pharmaceutically acceptable salt thereof or a morphic form thereof is used to treat a disorder that is mediated by two or more mutant proteins, for example a cancer mediated by a BRAF^{V600E}/NRAS^{Q61K} or a BRAF^{V600E}/NRAS^{Q61R} double mutant.

In certain embodiments a pharmaceutical composition comprising an effective amount of Compound 1 or a pharmaceutically acceptable salt thereof or a morphic form thereof is used to treat a cancer that has developed an escape mutation such as BRAF V600E NRAS^{Q61K} double mutant cancer.

In certain embodiments, a pharmaceutical composition comprising an effective amount of Compound 1 or a pharmaceutically acceptable salt thereof or a morphic form thereof is used to treat a cancer that is resistant to at least one BRAF inhibitor, for example a cancer that is resistant to or has acquired resistance to a BRAF inhibitor selected from dabrafenib, vemurafenib and encorafenib.

In certain embodiments a pharmaceutical composition comprising an effective amount of Compound 1 or a pharmaceutically acceptable salt thereof or a morphic form thereof is used to treat melanoma.

In certain embodiments a pharmaceutical composition comprising an effective amount of Compound 1 or a pharmaceutically acceptable salt thereof or a morphic form thereof is used to treat triple negative breast cancer, for example triple negative breast cancer with a G464V BRAF mutant.

In certain embodiments a pharmaceutical composition comprising an effective amount of Compound 1 or a pharmaceutically acceptable salt thereof or a morphic form thereof is used to treat lung cancer, for example lung adenocarcinoma with a G466V BRAF mutant.

In certain embodiments a pharmaceutical composition comprising an effective amount of Compound 1 or a pharmaceutically acceptable salt thereof or a morphic form thereof is used to treat melanoma with a V600 BRAF mutant.

In certain embodiments a pharmaceutical composition comprising an effective amount of Compound 1 or a pharmaceutically acceptable salt thereof or a morphic form thereof is used to treat cholangiocarcinoma.

In certain embodiments a pharmaceutical composition comprising an effective amount of Compound 1 or a pharmaceutically acceptable salt thereof or a morphic form thereof is used to treat erdeheim-chester disease.

In certain embodiments a pharmaceutical composition comprising an effective amount of Compound 1 or a pharmaceutically acceptable salt thereof or a morphic form thereof is used to treat Langerhans histiocytosis.

In certain embodiments a pharmaceutical composition comprising an effective amount of Compound 1 or a pharmaceutically acceptable salt thereof or a morphic form thereof is used to treat ganglioglioma.

In certain embodiments a pharmaceutical composition comprising an effective amount of Compound 1 or a pharmaceutically acceptable salt thereof or a morphic form thereof is used to treat glioma.

In certain embodiments a pharmaceutical composition comprising an effective amount of Compound 1 or a pharmaceutically acceptable salt thereof or a morphic form thereof is used to treat GIST.

In certain embodiments a pharmaceutical composition comprising an effective amount of Compound 1 or a pharmaceutically acceptable salt thereof or a morphic form thereof is used to treat glioblastoma.

In certain embodiments a pharmaceutical composition comprising an effective amount of Compound 1 or a pharmaceutically acceptable salt thereof or a morphic form thereof is used to treat hairy cell leukemia.

In certain embodiments a pharmaceutical composition comprising an effective amount of Compound 1 or a pharmaceutically acceptable salt thereof or a morphic form thereof is used to treat multiple myeloma.

In certain embodiments a pharmaceutical composition comprising an effective amount of Compound 1 or a pharmaceutically acceptable salt thereof or a morphic form thereof is used to treat non-small-cell lung cancer.

In certain embodiments a pharmaceutical composition comprising an effective amount of Compound 1 or a pharmaceutically acceptable salt thereof or a morphic form thereof is used to treat ovarian cancer.

In certain embodiments a pharmaceutical composition comprising an effective amount of Compound 1 or a pharmaceutically acceptable salt thereof or a morphic form thereof is used to treat pilomyxoid astrocytoma.

In certain embodiments a pharmaceutical composition comprising an effective amount of Compound 1 or a pharmaceutically acceptable salt thereof or a morphic form thereof is used to treat anaplastic pleomorphic xanthoastrocytoma.

In certain embodiments a pharmaceutical composition comprising an effective amount of Compound 1 or a pharmaceutically acceptable salt thereof or a morphic form thereof is used to treat astrocytoma.

In certain embodiments a pharmaceutical composition comprising an effective amount of Compound 1 or a pharmaceutically acceptable salt thereof or a morphic form thereof is used to treat thyroid cancer.

In certain embodiments a pharmaceutical composition comprising an effective amount of Compound 1 or a pharmaceutically acceptable salt thereof or a morphic form thereof is used to treat papillary thyroid cancer.

In certain embodiments a pharmaceutical composition comprising an effective amount of Compound 1 or a pharmaceutically acceptable salt thereof or a morphic form thereof is used to treat anaplastic thyroid cancer.

In certain embodiments a pharmaceutical composition comprising an effective amount of Compound 1 or a pharmaceutically acceptable salt thereof or a morphic form thereof is used to treat pancreatic cancer.

In certain embodiments a pharmaceutical composition comprising an effective amount of Compound 1 or a pharmaceutically acceptable salt thereof or a morphic form thereof is used to treat thoracic clear cell sarcoma.

In certain embodiments a pharmaceutical composition comprising an effective amount of Compound 1 or a pharmaceutically acceptable salt thereof or a morphic form thereof is used to treat salivary gland cancer.

In certain embodiments a pharmaceutical composition comprising an effective amount of Compound 1 or a pharmaceutically acceptable salt thereof or a morphic form thereof is used to treat colorectal cancer.

In certain embodiments a pharmaceutical composition comprising an effective amount of Compound 1 or a pharmaceutically acceptable salt thereof or a morphic form thereof is used to treat microsatellite stable colorectal cancer.

In certain embodiments a pharmaceutical composition comprising an effective amount of Compound 1 or a pharmaceutically acceptable salt thereof or a morphic form thereof is used to treat a disorder selected from cholangiocarcinoma, erdeheim-chester disease, Langerhans histiocytosis, ganglioglioma, glioma, GIST, glioblastoma, hairy cell leukemia, multiple myeloma, lung cancer, non-small-cell lung cancer, ovarian cancer, pilomyxoid astrocytoma, anaplastic pleomorphic xanthoastrocytoma, astrocytoma, thyroid cancer, papillary thyroid cancer, anaplastic thyroid cancer, pancreatic cancer, thoracic clear cell sarcoma, salivary gland cancer, colorectal cancer, and microsatellite stable colorectal cancer.

In certain aspects a method of treating a mutant BRAF mediated cancer that has metastasized to the brain or central nervous system (CNS) is provided comprising administering an effective amount of Compound 1, or a pharmaceutically acceptable salt or morphic form thereof, to a patient in need thereof. In certain embodiments the cancer that has metastasized to the brain or CNS is colorectal cancer, melanoma, non-small cell lung cancer, or other solid tumors bearing BRAF V600E. In certain embodiments of this aspect Compound 1 or a pharmaceutically acceptable salt thereof is administered to the patient in need thereof. In other embodiments of this aspect a morphic form of Compound 1 or a pharmaceutically acceptable salt thereof or a pharmaceutical composition prepared from a morphic form of Compound 1 is administered to the patient in need thereof.

In certain aspects, Compound 1 or a pharmaceutically acceptable salt thereof is used to treat a mutant BRAF mediated cancer that has metastasized to the brain or CNS, wherein the BRAF has mutated from the wild type. There are a number of possibilities for BRAF mutations. In certain non-limiting embodiments, the mutation is a, Class I mutation, a Class II mutation, or a Class III mutation, or any combination thereof. Non-limiting examples of Class I mutations include V600 mutations such as V600E, V600K, V600R, V600D, V600M and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N. In certain embodiments the BRAF mutation is a V600 mutation, for example a V600E BRAF that mediates a cancer that has metastasized to the brain or CNS.

In certain embodiments, Compound 1 or a pharmaceutically acceptable salt thereof, may treat a mutant BRAF mediated cancer that has metastasized to the brain or CNS wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments the disorder is cancer that has metastasized to the brain or CNS and the BRAF mutation is an exon 11 mutation.

In certain embodiments the disorder is cancer that has metastasized to the brain or CNS and the BRAF mutation is an exon 15 mutation.

In certain embodiments the disorder is cancer that has metastasized to the brain or CNS and the BRAF mutation is a G464 mutation.

In certain embodiments the disorder is cancer that has metastasized to the brain or CNS and the BRAF mutation is a G466 mutation.

In certain embodiments the disorder is cancer that has metastasized to the brain or CNS and the BRAF mutation is a G466R mutation.

In certain embodiments the disorder is cancer that has metastasized to the brain or CNS and the BRAF mutation is a G466E mutation.

In certain embodiments the disorder is cancer that has metastasized to the brain or CNS and the BRAF mutation is a G469 mutation.

In certain embodiments the disorder is cancer that has metastasized to the brain or CNS and the BRAF mutation is a G469E mutation.

In certain embodiments the disorder is cancer that has metastasized to the brain or CNS and the BRAF mutation is a D594 mutation.

In certain embodiments the disorder is cancer that has metastasized to the brain or CNS and the BRAF mutation is a D594A mutation.

In certain embodiments the disorder is cancer that has metastasized to the brain or CNS and the BRAF mutation is a L597 mutation.

In certain embodiments the disorder is cancer that has metastasized to the brain or CNS and the BRAF mutation is a L597R mutation.

In certain embodiments the disorder is cancer that has metastasized to the brain or CNS and the BRAF mutation is a L597S mutation.

In certain embodiments the disorder is cancer that has metastasized to the brain or CNS and the BRAF mutation is a L597Q mutation.

In certain embodiments the disorder is cancer that has metastasized to the brain or CNS and the BRAF mutation is a V600 mutation.

In certain embodiments the disorder is cancer that has metastasized to the brain or CNS and the BRAF mutation is a V600E mutation.

In certain embodiments the disorder is cancer that has metastasized to the brain or CNS and the BRAF mutation is a V600K mutation.

In certain embodiments the disorder is cancer that has metastasized to the brain or CNS and the BRAF mutation is a V600R mutation.

In certain embodiments the disorder is cancer that has metastasized to the brain or CNS and the BRAF mutation is a V600D mutation.

In certain embodiments the disorder is cancer that has metastasized to the brain or CNS and the BRAF mutation is a V600M mutation.

In certain embodiments the disorder is cancer that has metastasized to the brain or CNS and the BRAF mutation is a V600N mutation.

In certain embodiments the disorder is cancer that has metastasized to the brain or CNS and the BRAF mutation is a K601 mutation.

In certain embodiments the disorder is cancer that has metastasized to the brain or CNS and the BRAF mutation is a K601E mutation.

In certain embodiments the disorder is cancer that has metastasized to the brain or CNS and the BRAF mutation is a K601N mutation.

In certain embodiments, Compound 1 or a pharmaceutically acceptable salt thereof, may treat a mutant BRAF mediated cancer that has metastasized to the brain or CNS wherein the mutation is a splice variant, for example p61-BRAF^{V600E}.

In certain embodiments, Compound 1 or a pharmaceutically acceptable salt thereof, is used to treat a mutant BRAF mediated cancer that has metastasized to the brain or CNS, wherein the cancer is mediated by two or more mutant proteins, for example a cancer mediated by a BRAF^{V600E}/NRAS^{Q61K} or a BRAF^{V600E}/NRAS^{Q61R} double mutant. Non-limiting examples of double mutant cancers include colorectal cancer which is mediated by a BRAF mutation, for example BRAF^{V600E}, and a mutation of NRAS, MEK1, or PI3K, for example, BRAF^{V600E}/PIK3CA^{H1047R} or BRAF^{V600E}/PIK3CA^{P449T}.

In certain embodiments, Compound 1 or a pharmaceutically acceptable salt thereof, is used to treat a mutant BRAF mediated cancer that has metastasized to the brain or CNS, wherein the cancer is resistant to at least one BRAF inhibitor, for example a cancer that is resistant to or has acquired resistance to a BRAF inhibitor selected from dabrafenib, vemurafenib, and encorafenib.

In certain aspects the cancer that is resistant to treatment with a BRAF inhibitor has a RAF protein homo-dimerization or hetero-dimerization promoting mutation. For example, in certain embodiments Compound 1 or a pharmaceutically acceptable salt thereof, is used to treat a mutant BRAF mediated cancer that has metastasized to the brain or CNS wherein the cancer has one or more mutations that promote RAF protein dimerization. In certain embodiments the cancer with one or more RAF protein dimerization promoting mutations is resistant to treatment with a BRAF inhibitor for example dabrafenib, vemurafenib, or encorafenib. In certain embodiments the RAF protein dimer is a homo-dimer of (BRAF-BRAF) or a hetero-dimer with other RAF proteins (BRAF-RAF1 or BRAF-ARAF).

In certain embodiments, Compound 1 or a pharmaceutically acceptable salt thereof, is used to treat a cancer that has metastasized to the brain or CNS, wherein the cancer has developed an escape mutation such as BRAF V600E/NRAS^{Q61K} or BRAF V600E/NRAS^{Q61R} double mutant cancer.

In certain embodiments, Compound 1 or a pharmaceutically acceptable salt thereof, is used to treat a cancer that has metastasized to the brain or CNS, wherein the cancer has developed an amplification of a driving mutation.

In other embodiments, Compound 1 decreases phosphorylated ERK signal, indicating suppression of the EGFR-mediated MAPK pathway. In another embodiment, Compound 1 is used to treat a BRAF V600X colorectal cancer (CRC). In certain embodiments, Compound 1 is used to treat BRAF-V600X non-small cell lung cancer (NSCLC). In certain embodiments, Compound 1 shows activity against resistant tumor mutations such as point mutations in MEK, PI3K, splice variants such as p61-BRAF-V600E, BRAF kinase domain duplication, and BRAF amplifications.

In certain embodiments, Compound 1 or a pharmaceutically acceptable salt thereof, can be used to treat a colorectal cancer with a V600 mutation, for example, in certain embodiments Compound 1 or a pharmaceutically acceptable salt thereof can be used to decrease tumor growth in a colorectal cancer model with a BRAF V600 mutation than the combination of encorafenib and cetuximab.

In certain embodiments, Compound 1 or a pharmaceutically acceptable salt thereof, is used to treat melanoma that has metastasized to the brain or CNS. In other embodiments, Compound 1 or a pharmaceutically acceptable salt thereof, is used to treat colorectal cancer that has metastasized to the brain or CNS.

In certain embodiments Compound 1 or a pharmaceutically acceptable salt thereof is used to treat melanoma that has metastasized to the brain or CNS.

In certain embodiments Compound 1 or a pharmaceutically acceptable salt thereof is used to treat triple negative breast cancer that has metastasized to the brain or CNS, for example triple negative breast cancer with a G464V BRAF mutant that has metastasized to the brain or CNS.

In certain embodiments Compound 1 or a pharmaceutically acceptable salt thereof is used to treat lung cancer that has metastasized to the brain or CNS, for example lung adenocarcinoma with a G466V BRAF mutant that has metastasized to the brain or CNS.

In certain embodiments Compound 1 or a pharmaceutically acceptable salt thereof is used to treat melanoma that has metastasized to the brain or CNS with a V600 BRAF mutant.

In certain embodiments Compound 1 or a pharmaceutically acceptable salt thereof is used to treat cholangiocarcinoma that has metastasized to the brain or CNS.

In certain embodiments Compound 1 or a pharmaceutically acceptable salt thereof is used to treat erdeheim-chester disease that has metastasized to the brain or CNS.

In certain embodiments Compound 1 or a pharmaceutically acceptable salt thereof is used to treat langerhans histiocytosis that has metastasized to the brain or CNS.

In certain embodiments Compound 1 or a pharmaceutically acceptable salt thereof is used to treat ganglioglioma that has metastasized to the brain or CNS.

In certain embodiments Compound 1 or a pharmaceutically acceptable salt thereof is used to treat glioma that has metastasized to the brain or CNS.

In certain embodiments Compound 1 or a pharmaceutically acceptable salt thereof is used to treat GIST that has metastasized to the brain or CNS.

In certain embodiments Compound 1 or a pharmaceutically acceptable salt thereof is used to treat glioblastoma that has metastasized to the brain or CNS.

In certain embodiments Compound 1 or a pharmaceutically acceptable salt thereof is used to treat hairy cell leukemia that has metastasized to the brain or CNS.

In certain embodiments Compound 1 or a pharmaceutically acceptable salt thereof is used to treat multiple myeloma that has metastasized to the brain or CNS.

In certain embodiments Compound 1 or a pharmaceutically acceptable salt thereof is used to treat non-small-cell lung cancer that has metastasized to the brain or CNS.

In certain embodiments Compound 1 or a pharmaceutically acceptable salt thereof is used to treat ovarian cancer that has metastasized to the brain or CNS.

In certain embodiments Compound 1 or a pharmaceutically acceptable salt thereof is used to treat pilomyxoid astrocytoma that has metastasized to the brain or CNS.

In certain embodiments Compound 1 or a pharmaceutically acceptable salt thereof is used to treat anaplastic pleomorphic xanthoastrocytoma that has metastasized to the brain or CNS.

In certain embodiments Compound 1 or a pharmaceutically acceptable salt thereof is used to treat astrocytoma that has metastasized to the brain or CNS.

In certain embodiments Compound 1 or a pharmaceutically acceptable salt thereof is used to treat thyroid cancer that has metastasized to the brain or CNS.

In certain embodiments Compound 1 or a pharmaceutically acceptable salt thereof is used to treat papillary thyroid cancer that has metastasized to the brain or CNS.

In certain embodiments Compound 1 or a pharmaceutically acceptable salt thereof is used to treat anaplastic thyroid cancer that has metastasized to the brain or CNS.

In certain embodiments Compound 1 or a pharmaceutically acceptable salt thereof is used to treat pancreatic cancer that has metastasized to the brain or CNS.

In certain embodiments Compound 1 or a pharmaceutically acceptable salt thereof is used to treat thoracic clear cell sarcoma that has metastasized to the brain or CNS.

In certain embodiments Compound 1 or a pharmaceutically acceptable salt thereof is used to treat salivary gland cancer that has metastasized to the brain or CNS.

In certain embodiments Compound 1 or a pharmaceutically acceptable salt thereof is used to treat colorectal cancer that has metastasized to the brain or CNS.

In certain embodiments Compound 1 or a pharmaceutically acceptable salt thereof is used to treat microsatellite stable colorectal cancer that has metastasized to the brain or CNS.

In certain aspects Compound 1 or a pharmaceutically acceptable salt thereof is used to treat a mutant BRAF mediated cancer that has metastasized to the brain. In other aspects Compound 1 or a pharmaceutically acceptable salt thereof is used to treat a mutant BRAF mediated cancer that has metastasized to the CNS.

Another aspect of the present invention provides a method of treating or preventing a proliferative disease. The method comprises administering an effective amount of a pharmaceutical composition comprising an effective amount of Compound 1 or a Compound 1 morphic form as described herein, or an enantiomer, diastereomer, or stereoisomer, or isotopic derivative of Compound 1, or pharmaceutically acceptable salt, hydrate, or solvate thereof and optionally a pharmaceutically acceptable carrier to a patient in need thereof.

In certain embodiments, the disease or disorder is cancer or a proliferation disease.

In certain embodiments, the BRAF mediated disorder is an abnormal cell proliferation, including, but not limited to, a solid or hematological cancer.

In certain embodiments, the hematological cancer is acute myelogenous leukemia (AML), acute lymphoblastic leukemia (ALL), lymphoblastic T-cell leukemia, chronic myelogenous leukemia (CML), chronic lymphocytic leukemia (CLL), hairy-cell leukemia, chronic neutrophilic leukemia (CNL), acute lymphoblastic T-cell leukemia, acute monocytic leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukemia (MLL), erythroleukemia, malignant lymphoma, Hodgkin's lymphoma, non-Hodgkin's lymphoma, lymphoblastic T-cell lymphoma, Burkitt's lymphoma, follicular lymphoma, B cell acute lymphoblastic leukemia, diffuse large B cell lymphoma, Myc and B-Cell Leukemia (BCL)2 and/or BCL6 rearrangements/overexpression [double- and triple-hit lymphoma], myelodysplastic/myeloproliferative neoplasm, mantle cell lymphoma including bortezomib resistant mantle cell lymphoma.

Solid tumors that are sensitive to BRAF or mutant BRAF inhibition or degradation and thus can be treated with the compounds described herein include, but are not limited to lung cancers, including small cell lung cancer (SCLC) and non-small cell lung cancer (NSCLC), breast cancers including inflammatory breast cancer, ER-positive breast cancer including tamoxifen resistant ER-positive breast cancer, and triple negative breast cancer, colon cancers, midline carcinomas, liver cancers, renal cancers, prostate cancers including castrate resistant prostate cancer (CRPC), brain cancers including gliomas, glioblastomas, neuroblastoma, and medulloblastoma including MYC-amplified medulloblastoma, colorectal cancers, Wilm's tumor, Ewing's sarcoma, rhabdomyosarcomas, ependymomas, head and neck cancers, melanomas, squamous cell carcinomas, ovarian cancers, pancreatic cancers including pancreatic ductal adenocarcinomas (PDAC) and pancreatic neuroendocrine tumors (PanNET), osteosarcomas, giant cell tumors of bone, thyroid cancers, bladder cancers, urothelial cancers, vulval cancers, cervical cancers, endometrial cancers, mesotheliomas, esophageal cancers, salivary gland cancers, gastric cancers, nasopharyngeal cancers, buccal cancers, cancers of the mouth, GIST (gastrointestinal stromal tumors), NUT-midline carcinomas, testicular cancers, squamous cell carcinomas, hepatocellular carcinomas (HCC), MYCN driven solid tumors, and NUT midline carcinomas (NMC).

In further embodiments, the disease or disorder is sarcoma of the bones, muscles, tendons, cartilage, nerves, fat, or blood vessels.

In further embodiments, the disease or disorder is soft tissue sarcoma, bone sarcoma, or osteosarcoma.

In further embodiments, the disease or disorder is angiosarcoma, fibrosarcoma, liposarcoma, leiomyosarcoma, Kaposi's sarcoma, osteosarcoma, gastrointestinal stromal tumor, synovial sarcoma, pleomorphic sarcoma, chondrosarcoma, Ewing's sarcoma, reticulum cell sarcoma, hemangiosarcoma, botryoid sarcoma, rhabdomyosarcoma, or embryonal rhabdomyosarcoma.

In certain embodiments the disorder is a bone, muscle, tendon, cartilage, nerve, fat, or blood vessel sarcoma.

In other embodiments, the disease or disorder is a cancer which is sensitive to BRAF or mutant BRAF inhibition or degradation. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, solid tumors, hematological cancers or solid cancers.

The term “cancer” as used herein to refer to cancers that can be treated with Compound 1 refers to any cancer caused by the proliferation of malignant neoplastic cells, such as tumors, neoplasms, carcinomas, sarcomas, leukemias, lymphomas and the like wherein the disease is sensitive to BRAF or mutant BRAF inhibition or degradation. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers, such as oral, laryngeal, nasopharyngeal and esophageal, genitourinary cancers, such as prostate, bladder, renal, uterine, ovarian, testicular, lung cancer, such as small-cell and non-small cell, breast cancer, pancreatic cancer, melanoma and other skin cancers, stomach cancer, brain tumors, tumors related to Gorlin's syndrome, such as medulloblastoma or meningioma, and liver cancer.

One aspect of this application provides a pharmaceutical composition comprising an effective amount of Compound 1 or a Compound 1 morphic form that is useful for the treatment of diseases, disorders, and conditions characterized by excessive or abnormal cell proliferation that are sensitive to BRAF or mutant BRAF inhibition or degradation. Such diseases include, but are not limited to, a proliferative or hyperproliferative disease. Examples of proliferative and hyperproliferative diseases include, without limitation, cancer. The term “cancer” includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colorectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharyngeal, non-small cell lung cancer (NSCLC), endometrial, hepatocarcinoma, non-Hodgkin's lymphoma, and pulmonary.

Additional exemplary forms of cancer include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that can be treated by the present invention include colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing sarcoma, and plasmacytoma. In one aspect of the application, the present application provides for the use of one or more compound as described herein, in the manufacture of a medicament for the treatment of cancer, including without limitation the various types of cancer disclosed herein.

In some embodiments, a pharmaceutical composition or morphic form described herein is useful for treating a cancer which is sensitive to BRAF or mutant BRAF inhibition or degradation, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, a Compound 1 morphic form as described herein is useful for treating hematopoietic disorders which are sensitive to BRAF or mutant BRAF inhibition or degradation, in particular, acute-myelogenous leukemia (AML), chronic-myelogenous leukemia (CML), acute-promyelocytic leukemia, and acute lymphocytic leukemia (ALL).

In certain embodiments, a pharmaceutical composition or morphic form described herein can be used in an effective amount to treat a host, for example a human, with a lymphoma or lymphocytic or myelocytic proliferation disorder or abnormality. For example, a Compound 1 morphic form as described herein can be administered to a host suffering from a Hodgkin's Lymphoma or a Non-Hodgkin's Lymphoma. For example, the host can be suffering from a non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In other embodiments, a pharmaceutical composition or morphic form described herein can be used in an effective amount to treat a patient, for example a human, with a Hodgkin's lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin's Lymphoma; or Nodular Lymphocyte Predominant HL.

This application further embraces the treatment or prevention of cell proliferative disorders such as hyperplasias, dysplasias and pre-cancerous lesions which are sensitive to BRAF or mutant BRAF inhibition or degradation. Dysplasia is the earliest form of pre-cancerous lesion recognizable in a biopsy by a pathologist. A pharmaceutical composition or morphic form described herein may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

In certain aspects, a pharmaceutical composition comprising an effective amount of Compound 1 or a morphic form thereof is used to treat a BRAF mediated cancer, wherein the BRAF has mutated from the wild type. There are a number of possibilities for BRAF mutations.

In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, V600M and V600N. Non-limiting examples of Class II mutations include G469A, G469V, G469L, G469R, L597Q, and K601E. Non-limiting examples of Class III mutations include G466A, G466E, G466R, G466V, S467L, G469E, N581L, D594E, D594G, and D594N.

In certain embodiments, a pharmaceutical composition comprising an effective amount of Compound 1 or a morphic form thereof treats a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

Another aspect of the present invention provides a pharmaceutical composition comprising an effective amount of Compound 1 or a morphic form thereof, for use in the manufacture of a medicament for treating or preventing a disease mediated by BRAF.

In certain embodiments, a pharmaceutical composition comprising an effective amount of Compound 1 or a morphic form thereof is useful to treat a disorder comprising an abnormal cellular proliferation, such as a tumor or cancer, wherein BRAF is an oncogenic protein or a signaling mediator of the abnormal cellular proliferative pathway and its degradation decreases abnormal cell growth.

### Combination Therapy

A pharmaceutical composition comprising an effective amount of Compound 1 or a pharmaceutically acceptable salt thereof or a Compound 1 morphic form as described herein can be used alone or in combination or administered simultaneously or sequentially with another bioactive agent or second therapeutic agent to treat a patient such as a human with a mutant BRAF mediated disorder, including but not limited to those described herein.

The term “bioactive agent” or “additional active agent” is used to describe an agent, other than Compound 1 or a pharmaceutically acceptable salt thereof or a Compound 1 morphic form according to the present invention, which can be used in combination or alternation with a pharmaceutical composition comprising an effective amount of Compound 1 or a pharmaceutically acceptable salt thereof or a Compound 1 morphic form to achieve a desired result of therapy. In certain embodiments, a pharmaceutical composition comprising an effective amount of Compound 1 or a pharmaceutically acceptable salt thereof or a Compound 1 morphic form and the bioactive agent are administered in a manner that they are active in vivo during overlapping time periods, for example, have time-period overlapping Cₘₐₓ, Tₘₐₓ, AUC or another pharmacokinetic parameter. In another embodiment, a pharmaceutical composition comprising an effective amount of Compound 1 or a pharmaceutically acceptable salt thereof or a Compound 1 morphic form and the bioactive agent are administered to a patient in need thereof that do not have overlapping pharmacokinetic parameter, however, one has a therapeutic impact on the therapeutic efficacy of the other.

In certain aspects, a pharmaceutical composition comprising an effective amount of Compound 1 or a morphic form thereof is used in combination with a second active agent described herein to treat a mutant BRAF mediated cancer. Non-limiting examples of classes of molecules that can be used in combination with a pharmaceutical composition comprising an effective amount of Compound 1 or a morphic form thereof include MEK inhibitors, immune checkpoint inhibitors, and EGFR antibodies. In certain embodiments, a pharmaceutical composition comprising an effective amount of Compound 1 or a morphic form thereof is used in combination with trametinib for the treatment of a mutant BRAF mediated cancer, for example melanoma or non-small cell lung cancer. In certain embodiments, a pharmaceutical composition comprising an effective amount of Compound 1 or a morphic form thereof is used in combination with an immune checkpoint inhibitor to treat a mutant BRAF mediated cancer. In certain embodiments, a pharmaceutical composition comprising an effective amount of Compound 1 or a morphic form thereof is used in combination with cetuximab or panitumumab to treat a mutant BRAF mediated cancer, for example colorectal cancer. In certain embodiments, a pharmaceutical composition comprising an effective amount of Compound 1 or a morphic form thereof is used in combination with nivolumab, pembrolizumab, cemiplimab, ipilimumab, relatlimab, atezolizumab, avelumab, or durvalumab to treat a mutant BRAF mediated cancer, for example colorectal cancer, melanoma, or non-small cell lung cancer.

In other aspects, a pharmaceutical composition comprising an effective amount of Compound 1 or a morphic form thereof is used in combination with two or more additional active agents described herein to treat a mutant BRAF mediated cancer. In certain embodiments, a pharmaceutical composition comprising an effective amount of Compound 1 or a morphic form thereof is used in combination with a MEK inhibitor and an immune checkpoint inhibitor to treat melanoma or non-small cell lung cancer.

In some embodiments, Compound 1 or a pharmaceutically acceptable salt thereof or a Compound 1 morphic form is used in combination with another BRAF inhibitor such as sorafenib, vemurafenib (ZELBORAF®), dabrafenib (TAFINLAR®) or encorafenib (BRAFTOVI®).

In certain embodiments, the bioactive agent is a MEK inhibitor. MEK inhibitors are well known, and include, for example, trametinib/GSK1120212 (N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-l(2H-yl)phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA1 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 (I-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2-yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In certain embodiments the MEK inhibitor is trametinib.

In certain embodiments, a pharmaceutical composition comprising an effective amount of Compound 1 or a pharmaceutically acceptable salt thereof or a Compound 1 morphic form is used in combination with cetuximab or trametinib to treat colorectal cancer. In certain embodiments, a pharmaceutical composition comprising an effective amount of Compound 1 or a pharmaceutically acceptable salt thereof or a Compound 1 morphic form is used in combination with cetuximab and BYL719 to treat colorectal cancer. In certain embodiments, a pharmaceutical composition comprising an effective amount of Compound 1 or a pharmaceutically acceptable salt thereof or a Compound 1 morphic form is used in combination with cetuximab and irinotecan to treat colorectal cancer.

In certain embodiments, the bioactive agent is a SHP2 inhibitor. In certain embodiments, the SHP2 inhibitor is SHP099.

In certain embodiments, the bioactive agent is a RAF inhibitor. Non-limiting examples of Raf inhibitors include, for example, vemurafenib (N-[3-[[5-(4-chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), sorafenib N-oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (encorafenib (BRAFTOVI®)).

In certain embodiments the RAF inhibitor is encorafenib.

In certain embodiments the RAF inhibitor is vemurafenib.

In certain embodiments the RAF inhibitor is dabrafenib.

In certain embodiments, the bioactive agent is an EGFR inhibitor, including, for example gefitinib (IRESSA®), erlotinib (TARCEVA®), lapatinib (TYKERB®), osimertinib (TAGRISSO®), neratinib (NERLYNX®), vandetanib (CAPRELSA®), dacomitinib (VIZIMPRO®), rociletinib (XEGAFRI™), afatinib (GLOTRIF®, GIOTRIFF™, AFANIX™), lazertinib, or nazartib.

Additional examples of EGFR inhibitors include rociletinib (CO-1686), olmutinib (OLITA™), naquotinib (ASP8273), nazartinib (EGF816), PF-06747775, icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, dacomitinib (PF-00299804; Pfizer), brigatinib (ALUNBRIG®), lorlatinib, and PF-06747775 (PF7775).

In certain embodiments, the bioactive agent is a first-generation EGFR inhibitor such as erlotinib, gefitinib, or lapatinib. In certain embodiments, the bioactive agent is a second-generation EGFR inhibitor such as afatinib and/or dacomitinib. In certain embodiments, the bioactive agent is a third-generation EGFR inhibitor such as osimertinib.

In certain embodiments, a pharmaceutical composition comprising an effective amount of Compound 1 or a pharmaceutically acceptable salt thereof or a Compound 1 morphic form is administered to a patient in need thereof in combination with osimertinib.

In certain embodiments, a pharmaceutical composition comprising an effective amount of Compound 1 or a pharmaceutically acceptable salt thereof or a Compound 1 morphic form is administered to a patient in need thereof in combination with rociletinib.

In certain embodiments, a pharmaceutical composition comprising an effective amount of Compound 1 or a pharmaceutically acceptable salt thereof or a Compound 1 morphic form is administered to a patient in need thereof in combination with avitinib.

In certain embodiments, a pharmaceutical composition comprising an effective amount of Compound 1 or a pharmaceutically acceptable salt thereof or a Compound 1 morphic form is administered to a patient in need thereof in combination with lazertinib.

In certain embodiments, a pharmaceutical composition comprising an effective amount of Compound 1 or a pharmaceutically acceptable salt thereof or a Compound 1 morphic form is administered to a patient in need thereof in combination with nazartinib.

In certain embodiments, a pharmaceutical composition comprising an effective amount of Compound 1 or a pharmaceutically acceptable salt thereof or a Compound 1 morphic form is administered to a patient in need thereof in combination with an EGFR antibody, for example, cetuximab, panitumumab, or necitumumab.

In certain embodiments, a pharmaceutical composition comprising an effective amount of Compound 1 or a pharmaceutically acceptable salt thereof or a Compound 1 morphic form is administered to a patient in need thereof in combination with cetuximab.

In certain embodiments, a pharmaceutical composition comprising an effective amount of Compound 1 or a pharmaceutically acceptable salt thereof or a Compound 1 morphic form is administered to a patient in need thereof in combination with panitumumab.

In certain embodiments, a pharmaceutical composition comprising an effective amount of Compound 1 or a pharmaceutically acceptable salt thereof or a Compound 1 morphic form is administered to a patient in need thereof in combination with necitumumab.

In certain embodiments, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or another inhibitor. In certain aspects, the immune modulator is an antibody, such as a monoclonal antibody.

PD-1 inhibitors that block the interaction of PD-1 and PD-L1 by binding to the PD-1 receptor, and in turn inhibit immune suppression include, for example, nivolumab (OPDIVO®), pembrolizumab (KEYTRUDA®), pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (GlaxoSmithKline plc), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (TECENTRIQ®), durvalumab (AstraZeneca and MedImmune), KN035 (Alphamab Co. Ltd.), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors include, but are not limited to, BMS-986016 (Bristol-Myers Squibb), GSK2831781 (GlaxoSmithKline plc), IMP321 (Prima BioMed), LAG525 (Novartis), and the dual PD-1 and LAG-3 inhibitor MGD013 (MacroGenics). An example of a TIM-3 inhibitor is TSR-022 (GlaxoSmithKline plc).

In certain embodiments, the checkpoint inhibitor is selected from nivolumab (OPDIVO®); pembrolizumab (KEYTRUDA®); and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GAL9, LAG 3, VISTA, KIR, 2B4, CD160, CGEN-15049, CHK 1, CHK2, A2aR, B-7 family ligands, or a combination thereof.

In yet other embodiments, Compound 1 or a pharmaceutically acceptable salt thereof or a Compound 1 morphic form described herein can be administered in an effective amount for the treatment of abnormal tissue of the female reproductive system such as breast, ovarian, endometrial, or uterine cancer, in combination or alternation with an effective amount of an estrogen inhibitor including, but not limited to, a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors.

Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703, 810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In another embodiment, a pharmaceutical composition comprising an effective amount of Compound 1 or a pharmaceutically acceptable salt thereof or a Compound 1 morphic form can be administered in an effective amount for the treatment of abnormal tissue of the male reproductive system such as prostate or testicular cancer, in combination or alternation with an effective amount of an androgen (such as testosterone) inhibitor including, but not limited to a selective androgen receptor modulator, a selective androgen receptor degrader, a complete androgen receptor degrader, or another form of partial or complete androgen antagonist. In certain embodiments, the prostate or testicular cancer is androgen resistant.

Non-limiting examples of anti-androgen compounds are provided in WO 2011/156518 and U.S. Pat. Nos. 8,455,534 and 8,299,112. Additional non-limiting examples of anti-androgen compounds include enzalutamide, apalutamide, cyproterone acetate, chlormadinone acetate, spironolactone, canrenone, drospirenone, ketoconazole, topilutamide, abiraterone acetate, and cimetidine.

In certain embodiments, the bioactive agent is an ALK inhibitor. Examples of ALK inhibitors include but are not limited to crizotinib (XALKORI®), alectinib (ALECENSA®), ceritinib, TAE684 (NVP-TAE684), GSK1838705A, AZD3463, ASP3026, PF-06463922, entrectinib (RXDX-101), and AP26113.

In certain embodiments, the bioactive agent is an HER-2 inhibitor. Examples of HER-2 inhibitors include trastuzumab, lapatinib, ado-trastuzumab emtansine, and pertuzumab.

In certain embodiments, the bioactive agent is a CD20 inhibitor. Examples of CD20 inhibitors include obinutuzumab (GAZYVA®), rituximab (RITUXAN®), ofatumumab, ibritumomab, tositumomab, and ocrelizumab.

In certain embodiments, the bioactive agent is a JAK3 inhibitor. Examples of JAK3 inhibitors include tasocitinib.

In certain embodiments, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 (I-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (oblimersen).

In certain embodiments, the bioactive agent is a kinase inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include, but are not limited to, Wortmannin, demethoxyviridin, perifosine, idelalisib, pictilisib, palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 (I-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[l-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), LY294002, AZD8186, PF-4989216, pilaralisib, GNE-317, PI-3065, PI-103, NU7441 (KU-57788), HS 173, VS-5584(SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422).

Examples of BTK inhibitors include ibrutinib (also known as PCI-32765)(IMBRUVICA®) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 (I-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 (I-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, but are not limited to, cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (GLEEVEC®; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In certain embodiments, the bioactive agent is a c-MET inhibitor, for example, crizotinib (XALKORI®, CRIZONIX™), tepotinib (XL880, EXEL-2880, GSK1363089, GSK089), or tivantinib (ARQ197).

In certain embodiments, the bioactive agent is an AKT inhibitor, including, but not limited to, MK-2206, GSK690693, perifosine, (KRX-0401), GDC-0068, triciribine, AZD5363, honokiol, PF-04691502, and miltefosine, a FLT-3 inhibitor, including, but not limited to, P406, dovitinib, quizartinib (AC220), amuvatinib (MP-470), tandutinib (MLN518), ENMD-2076, and KW-2449, or a combination thereof.

In certain embodiments, the bioactive agent is an mTOR inhibitor. Examples of mTOR inhibitors include, but are not limited to, rapamycin and its analogs, everolimus (AFINITOR®), temsirolimus, ridaforolimus, sirolimus, and deforolimus.

In certain embodiments, the bioactive agent is a RAS inhibitor. Examples of RAS inhibitors include but are not limited to Reolysin and siG12D LODER.

In certain embodiments, the bioactive agent is a HSP inhibitor. HSP inhibitors include but are not limited to geldanamycin or 17-N-allylamino-17-demethoxygeldanamycin (17AAG), and radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhibitor, a c-MET inhibitor, a PARP inhibitor, a Cdk inhibitor, an IGFR-TK inhibitor, an anti-HGF antibody, a focal adhesion kinase inhibitor, a Map kinase (MEK) inhibitor, a VEGF trap antibody, pemetrexed, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IpdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, GLEEVEC®, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HNR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments, a pharmaceutical composition comprising an effective amount of Compound 1 or a pharmaceutically acceptable salt thereof or a Compound 1 morphic form is administered in combination with ifosfamide.

In certain embodiments, the bioactive agent is selected from, but are not limited to, imatinib mesylate (GLEEVEC®), dasatinib (SPRYCEL®), nilotinib (TASIGNA®), bosutinib (BOSULIF®), trastuzumab (HERCEPTIN®), trastuzumab-DM1, pertuzumab (PERJETA®), lapatinib (TYKERB®), gefitinib (IRESSA®), erlotinib (TARCEVA®), cetuximab (ERBITUX®), panitumumab (VECTIBIX®), vandetanib (CAPRELSA®), vemurafenib (ZELBORAF®), vorinostat (ZOLINZA®), romidepsin (ISTODAX®), bexarotene (TAGRETIN®), alitretinoin (PANRETIN®), tretinoin (VESANOID®), carfilizomib (KYPROLIS®), pralatrexate (FOLOTYN®), bevacizumab (AVASTIN®), ziv-aflibercept (ZALTRAP®), sorafenib (NEXAVAR®), sunitinib (SUTENT®), pazopanib (VOTRIENT®), regorafenib (STIVARGA®), and cabozantinib (COMETRIQ®).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: vincristine (ONCOVINE®) or liposomal vincristine (MARQIBO®), daunorubicin (daunomycin or CERUBIDINE®) or doxorubicin (ADRIAMYCIN®), cytarabine (cytosine arabinoside, ara-C, or CYTOSAR®), L-asparaginase (ELSPAR®) or PEG-L-asparaginase (pegaspargase or ONCASPAR®), etoposide (VP-16), teniposide (VUMON®), 6-mercaptopurine (6-MP or PURINETHOL®), methotrexate, cyclophosphamide (CYTOXAN®), prednisone, dexamethasone (DECADRON®), imatinib (GLEEVEC®), dasatinib (SPRYCEL®), nilotinib (TASIGNA®), bosutinib (BOSULIF®), and ponatinib (ICLUSIG®).

Examples of additional suitable chemotherapeutic agents include, but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytochalasin B, cytoxan, dacarbazine, dactinomycin, dactinomycin (formerly actinomycin), daunirubicin HCL, daunorucbicin citrate, denileukin diftitox, dexrazoxane, dibromomannitol, dihydroxy anthracin dione, docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, a pharmaceutical composition comprising an effective amount of Compound 1 or a pharmaceutically acceptable salt thereof or a Compound 1 morphic form is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), binutuzum, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, obinutuzumab, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; binutuzum; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; obinutuzumab, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France) binutuzumab; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with a pharmaceutical composition comprising an effective amount of Compound 1 or a pharmaceutically acceptable salt thereof or a Compound 1 morphic form. Suitable dosing regimens of combination chemotherapies are known in the ar. For example, combination dosing regimens are described in Saltz et al., Proc. Am. Soc. Clin. Oncol. 18:233a (1999) and Douillard et al., Lancet 355(9209): 1041-1047 (2000).

Additional therapeutic agents that can be administered in combination with a pharmaceutical composition comprising an effective amount of Compound 1 or a pharmaceutically acceptable salt thereof or a Compound 1 morphic form can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (ELOXATIN®), PD0332991, ribociclib (LEE011), amebaciclib (LY2835219), HDM201, fulvestrant (FASLODEX®), exemestane (AROMASIN®), PIM447, ruxolitinib (INC424), BGJ398, necitumumab, pemetrexed (ALIMTA®), and ramucirumab (IMC-1121B).

In certain embodiments, the additional therapy is a monoclonal antibody (Mab). Some Mabs stimulate an immune response that destroys cancer cells. Similar to the antibodies produced naturally by B cells, these Mabs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Mabs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g., a cyclosporin or an ascomycin, e.g., cyclosporin A (NEORAL®), FK506 (tacrolimus), pimecrolimus, a mTOR inhibitor, e.g., rapamycin or a derivative thereof, e.g., sirolimus (RAPAMUNE®), everolimus (CERTICAN®), temsirolimus, zotarolimus, biolimus-7, biolimus-9, a rapalog, e.g., ridaforolimus, azathioprine, campath 1H, a S1P receptor modulator, e.g., fingolimod or an analog thereof, an anti IL-8 antibody, mycophenolic acid or a salt thereof, e.g., sodium salt, or a prodrug thereof, e.g., mycophenolate mofetil (CELLCEPT®), OKT3 (ORTHOCLONE OKT3®), prednisone, ATGAM®, THYMOGLOBULIN®, brequinar sodium, OKT4, T10B9.A-3A, 33B3.1, 15-deoxyspergualin, tresperimus, leflunomide (ARAVA®), CTLAI-Ig, anti-CD25, anti-IL2R, basiliximab (SIMULECT), daclizumab (ZENAPAX®), mizorbine, methotrexate, dexamethasone, ISAtx-247, SDZ ASM 981 (pimecrolimus, ELIDEL®), CTLA4lg (abatacept), belatacept, LFA3lg, etanercept (sold as ENBREL® by Immunex), adalimumab (HUMIRA), infliximab (REMICADE®), an anti-LFA-1 antibody, natalizumab (ANTEGREN®), enlimomab, gavilimomab, antithymocyte immunoglobulin, siplizumab, alefacept, efalizumab, pentasa, mesalazine, asacol, codeine phosphate, benorylate, fenbufen, naprosyn, diclofenac, etodolac and indomethacin, aspirin and ibuprofen.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The combination therapy may include a therapeutic agent which is a non-drug treatment. For example, a pharmaceutical composition comprising an effective amount of Compound 1 or a pharmaceutically acceptable salt thereof or a Compound 1 morphic form could be administered in addition to radiation therapy, cryotherapy, hyperthermia, and/or surgical excision of tumor tissue.

### Pharmaceutical Compositions

New advantageous pharmaceutical compositions comprising an effective amount of Compound 1 or a pharmaceutically acceptable salt thereof or a Compound 1 morphic form according to the present invention that is suitable for administration to humans are provided. In certain embodiments, the pharmaceutical composition comprising Compound 1 or a pharmaceutically acceptable salt thereof is produced from a morphic form described herein, for example Compound 1 Form B. For example, Compound 1 Form B can be dissolved and then spray dried to form a solid spray dry dispersion with one or more pharmaceutically acceptable excipients.

Pharmaceutically acceptable excipients suitable for use as carriers or diluents are known to a skilled person and may be used in a variety of formulations. See, for example, Remington's Pharmaceutical Sciences, 23rd Edition, A. Adejare, Editor, Academic Press (2020); Handbook of Pharmaceutical Excipients, 6th Edition, R. C. Rowe, P. J. Sheskey, M. E. Quinn Editors, American Pharmaceutical Association, and Pharmaceutical Press (2009); and Handbook of Pharmaceutical Additives, 3rd Edition, compiled by Michael and Irene Ash, Synapse Information Resources (2007).

The pharmaceutical composition may be formulated as any pharmaceutically useful form, e.g., as a solid dosage form, liquid, an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and should be of sufficiently high purity and sufficiently low toxicity to render them suitable for administration in an effective amount to the patient being treated. The carrier can be inert, or it can possess pharmaceutical benefits of its own. The amount of carrier employed in conjunction with Compound 1 or a pharmaceutically acceptable salt thereof or a Compound 1 morphic form is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of Compound 1.

An effective amount of Compound 1 or a pharmaceutically acceptable salt thereof or a Compound 1 morphic form disclosed herein may be administered orally, topically, systemically, parenterally, by inhalation or spray, sublingually, via implant, for example an implant inserted into a tumor or abnormal cell proliferation, transdermally, via buccal administration, rectally, as an ophthalmic solution, injection, including intravenous, intra-aortal, intracranial, subdermal, intraperitoneal, subcutaneous, transnasal, sublingual, or rectal or by other means, in dosage unit formulations containing conventional pharmaceutically acceptable carriers.

In certain embodiments, a pharmaceutical composition prepared from or comprising Compound 1 or pharmaceutically acceptable salt thereof or a Compound 1 morphic form of the present invention is provided in a solid, gel, or liquid dosage form for oral delivery.

In certain embodiments the pharmaceutical composition comprises Compound 1 or a pharmaceutically acceptable salt thereof, a solubility enhancer, a permeation enhancer, a filler, one or more binders and or glidants, and one or more flow aids. Non-limiting examples of pharmaceutically acceptable excipients include hypromellose (for example, hypromellose acetate succinate), vitamin E (for example, d-alpha-tocopheryl polyethylene glycol succinate), mannitol, cellulose (for example microcrystalline cellulose), croscarmellose sodium, silicon dioxide (for example untreated fumed colloidal), and magnesium stearate. In certain embodiments, a pharmaceutical composition according to the present invention is formulated into a dosage unit form, such as an oral dosage unit form. In certain embodiments, a pharmaceutical composition according to the present invention is formulated into a tablet dosage form.

In certain embodiments, the pharmaceutical composition comprising Compound 1 or a pharmaceutically acceptable salt thereof comprises one or more of the following excipients.

| | | Percent |
|---|---|---|
| Class | Representative Components | (w/w) |
| Active | Compound 1, for example | about 5% to about 20% |
| Ingredient | Compound 1 prepared from | for example, about 10% |
| | Compound 1 Form B | |
| Solubility | Hypromellose, for example | about 15% to about 55% |
| enhancer | hypromellose acetate | for example, about 35% |
| | succinate | |
| Permeation | Vitamin E, for example d-α- | about 1% to about 10% |
| enhancer | tocopheryl polyethylene | for example, about 5% |
| | glycol succinate | |
| Filler | Mannitol | about 20% to about 40% |
| | | for example, about 28% |
| Binder/Glidant | Cellulose, for example | about 10% to about 30% |
| | microcrystalline cellulose | for example, about 20% |
| Mucoadhesive/ | Croscarmellose, for example | about 0.5% to about 5% |
| Disintegrant | croscarmellose sodium | for example, about 2% |

In certain embodiments, a process for manufacturing a pharmaceutical composition comprising Compound 1 or a pharmaceutically acceptable salt thereof or a Compound 1 morphic for according to the present invention is also provided. In certain embodiments, a process for manufacturing a pharmaceutical composition comprising Compound 1 includes: (i) a spray drying step to provide a spray-dried intermediate (SRI) containing Compound 1 and pharmaceutically acceptable excipients; (ii) a granulation step to provide a granulate containing Compound 1 and pharmaceutically acceptable excipients with a desired bulk density between about 0.4 to 0.6 g/mL for example about 0.48 to 0.54 g/mL; and (iii) a tableting step to provide a pharmaceutical composition comprising Compound 1 and pharmaceutically acceptable excipients in an oral dosage unit form.

In certain embodiments, the dose strength of the pharmaceutical composition is at least about 10 mg, 20 mg, 40 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 440 mg, 480 mg, 520 mg, 560 mg, 600 mg, 640 mg, 680 mg, 720 mg, 760 mg, or 800 mg, which may in nonlimiting aspects be given once weekly, twice weekly, three times weekly, four times weekly, five times weekly, six times weekly, once daily (QD), or twice daily (BID), optionally with treatment occurring on days 1-7, 1-14, 1-21, or 1-28 of a 28-day treatment cycle. In some embodiments, Compound 1 or a pharmaceutically acceptable salt thereof or a Compound 1 morphic form of the present invention is provided as a softshell capsule or tablet for oral administration. In certain embodiments, the dose strength of the solid or gel dosage form is at least about 10 mg, 20 mg, 40 mg, 80 mg, 140 mg, 240 mg, 360 mg, or 480 mg, 500 mg, 550 mg, 600 mg, 650 mg, 700 mg, 800 mg, which may in nonlimiting aspects be given once weekly, twice weekly, three times weekly, four times weekly, five times weekly, six times weekly, once daily (QD), or twice daily (BID), optionally with treatment occurring on days 1-7, 1-14, 1-21, or 1-28 of a 28-day treatment cycle.

In certain aspects the pharmaceutical composition is administered orally once or twice daily (BID) as needed.

Embodiments of the pharmaceutical composition include:
1. In certain embodiments, Compound 1 is provided in an effective amount in a pharmaceutical composition comprising at least three of, four of or all of a solubility enhancer, a permeation enhancer, a filler, a binder/glidant, and/or a mucoadhesive/disintegrant.
2. The pharmaceutical composition of embodiment 1, wherein the pharmaceutical composition is provided as a tablet dosage form.
3. The pharmaceutical composition of any one of embodiments 1-2, wherein the pharmaceutical composition is prepared from a Compound 1 morphic form for example by dissolving and then spray drying Compound 1 Form B.
4. The pharmaceutical composition of any one of embodiments 1-3, wherein the pharmaceutical composition further comprises a flow aid and a lubricant.
5. The pharmaceutical composition of any one of embodiments 1-4, wherein the pharmaceutical composition comprises from about 5% to about 20% of Compound 1 by weight.
6. The pharmaceutical composition of any one of embodiments 1-5, wherein the solubility enhancer is hypromellose or a hypromellose derivative.
7. The pharmaceutical composition of embodiment 6, wherein the hypromellose derivative is hypromellose acetate succinate.
8. The pharmaceutical composition of any one of embodiments 1-7, wherein the permeation enhancer is vitamin E or a vitamin E derivative.
9. The pharmaceutical composition of embodiment 8, wherein the vitamin E derivative is d-α-tocopheryl polyethylene glycol succinate.
10. The pharmaceutical composition of any one of embodiments 1-9, wherein the filler is mannitol.
11. The pharmaceutical composition of any one of embodiments 1-10, wherein the binder/glidant is cellulose.
12. The pharmaceutical composition of embodiment 11, wherein the binder/glidant is microcrystalline cellulose.
13. The pharmaceutical composition of any one of embodiments 1-12, wherein the mucoadhesive/disintegrant is croscarmellose or a croscarmellose derivative.
14. The pharmaceutical composition of embodiment 13, wherein the croscarmellose derivative is croscarmellose sodium.
15. The pharmaceutical composition of any one of embodiments 4-14, wherein the flow aid is a colloidal silicon dioxide.
16. The pharmaceutical composition of embodiment 15, wherein the colloidal silicon dioxide is an untreated fumed colloidal silicon dioxide.
17. The pharmaceutical composition of any one of embodiments 4-16, wherein the lubricant is magnesium stearate.
18. The pharmaceutical composition of any one of embodiments 1-17, wherein the pharmaceutical composition comprises from about 15% to about 55% of the solubility enhancer by weight.
19. The pharmaceutical composition of any one of embodiments 1-18, wherein the pharmaceutical composition comprises from about 1% to about 10% of the permeation enhancer by weight.
20. The pharmaceutical composition of any one of embodiments 1-19, wherein the pharmaceutical composition comprises from about 20% to about 40% of the filler by weight.
21. The pharmaceutical composition of any one of embodiments 1-20, wherein the pharmaceutical composition comprises from about 10% to about 30% of the binder/glidant by weight.
22. The pharmaceutical composition of any one of embodiments 1-21, wherein the pharmaceutical composition comprises from about 0.5% to about 5% of the mucoadhesive/disintegrant by weight.
23. The pharmaceutical composition of any one of embodiments 1-22, wherein the pharmaceutical composition comprises about 10% of Compound 1 by weight.
24. The pharmaceutical composition of any one of embodiments 1-23, wherein the pharmaceutical composition comprises about 35% of the solubility enhancer by weight.
25. The pharmaceutical composition of any one of embodiments 1-24, wherein the pharmaceutical composition comprises about 5% of the permeation enhancer by weight.
26. The pharmaceutical composition of any one of embodiments 1-25, wherein the pharmaceutical composition comprises about 28% of the filler by weight.
27. The pharmaceutical composition of any one of embodiments 1-26, wherein the pharmaceutical composition comprises about 20% of the binder/glidant by weight.
28. The pharmaceutical composition of any one of embodiments 1-27, wherein the pharmaceutical composition comprises about 2% of the mucoadhesive/disintegrant by weight.
29. The pharmaceutical composition of any one of embodiments 4-28, wherein the pharmaceutical composition comprises about 0.5% of the flow aid by weight.
30. The pharmaceutical composition of any one of embodiments 4-29, wherein the pharmaceutical composition comprises about 0.6% of the lubricant by weight.

### Pharmaceutical Compositions Comprising a Morphic Form of Compound 1

A Compound 1 morphic form can be administered as a neat chemical but is often administered as a pharmaceutical composition that includes an effective amount of a Compound 1 morphic form, to a host, typically a human, in need of such treatment for any of the disorders described herein. Accordingly, the disclosure provides pharmaceutical compositions comprising an effective amount of a Compound 1 morphic form together with at least one pharmaceutically acceptable carrier or excipient as described herein. The pharmaceutical composition may contain a Compound 1 morphic form as the only active agent, or, in alternative embodiments, a Compound 1 morphic form and at least one additional active agent.

In certain embodiments the pharmaceutical composition is in a dosage form that contains from about 0.001 mg to about 1000 mg, from about 0.01 mg to about 800 mg, from about 1 mg to about 800 mg, or from about 200 mg to about 600 mg of a Compound 1 morphic form and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least about, or no more than, 0.001, 0.005, 0.010, 0.10, 1, 5, 10, 25, 50, 100, 200, 250, 300, 400, 500, 600, 700, or 750 mg of a Compound 1 morphic form.

In certain embodiments, the pharmaceutical composition is in a dosage form that contains about 50 mg of a Compound 1 morphic form. In certain embodiments, the pharmaceutical composition is in a dosage form that contains about 100 mg of a Compound 1 morphic form. In certain embodiments, the pharmaceutical composition is in a dosage form that contains about 150 mg of a Compound 1 morphic form. In certain embodiments, the pharmaceutical composition is in a dosage form that contains about 200 mg of a Compound 1 morphic form. In certain embodiments, the pharmaceutical composition is in a dosage form that contains about 250 mg of a Compound 1 morphic form. In certain embodiments, the pharmaceutical composition is in a dosage form that contains about 300 mg of a Compound 1 morphic form. In certain embodiments, the pharmaceutical composition is in a dosage form that contains about 400 mg of a Compound 1 morphic form. In certain embodiments, the pharmaceutical composition is in a dosage form that contains about 500 mg of a Compound 1 morphic form. In certain embodiments, the pharmaceutical composition is in a dosage form that contains about 600 mg of a Compound 1 morphic form. In certain embodiments, the pharmaceutical composition is in a dosage form that contains about 700 mg of a Compound 1 morphic form. In certain embodiments the pharmaceutical composition is in a dosage form that contains about 800 mg of a Compound 1 morphic form.

In certain embodiments, a Compound 1 morphic form is administered once or twice per day to a patient in need thereof.

In certain embodiments, a pharmaceutical composition according to the present invention includes a Compound 1 morphic form and pharmaceutically acceptable excipients. In certain embodiments, pharmaceutically acceptable excipients include, but not limited to, hypromellose acetate succinate, vitamin E TPGS (d-α-tocopheryl polyethylene glycol succinate), mannitol, microcrystalline cellulose, croscarmellose sodium, untreated fumed colloidal silicon dioxide, and magnesium stearate.

The pharmaceutical compositions can be formulated for oral administration. These compositions can contain any amount of a Compound 1 morphic form that achieves the desired result, for example between 0.1 and 99 weight % (wt. %) of a Compound 1 morphic form and usually at least about 5 wt. % of a Compound 1 morphic form. Some embodiments contain from about 5 wt. % to about 30 wt. %, from about 25 wt. % to about 50 wt. % or from about 5 wt. % to about 75 wt. % of a Compound 1 morphic form.

### EXAMPLES

### General Synthesis

The compounds described herein can be prepared by methods known by those skilled in the art. In one non-limiting example, the disclosed compounds can be made using the schemes below.

Compounds of the present invention with stereocenters may be drawn without stereochemistry for convenience. One skilled in the art will recognize that pure enantiomers and diastereomers can be prepared by methods known in the art. Examples of methods to obtain optically active materials include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the enantiomer is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step in the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved by chiral catalysts or chiral auxiliaries;
vi) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer;
vii) first- and second-order asymmetric transformations—a technique whereby diastereomers from the racemate quickly equilibrate to yield a preponderance in solution of the diastereomer from the desired enantiomer of where preferential crystallization of the diastereomer from the desired enantiomer perturbs the equilibrium such that eventually in principle all the material is converted to the crystalline diastereomer from the desired enantiomers. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase (including vial chiral HPLC). The stationary phase can be made of chiral material, or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is place in contact with a thin membrane barrier. The barrier may separate two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through;
xiv) simulated moving bed chromatography is used in certain embodiments. A wide variety of chiral stationary phases are commercially available.

The term “nucleophilic aromatic substitution” (S_{N}Ar) means a substitution reaction in which a nucleophile displaces a good leaving group, such as a halide (F, Cl, Br, I), tosylate, mesylate, triflate or the like, on an aromatic ring. Examples of the nucleophile in the S_{N}Ar reaction include, but not limited to, an amine, including aromatic amine, alcohol, phenol and aromatic compounds containing hydroxyl group attached to the aromatic ring. The nucleophilic aromatic substitution may be performed at decreased temperature (below 0° C.), at room temperature (about 25° C.), or increased temperature (about 30-200° C.). The reaction is performed in any suitable solvent including, but not limited to, DMSO, N,N-dimethylformamide, THF, N,N-dimethylacetamide, 1,4-dioxane, and acetonitrile. The reaction is performed in the presence of a base, such as an inorganic or organic base, including but not limited to, NaOH, KOH, Na₂CO₃, Na₃PO₄, Cs₂CO₃, Li₂CO₃, DIEA (N,N-diisopropylethylamine, or Hunig's base).

Synthesis of compounds according to the present invention may include steps of protection and deprotection of some reactive groups, such as amino group and carboxylic hydroxyl. Methods of protection and deprotection of amino and hydroxyl groups and corresponding protective groups are known to the skilled person and described, for example, in Greene's Protective Groups in Organic Synthesis, 5th Edition, P. G. M. Wuts, Wiley (2014).

### Example 1: Synthesis of tert-butyl (R)-3-(6-(2-cyano-3,6-difluorophenoxy)-4-oxoquinazolin-3(4H)-yl)-1-oxa-8-azaspiro[4.5]decane-8-carboxylate—Route 1 (Via Transamination)

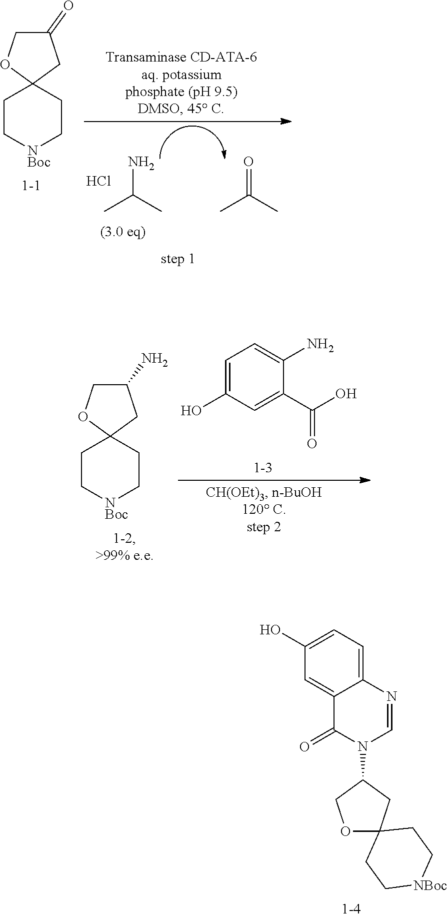

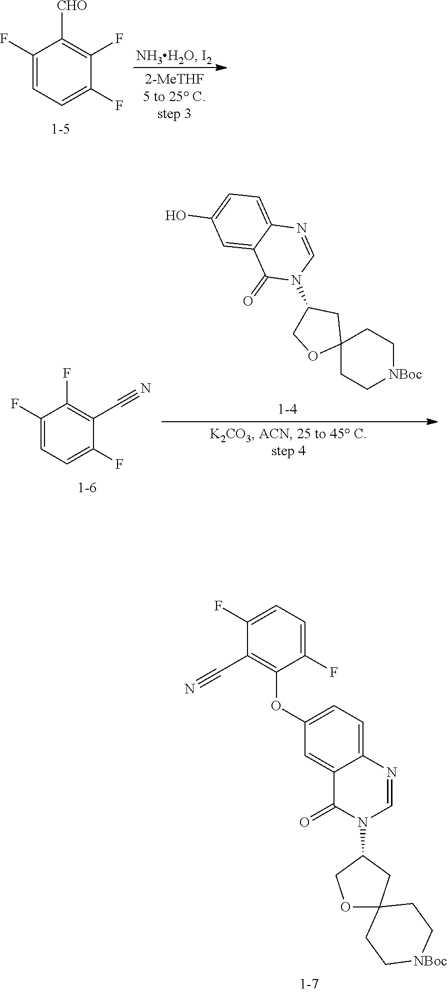 [SMILES: O=Cc1c(F)ccc(F)c1F ; N#Cc1c(F)ccc(F)c1F ; CC(C)(C)OC(=O)N1CCC2(CC1)C[C@@H](n1cnc3ccc(O)cc3c1=O)CO2 ; CC(C)(C)OC(=O)N1CCC2(CC1)C[C@@H](n1cnc3ccc(Oc4c(F)ccc(F)c4C#N)cc3c1=O)CO2]

Step 1 can be conducted at manufacturing scale to prepare large quantities of 1-2 for use in the preparation of Compound 1. A non-limiting example of this scaled up reaction and the raw material inputs is provided below.

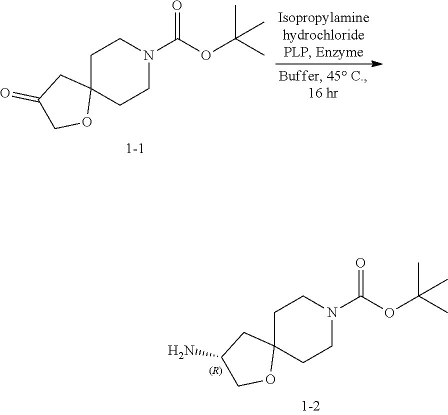

Raw Material List

| Material | CAS# | M.W. | Quantity | | Eq | |
|---|---|---|---|---|---|---|
| 1-1 | 954236-44-3 | 255.10 | 24.5 | kg | 1.00 | eq |
| propan-2-amine | 15572-56-2 | 95.57 | 26.8 | kg | 3.00 | eq |
| hydrochloride | | | | | | |
| PLP | 54-47-7 | 247.17 | 0.25 | kg | 1.03e−2 | eq |

| | | | | | |
|---|---|---|---|---|---|
| Enzyme: CD-ATA-6 | — | — | 1.3 | kg | — |
| KH₂PO₄ | 7778-77-0 | 136 | 6.8 | kg | — |
| KOH | 1310-58-3 | 56 | 3.1 | kg | — |
| Diatomaceous earth | — | — | 50.0 | kg | — |
| NaCl | 7647-14-5 | 58.4 | 20 | kg | — |
| (+)-D-pyroglutamic | 4042-36-8 | 129.1 | 23.8 | kg | — |
| NaOH | 1310-73-2 | 40.0 | 25 | kg | — |
| Sodium sulfate | 7757-82-6 | 142.0 | 50 | kg | — |
| DMSO | 67-68-5 | 78.1 | 100 | L | — |

Process Description
Operation Steps

| | |
|---|---|
| 1. | R1: enamel still (1000 L) with a mechanical stirring. |
| 2. | Charged water (500 L) into R1 at 25-30° C. |
| 3. | Charged KH₂PO₄(6.8 kg) into R1 at 25-30° C. |
| 4. | Charged KOH (3.1 kg) into R1 at 25-30° C. |
| 5. | The resulting mixture was heated to 45° C. under N₂ atmosphere. |
| 6. | Charged propan-2-amine hydrochloride (26.8 kg, 282.01 mol, 3 eq.) into R1 at 45° C. |
| 7. | Charged PLP (0.25 kg, 971.1 mmol, 1.03e−2 eq.) into R1 at 45° C. |
| 8. | The pH of reactions mixture was adjusted to 8.5 by aq. 1N KOH (20 L). |
| 9. | Charged Enzyme: CD-ATA-6 (1.3 kg) into resulting mixture. |
| 10. | Compound 1-1 (24.5 kg) was added the DMSO (100 L), then the mixture was |
| | added dropwise to the reaction mixture at 45° C. over 1 hr. |
| 11. | The resulting mixture was stirred at 45° C. (inner) for 16 hrs under N₂ atmosphere. |
| 12. | TLC (dichloromethane/methanol = 3/1, compound 1-1 (R_{f}) = 0.6, 1-2 (R_{f}) = 0.3) |
| | showed 1-2 was detected, and the compound 1-1 was consumed. |
| 13. | Two reactions were combined for work up. The reaction mixture was cooled to |
| | 20° C., after adjusted pH to 11 by 4N aq. KOH (20 L), and addition of |
| | dichloromethane (500 L), the mixture was vigorously stirred for 10 minutes. |
| 14. | The resulting mixture was centrifuged through diatomaceous earth (50.0 kg). |
| 15. | The filter was extracted two with dichloromethane (2 × 250 L) at pH was 11 by 4 |
| | Naq. KOH. |
| 16. | All the dichloromethane extracts were rinsed one time by aq. subsaturation NaCl |
| | 200 L (20 kg NaCl). |
| 17. | The organic phase was combined and dried over sodium sulfate (20.0 kg) with |
| | vigorous stirring. Filtration and concentration in vacuo afforded the crude product |
| | as brown oil. |
| 18. | R2: enamel still (2000 L) with a mechanical stirring. |
| 19. | Charged the ethyl alcohol (1000 L) into R2. |
| 20. | Charged all the crude product into R2. |
| 21. | The resulting mixture was added the (+)-D-pyroglutamic (23.8 kg) at 45° C. |
| | under N2 atmosphere. |
| 22. | Then the mixture was stirred for 1 hr at 65° C. under N₂ atmosphere. |
| 23. | The temperature was lowered to 20° C. at least and hold stirred always. After, the |
| | mixture was centrifuged, and afforded the filter cake as a white solid. |
| 24. | R3: enamel still (1000 L) with a mechanical stirring. |
| 25. | Charged the water (300 L) into R3. |
| 26. | Charged the NaOH (25 kg) into R3. |
| 27. | Charged the filter cake into R3. |
| 28. | The aqueous layer was extracted three with dichloromethane (3 × 100 L). |
| 29. | The organic phase was combined and dried over sodium sulfate (30.0 kg) with |
| | vigorous stirring. |
| 30. | The organic phase was filtered and concentrated in vacuo to afford Compound |
| | 1-2 (34.6 kg, 63.3% yield) as a pale-yellow solid. |

¹H NMR (400 MHz, CDCl₃), δ, ppm: 3.94 (m, 1H) 3.46-3.67 (m, 4H) 3.26-3.39 (m, 2H) 2.06 (m, 1H) 1.69-1.76 (m, 1H) 1.65 (m, 1H) 1.54-1.62 (m, 1H) 1.48-1.52 (m, 2H) 1.41-1.48 (m, 11H)

Step 2 can be conducted at manufacturing scale to prepare large quantities of 1-4 for use in the preparation of Compound 1. A non-limiting example of this scaled up reaction and the raw material inputs is provided below.

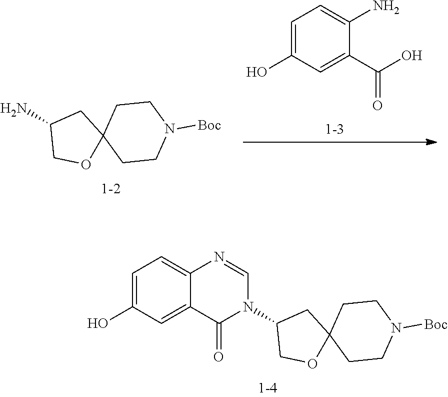 [SMILES: CC(C)(C)OC(=O)N1CCC2(CC1)C[C@@H](N)CO2 ; Nc1ccc(O)cc1C(=O)O ; CC(C)(C)OC(=O)N1CCC2(CC1)C[C@@H](n1cnc3ccc(O)cc3c1=O)CO2]

Raw Material List

| Material | CAS# | M.W. | Quantity | | Eq | |
|---|---|---|---|---|---|---|
| 1-2 | — | 256.34 | 33.0 | kg | 1.00 | eq |
| 1-3 | 394-31-0 | 153.14 | 20.7 | kg | 1.05 | eq |
| CH(OEt)₃ | 122-51-0 | 148.20 | 45.8 | kg | 2.40 | eq |
| NaHCO₃ | — | — | 18.0 | kg | — | |
| n-heptane | — | — | 667 | kg | 30.0 | V |
| n-BuOH | — | — | 267 | kg | 10.0 | V |

Process Description
Operation Steps

| | |
|---|---|
| 1. | R1: enamel still (1000 L) with a mechanical stirring and thermometer under N₂ |
| | protection at 25-30° C. |
| 2. | Charged n-BuOH (242 kg) into R1 at 25-30° C. |
| 3. | Charged Cpd. 1-2 (33.0 kg, 128 mol, 1.00 eq.) into R1 at 25-30° C. |
| 4. | Charged Cpd. 1-3 (20.7 kg, 135 mol, 1.05 eq.) into R1 at 25-30° C. |
| 5. | The resulting mixture was stirred at 110-115° C. (inner) for 30 min under N₂ |
| | atmosphere. |
| 6. | Charged CH(OEt)₃ (45.8 kg, 309 mol, 2.40 eq.) into R1 at 100-115° C. (inner). |
| 7. | The resulting mixture was stirred at 110-115° C. (inner) for 12 hrs under N₂ |
| | atmosphere. |
| 8. | GC showed that Cpd. 1-2 (Rt = 6.761 min) consumed and HPLC showed the |
| | desired peak (Rt = 8.054 min) was detected. |
| 9. | The mixture was concentrated to remove n-BuOH (187 kg) in vacuum at 55-60° C. |
| 10. | Charged sat. NaHCO₃ (18 kg NaHCO₃ dissolved in 165 kg H₂O) into R1 at 25- |
| | 30° C. and stirred at 25-30° C. for 1 hr. |
| 11. | R2: enamel still (2000 L) with a mechanical stirring and thermometer under N₂ |
| | protection at 25-30° C. |
| 12. | Charged the mixture into R2. |
| 13. | Charged n-heptane (703 kg) into R2 at 25-30° C. and stirred at 25-30° C. for 1 hr. |
| 14. | The mixture was filtered to give the filter cake. The filter cake was triturated |
| | with water (660 kg) at 25-30° C. for 0.5 hr and filtered to give the filter cake. |
| 15. | The cake was dried via vacuum oven at 45° C. for 48 hrs. Cpd. 1-4 (43.1 kg, 103 |
| | mol, 80.6% yield, 98.0% purity) was obtained as a brown solid and confirmed |
| | by HPLC. |

Step 3 can be conducted at manufacturing scale to prepare large quantities of 1-6 for use in the preparation of Compound 1. A non-limiting example of this scaled up reaction and the raw material inputs is provided below.

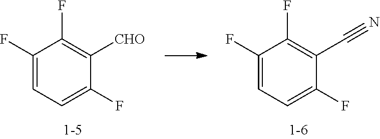 [SMILES: O=Cc1c(F)ccc(F)c1F ; N#Cc1c(F)ccc(F)c1F]

Raw Material List

| Material | CAS# | M.W. | Quantity | | Eq |
|---|---|---|---|---|---|
| Cpd. 1-5 | 104451-70-9 | 160.09 | 27.0 | kg | 1.00 eq |
| I₂ | 7553-56-2 | 153.81 | 47.0 | kg | 1.10 eq |
| NH₃•H₂O | 1336-21-6 | 35.05 | 393 | kg | 18.6 eq |
| Na₂SO₃ | — | — | 60.0 | kg | — |
| NaCl | — | — | 27.0 | kg | — |
| 2-MeTHF | — | — | 397 | kg | — |

Process Description
Operation Steps

| | |
|---|---|
| 1. | R1: enamel still (2000 L) with a mechanical stirring and thermometer under N₂ |
| | protection at 25-30° C. |
| 2. | Charged 2-MeTHF (232 kg) into R1 at 25-30° C. |
| 3. | Charged Cpd. 1-5 (27.0 kg, 168 mol, 1.00 eq.) into R1 at 25-30° C. |
| 4. | Charged I₂ (47.0 kg, 185 mol, 1.10 eq.) into R1 at 25-30° C. |
| 5. | The resulting mixture was cooled to 0-5° C. under N₂ atmosphere. |
| 6. | Charged NH₃•H₂O (393 kg, 3128 mol, 18.6 eq.) into R1 at 0-20° C. (inner) for 3 hrs. |
| 7. | The resulting mixture was stirred at 5-15° C. (inner) for 2 hrs under N₂ atmosphere. |
| 8. | HPLC showed that ~3% of Cpd. 1-5 (RT = 7.462 min) remained, and the desired |
| | peak (RT = 8.644 min) was detected. |
| 9. | Sat. Na₂SO₃ (60 kg Na₂SO₃ dissolved in 270 kg H₂O) was added to reaction |
| | mixture drop-wise at 0-10° C. (inner) and stirred at 0-10° C. (inner) for 0.5 hr. |
| 10. | The aqueous phase was extracted with 2-MeTHF (165 kg) at 0-10° C. The |
| | combined organic phases were adjusted to pH = 4 with 1M HCl and washed |
| | with brine (27 kg NaCl dissolved in 270 kg H₂O) at 0-10° C. to give a liquid. |
| 11. | Cpd. 1-6 (270 kg, 125 mol, 74.8% yield, 92.2% purity) was obtained as a yellow |
| | liquid, which was confirmed by HPLC and used in the next step directly. |

Step 4 can be conducted at manufacturing scale to prepare large quantities of 1-7 for use in the preparation of Compound 1. A non-limiting example of this scaled up reaction and the raw material inputs is provided below.

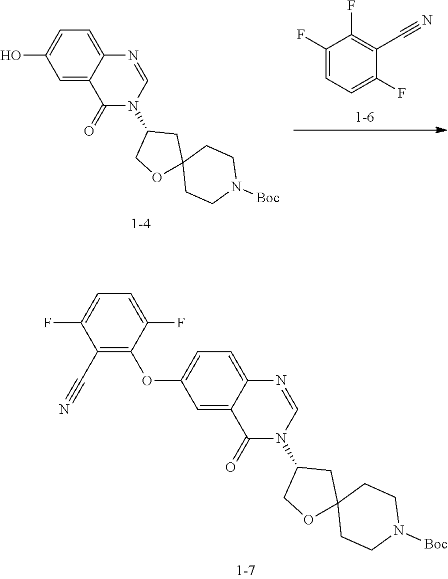 [SMILES: CC(C)(C)OC(=O)N1CCC2(CC1)C[C@@H](n1cnc3ccc(O)cc3c1=O)CO2 ; N#Cc1c(F)ccc(F)c1F ; CC(C)(C)OC(=O)N1CCC2(CC1)C[C@@H](n1cnc3ccc(Oc4c(F)ccc(F)c4C#N)cc3c1=O)CO2]

Raw Material List

| Material | CAS# | M.W. | Quantity | | Eq | |
|---|---|---|---|---|---|---|
| Cpd. 1-4 | | 256.34 | 43.1 | kg | 1.00 | eq |
| Cpd. 1-6 | 136514-17-5 | 157.09 | 222 | kg | 1.03 | eq |
| K₂CO₃ | 584-08-7 | 138.21 | 35.5 | | 2.50 | eq |
| MTEB | | | 960 | kg | | |
| DMSO | | | 99.0 | kg | | |
| NaCl | | | 40.0 | kg | | |
| ACN | | | 340 | kg | 10.0 | V |

Process Description
Operation Steps

| | |
|---|---|
| 1. | R1: enamel still (2000 L) with a mechanical stirring and thermometer under N₂ |
| | protection at 25-30° C. |
| 2. | Charged ACN (324 kg) into R1 at 25-30° C. |
| 3. | Charged Cpd. 1-4 (43.1 kg, 103 mol, 1.00 eq.) into R1 at 25-30° C. |
| 4. | Charged Cpd. 1-6 (222 kg, 106 mol, 1.03 eq.) into R1 at 25-30° C. |
| 5. | Charged K₂CO₃ (35.5 kg, 257 mol, 2.50 eq.) into R1 at 25-30° C. |
| 6. | The resulting mixture was stirred at 40-45° C. (inner) for 12 hrs under N₂ |
| | atmosphere. |
| 7. | HPLC showed that Cpd. 1-4 (RT = 8.034 min, 0.71%) remained and the desired |
| | peak (RT = 10.940 min) was detected. |
| 8. | The reaction mixture was cooled down 25-30° C., added into water (431 kg) and |
| | stirred at 25-30° C. for 0.5 hr. |
| 9. | The mixture was filtered through a pad of celite (30 kg). |
| 10. | The filtrate was extracted with MTBE (320 kg × 3). The combined extracts were |
| | washed with brine (40 kg NaCl dissolved in 400 kg H₂O), DMSO (99 kg) was |
| | added and concentrated to remove MTBE at 40-45° C. |
| 11. | Charged the mixture into water (900 kg) at 25-30° C. and stirred at 25-30° C. for |
| | 1 hr. |
| 12. | The mixture was filtered to give the filter cake. The filter cake was triturated |
| | with water (900 kg × 3) at 25-30° C. for 6 hrs and filtered to give the filter cake. |
| 13. | The cake was dried via vacuum oven at 45° C. for 48 hrs. |
| 14. | Compound 1-7 (39.0 kg, 69.0 mol, 73.3% yield, 95.3% purity) was obtained as a |
| | brown solid and confirmed by HPLC. |

### Example 2: Synthesis of tert-butyl (R)-3-(6-(2-cyano-3,6-difluorophenoxy)-4-oxoquinazolin-3(4H)-yl)-1-oxa-8-azaspiro[4.5]decane-8-carboxylate—Route 2 (Via Diastereomeric Crystallization)

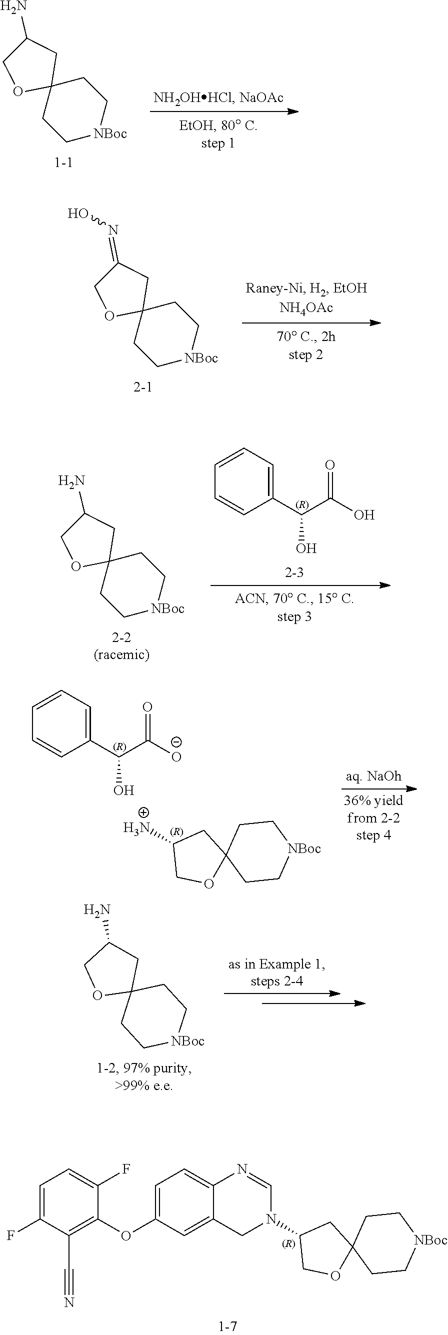

Step 1: Synthesis of tert-butyl 3-(hydroxyimino)-1-oxa-8-azaspiro[4.5]decane-8-carboxylate (2-1). To a solution of tert-butyl 3-oxo-1-oxa-8-azaspiro[4.5]decane-8-carboxylate (1-1, 30 g, 117.50 mmol) in ethanol (450 mL) was added sodium acetate (14.46 g, 176.26 mmol) and hydroxylamine hydrochloride (12.25 g, 176.26 mmol). The mixture was stirred at 70° C. for 1 h. The reaction mixture was concentrated to remove EtOH, diluted with ethyl acetate (300 mL) and water (300 mL). The aqueous layer was extracted with ethyl acetate (2×150 mL), the combined organic phases were washed with saturated sodium bicarbonate (aq., 300 mL), water (300 mL), brine (300 mL), dried over sodium sulfate, filtered and concentrated in vacuo to give tert-butyl 3-(hydroxyimino)-1-oxa-8-azaspiro[4.5]decane-8-carboxylate (2-1, 32 g, crude) as light yellow oil.

¹H NMR (400 MHz, CDCl₃) δ 8.71-7.60 (m, 1H), 4.52 (s, 1H), 4.38 (s, 1H), 3.74-3.57 (m, 2H), 3.41-3.26 (m, 2H), 2.58 (s, 1H), 2.50 (s, 1H), 1.77-1.66 (m, 2H), 1.65-1.54 (m, 2H), 1.46 (s, 9H).

Step 2: Synthesis of tert-butyl 3-amino-1-oxa-8-azaspiro[4.5]decane-8-carboxylate (2-2). To a solution of tert-butyl (3E)-3-hydroxyimino-1-oxa-8-azaspiro[4.5]decane-8-carboxylate (2-1, 32 g, 118.38 mmol) in ethanol (600 mL) was added Raney-Ni (6.00 g, 70.03 mmol) and ammonium acetate (19.1 g, 247.79 mmol). The mixture was stirred at 70° C. for 2 h under hydrogen atmosphere (15 psi). The reaction mixture was filtered through celite, and the filtrate was concentrated. The residue was dissolved in aq. hydrochloric acid (1M, 150 mL), the resulting mixture was washed with methyl tert-butyl ether (2×150 mL), then aq. sodium hydroxide (4 M) was added and adjusted to pH=12. The aqueous layer was extracted with dichloromethane (3×150 mL), the combined organic phases were washed with brine (100 mL), dried over sodium sulfate, filtered and concentrated to give tert-butyl 3-amino-1-oxa-8-azaspiro[4.5]decane-8-carboxylate (2-2, 22.2 g, 86.60 mmol, 73.16% yield) as a light yellow solid.

¹H NMR (400 MHz, CDCl₃) δ 4.02-3.85 (m, 1H), 3.70-3.43 (m, 4H), 3.39-3.21 (m, 2H), 2.05 (br dd, J=7.5, 12.7 Hz, 1H), 1.78-1.53 (m, 3H), 1.51-1.33 (m, 13H)

Step 3: Synthesis of (R)-8(tert-butoxycarbonyl)-1-oxa-8-azaspiro[4.5]decan-3-aminium (R)-2-hydroxy-2-phenylacetate (2-4) (D-Mandelic acid salt). To a mixture of tert-butyl 3-amino-1-oxa-8-azaspiro[4.5]decane-8-carboxylate (2-2, 25 g, 97.53 mmol) in acetonitrile (350 mL) was added (2R)-2-hydroxy-2-phenyl-acetic acid (2-3, 7.42 g, 48.76 mmol) in portions at 15° C. (ambient temperature), precipitates formed and then the mixture was stirred for 2 h at 70° C. After cooling slowly to 15° C., the suspension was stirred for further 12 h at 15° C. The precipitate was filtered, washed with acetonitrile (100 mL), and dried under vacuum. (R)-8-(tert-butoxycarbonyl)-1-oxa-8-azaspiro[4.5]decan-3-aminium (R)-2-hydroxy-2-phenylacetate (2-4, 19.5 g) was obtained.

¹H NMR (400 MHz, METHANOL-d4) δ=7.52-7.45 (m, 2H), 7.35-7.28 (m, 2H), 7.28-7.21 (m, 1H), 4.89 (br s, 1H), 4.01 (dd, J=5.4, 10.1 Hz, 1H), 3.91-3.79 (m, 2H), 3.65 (tdd, J=4.5, 8.7, 13.1 Hz, 2H), 3.32-3.21 (m, 2H), 2.24 (dd, J=8.0, 13.9 Hz, 1H), 1.80-1.62 (m, 4H), 1.55-1.49 (m, 1H), 1.48 (s, 9H).

¹H NMR (400 MHz, DMSO-d6) δ=7.37 (d, J=7.3 Hz, 2H), 7.28-7.21 (m, 2H), 7.20-7.13 (m, 1H), 4.57 (s, 1H), 3.86 (dd, J=6.0, 9.5 Hz, 2H), 3.71 (ddd, J=5.6, 8.0, 10.9 Hz, 2H), 3.62 (dd, J=4.9, 9.5 Hz, 2H), 3.42 (qd, J=4.2, 13.1 Hz, 3H), 3.19 (br d, J=10.3 Hz, 3H), 2.05 (dd, J=8.1, 13.4 Hz, 1H), 1.66-1.54 (m, 3H), 1.54-1.45 (m, 1H), 1.45-1.40 (m, 1H), 1.39 (s, 9H).

Step 4: Synthesis of tert-butyl (3R)-3-amino-1-oxa-8-azaspiro[4.5]decane-8-carboxylate (1-2). (R)-8-(tert-butoxycarbonyl)-1-oxa-8-azaspiro[4.5]decan-3-aminium (R)-2-hydroxy-2 phenylacetate 2-4 was suspended in water (200 mL), and neutralized with aq. sodium hydroxide (2M, 25 mL). The mixture was extracted with ethyl acetate (3×250 mL), the combined organic layers were washed with brine (3×100 mL), dried over sodium sulfate, filtered and concentrated under vacuum to give tert-butyl (3R)-3-amino-1-oxa-8-azaspiro[4.5]decane-8-carboxylate (1-2, 10.3 g, 95.3% e.e.) as white solid.

Steps 3 and 4 can be repeated to obtain 1-2 with higher enantiomeric purity To a mixture of tert-butyl (3R)-3-amino-1-oxa-8-azaspiro[4.5]decane-8-carboxylate (1-2, 10.3 g, 39 mmol) in acetonitrile (350 mL) was added (2R)-2-hydroxy-2-phenyl-acetic acid (2-3, 5.76 g, 37.8 mmol) in portions at 15° C. (ambient temperature), precipitates formed and then the mixture was stirred for 2 h at 70° C. After cooling slowly to 15° C., the suspension was stirred for further 12 h at 15° C. The precipitate was filtered, washed with acetonitrile (40 mL), and dried under vacuum. (3 g was kept in hand). The rest of D-Mandelic acid salt was suspended in water (80 mL), neutralized with aq. sodium hydroxide (2M, 20 mL) and extracted with ethyl acetate (3×150 mL). The combined organic layers were washed with brine (3×50 mL), dried over sodium sulfate, filtered and concentrated under vacuum to give tert-butyl (3R)-3-amino-1-oxa-8-azaspiro[4.5]decane-8-carboxylate (1-2, 6.5 g, 23.80 mmol, 99.5% e.e.) as white solid.

¹H NMR (400 MHz, CDCl₃) δ 3.93 (dd, J=5.8, 8.8 Hz, 1H), 3.72-3.43 (m, 4H), 3.41-3.23 (m, 2H), 2.05 (dd, J=7.3, 12.6 Hz, 1H), 1.79-1.53 (m, 4H), 1.52-1.34 (m, 13H).

### Stereochemistry Determination of Compound 2-4.

The mandelic acid salt 2-4 in Example 2 was determined by X-ray crystallography to be (R)-8-(tert-butoxycarbonyl)-1-oxa-8-azaspiro[4.5]decan-3-aminium (R)-2-hydroxy-2-phenylacetate. (See Example 32 for single crystal X-ray crystallography structure determination) This intermediate 2-4 was carried on in the synthesis of 1-7, which based on mechanism of the subsequent reactions was also the R-isomer.

The stereochemistry of Compound 1-7 obtained in Example 2(Route 2) was then compared to that from Example 1 (Route 1) using chiral SFC and polarimetry.

### Chiral SFC Method:

Column: CHIRALPAK® AD-3, 50×4.6 mm I.D., 3 um;
Mobile phase: Phase A for CO₂, and Phase B for MeOH (0.05% DEA);
Gradient elution: 40% MeOH (0.05% DEA) in CO₂;
Flow rate: 3 mL/min;
Detector: PDA;
Column Temp: 35° C.;
Back Pressure: 100 bar.
Retention time for 1-7 (Route 2)=1.70 min (late eluting peak).
Retention time for 1-7 (Route 1)=1.69 min (late eluting peak).
Retention time for racemic 1-7 (control)=0.96 min (early eluting peak) and 1.77 min (late eluting peak).
Specific rotation for 1-7 (Route 2) [α]_{D}²⁵=−67.310°.
Specific rotation for 1-7 (Route 1) [α]_{D}²⁵=−71.501°.

Since intermediate 1-7 from both Route 1 and Route 2 was the late eluting peak on the chiral SFC (identical method) and has the same negative sign for specific rotation, Compound 1-7 in Route 1 was determined to be the R-isomer. This also confirms that the absolute stereochemistry of Compound 1 is R as depicted below.

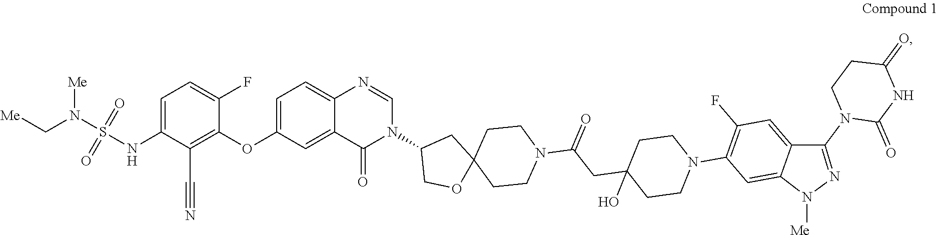 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

### Example 3: Synthesis of 2-(1-(3-(2,4-dioxotetrahydropyrimidin-1(2H)-yl)-5-fluoro-1-methyl-1H-indazol-6-yl)-4-hydroxypiperidin-4-yl)acetic acid

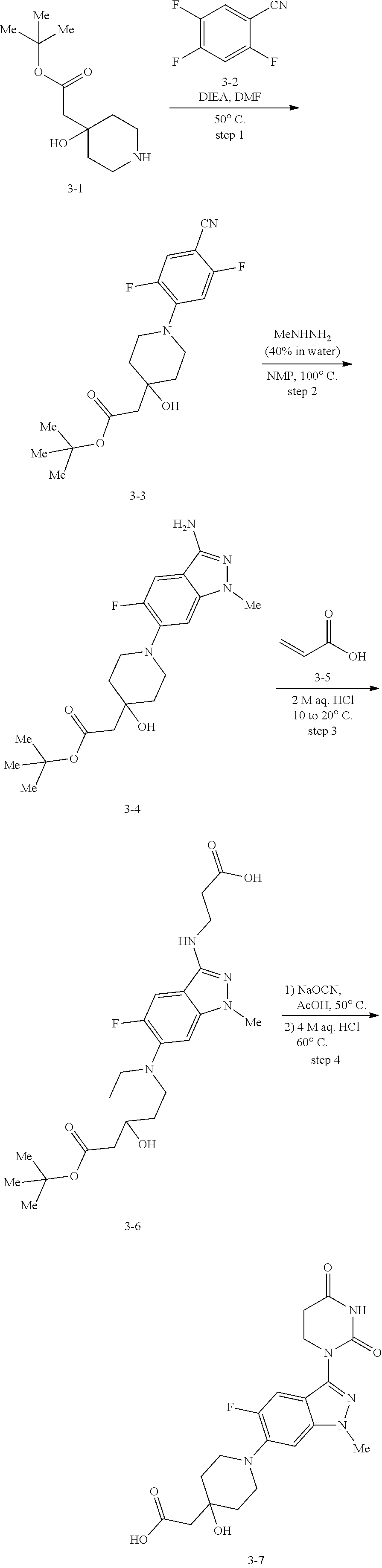

Step 1 can be conducted at manufacturing scale to prepare large quantities of 3-3 for use in the preparation of Compound 1. A non-limiting example of this scaled up reaction and the raw material inputs is provided below.

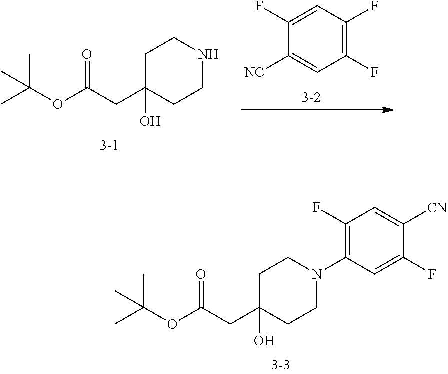 [SMILES: CC(C)(C)OC(=O)CC1(O)CCNCC1 ; N#Cc1cc(F)c(F)cc1F ; CC(C)(C)OC(=O)CC1(O)CCN(c2cc(F)c(C#N)cc2F)CC1]

Raw Material List

| Material | CAS# | M.W. | Quantity | | Eq |
|---|---|---|---|---|---|
| 3-1 | — | 215.29 | 34.0 | kg | 1.00 eq |
| 3-2 | 98349-22-5 | 157.09 | 24.8 | kg | 1.00 eq |
| DIEA | 7087-68-5 | 129.24 | 30.6 | kg | 1.50 eq |
| DMF | — | — | 180 | kg | — |

Process Description
Operation Steps

| | |
|---|---|
| 1. | Charged DMF (180 kg) into R1 at 25-30° C. |
| 2. | Charged 3-1 (34.0 kg, 157.93 mol; 1.00 eq.) into R1 at 25-30° C. |
| 3. | Charged DIEA (30.62 kg, 236.89 mol; 1.50 eq.) into R1 at 25-30° C. |
| 4. | Charged a solution of 3-2 (24.81 kg; 157.93 mol; 1.00 eq.) in DMF (20 kg) into R1 drop- |
| | wise at 30-40° C. |
| 5. | The resulting mixture was stirred at 50-70° C. (inner) for 2 hrs under N₂ atmosphere. |
| 6. | HPLC showed 3-2 (Rₜ = 8.39 min) was consumed and HPLC showed the desired peak (Rₜ = |
| | 11.1 min) was detected. |
| 7. | The resulting mixture was cooled to 25° C. |
| 8. | R2: enamel still (2000 L) with a mechanical stirring and thermometer under N₂ protection |
| | at 25-30° C. |
| 9. | Charged water (1000 kg) into R2 at 25-30° C. and stirred at 25-30° C. |
| 10. | Charged the reaction mixture into R2 at 25-30 ° C. and stirred at 25-30 ° C. for 1 hr. |
| 11. | The mixture was centrifuged and the filtered cake was washed by water (200 kg) to give the |
| | filter cake. |
| 12. | The cake was dried via vacuum oven at 55° C. for 48 hrs. |
| 13. | Cpd. 3-3 (61.8 kg, 140.31 mol, 90.0% yield, 96% purity, 20% water content) was obtained |
| | as a yellow solid. |

Step 2 can be conducted at manufacturing scale to prepare large quantities of 34 for use in the preparation of Compound 1. A non-limiting example of this scaled up reaction and the raw material inputs is provided below.

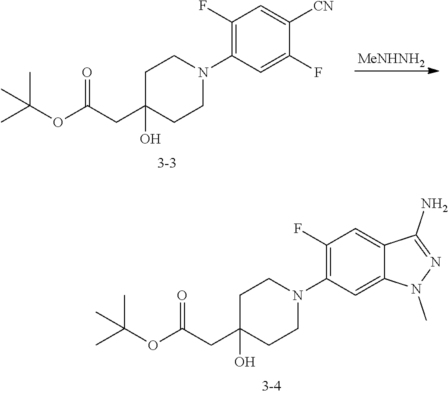 [SMILES: "MeNHNH2" ; CC(C)(C)OC(=O)CC1(O)CCN(c2cc(F)c(C#N)cc2F)CC1 ; Cn1nc(N)c2cc(F)c(N3CCC(O)(CC(=O)OC(C)(C)C)CC3)cc21]

Raw Material List

| Material | CAS# | M.W | Quantity | | Eq |
|---|---|---|---|---|---|
| Cpd. 3-3 | — | 352.37 | 61.8 | kg | 1.00 eq |
| MeNHNH2 | 60-34-4 | 46.07 | 161.6 | kg | 8.00 eq |
| NMP | 872-50-4 | — | 420 | kg | — |

Process Description
Operation Steps

| | |
|---|---|
| 1. | R1: enamel still (1000 L) with a mechanical stirring and thermometer under N₂ protection |
| | at 25-30° C. |
| 2. | Charged NMP (420 kg) into R1 at 25-30° C. |
| 3. | Charged Cpd. 3-3 (61.8 kg, 175.38 mol, 1.00 eq.) into R1 at 25-30° C. |
| 4. | Charged MeNHNH₂ (161.6 kg, 1400 mol, 8.00 eq.) into R1 at 25-30° C. |
| 5. | The resulting mixture was stirred at 110-120° C. (inner) for 24 hrs under N₂ atmosphere. |
| 6. | HPLC showed Cpd. 3-3 (Rt = 11.7 min) was consumed and one desired peak (Rt = 7.41 |
| | min) was detected. |
| 7. | The resulting mixture was cooled to 30° C. |
| 8. | R2: enamel still (3000 L) with a mechanical stirring and thermometer under N₂ protection |
| | at 25-30° C. |
| 9. | Charged water (1500 kg) into R2. Charge the mixture into R2 at 25-30° C. |
| 10. | Charged a solution of aq. citric acid (citric acid/H₂O = 98 kg/200 kg) into R2 drop-wise and |
| | stirred at 25-30° C. |
| 11. | Extracted with EtOAc (320 kg × 4). The combined organic phase was washed with aq. |
| | NaHCO₃ (NaHCO₃/water = 50 kg/400 kg). |
| 12. | The organic phase was concentrated in vacuum to 120~150 L at 50° C. |
| 13. | Charged solvent (MTBE/n-heptane = 10/1, 133 kg/13.7 kg) and concentrated in vacuum |
| | 120~150 L at 50° C., repeat 2 times. |
| 14. | Triturated with solvent (MTBE/n-heptane = 10/1, 66.6 kg/7 kg at 50° C. for 2 hrs. Cooled to |
| | 25° C. and filtered. |
| 15. | The cake was dried via vacuum oven at 50° C. for 12 hrs. |
| 16. | Cpd. 3-4 (37.8 kg, 99.88 mol, 71.0% yield, 97.0% purity) was obtained as a yellow solid. |

Step 3 can be conducted at manufacturing scale to prepare large quantities of 3-6 for use in the preparation of Compound 1. A non-limiting example of this scaled up reaction and the raw material inputs is provided below.

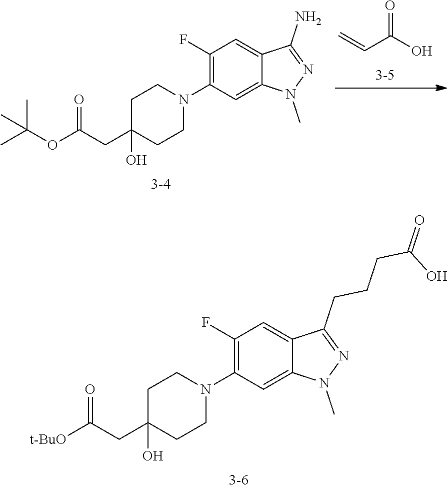

Raw Material List

| Material | CAS# | M.W. | Quantity | | Eq |
|---|---|---|---|---|---|
| Cpd. 3-4 | — | 378.44 | 32.0 | kg | 1.00 eq |
| acrylic acid | 25302-81-2 | 72.06 | 160 | kg | — |
| HCl | — | | 5.9 | kg | — |
| H₂O | — | — | 26.7 | kg | — |

Process Description
Operation Steps

| | |
|---|---|
| 1. | R1: enamel still (300 L) with a mechanical stirring and thermometer under N₂ protection at |
| | 25-30° C. |
| 2. | Charged acrylic acid (160 kg) into R1. |
| 3. | Charged HCl (5.9 kg)/H₂O (26.7 kg) into R1 at 10° C. |
| 4. | Then Cpd. 3-4 (32.0 kg, 84.6 mol, 1.00 eq.) was added in 5 portions for 30 min at 10~20° C. |
| 5. | The mixture was stirred at 15~20° C. for 31 hrs. |
| 6. | HPLC showed ~15% (Rt = 7.04 min) of Cpd. 3-4 remained. Several new peaks were shown |
| | on HPLC and ~70% (Rt = 7.16 min) of desired compound was detected. |
| 7. | The reaction mixture was cooled to 5~10° C., and 22.4 L ethyl acetate (0.7 V) was added. |
| 8. | R2: enamel still (1000 L) with a mechanical stirring and thermometer under N₂ protection |
| | at 25-30° C. |
| 9. | Charged the mixture into R2. The mixture was quenched by sat. Na₂CO₃ (Na₂CO₃/H₂O = |
| | 94 kg/400 kg) at 10~20° C. and stirred at 20° C. for 1 hr. |
| 10. | The resulting mixture was filtered, and the filtered cake was collected. The filtered cake |
| | was triturated with water (30 kg) at 25-30° C. for 0.5 hr and filtered to give the filter cake. |
| 11. | The filter cake was triturated with i-PrOH/i-PrOAc (4/1, 100 kg/33 kg) in R1 at 60° C. for |
| | 3 hrs. The resulting mixture was filtered, and the filter cake was collected. |
| 12. | The cake was dried via vacuum oven at 45° C. for 24 hrs. |
| 13. | Cpd. 3-6 (20.4 kg, 1.61 mol, 54% yield, 96.5% purity) was obtained as an off-white solid. |

Step 4 can be conducted at manufacturing scale to prepare large quantities of 3-7 for use in the preparation of Compound 1. A non-limiting example of this scaled up reaction and the raw material inputs is provided below.

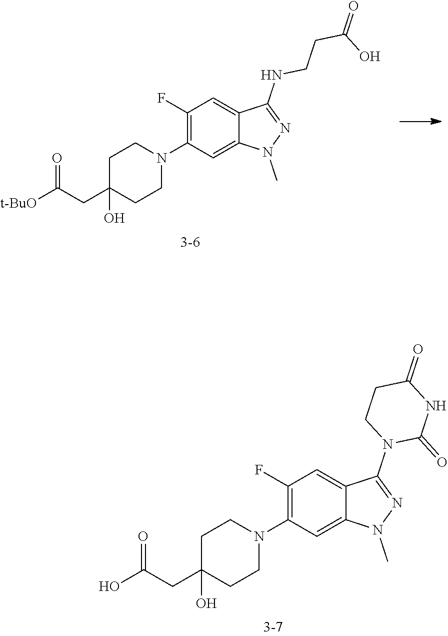 [SMILES: Cn1nc(NCCC(=O)O)c2cc(F)c(N3CCC(O)(CC(=O)OC(C)(C)C)CC3)cc21 ; Cn1nc(N2CCC(=O)NC2=O)c2cc(F)c(N3CCC(O)(CC(=O)O)CC3)cc21]

Raw Material List

| Material | CAS# | M.W. | Quantity | | Eq |
|---|---|---|---|---|---|
| Cpd. 3-6 | — | 450.50 | 20.4 | kg | 1.00 eq |
| AcOH | 64-19-7 | — | 107 | kg | — |
| NaOCN | — | 69.04 | 6.25 | kg | 2.00 eq |
| HCl | — | N/A | 8.0 | kg | — |
| H₂O | — | N/A | 13.6 | kg | — |

Process Description
Operation Steps

| | |
|---|---|
| 1. | R1: enamel still (300 L) with a mechanical stirring and thermometer under N₂ protection at |
| | 25-30° C. |
| 2. | Charged AcOH (107 kg) into R1 at 25° C. |
| 3. | Charged Cpd. 3-6 (20.4 kg; 45.28 mol; 1.00 eq.) into R1 at 25° C. |
| 4. | Charged NaOCN (6.25 kg, 90.57 mol, 2.00 eq. ) into R1 at 25° C. |
| 5. | The mixture was stirred at 50° C. for 16 hrs. |
| 6. | HPLC showed ~0.9% (Rₜ = 7.12 min) of Cpd. 3-6 remained. Several new peaks were shown |
| | on HPLC and ~68.9% (Rₜ = 6.90 min) of intermediate was detected. |
| 7. | Charged HCl (HCl/H₂O = 8 kg/13.6 kg) into R1 at 25° C. |
| 8. | The mixture was stirred at 60° C. for 48 hrs. |
| 9. | HPLC (reaction mixture) showed ~0% of intermediate remained. Several new peaks were |
| | shown on HPLC and ~86% (Rₜ = 4.98 min) of desired compound was detected. |
| 10. | The reaction mixture was cooled to 5~10° C. |
| 11. | R2: enamel still (2000 L) with a mechanical stirring and thermometer under N₂ protection |
| | at 25-30° C. |
| 12. | Charged the mixture into R2. The mixture was quenched by sat. NaHCO₃ (68.3 kg, 1.1M, |
| | 745 L, 18 eq.). The solution was added slowly for 45 min at 10~20° C. |
| 13. | The resulting mixture was filtered, and the filer cake was collected. |
| 14. | The powder was triturated with MeOH (40 L) in R1 at 60° C. for 4 hrs. The resulting mixture |
| | was filtered, and the filer cake was collected. |
| 15. | The powder was triturated with H₂O (80 L) in R1 at 100° C. for 4 hrs. The resulting mixture |
| | was filtered, and the filter cake was collected. |
| 16. | The cake was dried via vacuum oven at 45° C. for 48 hrs. |
| 17. | Compound 3-7 (13.2 kg, 1.20 mol, 72.0% yield, 98.7% purity) was obtained as an off-white |
| | solid. |

### Example 4: Synthesis of (3R)-3-[6-[2-cyano-3-[[ethyl(methyl)sulfamoyl]amino]-6-fluorophenoxy]-4-oxoquinazolin-3-yl]-8-[2-[1-[3-(2,4-dioxo-1,3-diazinan-1-yl)-5-fluoro-1-methylindazol-6-yl]-4-hydroxypiperidin-4-yl]acetyl]-1-oxa-8-azaspiro[4.5]decane (Compound 1)

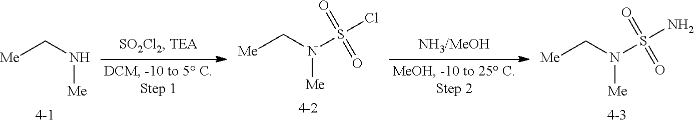

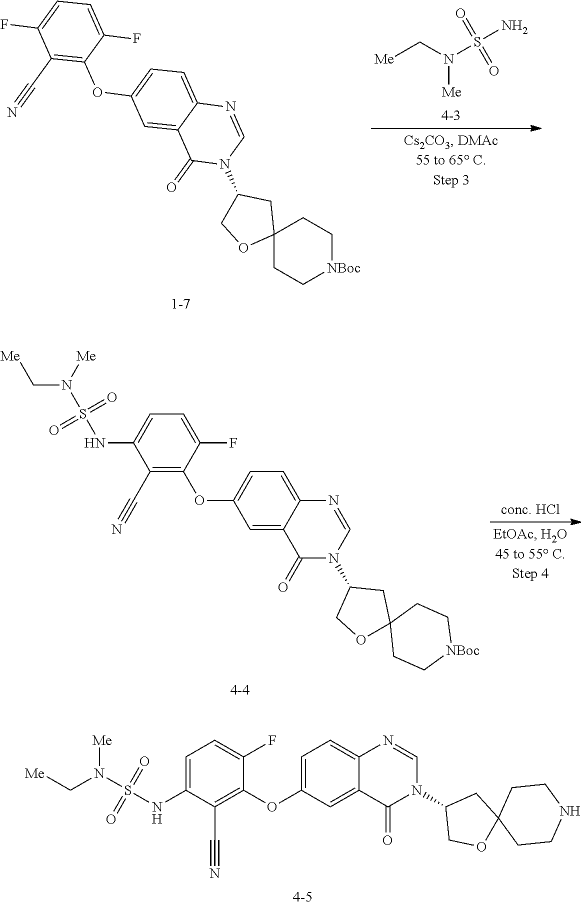 [SMILES: CC(C)(C)OC(=O)N1CCC2(CC1)C[C@@H](n1cnc3ccc(Oc4c(F)ccc(F)c4C#N)cc3c1=O)CO2 ; CCN(C)S(N)(=O)=O ; CCN(C)S(=O)(=O)Nc1ccc(F)c(Oc2ccc3ncn([C@H]4COC5(CCN(C(=O)OC(C)(C)C)CC5)C4)c(=O)c3c2)c1C#N ; CCN(C)S(=O)(=O)Nc1ccc(F)c(Oc2ccc3ncn([C@H]4COC5(CCNCC5)C4)c(=O)c3c2)c1C#N]

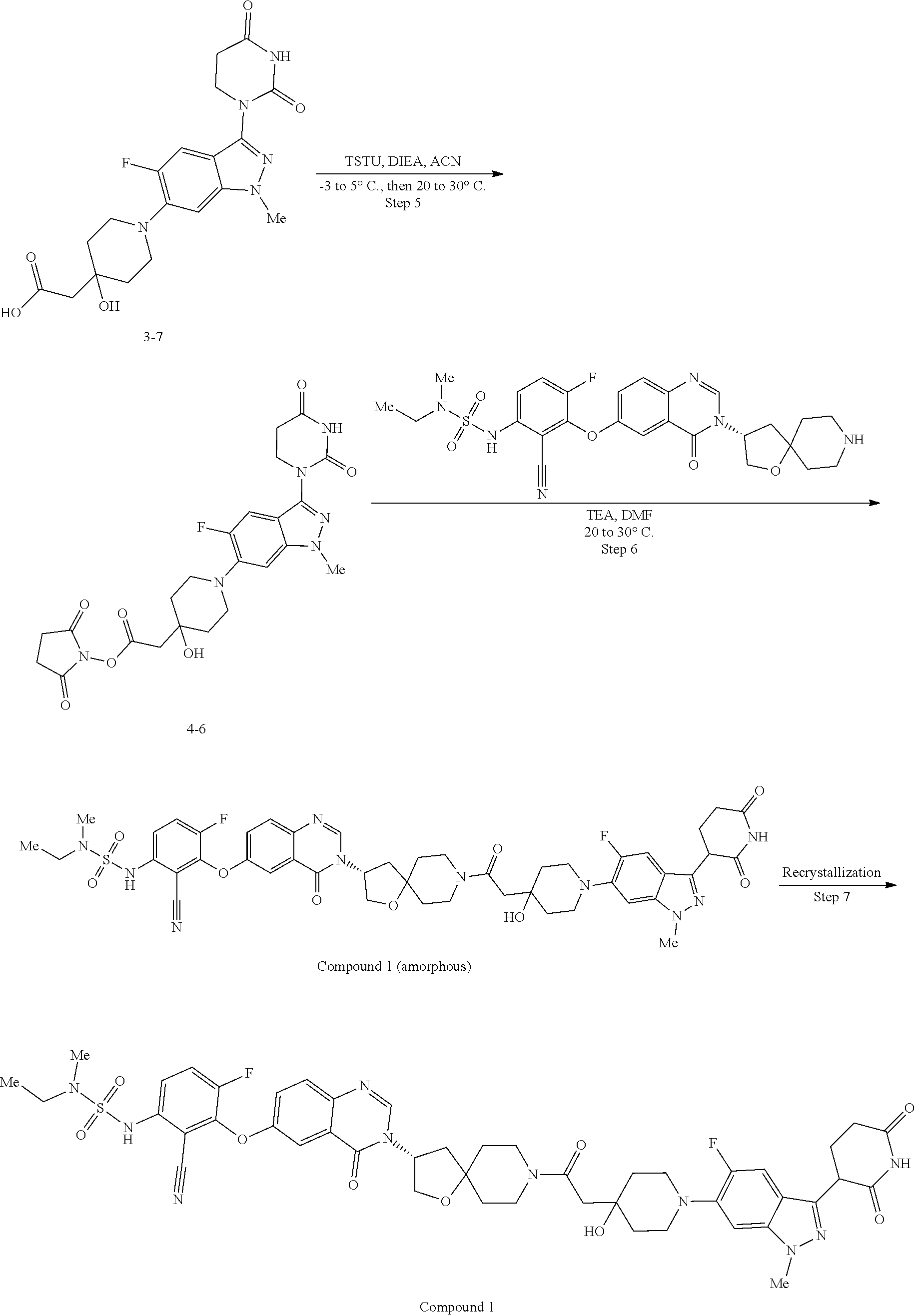 [SMILES: Cn1nc(N2CCC(=O)NC2=O)c2cc(F)c(N3CCC(O)(CC(=O)O)CC3)cc21 ; Cn1nc(N2CCC(=O)NC2=O)c2cc(F)c(N3CCC(O)(CC(=O)ON4C(=O)CCC4=O)CC3)cc21 ; CCN(C)S(=O)(=O)Nc1ccc(F)c(Oc2ccc3ncn([C@H]4COC5(CCNCC5)C4)c(=O)c3c2)c1C#N ; CCN(C)S(=O)(=O)Nc1ccc(F)c(Oc2ccc3ncn([C@H]4COC5(CCN(C(=O)CC6(O)CCN(c7cc8c(cc7F)c(C7CCC(=O)NC7=O)nn8C)CC6)CC5)C4)c(=O)c3c2)c1C#N ; CCN(C)S(=O)(=O)Nc1ccc(F)c(Oc2ccc3ncn([C@H]4COC5(CCN(C(=O)CC6(O)CCN(c7cc8c(cc7F)c(C7CCC(=O)NC7=O)nn8C)CC6)CC5)C4)c(=O)c3c2)c1C#N]

Compound 1 was prepared in seven steps from three starting materials, 4-1, 1-7, and 3-7, as shown in scheme above. Starting material 4-1 was treated with sulfuryl chloride (SO₂Cl₂) in the presence of triethylamine (TEA) to afford sulfamoyl chloride 4-2, which reacted with ammonia in the presence of methanol (NH₃/MeOH) to produce sulfamide 4-3. A nucleophilic aromatic substitution (S_{N}Ar) of 1-7 (>99% e.e.) with 4-3 afforded intermediate 4-4 with no loss of its enantiomeric purity (>99% e.e.). A deprotection of the Boc group in 4-4 using HCl afforded intermediate 4-5 as a free base. Treatment of 3-7 with TSTU produced its activated form 4-6. An amide bond formation between 4-5 and 4-6 in the presence of DIEA yielded Compound 1 as an amorphous solid. Recrystallization of the amorphous Compound 1 from a mixture of acetone and water affords Compound 1 drug substance in >99% e.e. as a crystalline solid.

Compound 1 drug substance was manufactured in seven steps and the general process is described below.

Step 1: Starting material 4-1 was treated with sulfuryl chloride and triethylamine in dichloromethane between −10 to 5° C. to generate sulfamoyl chloride 4-2. After the reaction was complete, water was slowly charged into the reaction mixture. The resulting organic layer was collected, washed with water, and concentrated under vacuum to afford 4-2 as a dichloromethane solution.

Step 2: Sulfamoyl chloride 4-2 in dichloromethane was added into a solution of ammonia in methanol between −10 to 0° C., followed by warming the reaction to 15 to 25° C., to produce sulfamide 4-3. After the reaction was complete, the reaction mixture was concentrated under vacuum and was subjected to an aqueous work-up with ethyl acetate and water. A solvent switch was performed to isolate 4-3 as a dimethylacetamide solution.

Step 3: A mixture of starting material 1-7, sulfamide 4-3, and cesium carbonate in dimethylacetamide was stirred at 55 to 65° C. to generate intermediate 4-4. After the reaction reached the target conversion, water was charged into the reaction mixture at 20 to 30° C. and filtration was performed to remove residual solid. The resulting aqueous solution was washed with methyl tert-butyl ether, neutralized with 1N hydrochloric acid, and extracted with ethyl acetate. The product-containing ethyl acetate layer was passed through a CUNO filter. The filtered solution was concentrated under vacuum to afford 4-4 as an ethyl acetate solution with the chiral purity intact (>99% e.e.).

Step 4: Intermediate 4-4 in ethyl acetate was treated with a hydrochloric acid aqueous solution at 45 to 55° C. to afford the deprotected form 4-5. After the reaction was complete, water was charged into the reaction mixture followed by the addition of a 20% sodium carbonate aqueous solution, adjusting pH of the reaction mixture to ˜9. Free base 4-5 was isolated as a solid by filtration.

Step 5: Starting material 3-7 was treated with N,N,N′N′-tetramethyl-O—(N-succinimidyl)uronium tetrafluoroborate (TSTU) in the presence of diisopropylethylamine (DIEA) in acetonitrile, first at −5 to 5° C. followed by warming to 20 to 30° C., to produce the activated form 4-6. After the reaction reached the target conversion, the resulting solid was collected by filtration. The filtered solid was washed by water followed by acetonitrile. The washed solid was dried to provide 4-6 as a white solid.

Step 6: A mixture of intermediates 4-5 and 4-6 in dimethylformamide was treated with triethylamine and was stirred at 20 to 30° C. to generate Compound 1. After the reaction reached the target conversion, the reaction mixture was added into a second reactor containing water. The resulting solid was first collected by filtration and was redissolved in dichloromethane. The product-containing dichloromethane layer was passed through a CUNO filter. The filtered solution was concentrated under vacuum. Trituration of the concentrated material from a dimethylformamide-water mixture afforded Compound 1 as an amorphous solid.

Step 7: Amorphous Compound 1 was recrystallized from a mixture of acetone, water, and ethanol to afford crystalline Compound 1 drug substance. Amorphous Compound 1 was first dissolved in a mixture of acetone and water at 48 to 58° C. and was then treated with a small amount of crystalline Compound 1 as the seed at 35 to 45° C. After stirring for several hours, ethanol was slowly added into the mixture. The resulting mixture was cooled to −3 to 3° C. and was stirred at that temperature for several hours. Filtration followed by washing with ethanol and drying afforded Compound 1 drug substance as a crystalline solid. In certain aspects the Compound 1 crystalline solid is Compound 1 Form B,

Step 1 can be conducted at manufacturing scale to prepare large quantities of 4-2 for use in the preparation of Compound 1. A non-limiting example of this scaled up reaction is provided below.

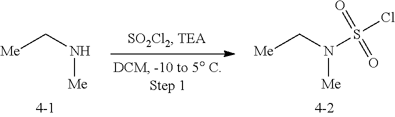

Process Description
Operation Steps

| | |
|---|---|
| 1. | Charged SO₂Cl₂ (7.53 kg, 1.0 eq.) into R1. |
| 2. | Charged DCM (16.5 L, 5 V) into R1. |
| 3. | Adjusted R1 to −10~0° C. |
| 4. | Charged DCM (16.5 L, 5 V) into R2. |
| 5. | Charged 4-1 (3.30 kg, 1.0 eq.) into R2. |
| 6. | Charged TEA (5.65 kg, 1.0 eq.) into R2. |
| 7. | Charged mixture solution in R2 into R1 dropwise over 8 h |
| | at −10~0° C. |
| 8. | Stirred R1 for 4 hr at −5~5° C. |
| 9. | Charged H₂O (16.5 L, 5 V) into R1 dropwise over 30 min. |
| 10. | Stirred R1 for 1 hr at 15-25° C. |
| 11. | Separated. |
| 12. | Washed the organic liquid with H₂O (9.9 L, 3 V). |
| 13. | Concentrated R1 to 2-3 V at 20-30° C. under vacuum. |
| 14. | Obtained 11.38 kg DCM solution of 4-2. |

R1 and R2 were glass-lined reactors with mechanical stirrers.

Step 2 can be conducted at manufacturing scale to prepare large quantities of 4-3 for use in the preparation of Compound 1. A non-limiting example of this scaled up reaction is provided below.

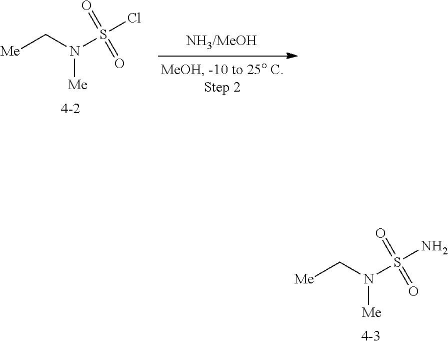 [SMILES: CCN(C)S(=O)(=O)Cl ; CCN(C)S(N)(=O)=O]

Process Description
Operation Steps

| | |
|---|---|
| 1. | Charged MeOH (63.5 kg, 8 V) into R1 |
| 2. | Charged NH₃ (net: 8.4 kg, 9.7eq.) into R1. |
| 3. | Adjusted R1 to −5-5° C. |
| 4. | Charged DCM solution of 4-2 (net: 8.0 kg, 1.0 eq.) into R1 by |
| | dropwise over 1 hr at −5-5° C. |
| 5. | Rinsed the drum of 4-2 with MeOH (2.0 kg, 0.3 V) into R1 |
| 6. | Adjusted R1 to 15-25° C. |
| 7. | Stirred R1 for 15 h at 15-25° C. |
| 8. | Concentrated R1 to 2 V below 40° C. (35~45° C.) under vacuum. |
| 9. | Charged EtOAc (21 kg, 3.0 V) into R1 |
| 10. | Concentrated R1 to 16 L below 40° C. (35~45° C.) under vacuum. |
| 11. | Charged EtOAc (21 kg, 3.0 V) into R1 |
| 12. | Concentrated R1 to 16 L below 40° C. (35~45° C.) under vacuum. |
| 13. | Charged EtOAc (21 kg, 3.0 V) into R1 |
| 14. | Washed the organic phase with H₂O (12 kg, 1.5 V). |
| 15. | Washed the organic phase with H₂O (4 kg, 0.5 V) |
| 16. | Concentrated organic phase to 16 L below 40° C. (35~45° C.) |
| | under vacuum. |
| 17. | Charged EtOAc (17 kg, 2.4 V) into R1. Transferred product- |
| | EtOAc solution into R2 |
| 18. | Concentrated R2 to 8 L below 40° C. (35~45° C.) under vacuum |
| 19. | Charged EtOAc (17 kg, 2.4 V) into R2 |
| 20. | Concentrated R2 to 8 L below 40° C. (35~45° C.) under vacuum |
| 21. | Charged DMAc (5 kg, 0.7 V) into R2 |
| 22. | Obtained 10.68 kg DMAc solution of 4-3. |

R1 was a stainless-steel reactor and R2 was a glass-lined reactor, both were equipped with mechanical stirrers.

Step 3 can be conducted at manufacturing scale to prepare large quantities of 4-4 for use in the preparation of Compound 1. A non-limiting example of this scaled up reaction is provided below.

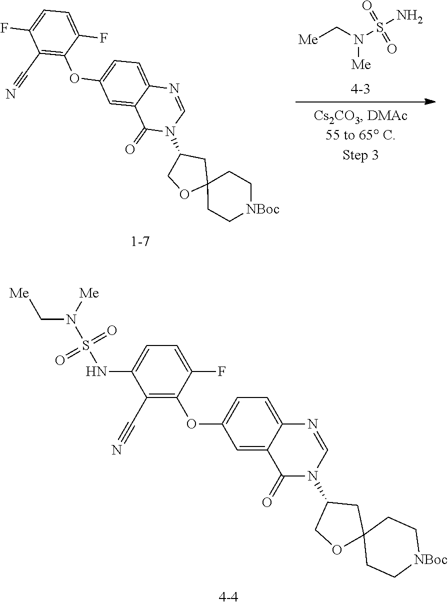 [SMILES: CC(C)(C)OC(=O)N1CCC2(CC1)C[C@@H](n1cnc3ccc(Oc4c(F)ccc(F)c4C#N)cc3c1=O)CO2 ; CCN(C)S(N)(=O)=O ; CCN(C)S(=O)(=O)Nc1ccc(F)c(Oc2ccc3ncn([C@H]4COC5(CCN(C(=O)OC(C)(C)C)CC5)C4)c(=O)c3c2)c1C#N]

Process Description
Operation Steps

| | |
|---|---|
| 1. | Charged 1-7 (11.75 kg, 1.0 eq.) into R1. |
| 2. | Charged DMAc (53.8 kg, 4.58X) into R1. |
| 3. | Charged Cs₂CO₃ (21.35 kg, 1.82X) into R1. |
| 4. | Charged DMAc (31.6 kg, 2.69X) into R1. |
| 5. | Charged 4-3 (10.65 kg, Net: 4.57 kg, 0.39X, solution |
| | of DMAc) into R1. |
| 6. | Adjusted R1 to 55-65° C. |
| 7. | Stirred R1 for 20 hr at 55-65° C. |
| 8. | Charged Process Water (358 kg, 30.4X) into R1 dropwise |
| | at 20~30° C. |
| 9. | Stirred R1 for 1.5 hr at 20~30° C. |
| 10. | Filtered and washed the cake with Process Water (45 kg, 3.8X). |
| 11. | Charged the filtrate into R2. |
| 12. | Charged MTBE (88 kg, 7.5X) into R2. |
| 13. | Stirred R2 for 1 hr at 20~30° C. |
| 14. | Let R2 stand for 2 hr. |
| 15. | Separated. |
| 16. | Charged the aqueous layer into R2. |
| 17. | Charged MTBE (89 kg, 7.6X) into R2. |
| 18. | Stirred R2 for 1 hr at 20~30° C. |
| 19. | Let R2 stand for 2 hr. |
| 20. | Separated. |
| 21. | Charged the aqueous layer into R2. |
| 22. | Charged MTBE (88 kg, 7.6X) into R2. |
| 23. | Stirred R2 for 1 hr at 20~30° C. |
| 24. | Let R2 stand for 3 hr. |
| 25. | Separated. |
| 26. | Charged the aqueous layer into R2. |
| 27. | Charged EtOAc (105 kg, 8.9X) into R2. |
| 28. | Charged 1N HCl (91 kg, 7.7X) to R2 over 1.0 h to adjust to pH 6-7 |
| | at 20-30° C. |
| 29. | Stirred R2 for 1.0 hr at 20~30° C. |
| 30. | Let R2 stand for 2 hr. |
| 31. | Separated. |
| 32. | Charged the aqueous layer into R2. |
| 33. | Charged EtOAc (106 kg, 9.0X) into R2. |
| 34. | Stirred R2 for 1.0 hr at 20~30° C. |
| 35. | Let R2 stand for 2 hr. |
| 36. | Separated. |
| 37. | Charged the aqueous layer into R2. |
| 38. | Charged EtOAc (106.5 kg, 9.0X) into R2. |
| 39. | Stirred R2 for 1.0 hr at 20~30° C. |
| 40. | Let R2 stand for 2 hr. |
| 41. | Separated. |
| 42. | Combined the organic layer to R2. |
| 43. | Decolored organic layer in R2 by CUNO at 20~30° C. for 3 hr. |
| 44. | Rinsed CUNO with EtOAc (168 kg). |
| 45. | Concentrated R2 below 45° C. under vacuum. |
| 46. | Obtained 85 kg EtOAc solution of 4-4. |

R1 and R2 were glass-lined reactors with mechanical stirrers.

Step 4 can be conducted at manufacturing scale to prepare large quantities of 4-5 for use in the preparation of Compound 1. A non-limiting example of this scaled up reaction is provided below.

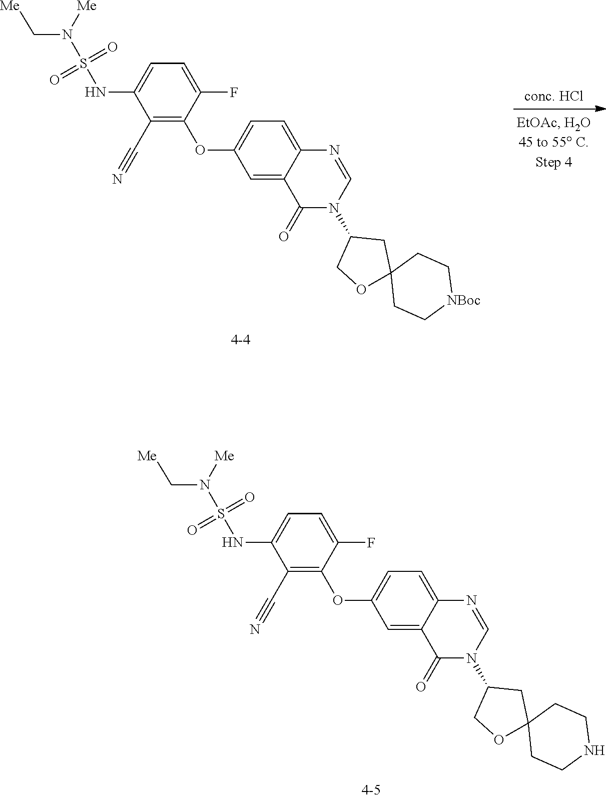 [SMILES: CCN(C)S(=O)(=O)Nc1ccc(F)c(Oc2ccc3ncn([C@H]4COC5(CCN(C(=O)OC(C)(C)C)CC5)C4)c(=O)c3c2)c1C#N ; CCN(C)S(=O)(=O)Nc1ccc(F)c(Oc2ccc3ncn([C@H]4COC5(CCNCC5)C4)c(=O)c3c2)c1C#N]

Process Description
Operation Steps

| | |
|---|---|
| 1. | Charged 35% hydrochloric acid 35% (5.85 kg, 0.72X) into R1 |
| 2. | Charged Process Water (24.15 kg, 2.97X) into R1 |
| 3. | Stirred R1 for 0.5 h at 20~30° C. |
| 4. | Loaded the material in reactor into steel-plastic composite drums. |
| 5. | Charged 4-4 EtOAc solution (84.75 kg, net: 8.14 kg) into R1 |
| 6. | Concentrated R1 below 45° C. under vacuum to ~4 V. |
| 7. | Adjusted R1 to 45~55° C. |
| 8. | Charged HCl aqueous solution into R1 slowly by dropwise |
| | at 45~55° C. |
| 9. | Stirred R1 for 4 hr at 45~55° C. |
| 10. | Adjusted R1 to 20~30° C. |
| 11. | Charged Process Water (25.4 kg, 3.12X) into R1. |
| 12. | Adjusted pH to 8.87 with 20% Na₂CO₃ solution. (62 kg, 7.6X) |
| 13. | Stirred for 2.0 hr at 20~30° C. |
| 14. | Filtered and washed the cake with Process Water (56 kg, 6.9X). |
| 15. | After drying, 6.4 kg product was obtained, HPLC purity = 98.2% |

R1 was a glass-lined reactor with mechanical stirrer.

Step 5 can be conducted at manufacturing scale to prepare large quantities of 4-6 for use in the preparation of Compound 1. A non-limiting example of this scaled up reaction is provided below.

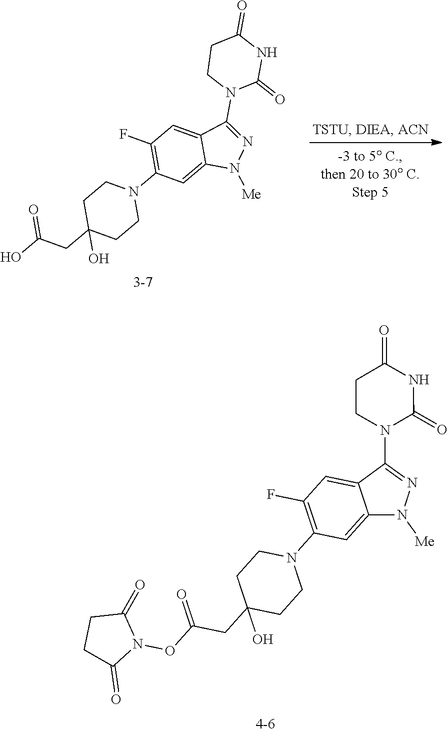 [SMILES: Cn1nc(N2CCC(=O)NC2=O)c2cc(F)c(N3CCC(O)(CC(=O)O)CC3)cc21 ; Cn1nc(N2CCC(=O)NC2=O)c2cc(F)c(N3CCC(O)(CC(=O)ON4C(=O)CCC4=O)CC3)cc21]

Process Description
Operation Steps

| | |
|---|---|
| 1. | Charged ACN (20.8 kg, 3.8X) into R1. |
| 2. | Charged 3-7 (5.40 kg, 1.0 eq.) into R1. |
| 3. | Charged Acetonitrile (20.8 kg, 3.8X) into R1 |
| 4. | Adjusted R1 to −5-5° C. |
| 5. | Charged DIEA (2.62 kg, 1.6 eq) into R1. |
| 6. | Stirred R1 for 1.5 hr (1~2 hr) at −5~5° C. |
| 7. | Charged TSTU (475.9 g, 1.6 eq) into R1. |
| 8. | Charged ACN (7.55 kg, 1.4X) into R1. |
| 9. | Adjusted R1 to 20-30° C. |
| 10. | Stirred R1 for 16 hr at 20~30° C. |
| 11. | Filtered and washed the cake with ACN (25.35 kg, 4.69X). |
| 12. | Rinsed the wet cake with H₂O (34 kg, 6.30X). |
| 13. | Rinsed the wet cake with acetonitrile (27.70 kg, 5.13X). |
| 14. | Dried at 40-50° C. for 10-20 h to obtain 5.65 kg of product. |

R1 was a glass-lined reactor with mechanical stirrer.

¹H NMR (400 MHz, DMSO-d₆): δ ppm 1.70-2.01 (m, 4H) 2.66-2.93 (m, 8H) 2.99-3.22 (m, 4H) 3.76-4.09 (m, 5H) 4.96 (br s, 1H) 7.13 (br d, J=1.10 Hz, 1H) 7.33 (br d, J=11.49 Hz, 1H) 10.54 (br s, 1H).

Step 6 can be conducted at manufacturing scale to prepare large quantities of Compound 1 (amorphous) for use in the preparation of Compound 1. A non-limiting example of this scaled up reaction is provided below.

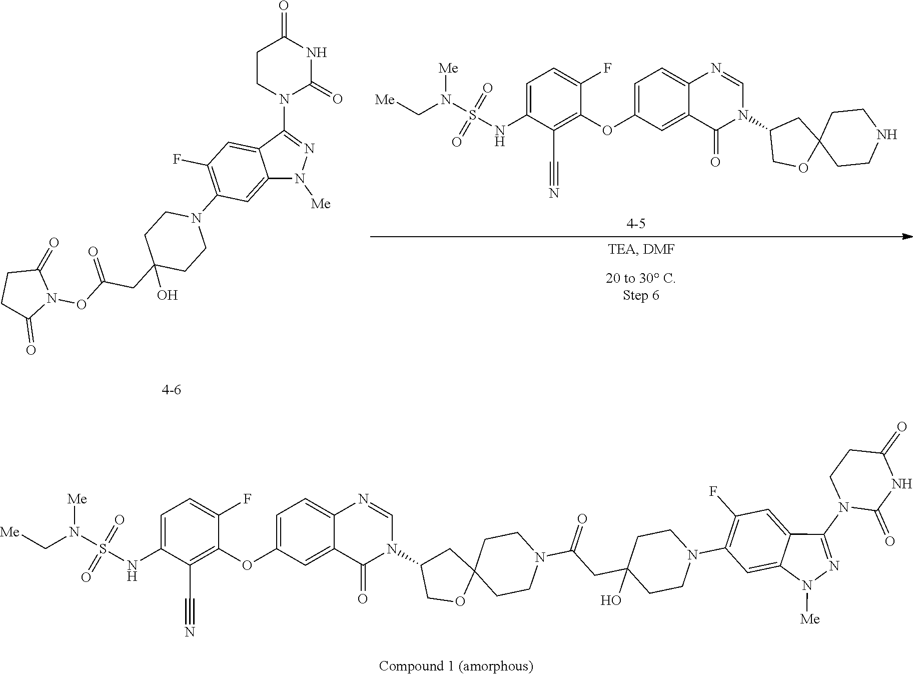 [SMILES: Cn1nc(N2CCC(=O)NC2=O)c2cc(F)c(N3CCC(O)(CC(=O)ON4C(=O)CCC4=O)CC3)cc21 ; CCN(C)S(=O)(=O)Nc1ccc(F)c(Oc2ccc3ncn([C@H]4COC5(CCNCC5)C4)c(=O)c3c2)c1C#N ; CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

Process Description
Operation Steps

| | |
|---|---|
| 1. | Charged DMF (8.2 kg) into R1. |
| 2. | Charged 4-5 (2.28 kg, net: 2.19 kg, 1.0 eq.) into R1. |
| 3. | Charged 4-6 (2.16 kg, net: 2.02 kg, 1.0 eq.) into R1. |
| 4. | Charged DMF (12.4 kg) into R1. |
| 5. | Charged TEA (0.201 kg, 0.5 eq.) into R1. |
| 6. | Adjusted R1 to 20-30° C. |
| 7. | Stir R1 for 24 hr at 20-30° C. |
| 8. | Charged water (110 kg) into R2. |
| 9. | Charged the reaction solution in R1 slowly into R2 with |
| | stirring at 20-30° C. |
| 10. | Stirred R2 for 2 hr at 20-30° C. |
| 11. | Filtered the reaction mixture and washed the cake with H₂O (25 kg). |
| 12. | Charged the filtered wet cake into R3. |
| 13. | Charge DCM (71 kg) into R3. |
| 14. | Stirred R3 for 1 hr at 20~30° C. |
| 15. | Let R3 stand for 4 hr at 20~30° C. |
| 16. | Separated the bottom organic layer and removed the upper aqueous |
| | layer. |
| 17. | Returned the organic layer to R3 and decolored the organic layer by |
| | circulating it through CUNO filter at 20-30° C. for 5.0 hr. |
| 18. | Concentrated R3 at 45° C. under vacuum to dryness. |
| 19. | Dissolved in DMF (12.8 kg). |
| 20. | Charged the DMF solution into H₂O (116 kg) at 20-30° C. |
| 21. | Adjusted the pH to 2~3 with 1N HCl (0.6 kg) and stirred for 4 hr. |
| 22. | Filtered and washed cake with H₂O (75 kg). |
| 23. | Obtained the wet cake. |
| 24. | Dried at 45~55° C. in vacuum oven to obtain 3.08 kg of product |
| | as a gray to white solid. XRPD: Purity: 97.7% |

R1 and R3 were glass-lined reactors with mechanical stirrers, and R2 was a stainless-steel reactor with a mechanical stirrer.

¹H NMR (400 MHz, DMSO-d₆): δ ppm 10.53 (s, 1H), 10.20 (br s, 1H), 8.36 (s, 1H), 7.95-7.75 (m, 2H), 7.70 (dd, J=3.2, 8.8 Hz, 1H), 7.50 (dd, J=4.4, 9.6 Hz, 1H), 7.41-7.26 (m, 2H), 7.11 (d, J=7.2 Hz, 1H), 5.40-5.19 (m, 1H), 5.03 (br s, 1H), 4.18-4.08 (m, 2H), 3.94 (s, 3H), 3.89 (t, J=6.7 Hz, 2H), 3.78 (br dd, J=4.8, 12.8 Hz, 1H), 3.68-3.58 (m, 1H), 3.56-3.39 (m, 2H), 3.19-3.14 (m, 4H), 3.10-3.00 (m, 2H), 2.79 (s, 3H), 2.73 (t, J=6.7 Hz, 2H), 2.57 (br s, 2H), 2.42-2.32 (m, 1H), 2.14-1.97 (m, 1H), 1.86-1.49 (m, 8H), 1.05 (t, J=7.2 Hz, 3H)

Step 7 can be conducted at manufacturing scale to prepare large quantities of crystalline Compound 1. A non-limiting example of this scaled up reaction is provided below.

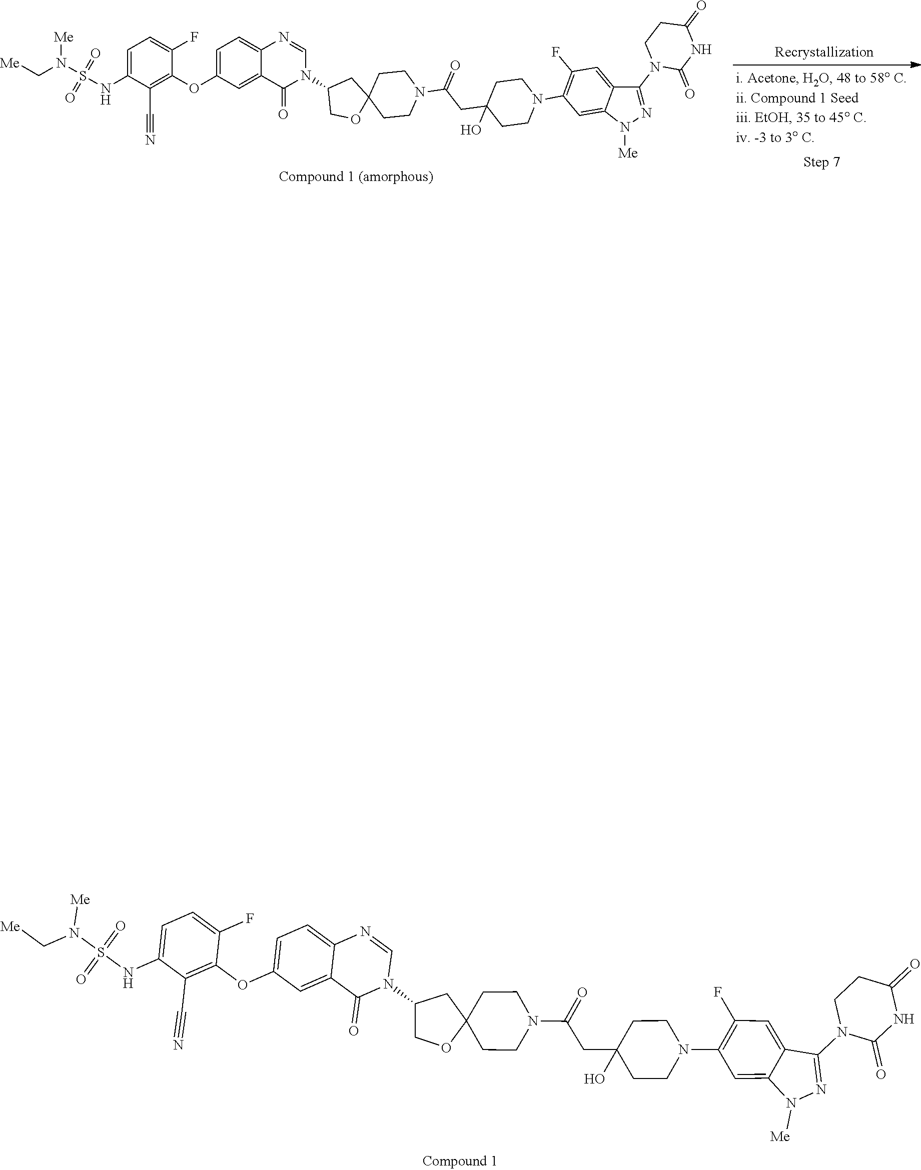 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N ; CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]

Process Description
Operation Steps

| | |
|---|---|
| 1. | Charged amorphous Compound 1 (net: 2.92 kg, 1.0 eq.) into R1. |
| 2. | Charged acetone/H₂O (9 V/1 V, 28 kg) into R1. |
| 3. | Adjusted R1 to 48-58° C. (Target:50° C.). |
| 4. | Stirred R1 for 0.5 hr at 48-58° C. (It started out as a sticky |
| | solid and then slowly dissolved.) |
| 5. | Filtered the solution and washed with acetone/H₂O = 1.8 kg at 53° C. |
| 6. | Transferred the solution into R2 |
| 7. | Stirred R2 for 0.5 hr at 48-58° C. |
| 8. | Adjusted R2 to 35-45° C. (Target temperature:36° C.) |
| 9. | Charged crystal seed (0.147 kg, 0.05X) at 35-45° C. under N₂. |
| 10. | Stirred R2 for 2.0 hr at 48-58° C. |
| 11. | Charged EtOH (59 kg, 25.6 V) into R2 dropwise over 10 hr at |
| | 35-45° C. |
| 12. | Adjusted R2 to −3-3° C. over 4 hr. |
| 13. | Stirred R2 for 12 hr at −3-3° C. |
| 14. | Continued to stir for 20-24 hr. |
| 15. | Filtered and washed the cake with 5 V EtOH. |
| 16. | Dried at 45~55° C. in vacuum oven for 24 hr to obtain 2.16 |
| | kg of crystalline Compound 1 was a white solid. Chiral purity: |
| | 99.8%, Purity: 98.9% |

R1 was a stainless-steel reactor and R2 was a Hastelloy reactor, both were equipped with mechanical stirrers.

In certain aspects Step 7 is modified, for example in certain embodiments the crystallization is conducted at a lower temperature such as a temperature between about 0° C. and about 35° C., a temperature between about 20° C. and about 35° C., a temperature between about 0° C. and about 20° C., or a temperature between about 10° C. and about 30° C. In certain embodiments the temperature is about 0° C., about 5° C., about 10° C., about 15° C. about 20° C., about 25° C., or about 30° C.

In certain embodiments the crystallization is conducted with a slow stir rate, for example with stirring between about 50 rpm and about 300 rpm, stirring between about 150 rpm and about 300 rpm, or stirring between about 200 rpm and about 300 rpm. In certain embodiments the stirring is about 50 rpm, about 60 rpm, about 70 rpm, about 80 rpm, about 90 rpm, about 100 rpm, about 110 rpm, about 120 rpm, about 130 rpm, about 140 rpm, about 150 rpm, about 160 rpm, about 170 rpm, about 180 rpm, about 190 rpm, about 200 rpm, about 210 rpm, about 220 rpm, about 230 rpm, about 240 rpm, about 250 rpm, about 260 rpm, about 270 rpm, about 280 rpm, about 290 rpm, or about 300 rpm.

In certain embodiments the crystallization is conducted for a period of more than ten hours, for example a crystallization time between about 10 hours and about 30 hours, a crystallization time between about 15 hours and about 30 hours, a crystallization time between about 20 hours and about 30 hours, a crystallization time between about 10 hours and about 25 hours, or a crystallization time between about 15 hours and about 25 hours. In certain embodiments the crystallization time is about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, or about 35 hours.

### Example 5: Synthesis of (3R)-3-[6-[2-cyano-3-[[ethyl(methyl)sulfamoyl]amino]-6-fluoro-phenoxy]-4-oxo-quinazolin-3-yl]-8-[2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetyl]-1-oxa-8-azaspiro[4.5]decane

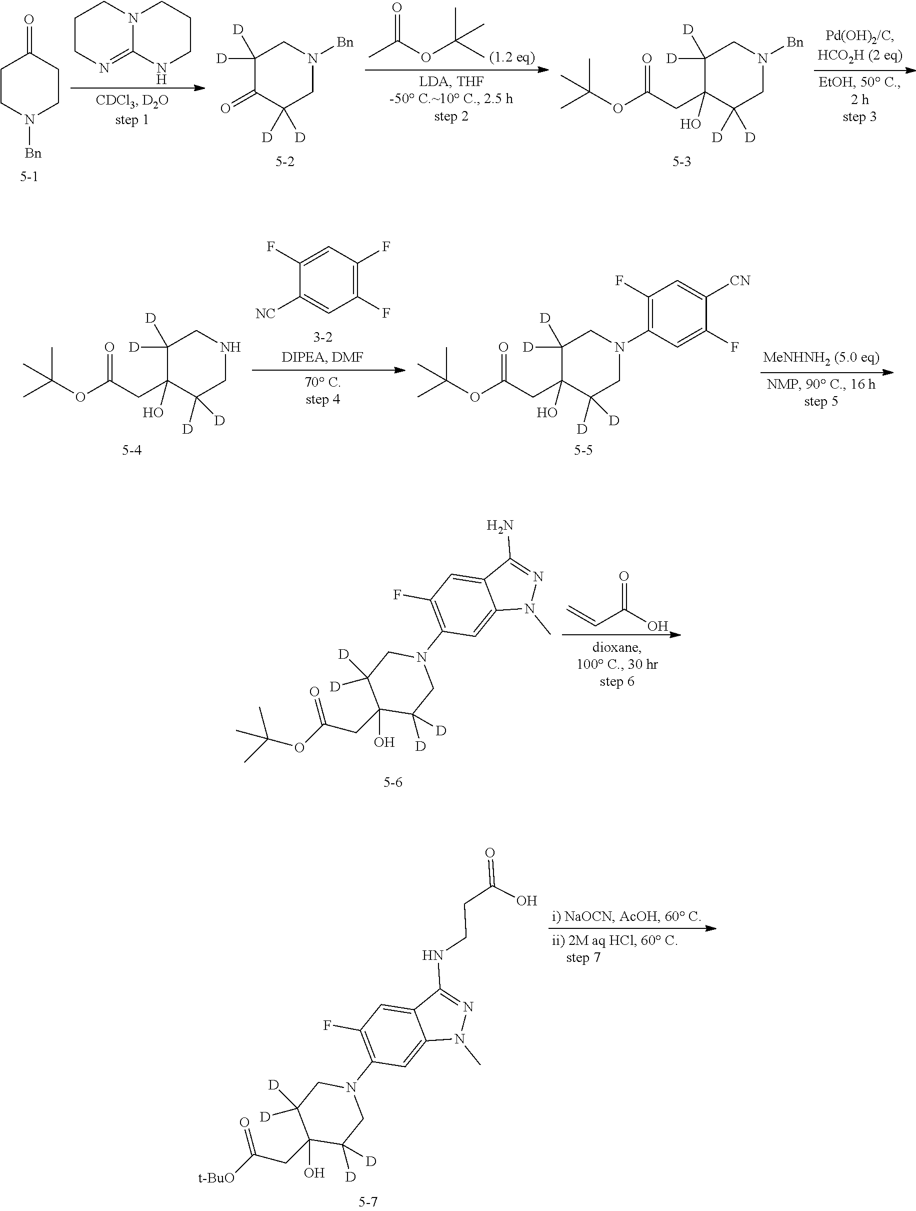

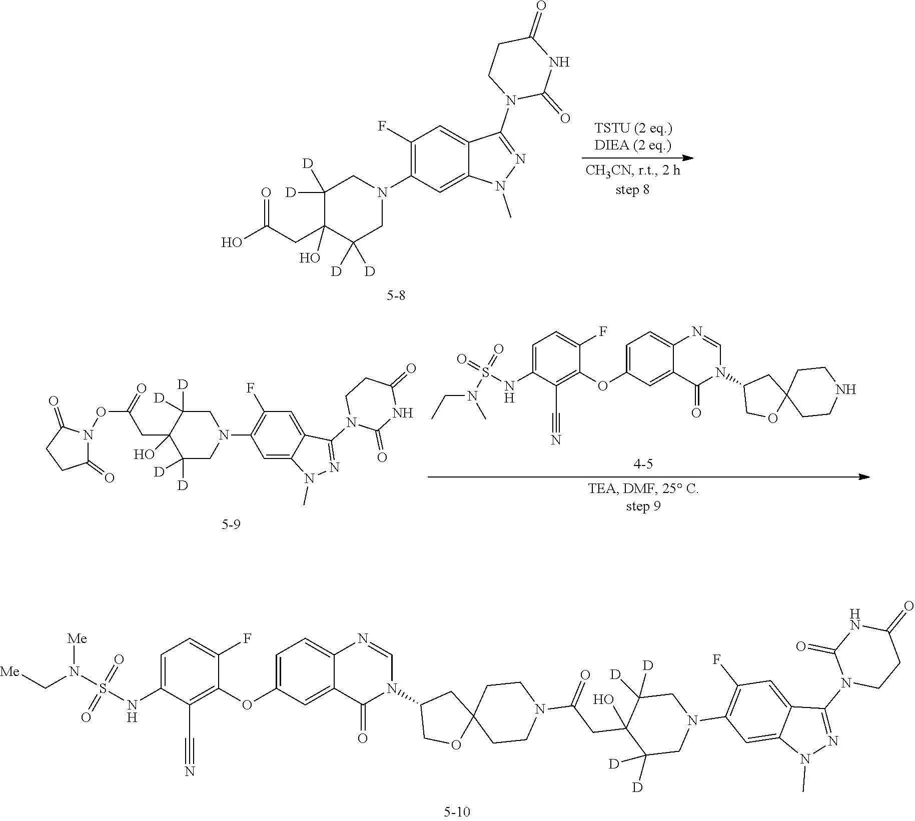

Step 1: To a solution of 1-benzylpiperidin-4-one (10 g, 52.84 mmol, 9.80 mL, 1 eq.) in CDCl₃ (50 mL) and D20 (50 mL) was added 3,4,6,7,8,9-hexahydro-2H-pyrimido[1,2-a]pyrimidine (1.40 g, 10.06 mmol, 0.19 eq.). The mixture was stirred at 15° C. for 16 hr in sealed tube. To the reaction mixture was added aq. HCl (1 M) to adjust pH to ˜7, the separated organic phase was washed with water (20 mL) and brine (20 mL), dried over Na₂SO₄, filtered and concentrated to give compound 1-benzyl-3,3,5,5-tetradeuterio-piperidin-4-one (9.8 g, crude) as a yellow oil. ¹H NMR (400 MHz, chloroform-d) δ=7.53-7.14 (m, 5H), 3.64 (s, 2H), 2.75 (s, 4H).

Step 2: To a solution of LDA (2 M, 30.11 mL, 1.2 eq.) was added a solution of tert-butyl acetate (7.00 g, 60.22 mmol, 8.08 mL, 1.2 eq.) in THF (75 mL) at −65° C., the mixture was stirred at −60° C. for 0.5 h, then a solution of 1-benzyl-3,3,5,5-tetradeuterio-piperidin-4-one (9.7 g, 50.19 mmol, 1.76 mL, 1 eq.) in THF (10 mL) was added at −60° C. The mixture was stirred at −60° C. for 1.5 h, then the mixture was stirred at 10° C. for 0.5 h. The reaction mixture was poured into sat. NH₄Cl (100 mL), extracted with EtOAc (100 mL×2), the combined organic phase washed with water (100 mL), brine (100 mL), dried over Na₂SO₄, filtered and concentrated to give compound tert-butyl 2-(1-benzyl-3,3,5,5-tetradeuterio-4-hydroxy-4-piperidyl)acetate (17 g, crude) as a yellow solid. LCMS m/z (ESI): 310.2 [M+H]⁺. ¹H NMR (400 MHz, chloroform-d) δ=7.28-7.14 (m, 5H), 3.53 (s, 1H), 3.45 (s, 2H), 2.49 (br d, J=11.4 Hz, 2H), 2.34 (br d, J=11.4 Hz, 2H), 2.30 (s, 2H), 1.39 (s, 9H).

Step 3: To a solution of tert-butyl 2-(1-benzyl-3,3,5,5-tetradeuterio-4-hydroxy-4-piperidyl)acetate (17 g, 54.94 mmol, 1 eq.) in EtOH (170 mL) was added Pd(OH)₂/C (3.40 g, 20% purity) and HCOOH (5.28 g, 109.88 mmol, 2 eq.). The mixture was stirred at 50° C. for 2 hr. The reaction mixture was filtered and concentrated. The residue was triturated with petroleum/EtOAc (10/1, 5V) at 25° C. for 2 h, the suspension was filtered, and the filtered cake was collected. Compound tert-butyl 2-(3,3,5,5-tetradeuterio-4-hydroxy-4-piperidyl)acetate (10 g, crude) was obtained as a yellow solid. ¹H NMR (400 MHz, chloroform-d) δ=3.02 (br d, J=12.4 Hz, 2H), 2.82 (d, J=12.4 Hz, 2H), 2.38 (s, 2H), 1.47 (s, 9H).

Step 4: To a solution of tert-butyl 2-(3,3,5,5-tetradeuterio-4-hydroxy-4-piperidyl)acetate (10 g, 45.60 mmol, 1 eq.) in DMF (40 mL) was added DIEA (11.79 g, 91.19 mmol, 15.88 mL, 2 eq.) and 2,4,5-trifluorobenzonitrile (6.80 g, 43.32 mmol, 0.95 eq.). The mixture was stirred at 70° C. for 1 hr. The reaction mixture was cooled to 25° C. and poured into water (250 mL). The resulting mixture was stirred at 25° C. for 2h, filtered and the filtered cake was collected. Compound tert-butyl 2-[1-(4-cyano-2,5-difluoro-phenyl)-3,3,5,5-tetradeuterio-4-hydroxy-4-piperidyl]acetate (12 g, crude) was obtained as a yellow solid. LCMS m/z (ESI): 357.1 [M+H]⁺. ¹H NMR (400 MHz, chloroform-d) δ=7.17 (dd, J=6.0, 12.4 Hz, 1H), 6.66 (dd, J=7.0, 11.0 Hz, 1H), 3.87 (s, 1H), 3.46-3.35 (m, 2H), 3.31-3.22 (m, 2H), 2.43 (s, 2H), 1.49 (s, 9H) Step 5: To a solution of tert-butyl 2-[1-(4-cyano-2,5-difluoro-phenyl)-3,3,5,5-tetradeuterio-4-hydroxy-4-piperidyl]acetate (12 g, 33.67 mmol, 1 eq.) in NMP (100 mL) was added methylhydrazine (19.43 g, 168.70 mmol, 22.21 mL, 40% purity, 5.0 eq.) slowly at 55° C. The mixture was stirred at 90° C. for 16 hrs. The reaction mixture was quenched by addition aq. citric acid to adjust pH to 6-7 at 25° C., then water was added into the mixture to 600 mL. The resulting mixture was stirred for 2 h at 20° C., filtered and the filtered cake was collected. Compound tert-butyl 2-[1-(3-amino-5-fluoro-1-methyl-indazol-6-yl)-3,3,5,5-tetradeuterio-4-hydroxy-4-piperidyl]acetate (11 g, crude) was obtained as a yellow solid. LCMS m/z (ESI): 383.2 [M+H]⁺. ¹H NMR (400 MHz, chloroform-d) δ=7.12 (d, J=11.8 Hz, 1H), 6.73-6.65 (m, 1H), 3.81 (s, 1H), 3.79 (s, 3H), 3.27-3.22 (m, 2H), 3.18-3.09 (m, 2H), 2.46 (s, 2H), 1.50 (s, 9H).

Step 6: To a solution of tert-butyl 2-[1-(3-amino-5-fluoro-1-methyl-indazol-6-yl)-3,3,5,5-tetradeuterio-4-hydroxy-4-piperidyl]acetate (11 g, 28.76 mmol, 1 eq.) in dioxane (110 mL) was added acrylic acid (6.22 g, 86.28 mmol, 5.92 mL, 3 eq.). The mixture was stirred at 100° C. for 30 hr. The reaction mixture was diluted with water (300 mL), extracted with 2-MeTHF/MeOH (5/1, 200×3), the organic phase was washed with brine (200 mL), dried over Na₂SO₄, filtered and concentrated. The residue was triturated with petroleum ether/EtOAc (1/1, 110 mL) at 20° C. for 2 h, then filtered. The filtered cake was collected, further triturated with i-PrOH/i-PrOAc (5/1, 10V) at 50° C. for 2 h. The suspension mixture was filtered at 50° C. Compound 3-[[6-[4-(2-tert-butoxy-2-oxo-ethyl)-3,3,5,5-tetradeuterio-4-hydroxy-1-piperidyl]-5-fluoro-1-methyl-indazol-3-yl]amino]propanoic acid (5.2 g, 10.53 mmol, 36.60% yield, 92% purity) was obtained as a white solid. LCMS m/z (ESI): 455.3 [M+H]⁺. ¹H NMR (400 MHz, DMSO-d₆) δ=7.36 (d, J=12.6 Hz, 1H), 6.86 (d, J=7.0 Hz, 1H), 3.70 (s, 3H), 3.40 (br t, J=6.8 Hz, 2H), 3.13-3.05 (m, 2H), 3.02-2.95 (m, 2H), 2.56 (t, J=6.8 Hz, 2H), 2.37 (s, 2H), 1.42 (s, 9H).

Step 7: To a solution of 3-[[6-[4-(2-tert-butoxy-2-oxo-ethyl)-3,3,5,5-tetradeuterio-4-hydroxy-1-piperidyl]-5-fluoro-1-methyl-indazol-3-yl]amino]propanoic acid (5.2 g, 11.44 mmol, 1 eq.) in AcOH (50 mL) was added sodium; cyanate (1.49 g, 22.88 mmol, 2 eq.). The mixture was stirred at 60° C. for 12 h, then HCl (2 M, 52.00 mL, 9.09 eq.) was added, and the mixture was stirred at 60° C. for 16h. To the reaction mixture was added saturated Na₂CO₃ and NaHCO₃ to adjust pH to ˜5, the mixture was stirred at 15° C. for 2h, solid precipitated out, and was filtered. The filtered cake was collected, triturated with water (1OV) at 100° C. for 16 h, then filtered. Compound 2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetic acid (2.4 g, 5.64 mmol, 49.30% yield, 99.5% purity) was obtained as a gray solid. LCMS m/z (ESI): 424.1 [M+H]⁺. ¹H NMR (400 MHz, DMSO-d₆) δ=10.53 (s, 1H), 7.33 (br d, J=12.8 Hz, 1H), 7.12 (br d, J=6.8 Hz, 1H), 4.03-3.81 (m, 5H), 3.15 (br d, J=11.6 Hz, 2H), 3.04 (br d, J=11.6 Hz, 2H), 2.74 (br t, J=6.4 Hz, 2H), 2.43 (s, 2H).

Step 8: To a solution of 2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetic acid (2.4 g, 5.67 mmol, 1 eq.) in ACN (24 mL) was added TSTU (3.41 g, 11.34 mmol, 2 eq.) and DIEA (1.47 g, 11.34 mmol, 1.97 mL, 2 eq.) at 15° C. The mixture was stirred at 15° C. for 2 hr. The reaction mixture was filtered, the filtered cake was washed with MeCN (10 mL), water (10 mL) and MeCN (10 mL). Compound (2,5-dioxopyrrolidin-1-yl) 2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetate (2.4 g, crude) was obtained as a white solid. LCMS m/z (ESI): 521.2 [M+H]⁺. ¹H NMR (400 MHz, DMSO-d₆) δ=10.54 (s, 1H), 7.33 (br d, J=12.8 Hz, 1H), 7.14 (br d, J=7.0 Hz, 1H), 4.94 (s, 1H), 4.01-3.78 (m, 5H), 3.18 (br d, J=11.6 Hz, 2H), 3.04 (br d, J=11.6 Hz, 2H), 2.83 (br d, J=9.0 Hz, 6H), 2.73 (br t, J=6.4 Hz, 2H).

Step 9: To a solution of (2,5-dioxopyrrolidin-1-yl) 2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetate (1 g, 1.92 mmol, 1 eq.) in DMF (10 mL) was added (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (1.07 g, 1.92 mmol, 1 eq.) and TEA (97.20 mg, 960.61 umol, 133.71 uL, 0.5 eq.). The mixture was stirred at 25° C. for 18 hr. The reaction mixture was added to water (80 mL) slowly, then adjusted pH to 6 with aq. HCl (1 M) and filtered. The filtered cake was collected, triturated with i-PrOH/i-PrOAc (5/1, 20 mL) at 25° C. for 2h, and then further triturated with MeOH (14 mL) at 25° C. for 12h. Compound (3R)-3-[6-[2-cyano-3-[[ethyl(methyl)sulfamoyl]amino]-6-fluoro-phenoxy]-4-oxo-quinazolin-3-yl]-8-[2-[3,3,5,5-tetradeuterio-1-[3-(2,4-dioxohexahydropyrimidin-1-yl)-5-fluoro-1-methyl-indazol-6-yl]-4-hydroxy-4-piperidyl]acetyl]-1-oxa-8-azaspiro[4.5]decane (1.4 g, 1.43 mmol, 74.69% yield, 98.6% purity) was obtained as a gray solid. LCMS m/z (ESI): 962.5 [M+H]⁺. ¹H NMR (400 MHz, DMSO-d₆) δ=10.52 (s, 1H), 10.18 (br s, 1H), 8.35 (s, 1H), 7.92-7.75 (m, 2H), 7.69 (dd, J=2.8, 9.0 Hz, 1H), 7.49 (br dd, J=3.9, 9.0 Hz, 1H), 7.42-7.26 (m, 2H), 7.11 (br d, J=7.0 Hz, 1H), 5.30 (br d, J=6.4 Hz, 1H), 5.00 (br s, 1H), 4.23-4.06 (m, 2H), 4.01-3.83 (m, 5H), 3.82-3.71 (m, 1H), 3.68-3.58 (m, 1H), 3.51 (br d, J=7.4 Hz, 1H), 3.46-3.38 (m, 1H), 3.20-3.10 (m, 4H), 3.04 (br d, J=11.6 Hz, 2H), 2.84-2.69 (m, 5H), 2.56 (br s, 2H), 2.41-2.34 (m, 1H), 2.14-2.00 (m, 1H), 1.84-1.48 (m, 4H), 1.05 (t, J=7.0 Hz, 3H).

### Example 6: Synthesis of (3R)-3-[6-[2-cyano-3-[[ethyl(methyl)sulfamoyl]amino]-6-fluoro-phenoxy]-5,7,8-trideuterio-4-oxo-quinazolin-3-yl]-8-[2-[1-[3-(2,4-dioxohexahydropyrimidin-1-yl)-5-fluoro-1-methyl-indazol-6-yl]-4-hydroxy-4-piperidyl]acetyl]-1-oxa-8-azaspiro[4.5]decane

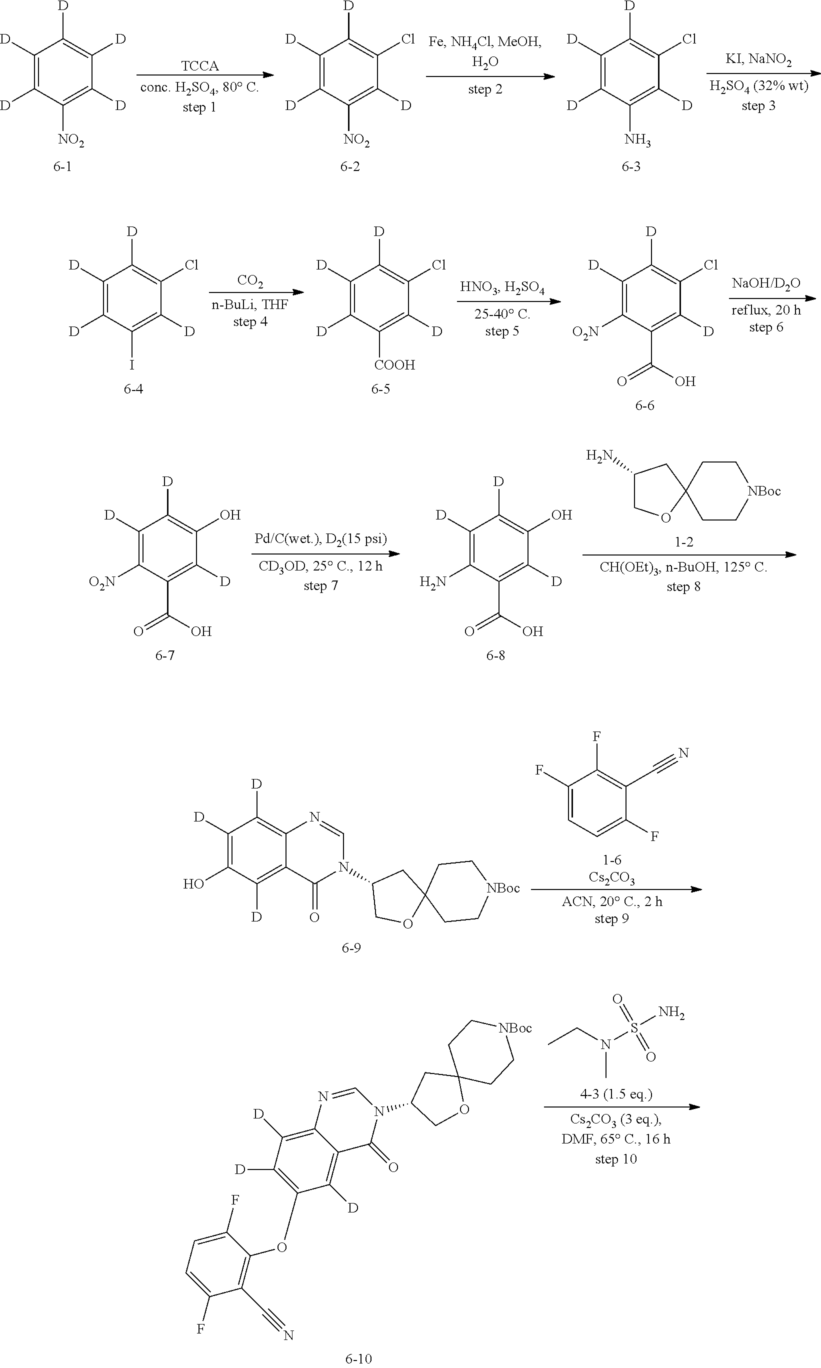

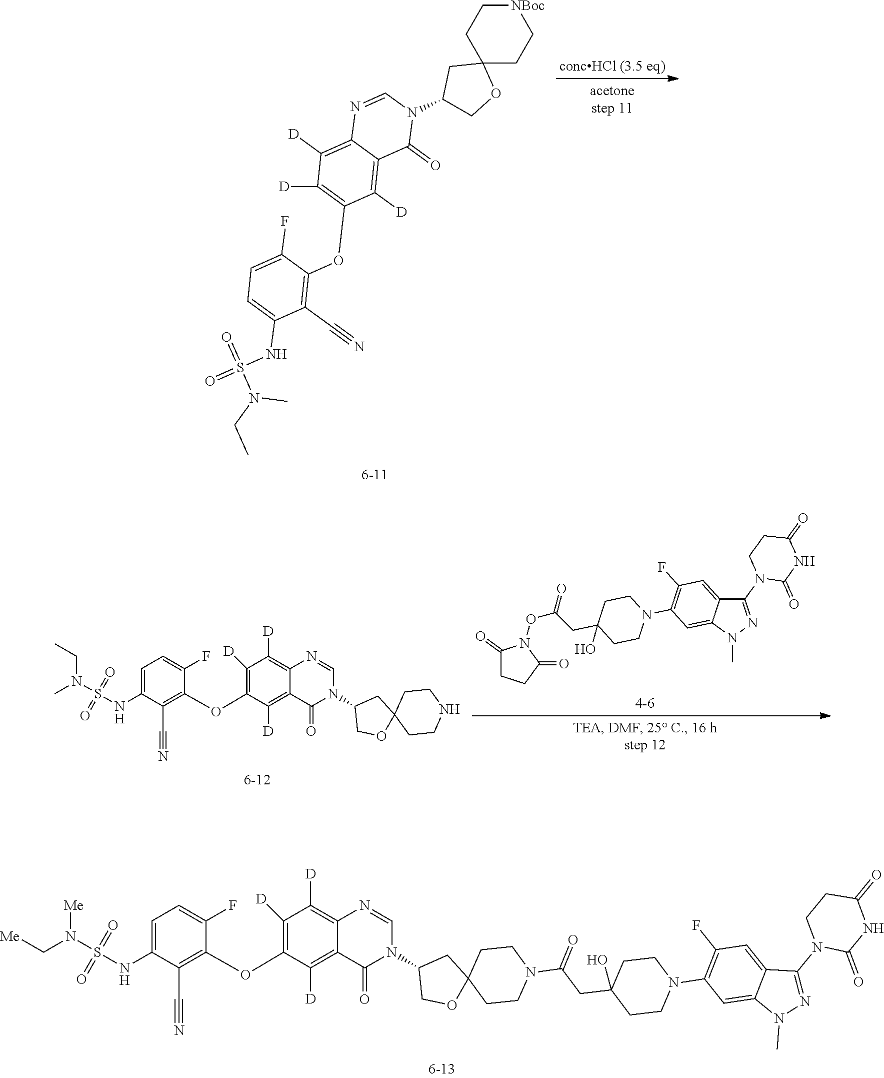 [SMILES: [2H]c1c(Oc2c(F)ccc(NS(=O)(=O)N(C)CC)c2C#N)c([2H])c2c(=O)n([C@H]3COC4(CCN(C(=O)OC(C)(C)C)CC4)C3)cnc2c1[2H] ; [2H]c1c(Oc2c(F)ccc(NS(=O)(=O)N(C)CC)c2C#N)c([2H])c2c(=O)n([C@H]3COC4(CCNCC4)C3)cnc2c1[2H] ; Cn1nc(N2CCC(=O)NC2=O)c2cc(F)c(N3CCC(O)(CC(=O)ON4C(=O)CCC4=O)CC3)cc21 ; [2H]c1c(Oc2c(F)ccc(NS(=O)(=O)N(C)CC)c2C#N)c([2H])c2c(=O)n([C@H]3COC4(CCN(C(=O)CC5(O)CCN(c6cc7c(cc6F)c(N6CCC(=O)NC6=O)nn7C)CC5)CC4)C3)cnc2c1[2H]]

Step 1: To a solution of 1,2,3,4,5-pentadeuterio-6-nitro-benzene (25 g, 195.10 mmol, 4.17 mL, 1 eq.) in H2S04 (140 mL, 98% purity) was added TCCA (15.42 g, 66.33 mmol, 0.34 eq.), and the mixture was stirred for 3 h at 80° C. The reaction mixture was cooled to room temperature, poured into ice/water (200 mL) and extracted with EtOAc (100 mL×3). The combined organic layers were washed with water (2×40 mL), brine (2×40 mL), dried over Na₂SO₄, filtered and concentrated under reduced pressure to give a residue. The residue was purified by flash silica gel chromatography (ISCO®; 220 g SepaFlash® Silica Flash Column, Eluent of 0% Ethyl acetate/Petroleum ether, gradient: 50 mL/min) to give 1-chloro-2,3,4,6-tetradeuterio-5-nitro-benzene (32 g, crude) as light brown oil. HPLC Rt=2.386 min. ²H NMR (400 MHz, DMSO) δ=8.72-8.21 (m, 2H).

Step 2: To a solution of 1-chloro-2,3,4,6-tetradeuterio-5-nitro-benzene (37 g, 228.99 mmol, 7.19 mL, 1 eq.) and NH₄Cl in MeOH (480 mL) and H₂O (80 mL) was added Fe (76.73 g, 1.37 mol, 6 eq.) (97.99 g, 1.83 mol, 8 eq.) in portions, and the mixture was stirred for 1 h at 80° C. The mixture was filtered through Celite, the filter cake was washed with MeOH (4×50 mL) and EtOAc (4×50 mL). The filtrate was concentrated under reduced pressure to give a residue. The residue was diluted with H₂O (50 mL) and extracted with EtOAc (50 mL×3). The combined organic layers were washed with brine (30 mL×2), dried over Na₂SO₄, filtered and concentrated under reduced pressure. The crude product was purified by reversed-phase HPLC (0.1% FA condition; H₂O/ACN=1/0-1/1) to give 3-chloro-2,4,5,6-tetradeuterio-aniline (17.8 g, 132.56 mmol, 57.89% yield, 98% purity) as light brown oil. LCMS m/z (ESI): 132.0 [M+H]⁺. ¹H NMR (400 MHz, DMSO-d₆) δ=5.36 (s, 2H). ²H NMR (400 MHz, DMSO) δ=7.53 (s, 1H), 7.12-7.04 (m, 3H).

Step 3: A solution of NaNO₂ (9.23 g, 133.74 mmol, 1.1 eq.) in H₂O (48 mL) was added to a solution of 3-chloro-2,4,5,6-tetradeuterio-aniline (16 g, 121.58 mmol, 1 eq.) in H2S04 (258.51 g, 843.44 mmol, 140.50 mL, 32% purity) at 0° C., and the mixture was stirred for 1 h at 0° C. Then a solution of KI (30.27 g, 182.38 mmol, 1.5 eq.) in H₂O (80 mL) was added at 0° C. The resulting mixture was stirred for 1 h at 0° C. H₂O (50 mL) was added at 0° C., the resulting mixture was extracted with EtOAc (80 mL×3), the combined organic layers were washed with brine (50 mL×2), dried over Na₂SO₄, filtered and concentrated under reduced pressure to give a residue. The residue was purified by flash silica gel chromatography (SepaFlash® Silica Flash Column; eluent: petroleum ether) to give 1-chloro-2,3,4,6-tetradeuterio-5-iodo-benzene (20 g, 78.77 mmol, 64.79% yield, 95.5% purity) as light brown oil. ²H NMR (400 MHz, DMSO) δ=7.71 (s, 1H), 7.58 (s, 1H), 7.31 (s, 1H), 7.702 (s, 1H).

Step 4: To a solution of 1-chloro-2,3,4,6-tetradeuterio-5-iodo-benzene (19 g, 78.36 mmol, 518.13 uL, 1 eq.) in THF (190 mL) was added n-BuLi (2.5 M, 34.48 mL, 1.1 eq.) at −65° C., and the mixture was stirred for 1 h at −65° C. The reaction mixture was poured into a large amount of dry ice and stirred at −65° C. for 1 h. Then the mixture was quenched by sat. NH₄Cl (50 mL), basified to pH=11, and extracted with MBTE (3×40 mL). The aqueous phase was adjusted to pH ˜4 with aq. HCl and extracted with EtOAc (3×150 mL). The combined organic layers were washed with brine (2×40 mL), dried under vacuum to give 3-chloro-2,4,5,6-tetradeuterio-benzoic acid (9.5 g, 57.26 mmol, 73.08% yield, 96.8% purity) as white solid. ¹H NMR (400 MHz, DMSO-d6) δ=13.46 (s, 1H). ²H NMR (400 MHz, DMSO) δ=8.44-8.08 (m, 4H).

Step 5: To a solution of 3-chloro-2,4,5,6-tetradeuterio-benzoic acid (8.6 g, 53.55 mmol, 103.63 uL, 1 eq.) in H2S04 (15 mL, 98% purity) was added the mixture of HNO₃ (7.60 g, 82.06 mmol, 5.43 mL, 68%, 1.53 eq.) and H2S04 (10.72 g, 107.10 mmol, 5.83 mL, 98%, 2 eq.) at 25° C. The resulting mixture was stirred for 2 h at 25° C., then stirred for 1 h at 40° C. The mixture was poured into ice/H₂O (180 mL) and extracted with DCM (3×200 mL). The organic layer was washed with 0.1% aq·HCl (160 mL), dried over Na₂SO₄ and evaporated to give 3-chloro-2,4,5-trideuterio-6-nitro-benzoic acid (10.2 g, 43.5 mmol, 79.68% yield, 87.3% purity) as light yellow solid. LCMS m/z (ESI): 203.0 [M−H]⁻. ²H NMR (400 MHz, DMSO) δ=8.84-8.70 (m, 3H).

Step 6: The mixture of 3-chloro-2,4,5-trideuterio-6-nitro-benzoic acid (8 g, 39.10 mmol, 103.63 uL, 1 eq.) and NaOH (12.51 g, 312.83 mmol, 8 eq.) in 20 (156.4 mL) was stirred for 20 h at 100° C. (inner). The mixture was cooled to room temperature, acidified with 6 N HCl and extracted with EtOAc (3×80 mL). The combined organic layer was dried over Na₂SO₄ and evaporated to dryness. The crude product was triturated with EtOAc/Tol (1/10, 1OV) at 25° C. for 12 h to give 2,4,5-trideuterio-3-hydroxy-6-nitro-benzoic acid (6.1 g, 30.48 mmol, 77.94% yield, 93% purity) as light yellow solid. LCMS m/z (ESI): 185.1 [M+H]⁻. ¹H NMR (400 MHz, DMSO-d) δ=14.30-13.13 (m, 1H), 11.91-10.84 (m, 1H). ²H NMR (400 MHz, DMSO) δ=8.80-8.29 (m, 1H), 7.72-7.31 (m, 2H).

Step 7: To a solution of 2,4,5-trideuterio-3-hydroxy-6-nitro-benzoic acid (2 g, 10.74 mmol, 1 eq.) in CD30D (20 mL) was added Pd/C (0.4 g, 10% purity). The mixture was stirred at 25° C. for 16 hr under D2 (15 psi) atmosphere. The reaction mixture was filtered, the filtrate was concentrated to give compound 2-amino-3,4,6-trideuterio-5-hydroxy-benzoic acid (1.2 g, crude) as a black solid. ¹H NMR (400 MHz, DMSO-d₆) δ=8.60 (s, 1H), 8.50-7.76 (m, 2H).

Step 8: To a solution of 2-amino-3,4,6-trideuterio-5-hydroxy-benzoic acid (1.2 g, 7.68 mmol, 1 eq.) in n-BuOH (12 mL) was added tert-butyl (3R)-3-amino-1-oxa-8-azaspiro[4.5]decane-8-carboxylate (1.97 g, 7.68 mmol, 1 eq.) in n-BuOH (12 mL) at 120° C., the mixture was stirred at 125° C. for 15 mins, then diethoxymethoxyethane (2.73 g, 18.44 mmol, 3.07 mL, 2.4 eq.) was added. The mixture was stirred at 125° C. for 16 hrs. The reaction mixture was diluted with water (50 mL), EtOAc (50 mL) and then filtered. The filtrate was washed with aq·HCl (1 M, 20 mL), aq·NaHCO₃ (20 mL), water (20 mL) and brine (20 mL), dried over Na₂SO₄, filtered and concentrated. The residue was triturated with petroleum/EtOAc (3/1, 15 mL) at 20° C. for 2 h, then filtered, and the filtered cake was collected. Compound tert-butyl (3R)-3-(5,7,8-trideuterio-6-hydroxy-4-oxo-quinazolin-3-yl)-1-oxa-8-azaspiro[4.5]decane-8-carboxylate (1.5 g, 3.40 mmol, 44.30% yield, 91.8% purity) was obtained as brown solid. LCMS m/z (ESI): 405.1 [M+H]⁺. ¹H NMR (400 MHz, DMSO-d₆) δ=10.49-9.97 (m, 1H), 8.19 (s, 1H), 5.34 (br s, 1H), 4.10 (br d, J=5.0 Hz, 2H), 3.42 (br s, 2H), 2.37 (br dd, J=9.0, 13.0 Hz, 1H), 1.98 (br dd, J=5.2, 13.4 Hz, 1H), 1.73-1.61 (m, 3H), 1.54 (br d, J=7.8 Hz, 1H), 1.39 (br s, 9H).

Step 9: To a solution of 2,3,6-trifluorobenzonitrile (704.22 mg, 4.08 mmol, 91% purity, 1.1 eq.) in ACN (15 mL) was added Cs₂CO₃ (3.02 g, 9.27 mmol, 2.5 eq.) and tert-butyl (3R)-3-(5,7,8-trideuterio-6-hydroxy-4-oxo-quinazolin-3-yl)-1-oxa-8-azaspiro[4.5]decane-8-carboxylate (1.5 g, 3.71 mmol, 1 eq.). The mixture was stirred at 20° C. for 2 hr. The reaction mixture was concentrated, then diluted EtOAc (20 mL) and water (20 mL). The organic phase was washed with water (20 mL), brine (20 mL), dried over Na₂SO₄, filtered and concentrated in vacuo to give compound tert-butyl (3R)-3-[6-(2-cyano-3,6-difluoro-phenoxy)-5,7,8-trideuterio-4-oxo-quinazolin-3-yl]-1-oxa-8-azaspiro[4.5]decane-8-carboxylate (1.4 g, crude) as a brown foam solid. LCMS m/z (ESI): 542.3 [M+H]⁺. ¹H NMR (400 MHz, CHLOROFORM-d) δ=8.33 (s, 1H), 7.46 (dt, J=5.0, 9.4 Hz, 1H), 7.12 (ddd, J=3.6, 7.6, 9.2 Hz, 1H), 5.50 (dt, J=4.0, 9.0 Hz, 1H), 4.27-4.10 (m, 2H), 3.66 (br s, 2H), 3.45-3.16 (m, 2H), 2.48 (dd, J=8.9, 14.0 Hz, 1H), 1.89 (dd, J=4.2, 14.0 Hz, 1H), 1.83-1.69 (m, 3H), 1.60-1.51 (m, 1H), 1.46 (s, 9H).

Step 10: To a solution of [methyl(sulfamoyl)amino]ethane (535.85 mg, 3.88 mmol, 1.5 eq.) in DMAc (12 mL) was added Cs₂CO₃ (2.53 g, 7.76 mmol, 3 eq.) and tert-butyl (3R)-3-[6-(2-cyano-3,6-difluoro-phenoxy)-5,7,8-trideuterio-4-oxo-quinazolin-3-yl]-1-oxa-8-azaspiro[4.5]decane-8-carboxylate (1.4 g, 2.59 mmol, 1 eq.). The mixture was stirred at 65° C. for 16 hr in sealed tube. The reaction mixture was poured into water (60 mL) and washed with MTBE (20 mL×3). The aqueous phase was adjusted pH to 6-7 by HCl (aq., 1 M), extracted with EtOAc (20 mL×2). The organic phase was washed brine (20 mL×2), dried over Na₂SO₄, filtered and concentrated to give compound tert-butyl (3R)-3-[6-[2-cyano-3-[[ethyl(methyl)sulfamoyl]amino]-6-fluoro-phenoxy]-5,7,8-trideuterio-4-oxo-quinazolin-3-yl]-1-oxa-8-azaspiro[4.5]decane-8-carboxylate (1.1 g, 1.47 mmol, 56.76% yield, 88% purity) as a brown foam solid. LCMS m/z (ESI): 660.3 [M+H]⁺. ¹H NMR (400 MHz, CHLOROFORM-d) δ=8.32 (s, 1H), 7.58-7.50 (m, 1H), 7.45-7.34 (m, 1H), 5.48 (br dd, J=4.6, 8.6 Hz, 1H), 4.22-4.04 (m, 2H), 3.64 (br s, 2H), 3.39-3.23 (m, 4H), 3.00 (s, 3H), 2.46 (dd, J=8.8, 14.0 Hz, 1H), 1.86 (dd, J=4.2, 14.0 Hz, 1H), 1.81-1.66 (m, 3H), 1.59-1.49 (m, 1H), 1.44 (s, 9H), 1.20-1.11 (m, 3H).

Step 11: To a solution of tert-butyl (3R)-3-[6-[2-cyano-3-[[ethyl(methyl)sulfamoyl]amino]-6-fluoro-phenoxy]-5,7,8-trideuterio-4-oxo-quinazolin-3-yl]-1-oxa-8-azaspiro[4.5]decane-8-carboxylate (1.1 g, 1.67 mmol, 1 eq.) in acetone (6 mL) was added HCl (12 M, 486.30 uL, 3.5 eq.). The mixture was stirred at 50° C. for 3 hr. The reaction mixture was concentrated. The residue was diluted with water (3 mL) and EtOAc (3 mL), then adjusted pH to 7-8 with aq. NaHCO₃, large amount of solid precipitated. The suspension mixture was stirred at 25° C. for 1 h, filtered and the filtered cake was collected. Compound (3R)-3-[6-[2-cyano-3-[[ethyl(methyl)sulfamoyl]amino]-6-fluoro-phenoxy]-5,7,8-trideuterio-4-oxo-quinazolin-3-yl]-1-oxa-8-azaspiro[4.5]decane (0.63 g, 1.07 mmol, 64.14% yield, 95% purity) was obtained as a gray solid. LCMS m/z (ESI): 560.3 [M+H]⁺. ¹H NMR (400 MHz, DMSO-d₆) δ=8.29 (s, 1H), 7.34-7.13 (m, 2H), 5.28 (br s, 1H), 4.27-4.01 (m, 2H), 3.09 (br d, J=1.2 Hz, 4H), 2.96 (q, J=7.0 Hz, 2H), 2.53 (s, 3H), 2.43-2.32 (m, 1H), 2.13 (br d, J=8.8 Hz, 1H), 2.01-1.68 (m, 4H), 1.01 (br t, J=7.0 Hz, 3H).

Step 12: To a solution of (2,5-dioxopyrrolidin-1-yl) 2-[1-[3-(2,4-dioxohexahydropyrimidin-1-yl)-5-fluoro-1-methyl-indazol-6-yl]-4-hydroxy-4-piperidyl]acetate (273.49 mg, 519.99 umol, 98.2% purity, 1 eq.) in DMF (3 mL) was added (3R)-3-[6-[2-cyano-3-[[ethyl(methyl)sulfamoyl]amino]-6-fluoro-phenoxy]-5,7,8-trideuterio-4-oxo-quinazolin-3-yl]-1-oxa-8-azaspiro[4.5]decane (0.3 g, 519.99 umol, 97% purity, 1 eq.) and TEA (26.31 mg, 259.99 umol, 36.19 uL, 0.5 eq.). The mixture was stirred at 25° C. for 16 hrs. The reaction mixture was added to water (20 mL) slowly, then adjusted pH to 6 by aq·HCl (1 M), the suspension mixture was stirred at 20° C. for 0.5h and filtered. The filtered cake was triturated with i-PrOH/i-PrOAc (5/1, 3 mL) at 25° C. for 2h, filtered and further triturated with MeOH (3 mL) at 25° C. for 2h, and filtered. Compound (3R)-3-[6-[2-cyano-3-[[ethyl(methyl)sulfamoyl]amino]-6-fluoro-phenoxy]-5,7,8-trideuterio-4-oxo-quinazolin-3-yl]-8-[2-[1-[3-(2,4-dioxohexahydropyrimidin-1-yl)-5-fluoro-1-methyl-indazol-6-yl]-4-hydroxy-4-piperidyl]acetyl]-1-oxa-8-azaspiro[4.5]decane (250 mg, 242.71 umol, 46.68% yield, 93.3% purity) was obtained as a gray solid. LCMS m/z (ESI): 961.5 [M+H]⁺. ¹H NMR (400 MHz, DMSO-d6) δ=10.52 (br s, 1H), 10.18 (br d, J=0.9 Hz, 1H), 8.35 (br s, 1H), 7.85 (br t, J=9.1 Hz, 1H), 7.49 (br d, J=5.6 Hz, 1H), 7.40-7.24 (m, 1H), 7.11 (br d, J=6.0 Hz, 1H), 5.29 (br s, 1H), 5.03 (br s, 1H), 4.14 (br s, 2H), 4.02-3.83 (m, 5H), 3.78 (brd, J=4.8 Hz, 1H), 3.62 (brd, J=3.6 Hz, 1H), 3.56-3.44 (m, 2H), 3.16 (brd, J=6.4 Hz, 4H), 3.07 (br d, J=10.0 Hz, 2H), 2.86-2.67 (m, 5H), 2.56 (br s, 2H), 2.37 (br s, 1H), 2.07 (br dd, J=5.4, 6.8 Hz, 1H), 1.88-1.46 (m, 8H), 1.05 (br t, J=6.4 Hz, 3H).

### Evaluation of Compound 1 Free Morphic Forms

The following techniques were used to characterize Compound 1 Free Morphic Forms.

### Example 7: Approximate Solubility at 25° C. And 50° C.

About 5 mg of Compound 1 Form A was weighed to a 2 mL glass vial. 20 μL aliquots of each solvent were added to dissolve the drug substance at 25° C. About 10 mg of Compound 1 Form A was weighed to a 2 mL glass vial. 20 μL aliquots of each solvent were added to dissolve the drug substance at 50° C. Vortex and sonication were applied to assist dissolution. Maximum volume of each solvent added was 1 mL. Approximate solubility was determined by visual observation.

About 5 mg of Compound 1 Form B was weighed to a 2 mL glass vial. 20 μL aliquots of each solvent were added to dissolve the drug substance at 25° C. About 10 mg of Compound 1 Form B was weighed to a 2 mL glass vial. 20pL aliquots of each solvent were added to dissolve the drug substance at 50° C. Vortex and sonication were applied to assist dissolution. Maximum volume of each solvent added is 1 mL. Approximate solubility was determined by visual observation.

The results are summarized in Table 1.

TABLE 1
Approximate solubility of Form A and Form
B at 25° C. and 50° C.

| | Solubility (mg/mL) | | | |
|---|---|---|---|---|
| | Form A | | Form B | |
| Solvent | 25° C. | 50° C. | 25° C. | 50° C. |
| Water | <5 | <10 | <5 | <5 |
| Methanol | ~3 | 5-10 | <5 | <5 |
| Ethanol | <5 | <10 | <5 | <5 |
| Isopropanol | <5 | <10 | <5 | <5 |
| Acetone | 13-17 | 25-29 | <5 | <5 |
| Acetonitrile | 13-17 | 25-33 | <5 | <5 |
| THF | >250 | >500 | 10~13 | 16~20 |
| Ethyl acetate | <5 | <10 | <5 | <10 |
| Isopropyl acetate | <5 | <10 | <5 | <10 |
| DCM | >250 | Not carried out | 83~125 | Not carried out |
| Methyl ethyl | 14-17 | 25-33 | 14-17 | 25-33 |
| ketone | | | | |
| MTBE | <5 | <10 | <5 | <5 |
| Heptane | <5 | <10 | <5 | <5 |
| 2-MeTHF | 5-7 | <10 | <5 | <5 |
| Toluene | <5 | <10 | <5 | <5 |
| 1,4-Dioxane | >250 | >500 | 20~25 | 28~40 |
| DMSO | >250 | >500 | >250 | >500 |

### Example 8: Equilibration with Solvents at 25° C. For 4 Weeks

About 50 mg of Compound 1 free Form A was equilibrated in 0.1-1 mL of solvents at 25° C. for 4 weeks with a stirring bar on a magnetic stirring plate at a rate of 300 rpm. The obtained suspensions were filtered through a 0.45 μm nylon membrane filter by centrifugation at 14,000 rpm. Solid parts (wet cakes) were investigated by XRPD. The results are summarized in Tables 2 and 3.

TABLE 2
Equilibration of Form A with solvents at 25° C. for 4 weeks

| Solvent | XRPD | Comments |
|---|---|---|
| Methanol | Form B | Low crystallinity |
| Acetone | Form B | Medium crystallinity |
| Methyl ethyl ketone | Form B | Medium crystallinity |
| Acetonitrile | Form B | Medium crystallinity |
| THF/heptane (v:v = 1:1) | Form B | Medium crystallinity |
| Ethyl acetate | Form C* | Medium crystallinity |
| Isopropanol | Form A | Low crystallinity |
| MTBE | Form A | Low crystallinity |
| 1,4-Dioxane/heptane | Not carried out | Sticky material |
| (v:v = 1:1) | | |
| DMF | Not carried out | Clear solution |
| | | (about 500 mg/mL) |
| 2-MeTHF | Form C | Medium crystallinity |
| Ethanol | Form A | Low crystallinity |
| Isopropyl acetate | Form A | Low crystallinity |
| Toluene | Form A | Low crystallinity |

*Form C was additionally analyzed by DSC: endothermic peak from 25.9° C.; Tₒₙₛₑₜ 185.3° C.; TGA: 2.2% at 180° C.; and ¹H NMR: 2.7% ethyl acetate by weight (0.2 equivalent by molar ratio).

TABLE 3
Equilibration of Form A with solvents at 25° C. for 4 weeks

| Solvent | XRPD | Comments |
|---|---|---|
| DCM/heptane | Not carried out | Sticky material |
| (v:v = 1:1) | | |
| DCM/methanol | Not carried out | Clear solution |
| (v:v = 1:1) | | (about 500 mg/mL) |
| Methanol/water | Almost amorphous | — |
| (v:v = 22:78) | form | |
| a.w. = 0.9* | | |
| Acetone/water | Almost amorphous | — |
| (v:v = 36:64) | form | |
| a.w. = 0.9* | | |
| Acetonitrile/water | Not carried out | Sticky material |
| (v:v = 42:58) | | |
| a.w. = 0.9* | | |
| THF/water | Not carried out | Clear solution |
| (v:v = 82:18) | | (about 500 mg/mL) |
| a.w. = 0.9* | | |
| DMSO/water | Almost amorphous | — |
| (v:v = 23:77) | form | |
| a.w. = 0.9* | | |
| DMF/water | Almost amorphous | — |
| (v:v = 30:70) | form | |
| a.w. = 0.9* | | |
| Methanol/water | Form B | Low crystallinity |
| (v:v = 69:31) | | |
| a.w. = 0.6* | | |
| Acetone/water | Not carried out | Clear solution |
| (v:v = 86:14) | | (about 500 mg/mL) |
| a.w. = 0.6* | | |
| Acetonitrile/water | Form B | Medium |
| (v:v = 96:4) | | crystallinity |
| a.w. = 0.6* | | |
| THF/water | Not carried out | Clear solution |
| (v:v = 92:8) | | (about 500 mg/mL) |
| a.w. = 0.6* | | |
| DMSO/water | Almost amorphous | — |
| (v:v = 57:43) | form | |
| a.w. = 0.6* | | |
| DMF/water | Not carried out | Clear solution |
| (v:v = 73:27) | | (about 500 mg/mL) |
| a.w. = 0.6* | | |

*The water activity of a binary solvent system is calculated based on UNIFAC method (UNIQUAC Functional-group Activity Coefficients).

FIG. 1 depicts the XRPD pattern of Compound 1 Form B, FIG. 7 depicts the XRPD pattern of Compound 1 Form A, and FIG. 10 depicts the XRPD pattern of Compound 1 Form C.

### Example 9: Equilibration Under a Temperature Cycle

About 50 mg of Compound 1 Form A was equilibrated in 0.1-1 mL of solvents under a temperature cycle between 5° C. to 40° C. at a heating/cooling rate of 0.1° C./min for 16 cycles. The equilibration was executed with a stirring bar on a magnetic stirring plate at a rate of 300 rpm. The obtained suspensions were filtered through a 0.45 pm nylon membrane filter by centrifugation at 14,000 rpm. Solid parts (wet cakes) were investigated by XRPD. The results are summarized in Tables 4 and 5.

TABLE 4
Equilibration of Compound 1 Form A under a temperature cycle

| Solvent | XRPD | Comments |
|---|---|---|
| Methanol | Form B | Low crystallinity |
| Acetone | Form B | Low crystallinity |
| Methyl ethyl ketone | Almost amorphous | — |
| | form | |
| Acetonitrile | Form B | Medium |
| | | crystallinity |
| THF/heptane | Form B | Low crystallinity |
| (v:v = 1:1) | | |
| Ethyl acetate | Form B | Low crystallinity |
| Isopropanol | Form A | Low crystallinity |
| MTBE | Almost amorphous | — |
| | form | |
| 1,4-Dioxane/heptane | Not carried out | Sticky material |
| (v:v = 1:1) | | |
| DMF | Not carried out | Clear solution |
| | | (About 500 mg/mL) |
| 2-MeTHF | Form C (about 20 | After about 8 |
| | cycles) | more cycles: |
| | | Form C converted |
| | | to Form B |
| DCM/heptane | Not carried out | Sticky material |
| (v:v = 1:1) | | |
| Ethanol | Almost amorphous | — |
| | form | |
| Isopropyl acetate | Form A | Low crystallinity |
| Toluene | Form A | Low crystallinity |

TABLE 5
Equilibration of Compound 1 Form A under a temperature cycle

| Solvent | XRPD | Comments |
|---|---|---|
| DCM/methanol (v:v = 1:1) | Not carried out | Clear solution |
| | | (About 500 mg/mL) |
| Methanol/water (v:v = 22:78) | Almost | — |
| a.w. = 0.9* | amorphous form | |
| Acetone/water | Not carried out | Sticky material |
| (v:v = 36:64) | | |
| a.w. = 0.9* | | |
| Acetonitrile/water | Not carried out | Sticky material |
| (v:v = 42:58) | | |
| a.w. = 0.9* | | |
| THF/water (v:v = 82:18) | Not carried out | Clear solution |
| a.w. = 0.9* | | (About 500 mg/mL) |
| DMSO/water (v:v = 23:77) | Form A | Low crystallinity |
| a.w. = 0.9* | | |
| DMF/water (v:v = 30:70) | Form B | Low crystallinity |
| a.w. = 0.9* | | |
| Methanol/water (v:v = 69:31) | Form B | Low crystallinity |
| a.w. = 0.6* | | |
| Acetone/water (v:v = 86:14) | Not carried out | Clear solution |
| a.w. = 0.6* | | (About 500 mg/mL) |
| Acetonitrile/water | Form B** | Medium crystallinity |
| (v:v = 96:4) | | |
| a.w. = 0.65* | | |
| THF/water (v:v = 92:8) | Not carried out | Clear solution |
| a.w. = 0.6* | | (About 500 mg/mL) |
| DMSO/water (v:v = 57:43) | Form A | Low crystallinity |
| a.w. = 0.6* | | |
| DMF/water (v:v = 73:27) | Form B | Medium crystallinity |
| a.w. = 0.6* | | |

*The water activity of a binary solvent system at 5° C. is calculated based on UNIFAC method (UNIQUAC Functional-group Activity Coefficients).
**After about 8 cycles the XRPD: Medium crystallinity, Form B; DSC: melting Tₒₙₛₑₜ 195.4° C.; TGA: 1.9% at 170° C.; ¹H NMR: No detectable residual solvent.

### Example 10: Crystallization at Room Temperature by Slow Evaporation

About 30 mg of Compound 1 Form A was dissolved in 0.2-10 mL of solvents. Obtained solutions were filtered through a 0.45 μm syringe membrane filter. The clear solutions were slowly evaporated in ambient condition (about 20-25° C., 40-80% RH). Solid residues were investigated by XRPD. The results are summarized in Table 6.

TABLE 6
Crystallization at room temperature by slow evaporation

| Solvent | XRPD | Comments |
|---|---|---|
| Methanol | Almost amorphous form | — |
| Acetone | Not carried out | Glassy material |
| Methyl ethyl ketone | Not carried out | Glassy material |
| Acetonitrile | Amorphous form | — |
| Ethyl acetate | Amorphous form | — |
| THF | Not carried out | Glassy material |
| 1,4-Dioxane | Not carried out | Glassy material |
| DCM | Almost amorphous form | — |
| DCM/methanol (v:v = 1:1) | Almost amorphous form | — |

### Example 11: Crystallization from Hot Saturated Solutions by Slow Cooling

About 50 mg of Compound 1 Form A was dissolved in the minimal amount of selected solvents at 50° C. Obtained solutions were filtered through a 0.45 μm syringe membrane filter. The clear solutions were cooled to 5° C. at 0.1° C./min. Samples without precipitates at 5° C. were further cooled to −20° C. Precipitates were collected by centrifugation filtration through a 0.45 μm nylon membrane filter at 14,000 rpm. Solid parts (wet cakes) were investigated by XRPD.

To find out whether Compound 1 Form B can be obtained from clear solution, cooling was also tried to prepare crystalline Form B. About 50 mg of Compound 1 Form A was dissolved in the minimal amount of ACN or acetone at 50° C. Obtained solutions were filtered through a 0.45 μm syringe membrane filter. The clear solutions were cooled to 5° C. at 0.1° C./min. About 2 mg seeds were added into the clear solutions when cooled to about 24° C. Obtained suspensions were cooled to 5° C. at 0.1° C./min and kept at 5° C. for about 12 h. Precipitates were collected by centrifugation filtration through a 0.45 μm nylon membrane filter at 14,000 rpm. Solid parts (wet cakes) were investigated by XRPD. Results showed that crystalline Form B can be obtained from acetone clear solution directly by cooling with the addition of Form B crystalline seeds. The results are summarized in Table 7.

TABLE 7
Crystallization from hot saturated solutions by slow cooling

| Solvent | XRPD | Result |
|---|---|---|
| Methanol | Almost | — |
| | amorphous form | |
| Ethanol | Not carried out | Placed at −20° C. |
| | | for 10 days: few solids |
| Acetone | Not carried out | Placed at −20° C. |
| | | for 11 days: clear solution |
| Methyl ethyl ketone | Not carried out | Placed at −20° C. |
| | | for 11 days: clear solution |
| Acetonitrile | Not carried out | Placed at −20° C. |
| | | for 11 days: clear solution |
| THF | Not carried out | Oil material |
| Ethyl acetate | Not carried out | Placed at −20° C. |
| | | for 11 days: clear solution |
| DCM | Not carried out | Sticky material |
| Ethanol/water | Almost | — |
| (v:v = 1:1) | amorphous form | |
| Acetone/water | Not carried out | Sticky material |
| (v:v = 1:1) | | |
| Acetonitrile/water | Not carried out | Placed at −20° C. |
| (v:v = 1:1) | | for 11 days: clear solution |
| THF/water | Not carried out | Placed at −20° C. |
| (v:v = 1:1) | | for 11 days: clear solution |
| ACN | Almost | — |
| | amorphous form | |
| Acetone | Form B | Few solids were obtained |
| | | at 5° C. Then placed the |
| | | sample at −20° C. for |
| | | 3 days to afford Form B. |

### Example 12: Crystallization by Addition Anti-Solvent

About 50 mg of Compound 1 Form A was dissolved in the minimal amount of selected good solvents at ambient temperature (about 20-25° C.). Obtained solutions were filtered through a 0.45 μm syringe membrane filter. 1-4 folds of anti-solvent were added into the clear solutions slowly until a large amount of solids precipitated out. Precipitates were collected by centrifugation filtration through a 0.45 μm nylon membrane filter at 14,000 rpm. Solid parts (wet cakes) were investigated by XRPD. The results are summarized in Table 8.

TABLE 8
Crystallization by addition of anti-solvent

| | Anti- | | Results |
|---|---|---|---|
| Solvent (mL) | solvent (mL) | XRPD | (After 2 hours) |
| DMSO (0.2) | Water (0.2) | Almost | — |
| | | amorphous form | |
| Acetone (3) | Water (4) | Not carried out | From suspension |
| | | | to sticky material |
| Acetone (3) | Heptane (2) | Form A | Low crystallinity |
| Acetonitrile (3) | Water (5) | Not carried out | From suspension |
| | | | to oil material |
| THF (0.2) | Heptane (0.2) | Almost | Sticky material + |
| | | amorphous form | suspension |
| THF (0.2) | Water (0.2) | Not carried out | Oil material |
| DCM (0.2) | Heptane (0.2) | Not carried out | Sticky material |
| 2-MeTHF (8) | MTBE (8) | Almost | — |
| | | amorphous form | |
| 1,4-Dioxane (0.1) | Water (0.2) | Not carried out | Sticky material |

### Example 13: Crystallization by Vapor Diffusion

About 50 mg of Compound 1 Form A was dissolved in the minimal amount of selected solvents at ambient temperature (about 20-25° C.). Obtained solutions were filtered through a 0.45 μm syringe membrane filter. The clear solutions were transferred into 8 mL glass vials without lids. Then these 8 mL lid less vials were placed to 40 mL glass vials. To the 40 mL vials were added anti-solvents. Then these 40 mL vials were capped tightly and placed at ambient condition for up to 21 days. Precipitates were collected by centrifugation filtration through a 0.45 μm nylon membrane filter at 14,000 rpm. Solid parts (wet cakes) were investigated by XRPD. The results are summarized in Table 9.

TABLE 9
Crystallization by vapor diffusion

| | Anti- | | |
|---|---|---|---|
| Solvents(mL) | solvent(mL) | XRPD | Results |
| DMSO (0.2) | Ethanol (0.8) | Not carried out | Oil material |
| Acetone (2.5) | MTBE (10.0) | Not carried out | Oil material |
| Acetonitrile (3.0) | MTBE (12.0) | Not carried out | Oil material |
| THF (0.2) | MTBE (0.8) | Almost | — |
| | | amorphous form | |
| 2-MeTHF (8.0) | MTBE (32.0) | Not carried out | Few solids |
| 1,4-Dioxane (0.2) | MTBE (0.8) | Almost | — |
| | | amorphous form | |

### Example 14: Crystallization by Heat-Cool DSC

Polymorphic behavior of Compound 1 Form A was investigated by two different heat-cool DSC cycles. A Tzero pan and a Tzero hermetic lid with a pin hole were used for this experiment. No new crystalline form was obtained by heat-cool DSC. The results are summarized in Table 10.

TABLE 10
Crystallization by heat-cool DSC

| Heat-cool cycles | Thermal events |
|---|---|
| Cycle 1 | Heated to 185° C.: Endothermic |
| Step 1: 30° C. to 185° C. | peak from about 30° C.; Melting |
| at 10° C./min; | Tₒₙₛₑₜ 167.7° C., enthalpy about |
| Step 2: 185° C. to −20° C. | 16 J/g |
| at 20° C./min; | Heated to 250° C.: Tg 149.8° C., |
| Step 3: reheat to 250° C. | Delta Cp 0.3 J(g. ° C.) |
| at 10° C./min. | |
| Cycle 2 | Heated to 185° C.: Endothermic |
| Step 1: 30° C. to 185° C. | peak from about 30° C.; Melting |
| at 10° C./min; | Tₒₙₛₑₜ 167.7° C., enthalpy about |
| Step 2: 185° C. to −20° C. | 17 J/g |
| at 2° C./min; | Heated to 250° C.: Tg 145.9° C., |
| Step 3: reheat to 250° C. | Delta Cp 0.2 J(g. ° C.) |
| at 10° C./min. | |

### Example 15: Bulk Stability

Compound 1 Form B was placed at 25° C./92% RH in an open container, at 40° C./75% RH in an open container and at 60RC in a closed container for 1 week. Samples after the stress were characterized by XRPD and HPLC and inspected for color change. The results are summarized in Table 11.

TABLE 11
Bulk stability

| Initial purity | |
|---|---|
| 99.3% | |
| Purity | Color |

Solid state, 25° C./92% RH, open container, 1 week

| | | |
|---|---|---|
| Bulk (HPLC) | 99.2% | No change of color |

| | |
|---|---|
| Bulk (XRPD) | Form B |
| | No change in crystallinity |

Solid state, 40° C./75% RH, open container, 1 week

| | | |
|---|---|---|
| Bulk (HPLC) | 99.3% | No change of color |

| | |
|---|---|
| Bulk (XRPD) | Form B |
| | No change in crystallinity |

Solid state, 60° C., tight container, 1 week

| | | |
|---|---|---|
| Bulk (HPLC) | 99.3% | No change of color |

| | |
|---|---|
| Bulk (XRPD) | Form B |
| | No change in crystallinity |

Solid state, photo (visible light, 1.2 million lux-hrs)

| | | |
|---|---|---|
| Bulk (HPLC) | 96.9% | No change of color |
| Control (HPLC) | 99.4% | |

| | |
|---|---|
| Bulk (XRPD) | Form B |
| | Slight decrease in crystallinity |

### Example 16: Water Sorption and Desorption Experiments

Water sorption and desorption behavior of Compound 1 Form B was investigated by DVS at 25° C. with a cycle of 40-0-95-0-40% RH, dm/dt 0.002, min. equilibration time 60 min and maximum equilibration time 360 min XRPD was measured after the DVS test to determine form change. The results are summarized in Table 12.

TABLE 12
Water sorption and desorption experiments

| | Method | | | |
|---|---|---|---|---|
| | 40-0-95-0-40% RH, dm/dt 0.002, min. equilibration | | | |
| | time 60 min, max. equilibration time 360 min, 25° C. | | | |
| Relative | 1^{st} sorp. | 1^{st} desorp. | 2^{nd} sorp. | 2^{nd} desorp. |
| humidity | Weight % | Weight % | Weight % | Weight % |
| at 25° C. | change | change | change | change |
| 0% | 0.0 | 0.0 | 0.0 | 0.0 |
| 10% | 0.1 | 0.1 | 0.1 | 0.1 |
| 20% | 0.2 | 0.3 | 0.2 | 0.3 |
| 30% | 0.3 | 0.4 | 0.3 | 0.4 |
| 40% | 0.4 | 0.5 | 0.4 | 0.5 |
| 50% | 0.5 | N/A* | N/A | 0.8 |
| 60% | 0.6 | N/A | N/A | 0.9 |
| 70% | 0.7 | N/A | N/A | 1.0 |
| 80% | 0.9 | N/A | N/A | 1.2 |
| 90% | 1.2 | N/A | N/A | 1.4 |
| 95% | 1.6 | N/A | N/A | 1.6 |

XRPD after DVS test
No form change, Form B
DVS
1.6% water uptake at 95% RH
*N/A: not applicable

### Example 17: Compression Simulation Experiments

About 20 mg of Compound 1 Form B was compressed for 5 minutes under 2 MPa and 10 MPa with a hydraulic press. Potential form change and degree of crystallinity were evaluated by XRPD. The results are summarized in Table 13.

TABLE 13
Compression simulation experiments

| Pressure | XRPD | Comments |
|---|---|---|
| 2 MPa | Form B | Slight decrease in crystallinity; |
| | | Slight discoloration. |
| 10 MPa | Form B | Slight decrease in crystallinity; |
| | | Slight discoloration. |

### Example 18: Dry Grinding Simulation Experiments

About 20 mg of Compound 1 Form B was ground manually with a mortar and a pestle for 1, 2 and 5 min. Potential form change, and degree of crystallinity were evaluated by XRPD. The results are summarized in Table 14.

TABLE 14
Dry grinding simulation experiments

| Grinding time | XRPD | Comments |
|---|---|---|
| 1 min | Form B | Slight decrease in crystallinity. |
| 2 min | Form B | Slight decrease in crystallinity. |
| 5 min | Form B | Slight decrease in crystallinity. |

### Example 19: Wet Granulation Simulation Experiments

Water or ethanol was added drop wise to about 20 mg of Compound 1 Form B until the sample was wetted sufficiently. Wet sample was ground gently with in a mortar and a pestle. Post granulation sample was dried under ambient condition for 10 min. Potential form change and degree of crystallinity were evaluated by XRPD. The results are summarized in Table 15.

TABLE 15
Wet granulation simulation experiments

| Granulation solvents | XRPD |
|---|---|
| Water | Form B; no crystallinity decrease. |
| Ethanol | Form B; no crystallinity decrease. |

### Example 20: Preparation of Compound 1 Form B

Form B was prepared using the procedure below.

Experiment 1. About 500 mg of Compound 1 Form A was weighed into a 20 mL glass vial. Then, 3 mL of ACN/H₂O (v:v=96:4) was added into the vial under stirring at 40° C. for about 5 min to obtain a suspension.

About 2 mg of Form B seeds was added into above suspension.

The suspension was equilibrated under a temperature cycle between 5° C. to 40° C. at a heating/cooling rate of 0.1° C./min for about 9 cycles.

Solids were collected by suction filtration and then dried at 35° C. under vacuum for about 1 hour.

400 mg of Form B was obtained as off-white solid in 80% yield.

Experiment 2. About 300 mg of Compound 1 Form A was weighed into a 20 mL glass vial. Then, 3 mL of ACN/H₂O (v:v=96:4) was added into the vial under stirring at 40° C. for about 5 min to obtain a sticky material. (Sticky material)

About 5 mg of Form B seeds was added into above sticky material. After 7 hours, it converted to suspension. (Sticky material to Suspension)

The equilibration was executed with a stirring bar on a magnetic stirring plate at a rate of 300 rpm under a temperature cycle between 5° C. to 40° C. at a heating/cooling rate of 0.1° C./min for about 8 cycles. (Suspension).

Solids were collected by suction filtration and then dried at 30° C. under vacuum for about 4 hours.

174 mg of Form B was obtained as off-white solid in 58% yield.

In other aspects Compound 1 Form B is obtained using methods described herein with modification of the temperature, stir rate, or crystallization time. For example, in certain embodiments Compound 1 Form B is formed without using a temperature cycle and is instead crystallized from a solution with stirring at a temperature of between about 0° C. and about 50° C., a temperature between about 20° C. and about 35° C., a temperature between about 0° C. and about 20° C., or a temperature between about 10° C. and about 30° C. In certain embodiments the temperature is about 0° C., about 5° C., about 10° C., about 15° C. about 20° C., about 25° C., about 30° C., about 35° C., about 40° C., about 45° C., or about 50° C.

In certain embodiments the method described herein is modified to have a slow stir rate, for example with stirring between about 50 rpm and about 300 rpm, stirring between about 150 rpm and about 300 rpm, or stirring between about 200 rpm and about 300 rpm. In certain embodiments the stirring is about 50 rpm, about 60 rpm, about 70 rpm, about 80 rpm, about 90 rpm, about 100 rpm, about 110 rpm, about 120 rpm, about 130 rpm, about 140 rpm, about 150 rpm, about 160 rpm, about 170 rpm, about 180 rpm, about 190 rpm, about 200 rpm, about 210 rpm, about 220 rpm, about 230 rpm, about 240 rpm, about 250 rpm, about 260 rpm, about 270 rpm, about 280 rpm, about 290 rpm, or about 300 rpm.

In certain embodiments the method described herein is modified to have a long crystallization period, for example a crystallization time of between about 10 hours and about 30 hours, a crystallization time between about 15 hours and about 30 hours, a crystallization time between about 20 hours and about 30 hours, a crystallization time between about 10 hours and about 25 hours, or a crystallization time between about 15 hours and about 25 hours. In certain embodiments the crystallization time is about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, or about 35 hours. In other embodiments the crystallization time is more than about 30 hours.

In certain aspects a temperature or temperature range without a temperature cycle, slow stir rate, and prolonged crystallization time are used to prepare Compound 1 Form B. In certain aspects a temperature cycle, slow stir rate, and prolonged crystallization time are used to prepare Compound 1 Form B.

### Evaluation of Compound 1 Salt Morphic Forms

### Example 21: Slurry Equilibration

About 30 mg of Compound 1 Form A and 0.5 or 1 equivalent of base were added into a solvent in a 2 mL glass vial. For the experiments with NaHCO₃, Ca(OH)₂ and Mg(OH)₂, 5% water by volume was added to improve solubility of counter ions in tested solvents. Obtained mixtures were stirred at 25° C. for at least 48 hours.

Obtained suspensions were filtered through a 0.45 μm nylon membrane filter by centrifugation at 14,000 rpm. After being dried at 25° C. under vacuum for 2 h, solid parts were analyzed by XRPD. The results are summarized in Table 16.

TABLE 16
Slurry equilibration

| | Solvent | | |
|---|---|---|---|
| Counter ions | Methanol | Acetone | Acetonitrile |
| Free form only | Almost amorphous | Form B | Form B |
| | form | | |
| NaOH (1 equiv.) | Sodium salt Form D, | Almost amorphous | Almost amorphous |
| | medium crystallinity | form | form |
| KOH (1 equiv.) | Almost amorphous | Potassium salt Form | Potassium salt Form |
| | form | G, low crystallinity | H, medium |
| | | | crystallinity |
| Erbumine (1 equiv.) | Few solids | Almost amorphous | Almost amorphous |
| | | form | form |
| NH₃ (1 equiv.) | Almost amorphous | Hazy suspension | Form I* |
| | form | | |
| Choline (1 equiv.) | Clear solution | Sticky material | Sticky material |
| L-Lysine (1 equiv.) | Almost amorphous | Almost amorphous | Form B, low |
| | form | form | crystallinity |
| L-arginine (1 equiv.) | Almost amorphous | Almost amorphous | Almost amorphous |
| | form | form | form |
| Methylglucamine (1 | Sticky material | Almost amorphous | Almost amorphous |
| equiv.) | | form | form |
| TRIS (1 equiv.) | Few solids | Form B, low | Form B, low |
| | | crystallinity | crystallinity |
| Betaine (1 equiv.) | Almost amorphous | Form B, low | Form B, low |
| | form | crystallinity | crystallinity |
| Ca(OH)₂ (0.5 equiv.) | Almost amorphous | Sticky material | Sticky material |
| | form | | |
| Mg(OH)₂ (0.5 equiv.) | Form B, low | Few solids | Form B |
| | crystallinity | | |
| NaHCO₃ (1 equiv.) | Sodium salt Form E, | Sodium salt Form F, | Sodium salt Form F, |
| | medium crystallinity | medium crystallinity | medium crystallinity |

*Free form according to IC, the ratio of free form to NH₄⁺ is 1:0.1 (FIG. 37).

FIG. 23 depicts the XRPD pattern of Compound 1 sodium salt Form D, FIG. 26 depicts the XRPD pattern of Compound 1 sodium salt Form E, FIG. 17 depicts the XRPD pattern of Compound 1 sodium salt Form F, FIG. 29 depicts the XRPD pattern of Compound 1 potassium salt Form G, and FIG. 32 depicts the XRPD pattern of Compound 1 potassium salt Form H.

### Example 22: Anti-Solvent Addition

Clear solution obtained from slurry equilibration was treated by addition of antisolvent (Table 17).

TABLE 17
Anti-solvent addition

| Counter ion | Solvent/mL | Anti-solvent/mL | Notes |
|---|---|---|---|
| Choline | MeOH/0.2 | MTBE/0.3 | Sticky material |

### Example 23: Re-Slurry

Sticky material or clear solution obtained from slurry equilibration was treated by slow evaporation under ambient condition and by addition of MTBE, toluene and ethyl acetate (EA) (Table 18).

TABLE 18
Re-slurry

| Counter ion | MTBE | Toluene | Ethyl Acetate |
|---|---|---|---|
| Choline | Sticky material | Sticky material | Sticky material |
| Methylglucamine | Not carried out | Not carried out | Form A |
| Ca(OH)₂ | Not carried out | Not carried out | Almost amorphous |

Salt Forms identified in the investigation (Table 16) were further characterized by XRPD, DCS, TGA, KF, HPLC and ¹H NMR/IC (Table 19).

TABLE 19
Characterization of Salt Forms

| | Sodium salt | Sodium salt | Sodium salt | Potassium | Potassium |
|---|---|---|---|---|---|
| | Form D, | Form E, | Form F, | salt Form G, | salt Form H, |
| Property | hydrate | hydrate | hydrate | solvate | hydrate |
| Preparation | MeOH | MeOH | ACN | Acetone | ACN |
| solvent | | | | | |
| Purity (HPLC) | 97.4% | 98.3% | 98.9% | 98.9% | 99.0% |
| Crystallinity | Medium | Medium | Medium | Low | Medium |
| (XRPD) | crystallinity | crystallinity | crystallinity | crystallinity | crystallinity |
| Melting onset | Dehydration | Dehydration | Dehydration | Multiple | Dehydration |
| (DCS, ° C.) | from 4.5° C., | from 5.6° C., | from 59.8° C., | thermal events | from 4.2° C., |
| | 67.5° C., | 61.1° C., | 132.5° C., | | 87.2° C., |
| | endothermic | Tₒₙₛₑₜ 228.7° C. | Tₒₙₛₑₜ 197.7° C. | | endothermic |
| | peak from | | | | peak from |
| | 117.9° C., | | | | 123.0° C., |
| | Tₒₙₛₑₜ 229.5° C. | | | | Tₒₙₛₑₜ 221.7° C. |
| Enthalpy | 27.83 | 26.01 | 26.41; 41.85 | — | 33.48 |
| (DSC, J/g) | | | | | |
| Weight loss | 6.9% at | 10.8% at | 2.7% at 100° C., | 4.5% at 170° C., | 6.6% at |
| (TGA) | 150° C. | 180° C. | 3.2% at 100° C.- | 4.8% at 170° C.- | 170° C. |
| | | | 170° C. | 230° C. | |
| Stoichiometric | Compound: | Compound: | Compound: | Compound: | Compound: |
| ratio (¹H | Na⁺ = 1:1 | Na⁺ = 1:1 | Na⁺ = 1:1.2 | K⁺ = 1:1 | K⁺ = 1:1.2 |
| NMR or IC) | | | | | |
| Residual | No detectable | No detectable | No detectable | 2.5% acetone | No detectable |
| solvent (¹H | residual | residual | residual | by weight (0.42 | residual |
| NMR) | solvent | solvent | solvent | equiv. by molar | solvent |
| | | | | ratio) | |
| Water content | Not carried | 10.2% by | 6.2% by | Not carried | Not carried |
| (by KF) | out | weight | weight | out | out |

### Example 24: Preparation of Compound 1 Sodium Salt Form F

Sodium salt Form F was prepared using the procedure below.

About 500 mg of Compound 1 Form A and 47 mg of NaHCO₃(about 1.0 equivalent) were weighed into a 20 mL glass vial. Then, 8 mL of ACN and 100 μL of water were added into the vial under stirring at 25° C. at a rate of 400 rpm for about 1 min to obtain a suspension. (Suspension)

About 2 mg of sodium salt Form F seeds was added into above suspension. (Suspension)

The suspension converted to a sticky material after about 5 minutes. (Suspension to Sticky material)

The sticky material was converted to suspension after equilibrated at 25° C. for about 6 hours. (Sticky material to Suspension)

Solids were collected by suction filtration after equilibrated at 25° C. for about 9 days and then dried at 30° C. under vacuum for about 3 hours.

About 419 mg of sodium salt Form F was obtained as off-white solid in 76% yield. The chemical and physicochemical proposed of Compound 1 sodium salt Form F are summarized in Table 20.

TABLE 20
Chemical and Physicochemical Properties of Sodium Salt Form F

| | Physical Form |
|---|---|
| Property | Sodium salt Form F, hydrate |
| HPLC purity | 98.9% |
| Stoichiometry by HPLC or IC | 1:1.2 |
| Residual solvent by ¹H NMR, weight % | No detectable residual solvent |
| Water content by Karl Fischer, weight % | 4.6% by weight (2.5 equiv. water) |
| Crystallinity by XRPD | Medium crystallinity |
| DSC, heating rate [10° C./min] | Dehydration from 13.7° C., 64.7° C., 116.5° C. |
| | Tₒₙₛₑₜ 194.7° C. |
| Melting enthalpy (J/g) | 32.4 J/g |
| Weight loss in % at ° C. (Thermogravimetry, | 4.8% at 180° C. |
| heating rate [10° C./min]) | |

### Example 25: Bulk Stability of Compound 1 Sodium Salt Form F

Sodium salt Form F was placed at 25° C./92% RH in an open container, at 40° C./75% RH in an open container and at 60RC in a closed container for 1 week. Samples after the stress were characterized by XRPD and HPLC and inspected for color change. The results are summarized in Table 21.

TABLE 21
Bulk Stability of Sodium Salt Form F

| Initial purity | |
|---|---|
| 98.9% | |
| Initial color | |
| Off-white | |
| Purity | Color |

Solid state, 25° C./92% RH, open container, 1 week

| | | |
|---|---|---|
| Bulk (HPLC) | 97.7% | No change of color |

| | |
|---|---|
| Bulk (XRPD) | No form change |

Solid state, 40° C./75% RH, open container, 1 week

| | | |
|---|---|---|
| Bulk (HPLC) | 98.5% | No change of color |

| | |
|---|---|
| Bulk (XRPD) | No form change |

Solid state, 60° C., tight container, 1 week

| | | |
|---|---|---|
| Bulk (HPLC) | 98.9% | No change of color |

| | |
|---|---|
| Bulk (XRPD) | No form change, slight crystallinity decrease |

Solid state, photo (visible light, 1.2 million lux-hrs)

| | | |
|---|---|---|
| Bulk (HPLC) | 97.9% | No change of color |
| Control (HPLC) | 98.9% | |

| | |
|---|---|
| Bulk (XRPD) | No form change |

### Example 26: Solubility of Compound 1 Form B and Compound 1 Sodium Salt Form F

Solubility of the Form B and sodium salt Form F was measured in 7 aqueous pH buffers and bio-relevant fluids including pH 1.2 HCl solution (0.2N), pH 4.5 acetate buffer (50 mM), pH 6.8 phosphate buffer (50 mM), pH 2.0 SGF, pH 6.5 FaSSIF-v1, pH 5.0 FeSSIF-v1 and water at 37° C. for 2 h and 24 h, respectively.

Accurate 20 mg of the Form B was weighed into a 20 mL glass vial. Then, 10 mL of solubility medium was added. The sodium salt Form F amount used was equivalent to 20 mg anhydrous form. Obtained suspensions were stirred at 37° C. at 400 rpm and sampled at 2 hours and at 24 hours, respectively. The samples were centrifuged at 37° C. at 4,000 rpm for 4 min. Supernatants were analyzed by HPLC and pH meter for solubility and pH value, respectively. Residual solids (wet cakes) from the 24 hours samples were also characterized by XRPD to determine physical form. The results are shown in Table 22.

TABLE 22
Solubility at 37° C., target concentration 2 mg/mL (in form), equilibration for 24
hours, LOQ: 1.5 μg/mL

| | Form B | | | Sodium salt Form F | | |
|---|---|---|---|---|---|---|
| Solubility | Solubility | | XRPD | Solubility | | XRPD |
| media | 2 h | 24 h (pH) | 24 h | 2 h | 24 h (pH) | 24 h |
| pH 1.2 | 11.7 μg/mL | 28.0 | No form | 19.3 μg/mL | 51.1 | No form change, |
| HCl solution | | μg/mL | change | | μg/mL | crystallinity |
| (0.2N) | | (pH = 0.9) | | | (pH = 1.0) | decrease. |
| | | | | | | IC: Compound: |
| | | | | | | :Na⁺ = 1:0.2 |
| pH 4.5 | <LOQ | <LOQ | No | <LOQ | 3.7 | No form change |
| acetate buffer | | (pH = 4.5) | form | | μg/mL | |
| (50 mM) | | | change | | (pH = 4.6) | |
| pH 6.8 | <LOQ | <LOQ | No | <LOQ | <LOQ | No form change |
| phosphate | | (pH = 6.8) | form | | (pH = 6.9) | |
| buffer | | | change | | | |
| (50 mM) | | | | | | |
| SGF, pH 2.0 | 3.8 μg/mL | 5.2 | No | 10.9 μg/mL | 8.6 | No form change, |
| | | μg/mL | form | | μg/mL | slight crystallinity |
| | | (pH = 2.1) | change | | (pH = 2.2) | decrease. |
| | | | | | | IC: Compound: |
| | | | | | | :Na⁺ = 1:0.3 |
| FaSSIF-v1, | <LOQ | <LOQ | No | <LOQ | <LOQ | No form change |
| pH 6.5 | | pH = 6.5 | form | | (pH = 6.7) | |
| | | | change | | | |
| FeSSIF-v1, | 4.8 μg/mL | 6.7 | No | 6.7 μg/mL | 10.4 | No form change |
| pH 5.0 | | μg/mL | form | | μg/mL | |
| | | (pH = 5.0) | change | | (pH = 5.1) | |
| Water | <LOQ | 4.6 | No | 98.8 μg/mL | 73.0 | No form change |
| | | μg/mL | form | | μg/mL | |
| | | (pH = 7.0) | change | | (pH = 8.7) | |

Both the sodium salt Form F and Form B showed pH dependent solubility in aqueous media. Sodium salt Form F showed better solubility than that of the Form B, especially in pH 1.2 HCl solution, in pH 4.5 acetate buffer, in SGF, and in FeSSIF-v1. Form B showed no form change after solubility test. Sodium salt Form F showed crystallinity decrease after solubility test in pH 1.2 HCl solution and in SGF, respectively. IC showed that sodium salt Form F partially disproportionated to form after solubility test.

### Example 27: Hygroscopicity of Compound 1 Sodium Salt Form F

Hygroscopicity of sodium salt Form F was evaluated by dynamic vapor sorption (DVS) test at 25° C. The results are summarized in Table 23.

Sodium salt Form F is slightly hygroscopic below 80% RH. Then it becomes hygroscopic and shows 12.6% water uptake from 80% RH to 95%/RH at 25° C. After the DVS test, sodium salt Form F showed no form change and no crystallinity decrease.

TABLE 23
Hygroscopicity by DVS at 25° C. dm/dt = 0.002%

| Relative | 1^{st} sorp. | 1^{st} desorp. | 2^{nd} sorp. | 2^{nd} desorp. |
|---|---|---|---|---|
| humidity | Weight % | Weight % | Weight % | Weight % |
| at 25° C. | change | change | change | change |
| 0% | N/A | 0.0 | 0.0 | N/A |
| 10% | N/A | 0.6 | 0.5 | N/A |
| 20% | N/A | 1.2 | 0.8 | N/A |
| 30% | N/A | 2.2 | 1.2 | N/A |
| 40% | 3.1 | 3.7 | 1.5 | 3.9 |
| 50% | 3.3 | 5.0 | 2.2 | 5.2 |
| 60% | 3.4 | 6.1 | 3.5 | 6.2 |
| 70% | 3.7 | 7.5 | 5.8 | 7.7 |
| 80% | 4.1 | 9.7 | 7.9 | 9.9 |
| 90% | 7.0 | 15.5 | 13.1 | 16.4 |
| 95% | 16.7 | 16.7 | 19.6 | 19.6 |

XRPD after DVS test
No form change, Sodium Salt Form F
DVS
1.0% water uptake from 40% RH to 80% RH, 12.6% water uptake from 80% RH to 95% RH

### Example 28: Instrumental Methods

X-ray Powder Diffractometer (XRPD)

| | |
|---|---|
| Instrument | Bruker D8 Advance |

XRPD Method 1

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Monocrystalline silicon, flat surface, without kapton film |

XRPD Method 2

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Monocrystalline silicon, covered by kapton film |

XRPD Method 3

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.06 second per step |
| Scan range | 3° to 40° |
| Sample rotation speed | 15 rpm |
| Sample holder | Monocrystalline silicon, covered by kapton film |

Single Crystal X-Ray Diffraction

| | |
|---|---|
| Instrument/Diffractometer | Rigaku Oxford Diffraction XtaLAB Synergy |
| | four-circle diffractometer equipped with a |
| | HyPix-6000HE area detector. |
| Cryogenic system | Oxford Cryostream 800 |
| Cu | λ = 1.54184 Å, 50 W, Micro focus source with |
| | multilayer mirror (μ-CMF). |
| Distance from the crystal to the CCD detector | d = 35 mm |
| Tube Voltage | 50 kV |
| Tube Current | 1 mA |

Differential Scanning Calorimetric (DSC)

| | |
|---|---|
| Instrument | TA Discovery 2500 or Q2000 |

Method

| | |
|---|---|
| Sample pan | Tzero pan and Tzero hermetic lid with a pin hole of 0.7 mm in |
| | diameter |
| Temperature range | 0 to 250° C. or before decomposition |
| Heating rate | 10° C./min |
| Nitrogen flow | 50 mL/min |
| Sample mass | About 0.5-1.5 mg |

Thermal Gravimetric Analysis (TGA)

| | |
|---|---|
| Instrument | Discovery 5500 or Q5000 |
| Sample pan | Aluminum, sealed |
| Start temperature | Ambient condition (below 35° C.) |
| Final temperature | 300° C. or abort next segment if weight <80% (w/w) |
| Heating rate | 10° C./min |
| Nitrogen flow | Balance 10 mL/min; sample chamber 25 mL/min |
| Sample mass | About 2 mg |

Dynamic Vapor Sorption (DVS)
DVS Method 1

| | |
|---|---|
| Instrument | Intrinsic |
| Total gas flow | 200 sccm |
| Oven temperature | 25° C. |
| Solvent | Water |
| Method | Cycle: 40-0-95-0-40% RH |
| | Stage Step: 10% |
| | Equilibrium: 0.002 dm/dt (%/min) |
| | Minimum dm/dt stability duration: 60 min |
| | Maximum dm/dt stage time: 360 min |
| Sample mass | About 20 mg |

DVS Method 2

| | |
|---|---|
| Instrument | Advantage |
| Total gas flow | 200 sccm |
| Oven temperature | 25° C. |
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |
| Sample mass | About 20 mg |

Karl Fischer (KF)

| | |
|---|---|
| Instrument | Mettler Toledo Coulometric KF Titrator C30 |
| Method | Coulometric |
| Sample mass | About 5-10 mg |

Polarized Light Microscope (PLM)

| | |
|---|---|
| Instrument | Nikon LV100POL |
| Method | Crossed polarizer, silicone oil added |

Nuclear Magnetic Resonance (NMR)

| | |
|---|---|
| Instrument | Bruker Avance-AV 400M (for ¹H-NMR) |
| Frequency | 400 MHz |
| Probe | 5 mm PABBO BB/19F-1H/D Z-GRD Z108618/0406 (for ¹H- |
| | NMR) |
| Number of scan | 8 |
| Temperature | 297.6 K |
| Relaxation delay | 1 second |

Ion Chromatography (IC)

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L |
| | NaHCO₃ |
| Eluent (cation) | 2.5 mmol/L MSA |
| Suppressor solutions | 0.5% H₂SO₄ |
| Column: | Cation Column C4-150 |
| Column temperature: | 30° C. |
| Flow rate: | 0.9 mL/min (cation) |
| Diluent: | ACN:Water = 1:1 |
| Injection volume: | 20 μL |

Ultra Performance Liquid Chromatography (UPLC)

| | |
|---|---|
| Instrument | Agilent 1290 infinity II |
| UPLC method | Wave length: 250 nm |
| | Column: C18, 2.1 mm × 100 mm 1.7 μm |
| | Detector: DAD |
| | Column temperature: 40° C. |
| | Flow rate: 0.4 mL/min |
| | Diluent: ACN/H₂O(8:2, v:v) |
| | Mobile phase A: 0.05% TFA in water |
| | Mobile phase B: 0.05% TFA in ACN |
| | Injection volume: 1 μL |
| | Gradient: |

| Time (min) | Mobile Phase A (%) | Mobile Phase B (%) |
|---|---|---|
| 0 | 95 | 5 |
| 1 | 95 | 5 |
| 8 | 20 | 80 |
| 10 | 20 | 80 |
| 10.1 | 95 | 5 |
| 12 | 95 | 5 |

High Performance Liquid Chromatograph (HPLC)

| | |
|---|---|
| Instrument | Agilent 1260 Infinity II Binary Pump |
| HPLC method | Wave length: 254 nm |
| | Column: XbridgeC18, 3.5 μm, 4.6 × 150 mm |
| | Detector: DAD |
| | Column temperature: 40° C. |
| | Flow rate: 1 mL/min |
| | Mobile phase A: 0.05% TFA in water |
| | Mobile phase B: 0.05% TFA in ACN |
| | Diluent: MeOH/H₂O(8:2, v:v) |
| | Injection volume: 20 μL |
| | Gradient: |

| Time (min) | Mobile Phase A (%) | Mobile Phase B (%) |
|---|---|---|
| 0 | 90 | 10 |
| 5 | 50 | 50 |
| 10 | 50 | 50 |
| 17 | 20 | 80 |
| 17.1 | 90 | 10 |
| 22 | 90 | 10 |
| 0 | 90 | 10 |

### Example 29: Stability Study for Compound 1 Form A

TABLE 24
Results of stability study of Compound 1 Form A under
5 ± 3° C. storage conditions

Test Result (5 ± 3° C.)

| Testing Item | | Initial | 6 M |
|---|---|---|---|
| Appearance | | white solid | white solid |
| Water Content by KF (% w/w) | | 0.59% | 4.7% |
| Assay by UPLC (% w/w) | | 97.3% | 92.0% |
| Related Substances | Total | 2.6% | 2.8% |
| by UPLC (% area) | Impurities | | |

| | | |
|---|---|---|
| Chiral purity by HPLC (% area) | 99.2% | 99.2% |
| XRPD | Form A | Form A |

TABLE 25
Results of stability study of Compound 1 Form A under 25° C. ±
2° C./60% RH ± 5% RH storage conditions

| | | Test Result | | |
|---|---|---|---|---|
| | | (25° C. ± 2° C./60% RH ± 5% RH) | | |
| Testing Item | | Initial | 3 M | 6 M |
| Appearance | | white | white | white |
| | | solid | solid | solid |
| Water Content by KF (% w/w) | | 0.59% | 3.6% | 4.2% |
| Assay by UPLC (% w/w) | | 97.3% | 93.3% | 92.9% |
| Related Substances, | Total | 2.6% | 2.6% | 2.9% |
| UPLC (% area) | Impurities | | | |

| | | | |
|---|---|---|---|
| Chiral purity by HPLC (% area) | 99.2% | 99.2% | 99.2% |
| XRPD | Form A | Form A | Form A |

TABLE 26
Results of stability study of Compound 1 Form A under 30 ±
2° C./65 ± 5% RH storage conditions

| | | Test Result (30 ± 2° C./65 ± 5% RH) | |
|---|---|---|---|
| Testing Item | | Initial | 6 M |
| Appearance | | white solid | white solid |
| Water Content by KF (% w/w) | | 0.59% | 3.8% |
| Assay by UPLC (% w/w) | | 97.3% | 92.4% |
| Related Substances, | Total | 2.6% | 3.0% |
| UPLC (% area) | Impurities | | |

| | | |
|---|---|---|
| Chiral purity by HPLC (% area) | 99.2% | 99.2% |
| XRPD | Form A | Form A |

TABLE 27
Results of stability study of Compound 1 Form A under 40° C. ±
2° C./75% RH ± 5% RH storage conditions

| | | Test Result | | |
|---|---|---|---|---|
| | | (40° C. ± 2° C./75% RH ± 5% RH) | | |
| Testing Item | | Initial | 3 M | 6 M |
| Appearance | | white | white | white |
| | | solid | solid | solid |
| Water Content by KF (% w/w) | | 0.59% | 3.9% | 4.5% |
| Assay by UPLC (% w/w) | | 97.3% | 92.2% | 92.5% |
| Related Substances, | Total | 2.6% | 2.7% | 3.3% |
| UPLC (% area) | Impurities | | | |

| | | | |
|---|---|---|---|
| Chiral purity by HPLC (% area) | 99.2% | 99.2% | 99.2% |
| XRPD | Form A | Form A | Form A |

As shown in the foregoing stability study results for Compound 1 Form A, all the tested parameters, including appearance, water content, chiral purity, assay and related substance meet the acceptance criteria. The water content increased overtime in each stability condition.

### Example 30: Stability Study for Compound 1 Form B

TABLE 28
Results of stability study of Compound 1 Form B under
5 ± 3° C. storage conditions

Test Result (5 ± 3° C.)

| Testing Item | | Initial | 6 M |
|---|---|---|---|
| Appearance | | white solid | white solid |
| Water Content by KF (% w/w) | | 0.4% | 1.2% |
| Purity by UPLC (% area) | | 98.9% | 99.0% |
| Assay by UPLC (% w/w) | | 99.4% | 97.7% |
| Related Substances, | Total | 1.1% | 1.0% |
| UPLC (% area) | Impurities | | |

| | | |
|---|---|---|
| Chiral purity by HPLC (% area) | 99.8% | 99.9% |
| XRPD | Form B | Form B |

TABLE 29
Results of stability study of Compound 1 Form B under 25° C. ±
2° C./60% RH ± 5% RH storage conditions

| | | Test Result | | |
|---|---|---|---|---|
| | | (25° C. ± 2° C./60% RH ± 5% RH) | | |
| Testing Item | | Initial | 3 M | 6 M |
| Appearance | | white | white | white |
| | | solid | solid | solid |
| Water Content by KF (% w/w) | | 0.4% | 0.7% | 0.9% |
| Purity by UPLC (% area) | | 98.9 | 98.9 | 99.1 |
| Assay by UPLC (% w/w) | | 99.4% | 97.2% | 97.8% |
| Related Substances, | Total | 1.1% | 1.1% | 0.90% |
| UPLC (% area) | Impurities | | | |

| | | | |
|---|---|---|---|
| Chiral purity by HPLC (% area) | 99.8% | 99.9% | 99.9% |
| XRPD | Form B | Form B | Form B |

TABLE 30
Results of stability study of Compound 1 Form B under 40° C. ±
2° C./75% RH ± 5% RH storage conditions

| | | Test Result | | |
|---|---|---|---|---|
| | | (40° C. ± 2° C./75% RH ± 5% RH) | | |
| Testing Item | | Initial | 3 M | 6 M |
| Appearance | | white | white | white |
| | | solid | solid | solid |
| Water Content by KF (% w/w) | | 0.4 | 0.9 | 1.0 |
| Purity by UPLC (% area) | | 97.3% | 92.2% | 92.5% |
| Assay by UPLC (% w/w) | | 97.3% | 92.2% | 92.5% |
| Related Substances, | Total | 1.1% | 1.2% | 1.1% |
| UPLC (% area) | Impurities | | | |

| | | | |
|---|---|---|---|
| Chiral purity by HPLC (% area) | 99.8% | 99.9% | 99.9% |
| XRPD | Form B | Form B | Form B |

According to the stability study results for Compound 1 Form B, all the tested parameters, including appearance, purity, assay, related substance, chiral purity, water content, and X-ray powder diffraction (XRPD) meet the acceptance criteria at 6M point (storage conditions: 5±3° C., 25° C.±2° C./60% RH±5% RH, and 40° C.±2° C./75% RH±5% RH). The water content showed an increasing trend under 5±3° C., 25° C.±2° C./60% RH±5% RH, and 40° C.±2° C./75% RH±5% RH conditions.

### Example 31: Drug Product Formulation

Description and composition of the drug product (Compound 1, Tablet) Compound 1 Tablets for oral administration were manufactured in 10 mg, 40 mg, and 80 mg active moiety dose strengths. The composition of each tablet strength is presented in Table 31.

TABLE 31
Composition of the Drug Product

| | Composition | | | | |
|---|---|---|---|---|---|
| | Quantity | Quantity | Quantity | | |
| | per 10 mg | per 40 mg | per 80 mg | | |
| | tablet | tablet | tablet | Percent | |
| Ingredient | (mg/tablet) | (mg/tablet) | (mg/tablet) | (w/w) | Function |
| Compound 1 | 10.00 | 40.00 | 80.00 | 10.00 | Active Ingredient |
| HPMCAS-LG* | 35.00 | 140.00 | 280.00 | 35.00 | Solubility enhancer |
| Vitamin E TPGS* | 5.00 | 20.00 | 40.00 | 5.00 | Permeation enhancer |
| Mannitol, | 27.90 | 111.60 | 223.20 | 27.90 | Filler |
| Intragranular | | | | | |
| Microcrystalline | 14.00 | 56.00 | 112.00 | 14.00 | Binder/Glidant |
| Cellulose, | | | | | |
| Intragranular | | | | | |
| Croscarmellose | 1.00 | 4.00 | 8.00 | 1.00 | Mucoadhesive/ |
| Sodium, | | | | | Disintegrant |
| Intragranular | | | | | |
| Intragranular | 0.50 | 2.00 | 4.00 | 0.50 | Flow aid |
| Untreated Fumed | | | | | |
| Colloidal Silicon | | | | | |
| Dioxide | | | | | |
| Magnesium Stearate, | 0.30 | 1.20 | 2.40 | 0.30 | Lubricant |
| Intragranular | | | | | |
| Microcrystalline | 5.00 | 20.00 | 40.00 | 5.00 | Binder/Glidant |
| Cellulose, | | | | | |
| Extragranular | | | | | |
| Croscarmellose | 1.00 | 4.00 | 8.00 | 1.00 | Mucoadhesive/ |
| Sodium, | | | | | Disintegrant |
| Extragranular | | | | | |
| Magnesium Stearate, | 0.30 | 1.20 | 2.40 | 0.30 | Lubricant |
| Extragranular | | | | | |

*HPMCAS: Hypromellose Acetate Succinate; TPGS: d-α-tocopheryl polyethylene glycol succinate

### Components of the Drug Product

### Drug Substance

Solubility data for Compound 1 drug substance is shown in Table 32.

TABLE 32
Solubility Data for Compound 1

| | | Solubility |
|---|---|---|
| Media | pH | (mg/mL) |
| pH 1.2 HCl/KCl solution | 1.17 | 22.95 |
| pH 2.0 HCl/KCl solution | 1.95 | 4.35 |
| 100 mM pH 3.0 citrate buffer solution | 3.02 | 2.09 |
| 100 mM pH 4.5 citrate buffer solution | 4.53 | 3.16 |
| 50 mM pH 6.8 phosphate buffer | 6.76 | 1.53 |
| solution | | |
| 50 mM pH 7.4 phosphate buffer | 7.40 | 8.79 |
| solution | | |
| pH 9.0 NaOH solution | 8.55 | 21.60 |
| Purified water | 8.90 | 42.44 |
| SGF | 1.74 | 6.58 |
| FaSSIF | 6.48 | 5.84 |
| FeSSIF | 4.86 | 1.35 |

### Excipients

The following excipients were used to develop the clinical formulation for the drug product: HPMCAS-LG, vitamin E TPGS, mannitol, microcrystalline cellulose, croscarmellose sodium, untreated fumed colloidal silicon dioxide, and magnesium stearate. Excipient compatibility studies were performed and demonstrated no compatibility concerns with components selected for the formulation.

### Drug Product

The formulation development for Compound 1 tablets allowed the development of a spray dried intermediate (SDI) to increase the permeability of the drug product. Dispersion excipients and a permeation enhancer were discovered that provided a SDI with improved solubility and permeability compared to neat API. From the formulations tested, the SDI formulation that was used to develop the manufacturing process was 20:70:10 Compound 1: HPMCAS-L: vitamin E TPGS. The 20:70:10 Compound 1: HPMCAS-L: vitamin E TPGS SDI showed improved solubility in FaSSIF biorelevant media as compared to neat API.

### Physicochemical and Biological Properties

Initial studies indicate Compound 1 is a Biopharmaceutics Classification System (BCS) Class II and a Developability Classification System (DCS) Class IIb molecule. Dissolution studies performed on 20:70:10 Compound 1: HPMCAS-L: vitamin E TPGS SDI showed improved dissolution profile than the drug substance itself with solubility maintaining at 52.4 pg/mL in FaSSIF at 210 minutes. The solubility of the SDI in 0.01N HCl is 11 μg/mL, which is similar to the solubility of the drug substance.

### Manufacturing Process Development

The manufacturing process consists of three stages:
(i) manufacture of spray dried intermediate;
(ii) granulation; and
(iii) tableting.

### Spray Dried Intermediate (SDI)

A solvent system of 80:20 dichloromethane:methanol was selected for spray drying as it offers adequate solubility for Compound 1 drug substance and the excipients (>100 mg/mL). The total solid concentration of the spray solution was 8 wt %, which provides a good balance between spray drying capacity and solution viscosity dictated by HPMCAS-L. The wet SDI, prior to tray drying, was shown to be both physically and chemically stable at room temperature. The stability of the SDI was supported by XRPD and HPLC purity and assay data. The secondary drying parameters were established to ensure adequate SDI stability during drying and that the ICH limits for residual dichloromethane and methanol can be met. Through a feasibility batch and an engineering batch, an SDI manufacturing process is established.

### Granulation

A granulation process, which consists of blending between Compound 1 SDI and intragranular excipients, producing ribbons through roller compaction, milling the ribbons, and final blending of the milled granules with extragranular excipients, is developed through efforts in prototyping batches, a scale-up batch, and an engineering batch. Appropriate amount of croscarmellose sodium (disintegrant) in intragranular and extragranular blends (a total of 2.0 wt %) was defined to ensure good disintegration profile for all tablet strengths. Blend speed and time were defined and demonstrated through scale-up and engineering batches. Roller compaction parameters such as roller speed, screw speed, and roller pressure, and milling mesh size (18 mesh) were defined to target a desired bulk density of approximately 0.48 to 0.54 g/mL.

### Tableting

Tablet tooling was defined and confirmed through engineering batches to provide desired tablet sizes and shapes (round tablets for 10 mg and 40 mg strengths, oval tablets for 80 mg strength). Compression parameters such as fill cam sizes, feed frame speed, and compression forces were defined for each strength to achieve desired tablet hardness and friability.

### Container Closure System

The engineering batches were packed in 30 cc (10 mg and 40 mg strength tablets) and 60 cc (80 mg strength tablets) HDPE bottles with either a 28 mm or 33 mm child resistant closure with induction sealing.

### Microbiological Attributes

Compound 1 Tablets are intended for oral administration. Microbiological quality complies with the USP recommended acceptance criteria for the quality of nonaqueous preparations of nonsterile dosage forms of drugs for oral use listed in USP<1111>. The quality standards that provide appropriate limits for microbial content of oral dosage forms include USP<61> and USP <62> limits for Microbial Enumeration and Specific Microorganisms (Escherichia coli).

### Batch Formula (Compound 1, Tablets)

The batch formulae for the dose strengths manufactured are shown in Table 33.

TABLE 33
Batch formula for the Manufacture of Compound 1 Tablets

| | Amount | Amount | Amount | |
|---|---|---|---|---|
| | per 10 mg | per 40 mg | per 80 mg | % of |
| Component/ | Batch | Batch | Batch | Total |
| Ingredient | (g) | (g) | (g) | Weight |
| Compound 1 | 266.50* | 825.60* | 1354.00* | 10.00 |
| HPMCAS-LG | 932.75* | 2889.60* | 4739.00* | 35.00 |
| Vitamin E TPGS | 133.25* | 412.80* | 677.00* | 5.00 |
| Mannitol, Intragranular | 743.54* | 2303.42* | 3777.66* | 27.90 |
| Microcrystalline | 373.10 | 1155.84 | 1895.60 | 14.00 |
| Cellulose, Intragranular | | | | |
| Croscarmellose Sodium, | 26.65 | 82.56 | 135.40 | 1.00 |
| Intragranular | | | | |
| Intragranular Untreated | 13.32 | 41.28 | 67.70 | 0.50 |
| Fumed Colloidal Silicon | | | | |
| Dioxide | | | | |
| Magnesium Stearate, | 8.00 | 24.77 | 40.62 | 0.30 |
| Intragranular | | | | |
| Microcrystalline | 133.25 | 412.80 | 677.00 | 5.00 |
| Cellulose, Extragranular | | | | |
| Croscarmellose Sodium, | 26.65 | 82.56 | 135.40 | 1.00 |
| Extragranular | | | | |
| Magnesium Stearate, | 8.00 | 24.77 | 40.62 | 0.30 |
| Extragranular | | | | |
| Total weight | 2,665.0 | 8256.0 | 13540.0 | 100 |

*The weight is adjusted based on assay of the SDI

### Description of Manufacturing Process and Process Controls (Compound 1, Tablet)

### Summary of Manufacture

Compound 1 Tablets are manufactured in three stages:

### 1. Spray Dried Intermediate (SDI)

A spray dried intermediate is manufactured using Compound 1, HPMC AS-LG, vitamin E TPGS, dichloromethane and methanol.

The first stage of the Compound 1 drug product process is the production of a Compound 1: HPMCAS-LG: vitamin E TPGS spray dried intermediate. Compound 1 is dissolved in dichloromethane and methanol. Vitamin E TPGS and HPMCAS-LG are added to the solution and mixed until both excipients are dissolved. The solution is the spray dried while controlling the solution feed rate, outlet temperature and inlet pressure of the spray dryer. The SDI is then dried in a tray dryer until in-process samples tested for residual solvents show residual solvent level below the in-process controls.

Equipment: spray dryer; tray dryer.

Material input: Compound 1; HPMCAS-LG; vitamin E TPGS; methylene chloride; methanol.

Process Controls: inlet temperature; outlet temperature; solution feed rate; drying time; drying temperature.

### 2. Granulation

A granulation is produced for the tableting process. All strengths are manufactured using 100 mg/g final blend.

A granulation process is used for all strengths of the Compound 1 drug product. Mannitol, Compound 1, HPMCAS-LG, vitamin E TPGS SDI, croscarmellose sodium, untreated fumed colloidal silicon dioxide, and microcrystalline cellulose are added to a V-shell blender. The mixture is blended and milled through a ˜20 mesh screen. The milled material is added back into the V-shell blender along with magnesium stearate. The mixture is blended and loaded into a roller compactor hopper. The granulated blend is roller compacted into ribbons while controlling the roll pressure, roll speed, and screw speed. The ribbons are milled through an 18-mesh screen and placed back into the V-shell blender. The extragranular excipients (microcrystalline cellulose, croscarmellose sodium, and magnesium stearate) are added to the blender and blended to produce the final granulation material.

Equipment: blender; mill; roller compactor.

Material input: Compound 1 SDI intermediate; mannitol; microcrystalline cellulose; croscarmellose sodium; colloidal silicon dioxide; magnesium stearate.

Process controls: blend speed and time; mill speed; mesh size; roll speed; screw speed; roller pressure.

### 3. Compression

The granulation is compressed to produce 10 mg, 40 mg, and 80 mg tablet strengths.

Compound 1 Tablets were manufactured using the granulation described above. Each strength was manufactured by loading the granulation into the hopper of the tablet press and compressing the material while monitoring the press speed, feed frame speed, pre-compression force, compression force and fill depth. At pre-defined intervals during the tableting process, tablets are checked for tablet weight, tablet thickness, hardness and visual appearance. The compressed tablets are passed through a deduster and metal detector. Samples are removed for testing and the remaining tablets are bulk packaged prior to primary packaging.

Equipment: tablet press.

Process controls: tablet weight; tablet hardness; visual inspection.

### Example 32. Single Crystal Structure of (R)-8-(tert-butoxycarbonyl)-1-oxa-8-azaspiro[4.5]decan-3-aminium (R)-2-hydroxy-2-phenylacetate (2-4) from Example 2

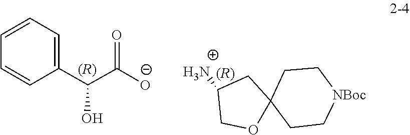

Compound (R)-8-(tert-butoxycarbonyl)-1-oxa-8-azaspiro[4.5]decan-3-aminium (R)-2-hydroxy-2-phenylacetate (2 mg) was dissolved in 0.2 mL acetone/H₂O (4:1) and kept in a half sealed 4 mL vial. The solution was evaporated slowly at room temperature. Crystals were observed and collected the second day for single-crystal X-ray diffraction.

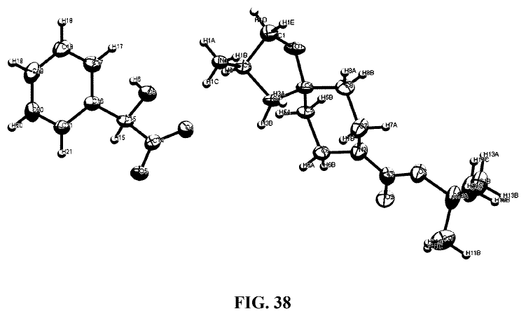

A total of 16982 reflections were collected in the 2θ range from 4.778 to 133.152. The limiting indices were: −5≤h≤7, −10≤k≤11, −43≤l≤44; which yielded 3843 unique reflections (Rᵢₙₜ=0.0769). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 267, compared with 3843 data. All reflections were included in the refinement. The goodness of fit on F² was 1.083 with a final R value for [I >2σ (I)]R1=0.0780 and wR₂=0.2037. The largest differential peak and hole were 0.47 and −0.26 ∈⁻³, respectively. ORTEP structure is depicted in FIG. 38.

Crystallographic data are presented in Table 34 and atomic coordinates are presented in Table 35.

TABLE 34
Crystallographic data and the parameters of the single
crystal X-ray diffraction experiment for Compound 2-4

| Crystal Size | 0.30 × 0.10 × 0.04 mm³ |
|---|---|
| Radiation Type | Cu Kα (λ = 1.54184 Å) |
| Crystal system | orthorhombic |
| Space Group | P2₁2₁2₁ |
| Cell Size | a = 6.0977(2) Å |
| | b = 9.8255(3) Å |
| | c = 36.9923(13) Å |
| | α = 90° |
| | β = 90° |
| | γ = 90° |
| Cell Volume | V= 2216.32(13) Å³ |
| Cell Formula Units | Z = 4 |
| Crystal Density | Dc = 1.224 g/cm³ |
| Crystal F(000) | 880.0 |
| Absorption Coefficient u | μ(Cu Ka) = 0.736 mm⁻¹ |
| Limiting Indices | −5 <= h <= 7 |
| | −10 <= k <= 11 |
| | −43 <= 1 <= 44 |
| Cell Measurement Temperature | T = 149.99(13) K. |
| 2θ range for data collection | 4.778 to 133.152° |
| Goodness-of-fit on F² | 1.083 |
| Final R indices [I > 2sigma(I)] | R1 = 0.0780, wR₂ = 0.2037 |
| R indices (all data) | R1 = 0.0848, wR₂ = 0.2073 |
| Largest diff. peak and hole | 0.47/−0.26 Å⁻³ |
| Reflections collected/unique | 16982/3843 [Rᵢₙₜ = 0.0769] |
| Flack parameter | −0.28(19) |

TABLE 35
Atomic coordinates (× 10\hat{ }4) and equivalent isotropic
displacement parameters (A\hat{ }2 × 10\hat{ }3) for Compound 2-4 from
the single crystal X-ray diffraction experiment.

| Atom | x | y | z | U(eq) |
|---|---|---|---|---|
| O(4) | 2100(7) | 6685(4) | 4622.6(11) | 33.9(10) |
| O(1) | 8840(7) | 3841(4) | 5405.0(13) | 35.4(10) |
| O(6) | 4490(7) | 5333(5) | 4163.3(12) | 35.1(10) |
| O(5) | −803(7) | 6716(5) | 4261.7(12) | 41.3(12) |
| O(3) | 2993(9) | 2713(5) | 6701.0(12) | 44.8(12) |
| O(2) | 2079(9) | 876(5) | 6362.3(12) | 43.5(12) |
| N(1) | 6360(8) | 6430(5) | 4829.8(13) | 27.9(11) |
| N(2) | 3835(10) | 2680(5) | 6114.7(14) | 37.0(13) |
| C(14) | 1185(10) | 6471(6) | 4328.9(16) | 28.6(13) |
| C(4) | 6645(10) | 3840(6) | 5558.1(17) | 31.0(14) |
| C(1) | 9023(11) | 5022(6) | 5187.2(17) | 32.3(14) |
| C(6) | 3722(11) | 2154(6) | 5748.8(16) | 33.4(14) |
| C(9) | 2907(12) | 2024(8) | 6389.5(18) | 40.9(16) |
| C(15) | 2515(9) | 5836(6) | 4015.7(16) | 27.5(13) |
| C(16) | 2839(10) | 6886(6) | 3722.6(16) | 27.5(12) |
| C(2) | 6819(10) | 5107(6) | 5000.5(16) | 28.3(13) |
| C(21) | 1082(11) | 7185(7) | 3488.1(16) | 34.8(15) |
| C(17) | 4789(11) | 7589(7) | 3688.9(17) | 35.7(14) |
| C(18) | 5042(13) | 8586(7) | 3423.3(18) | 41.4(16) |
| C(19) | 3294(13) | 8876(7) | 3194.2(18) | 42.9(17) |
| C(5) | 5882(10) | 2350(6) | 5546.1(17) | 29.4(13) |
| C(8) | 6757(11) | 4280(7) | 5947.0(18) | 38.9(16) |
| C(3) | 5255(10) | 4767(6) | 5315.0(18) | 33.0(14) |
| C(20) | 1349(13) | 8170(8) | 3227.1(17) | 42.6(17) |
| C(10) | 2078(15) | 2175(9) | 7037.4(18) | 51.7(19) |
| C(7) | 4571(13) | 4111(7) | 6138.2(18) | 41.8(17) |
| C(13) | 2606(17) | 3287(9) | 7303(2) | 61(2) |
| C(12) | 3279(17) | 862(9) | 7137(2) | 62(2) |
| C(11) | −362(15) | 1950(10) | 6999(2) | 64(2) |
| O(4) | C(14) | 1.240(8) | C(4) | C(3) |
| O(1) | C(4) | 1.454(7) | C(1) | C(2) |
| O(1) | C(1) | 1.417(7) | C(6) | C(5) |
| O(6) | C(15) | 1.412(7) | C(15) | C(16) |
| O(5) | C(14) | 1.261(8) | C(16) | C(21) |
| O(3) | C(9) | 1.337(8) | C(16) | C(17) |
| O(3) | C(10) | 1.463(8) | C(2) | C(3) |
| O(2) | C(9) | 1.240(9) | C(21) | C(20) |
| N(1) | C(2) | 1.473(7) | C(17) | C(18) |
| N(2) | C(6) | 1.450(8) | C(18) | C(19) |
| N(2) | C(9) | 1.330(9) | C(19) | C(20) |
| N(2) | C(7) | 1.479(9) | C(8) | C(7) |
| C(14) | C(15) | 1.545(8) | C(10) | C(13) |
| C(4) | C(5) | 1.536(8) | C(10) | C(12) |
| C(4) | C(8) | 1.504(9) | C(10) | C(11) |

### Example 33: Cell Viability Assay (CTGlo) in HT29 Model of Colorectal Carcinoma

The effect of Compound 1 on cell viability was assessed based on the quantification of ATP using the CellTiter-Glo® 2.0 (referred to here as CTGlo) Cell Viability Assay (Catalog No. G9243, Promega, Madison, WI, USA), which signals the presence of metabolically active cells. The HT29 human colorectal adenocarcinoma (CRC) cell line with epithelial morphology, was used in these assays.

HT29 cells were obtained from ATCC (Catalog No. HTB-38, Manassas, VA, USA) (also referred to here as HT29.1) and were maintained in McCoy's 5a Medium Modified, (Catalog No. 30-2007, ATCC, Manassas, VA, USA) supplemented with 10% fetal bovine serum (FBS; Catalog No. 16000044, Gibco, Grand Island, NY, USA) at 37° C. in an atmosphere of 5% CO₂ in air. The cells were routinely sub-cultured to maintain cell density between 3×10⁵-1.5×10⁶ cells/mL. Cells were washed with phosphate buffered saline, pH 7.4 (PBS; Catalog No. 10010049, ThermoFisher Scientific, Waltham, MA, USA), trypsinized with Trypsin-EDTA (0.25%), phenol red (Catalog No. 25200056, ThermoFisher Scientific, Waltham, MA, USA) for 5 min at 37° C., and resuspended in growth media. An aliquot was diluted 2× with a 0.4% Trypan Blue solution (Catalog No. 15250061, ThermoFisher Scientific, Waltham, MA, USA) and cell count was determined. Cell concentration was adjusted with growth media to 2.0×10⁴ cells/mL.

To determine the effect of Compound 1 on cell viability, 50 μL HT29 cells suspended in growth media at 2.0×10⁴ cells/mL (for a cell density of 1000 cells/well) were dispensed to each well of a 384-well, black TC-treated microplates (Catalog No. 3571, Corning, Glendale, CA, USA).

Test compounds were prepared by dissolving neat compounds in dimethyl sulfoxide (DMSO; Catalog No. D8418, Sigma-Aldrich, Inc., St. Louis, MO, USA) to generate 10 mM stock solution and stored at −20° C. The 10 mM DMSO stock solutions were serially diluted (half log titration) in DMSO to generate a 10-point dose series with a final concentration of 10 μM, in duplicate. Using an Echo 550 Acoustic Liquid Handler (Beckman Coulter Life Sciences, Indianapolis, IN, USA), plated cells were treated with fifty nL of serially diluted test compound solutions in duplicate. fifty nL DMSO was transferred to control wells. Plates were incubated at 37° C., 5% CO₂ for seventy-two hours. CellTiter-Glo® reagent was then added to the cells and luminescence signals were measured after four hours using an EnVision™ Multimode plate reader (Perkin Elmer, Santa Clara, CA).

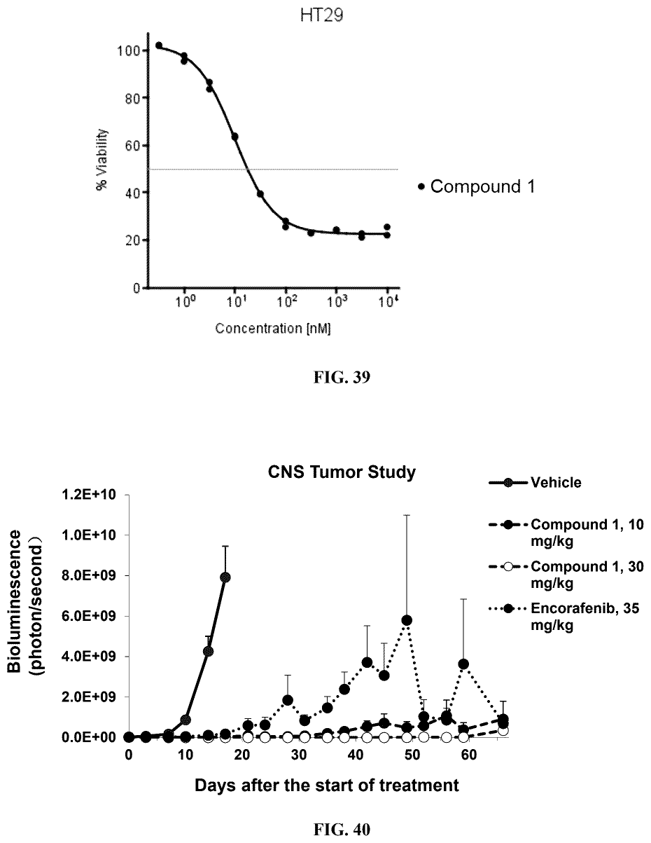

For data normalization, cells untreated with test compounds at Time=seventy-two hours were set to 100% (equivalent to maximal cell growth after ninety-six hours). Media-only wells were set to −100% (equivalent to cytotoxicity). Percent Viability was determined by normalizing the signal with positive and DMSO treated negative controls on the same microtiter plate. The half-maximal inhibition of cell growth (GI50) was computed from where the fitted curve crosses 50%. The Emax, or maximum effect of each compound, represents the lowest % viability achieved following compound treatment. HT29 viability following seventy-two hours after treatment with Compound 1 is shown in FIG. 39.

### Example 34: Efficacy, Pharmacokinetics, and Pharmacodynamics of Compound 1 in Model of CNS Activity

For the efficacy study, agents were tested in A375 human melanoma cancer cell derived xenografts (CDX). This cell line was isolated from the primary melanoma of a 54-year-old female and is known to have homozygous BRAF V600E mutation. The A375 tumor cells were maintained in vitro in DMEM medium supplemented with 10% fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation.

For this CNS study, A375 cells with a luciferase tag for tracking tumor cell signal, were injected intracranially into 6-8 week old female BALB/c nude mice. The mice were procured from Zhejiang Vital River Laboratory Animal Co., LTD. Beijing, China. The mice were anaesthetized, restrained, and sterilized prior to surgery. A sagittal cut was made above the anterior parietal and occipital bone with sterilized scalpel, the skull was revealed and cleaned with cotton bud until the anterior fontanel was visible. A 2 mm hole was created to the right of the anterior fontanel and 0.5 mm above the coronal on the skull with a cranial drill. Cell suspensions in 20% Matrigel were mixed and aspirated into the injector carefully to avoid bubbles and wiped to avoid extracranial tumorigenesis. The needle was inserted vertically into the cranial hole and the cell mixture was slowly injected for two minutes and held for another minute to decrease the drainage of the cell suspension. The wound was closed, and a subcutaneous injection of 5 mg/kg Meloxicam was given to relieve the pain and continued for three days. The mice were monitored closely until they woke up after the surgery. After inoculation, the animals were checked daily for morbidity and mortality.

The administration of the test agents began three days after inoculation. CNS tumor-bearing mice were randomly assigned to groups of eight mice and administered Compound 1 at 10 mg/kg orally (PO) twice a day (BID), Compound 1 at 30 mg/kg PO/BID, encorafenib at 35 mg/kg once a day orally, (PO/QD), or the vehicle alone PO/BID. Measurements for the presence of the A375 luciferase signal within the CNS were done with IVIS Lumina III machine. Images were collected twice weekly to follow the growth of the models. The tumor-bearing mice were weighed and intraperitoneally administered luciferin (Perkin Elmer Inc-122799) at a dosage of 150 mg/kg. After ten minutes, mice were pre-anesthetized with the mixture gas of oxygen and isoflurane. The bioluminescence value at fifteen minutes post luciferin injection was recorded as the final value and plotted. The endpoint for individual mice was reached when the mouse body weight decreased by 20% when compared to their starting weight.

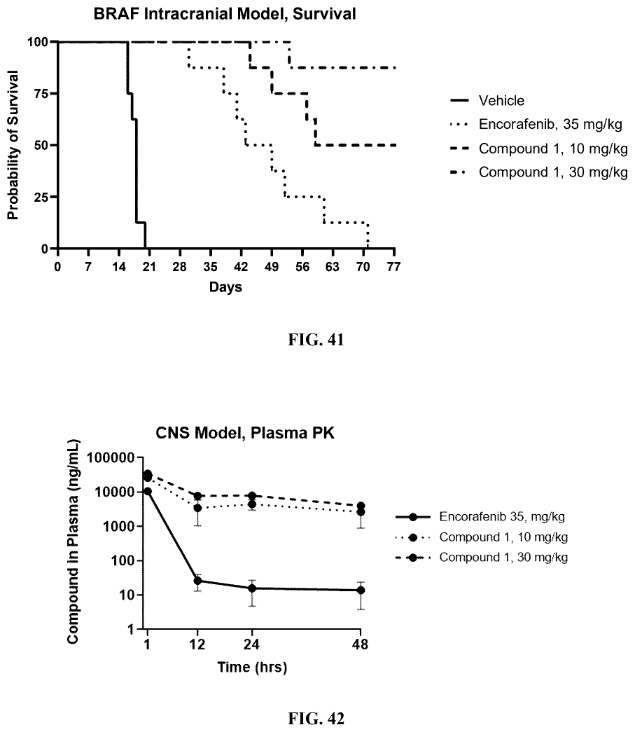

The mice in the vehicle control group all reached their endpoint within the first three weeks of study with a median survival of 18 days. Encorafenib prolonged the survival to an average of 46 days. As of day 75, four mice in the 10 mg/kg group and 1 mouse in the 30 mg/kg group of Compound 1 have demonstrated end point cut offs and were terminated. As of day 75, the survival rate for mice treated with 10 mg/kg of Compound 1 had a 50% survival rate and the 30 mg/kg treatment had a survival rate of 87.5%. The luciferase signal increased concordantly with the increase in symptoms of the CNS tumors. The luciferase signal for the mice removed from study were not carried forward, so a sudden drop in luciferase signal over time is due to mice with a heavy tumor burden being terminated and their high luciferase signal not being carried forward. The compounds were tolerated and none of the groups showed body weight loss prior to an increase in luciferase signal. The change in luciferase signal over time, representing tumor burden, is shown in FIG. 40. The survival curve for the mice on study is shown in FIG. 41.

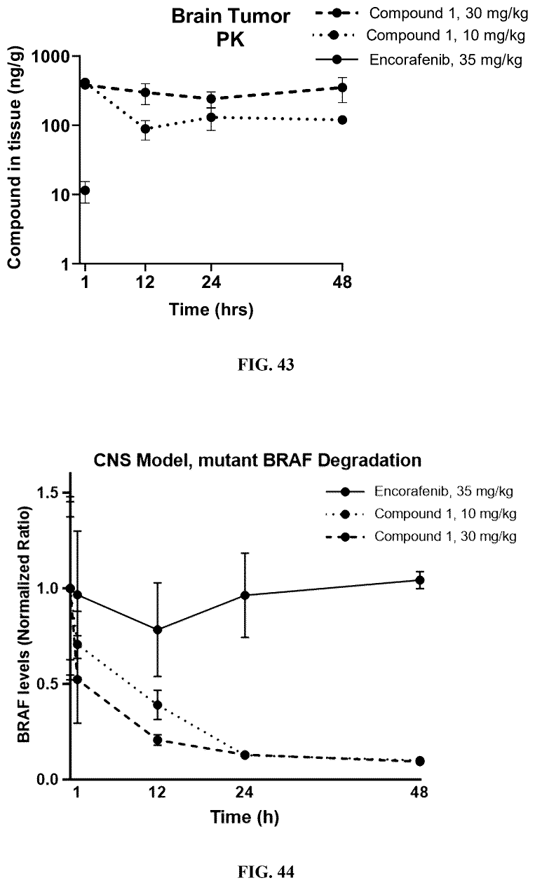

To determine the level of CNS and plasma exposure of Compound 1 compared to encorafenib, a separate pharmacokinetics/pharmacodynamics (PK/PD) study was conducted. Mice were inoculated with luciferase tagged A375 cells as stated above. The tumors were left to establish and expand for 14 days before the study. Mice were randomized and treated with Compound 1 at 10 mg/kg orally (PO), Compound 1 at 30 mg/kg PO, encorafenib at 35 mg/kg, or the vehicle alone. Samples from 3 mice per treatment group were collected at 1, 12, 24, and 48 hours. To test the plasma samples, the mice were anesthetized, and blood was collected into a tube with EDTA-K2, centrifuged, transferred to a fresh tube, and stored frozen. Following blood collection, the mice were perfused for fifteen minutes, decapitated, and the brain was collected and frozen. The harvested plasma and brain were processed for pharmacokinetics analysis by LC/MS/MS with a concentration range of 1-1,000 ng/mL and pharmacodynamics analysis by Western Blot. The concentrations of Compound 1 and encorafenib found in the plasma is shown in FIG. 42, and the concentration found in the brain is shown in FIG. 43. The pharmacodynamics of BRAF protein degradation in the inoculated CNS tumor was quantified and shown in FIG. 44.

### Example 35: Efficacy of Compound 1 in Combination with Cetuximab in CDX Model of Colorectal Cancer

For the efficacy study, agents were tested in the HT-29 human colorectal cancer cell derived xenografts (CDX). This cell line was isolated from the primary colorectal adenocarcinoma of a 44-year-old female and is known to have a heterozygous BRAF V600E mutation and an oncogenic heterozygous PIK3CA mutation. The HT-29 tumor cells were maintained in vitro in McCoy's 5a medium supplemented with 10% fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. For this CDX study, HT-29 cell pellets were implanted on the left flank of 6-8 week old female BALB/c nude mice.

When tumors reached an average volume of 125-200 mm³, animals were assigned to treatment or control groups with 8 mice in each group. Tumor volumes were stratified to result in similar mean tumor volumes in each treatment and control group; treatment began on Day 0. The endpoint for each individual mouse was reached when the mouse weight decreased by 20%.

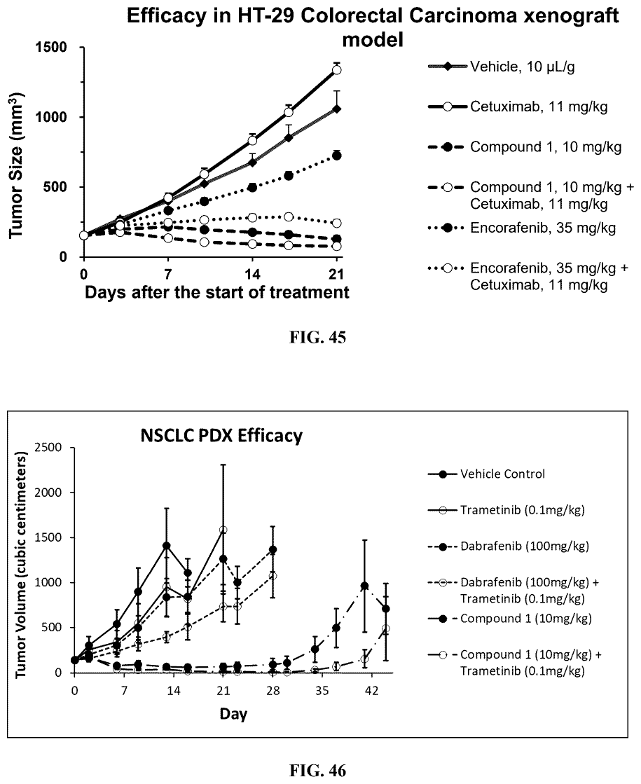

This CRC xenograft model was used for testing the efficacy of Compound 1 compared to the standard of care BRAF inhibitor encorafenib as a single agent, and in combination with the EGFR monoclonal antibody cetuximab. CDX tumor mice were administered Compound 1 at 10 mg/kg orally (PO) twice a day (BID), cetuximab was dosed once every three days (Q3D) from day 1 and dosed at 11 mg/kg via intraperitoneal (IP) injection for 28 days, the combination of Compound 1 at 10 mg/kg PO/BID plus cetuximab at 11 mg/kg IP/Q3, encorafenib at 35 mg/kg, the combination of encorafenib at 35 mg/kg PO/QD plus cetuximab at 11 mg/kg IP/Q3, or the vehicle alone PO/BID. Compound 1 with or without cetuximab, demonstrated tumor regression at end of study. Test compounds were made fresh weekly for the study. Tumor volumes and animal body weight were measured twice weekly. All compounds were well tolerated and none of the groups showed more than 10% body weight loss during the study. The change in tumor volume over time is shown in FIG. 45.

### Example 36: Efficacy of Compound 1 in Combination with Trametinib in a PDX Model of NSCLC

For this efficacy study, a patient-derived xenograft (PDX) mouse model was established using tumor fragments acquired during biopsy of a tumor from a human patient with non-small cell lung carcinoma (NSCLC) genotyped to have the BRAF V600E mutation through serial passages on the flank of immunocompromised mice. For this PDX study, established tumor fragments were implanted on the left flank of 6-8 week old female Athymic Nude-Foxn1nu (Immune-compromised) mice (Envigo; Indianapolis, Indiana).

When tumors reached an average tumor volume of 125-225 mm³ animals were assigned to treatment or control groups with 6 mice in each group. Tumor volumes were stratified to result in approximately equal average tumor sizes in each treatment and control group; treatment began on Day 0. The endpoint for each group was reached when the mean tumor volume of the group reached 1500 mm³.

This NSCLC xenograft model was used for testing the efficacy of Compound 1 compared to the standard of care BRAF inhibitor dabrafenib as a single agent, and in combination with the MEK inhibitor trametinib. PDX tumor mice were administered Compound 1 at 10 mg/kg orally (PO) twice a day (BID), dabrafenib at 100 mg/kg PO once a day (QD), trametinib at 0.1 mg/kg PO/QD, the combination of Compound 1 at 10 mg/kg PO/BID plus trametinib at 0.1 mg/kg PO/QD, the combination of dabrafenib at 100 mg/kg PO/QD plus trametinib at 0.1 mg/kg PO/QD, or the vehicle alone PO/BID. Mice were treated until day 28, at which point the vehicle control, and mice treated with trametinib, dabrafenib, or the combination of trametinib and dabrafenib had already reached tumor burden endpoint and were terminated. The remaining treatment groups, Compound 1 with or without trametinib, demonstrated tumor regression and were taken off treatment and monitored for tumor outgrowth until day 45 without additional administration of drug treatment. Test compounds were made fresh weekly for the study. Tumor volumes and animal body weight were measured twice weekly. All compounds were well tolerated and none of the groups showed more than 3% body weight loss during the study. The change in tumor volume over time is shown in FIG. 46.

### Example 37: Efficacy of Compound 1 in Combination with Trametinib PDX Model of Resistant Melanoma with a BRAF Kinase Domain Duplication

A biopsy from the melanoma tumor of a patient that progressed while on BRAF inhibitor was established as a patient-derived xenograft (PDX) through serial passages on the flank of immunocompromised mice. The model was determined to carry a mutation in BRAF that led to the duplication of the kinase domain, a known splice variant resistance mechanism to BRAF inhibitors. For this efficacy study 70 mg PDX tumor fragments approximately 70 mg in size were implanted on the left flank of 6-12 week old female Athymic Nude, Outbred Homozygous (Crl:NU(NCr)-Foxn1nu), Strain #: 490 mice. When tumors reached an average tumor volume of 125-225 mm³ animals were assigned to treatment or control groups with eight mice in each group. Tumor volumes were stratified to result in approximately equal average tumor sizes in each treatment and control group; treatment began on Day 0. During the study, animals exhibiting >10% weight loss when compared to Day 0 were provided supplemented food ad libitum, along with all animals in the group. Any animal exhibiting >20% net weight loss for a period lasting 7 days or if mice display >30% net weight loss when compared to Day 0 were considered moribund and euthanized. Individual animals reached their tumor burden endpoint when the tumor volume exceeded 2.5 cm³.

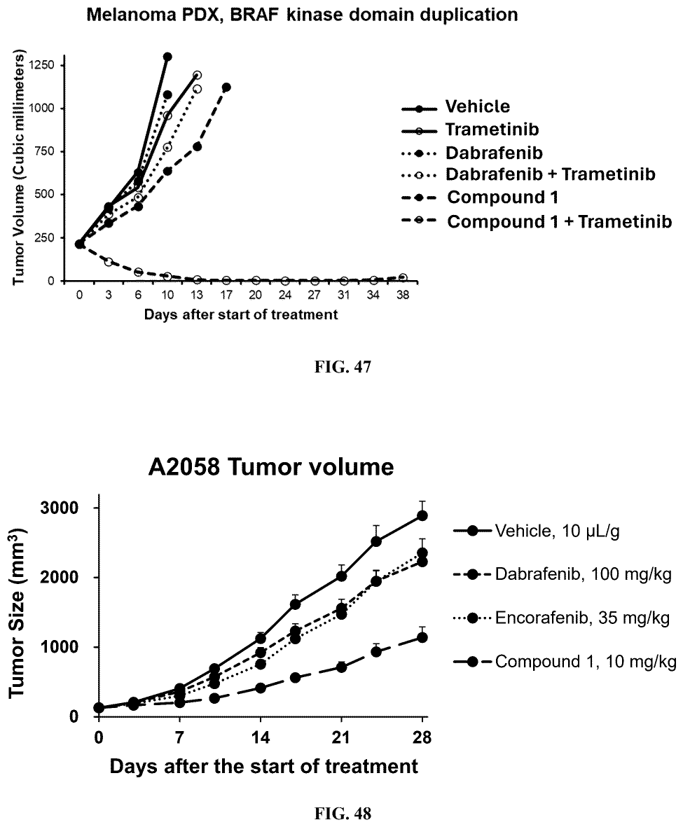

This melanoma BRAF kinase domain duplication xenograft model was used for testing the efficacy of Compound 1 compared to the standard of care BRAF inhibitor as single agents, and in combination with the MEK inhibitor trametinib. Tumor-bearing mice were administered Compound 1 at 10 mg/kg orally (PO) twice a day (BID), dabrafenib at 100 mg/kg PO once a day (QD), trametinib at 0.1 mg/kg PO/QD, the combination of Compound 1 at 10 mg/kg PO/BID plus trametinib at 0.1 mg/kg PO/QD, the combination of dabrafenib at 100 mg/kg PO/QD plus trametinib at 0.1 mg/kg PO/QD, or the vehicle alone PO/BID. Mice were treated until day 21, at which point the remaining treatment group, Compound 1 in combination with trametinib, was taken off treatment and monitored for tumor outgrowth until day 38 without additional drug treatment. Tumor volumes and animal body weight were measured twice weekly. All compounds were well tolerated and none of the groups showed more than 10% body weight loss during the study. The change in tumor volume over time is shown in FIG. 47.

### Example 38: Efficacy of Compound 1 in Combination with Cetuximab in CDX Model of BRAF Inhibitor Resistant Melanoma with Mutant BRAF and MEK1

For the efficacy study, agents were tested in the A2058 human melanoma cancer cell derived xenografts (CDX). This cell line was isolated from the metastatic site in a 43-year-old male patient with melanoma, and is known to have a heterozygous BRAF V600E mutation and an oncogenic MEK1 P124S mutation. The A2058 tumor cells were maintained in vitro in EMEM medium supplemented with 10% fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. For this CDX study, A2058 cell pellets were implanted on the left flank of 6-8 week old female BALB/c nude mice.

When tumors reached an average volume of 125-200 mm³, animals were assigned to treatment or control groups with 8 mice in each group. Tumor volumes were stratified to result in similar mean tumor volumes in each treatment and control group; treatment began on Day 0. The endpoint for each individual mouse was reached when the mouse weight decreased by 20%.

This melanoma xenograft model was used for testing the efficacy of Compound 1 compared to the standard of care BRAF inhibitors dabrafenib and encorafenib as a single agent. PDX tumor mice were administered Compound 1 at 10 mg/kg orally (PO) twice a day (BID), encorafenib at 35 mg/kg PO once a day (QD), dabrafenib at 100 mg/kg PO/QD, or the vehicle alone PO/BID. Mice were treated until day 28. Tumor Growth Inhibition (TGI) was calculated using the formula: TGI (%)=(1−(T−T₀)/(C−C₀))*100, where T=mean tumor size at the terminal end of treatment group; T₀=mean tumor size at day 0 of treatment group; C=mean tumor size at the terminal end of control group; and C₀=mean tumor size at day 0 of control group. Compound 1 demonstrated a TGI of 63%, while encorafenib and dabrafenib were 19% and 23% respectively. Test compounds were made fresh weekly for the study. Tumor volumes and animal body weight were measured twice weekly. All compounds were well tolerated and none of the groups showed more than 10% body weight loss during the study. The change in tumor volume over time is shown in FIG. 48.

### Example 39: A2058 Efficacy, and PKPD of Compound 1 in a BRAF and MEK1 Mutant Model

### Methods

For the efficacy study, agents were tested in the A2058 human melanoma model. Compound 1, encorafenib and dabrafenib were administered to BALB/c nude mice bearing A2058 xenografts orally for 28 days. Compound 1 was dosed twice per day (BID) and dosed at 10 mg/kg from day 0. Encorafenib and dabrafenib were dosed daily (QD) from day 1. Encorafenib was dosed at 35 mg/kg. Dabrafenib was dosed at 100 mg/kg.

For the PKPD study, agents were administered to BALB/c nude mice bearing A2058 human melanoma xenografts. Compound 1, encorafenib and dabrafenib were dosed once orally on day 1. Compound 1 was dosed at 10 mg/kg, encorafenib was dosed at 35 mg/kg and dabrafenib was dosed at 100 mg/kg.

### CONCLUSION

For efficacy study, Compound 1 at 10 mg/kg BID (TV=933 mm³, T/C=37.06%, TGI=66.37%, p<0.0001) exhibited significant antitumor activity. Encorafenib at 35 mg/kg QD (TV=1944 mm³, T/C=77.22%, TGI=24.04%, p>0.05) and dabrafenib at 100 mg/kg QD (TV=1948 mm³, T/C=77.37%, TGI=23.87%, p>0.05) had no significant antitumor activity.

For the PK study the concentration of Compound 1, encorafenib and dabrafenib peaked at the collection timepoints of one hour in plasma and six hours in tumor.

The Western Blotting results showed that Compound 1, encorafenib and dabrafenib inhibited the expression of p-ERK from six to twenty-four hours post dose. Compound 1 inhibited the expression of BRAF from six to forty-eight hours post dose. Encorafenib and dabrafenib had no effect on the expression of BRAF.

### Experimental Design

### Groups and Treatments for the Study

TABLE 36
Study Design for Efficacy Study

| | | | Weeks 1-4 | | | Time point | Time point |
|---|---|---|---|---|---|---|---|
| | | | Dose | Dosing | | for Plasma | for Tumor |
| Group | N^{a} | Treatment^{b} | (mg/kg) | Route | Schedule^{c} | Collection^{d} | Collection^{e} |
| 1 | 8 | Vehicle | — | p.o. | BID × 28 | — | 6 h |
| | | | | | days | | |
| 2 | 8 | Compound | 10 | p.o. | BID × 28 | 6 h | 6 h |
| | | 1 | | | days | | |
| 3 | 8 | Encorafenib | 35 | p.o. | QD × 28 days | 6 h | 6 h |
| 4 | 8 | Dabrafenib | 100 | p.o. | QD × 28 days | 6 h | 6 h |

^{a}N: animal number; Dosing volume: adjusted dosing volume based on body weight 10 μL/g.
^{b}The formulations for vehicle and Compound 1 were 20% PEG400 + 80% (25% SBE-β-CD in ddH₂O). The formulation for encorafenib was 0.5% CMC-Na + 0.5% Tween 80 in ddH₂O. The formulation for dabrafenib was 10% PEG400 + 90% (11.1% HP-β-CD in ddH₂O).
^{c}Mouse#1-4 was euthanized on PG-D24 due to the tumor size over 3000 mm³. Mouse#8-3 was found dead on PG-D26.
^{d}Plasma samples were collected at a predetermined time for potential PK analysis.
^{e}Tumor samples were collected at a predetermined time for potential PK and WB analysis.

TABLE 37
Study Design for PKPD Study

| | | | | | | Time point | Time point |
|---|---|---|---|---|---|---|---|
| | | | Dose | Dosing | | for Plasma | for Tumor |
| Group | N^{a} | Treatment^{b} | (mg/kg) | Route | Schedule | Collection^{c} | Collection^{d} |
| 5 | 3 | Vehicle | — | p.o. | Single dose | — | 6 h |
| 6 | 3 | Compound 1 | 10 | p.o. | Single dose | 1 h, 6 h | 6 h |
| | 3 | | | | | 12 h | 12 h |
| | 3 | | | | | 24 h | 24 h |
| | 3 | | | | | — | 48 h |
| 7 | 3 | Encorafenib | 35 | p.o. | Single dose | 1 h, 6 h | 6 h |
| | 3 | | | | | 12 h | 12 h |
| | 3 | | | | | 24 h | 24 h |
| | 3 | | | | | — | 48 h |
| 8 | 3 | Dabrafenib | 100 | p.o. | Single dose | 1 h, 6 h | 6 h |
| | 3 | | | | | 12 h | 12 h |
| | 3 | | | | | 24 h | 24 h |
| | 3 | | | | | — | 48 h |

^{a}N: animal number; Dosing volume: adjusted dosing volume based on body weight 10 μL/g.
^{b}The formulations for vehicle and Compound 1 were 20% PEG400 + 80% (25% SBE-β-CD in ddH₂O). The formulation for encorafenib was 0.5% CMC-Na + 0.5% Tween 80 in ddH₂O. The formulation for dabrafenib was 10% PEG400 + 90% (11.1% HP-β-CD in ddH₂O).
^{c}Plasma samples were collected at a predetermined time for PK analysis.
^{d}Tumor samples were collected at a predetermined time for PK, WB and FFPE analysis

### Materials

### Animals and Housing Condition

### Animals

Species: Mus Musculus
Strain: BALB/c nude
Age: 6-8 weeks
Gender: Female
Body weight: 18-22 g
Number of animals: 71 mice plus spare
Animal supplier: Shanghai Lingchang biological science and technology Co., Ltd.

### Housing Condition

The mice were kept in individual ventilation cages at constant temperature and humidity with 3 or 4 animals in each cage.

Temperature: 20˜26° C.

Humidity: 40-70%.

Cages: Made of polycarbonate. The size is 375 mm×215 mm×180 mm. The bedding material was corn cob and changed twice per week.

Diet: Animals had free access to irradiation sterilized dry granule food during the entire study period.

Water: Animals had free access to sterile drinking water.

Cage identification: The identification labels for each cage contained the following information: number of animals, gender, strain, date received, treatment, study number, group number and the starting date of the treatment.

Animal identification: Animals were marked by ear coding.

### Test Compounds

| | | | Mole- | For- | |
|---|---|---|---|---|---|
| | | | cular | mula | |
| Compound | Manufacturer | Batch | Weight | Weight | Purity |
| Compound 1 | C4 Therapeutics | EW31581- | 958.01 | 958.01 | 96.73% |
| | | 15-P1 | | | |
| Encorafenib | MedChemExpress | HY-15605- | 540.01 | 540.01 | 99.05% |
| | | 228439 | | | |
| Dabrafenib | MedChemExpress | HY-14660- | 519.56 | 519.56 | 99.90% |
| | | 153495 | | | |

### Reagents and Materials for PD

### Reagents and Consumables

| No. | Reagents/Consumables | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS | Invitrogen | NP0007 |
| | Sample Buffer (4×) | | |
| 6 | NuPAGE ™ Sample | Invitrogen | NP0009 |
| | Reducing Agent (10×) | | |
| 7 | NuPAGE ® MES SDS | Invitrogen | NP0002 |
| | Running Buffer (20×) | | |
| 8 | 20x TBS | Bio-Serve | BS-P-15 |
| 9 | Tween 20 | Sigma | P2287 |
| 10 | PageRuler ™ Prestained | Thermo | 26616 |
| | Protein Ladder | Scientific | |
| 11 | iBlot ® 2 Transfer Stack Regular | Invitrogen | IB23001 |
| 12 | NuPAGE, Novex 4-12% | Invitrogen | WG1403BO |
| | Bis-Tris Gel, 26well | | X |
| 13 | Bovine Serum Albumin | BBI life | A600332-0100 |
| | | sciences | |
| 14 | SuperSignal ™ West Femto | Thermo | 34096 |
| | Maximum Sensitivity Substrate | Scientific | |
| 15 | Nonfat Milk | BD Difco | 232100 |
| 16 | SuperSignal West Pico | Thermo | 34078 |
| | Chemiluminescent Substrate | Scientific | |

To prepare 1×TBST, 50 mL of 20×TBS was added to 949 mL double distilled H₂O, followed by the addition of 1 mL Tween 20. Formulation was mixed well and used in the next step. Running buffer was prepared by adding 50 mL of 20×MES SDS Running Buffer to 950 mL of double distilled H₂O. Formulation was mixed well and used in the next step.

### Instruments

| No. | Instruments | Vendor | Model # |
|---|---|---|---|
| 1 | Tissuelyser | Shanghai Jingxin | JXFSTPRP-CL |
| 2 | Electro- thermal incubator | Boxun | HPX-9162 MBE |
| 3 | SpectraMax | Molecular Devices | M5e |
| 4 | Dry baths | Hangzhou All | MK-10 |
| | | sheng Instruments | |
| | | Co., Ltd | |
| 5 | Xcell4 SureLock ® Midi-cell | Invitrogen | WR0100 |
| 6 | PowerPac ™ Basic | Bio-Rad | 1645050 |
| | Power Supply | | |
| 7 | Orbital Shaker | Qiling | TS-1 |
| 8 | iBlot ® 2 Gel Transfer Device | Invitrogen | iBlot 2 |
| 9 | Beads | Qiagen | 69989 |
| 10 | Amersham Imager 680 | GE Healthcare | AI680QC |
| 11 | Vortex Shaker | Kylin-Bell | Vortex-6 |
| 12 | Centrifuge | Eppendorf | 5424R |

### Antibodies

| No. | Antibodies | Vendor | Catalog # |
|---|---|---|---|
| 1 | BRAF V600E Rabbit | RevMab | RevMab-31- |
| | Monoclonal Antibody [RM8] | | 1042-00 |
| 2 | Phospho-p44/42 MAPK | Cell Signal | CST-4370S |
| | XP ® Rabbit mAb | Technology | |
| 3 | GAPDH antibody | Cell Signal | CST-5174S |
| | | Technology | |
| 4 | p44/42 MAPK (Erk1/2) | Cell Signal | CST-4696S |
| | (L34F12) Mouse mAb | Technology | |
| 5 | Goat Anti-Rabbit IgG H&L (HRP) | Abcam | ab6721 |
| 6 | Goat Anti-Mouse IgG H&L(HRP) | Abcam | ab97230 |

### Experimental Methods and Procedures

### Cell Culture

The A2058 tumor cells were maintained in vitro in EMEM medium supplemented with 10% fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation.

TABLE 38
Vendor and Cat No. of Cell Line and Reagents

| Cell Line/Reagent | Vendor | Catalog # |
|---|---|---|
| A2058 | ATCC, Manassas, VA, USA | CRL-11147 |
| EMEM | Gibco | 11995-065 |
| Penicillin-Streptomycin | Gibco | 15240-062 |
| 0.25% Trypsin-EDTA | Gibco | 25200-072 |
| FBS | Corning | 35-081-CV |
| DPBS | Corning | 21-031-CVC |
| Matrigel Matrix | Corning | 354234 |

### Tumor Inoculation

Each mouse was inoculated subcutaneously at the right flank with A2058 cells (5×106) in 0.2 mL of PBS supplemented with Matrigel (PBS:Matrigel=1:1) for tumor development. Animals were grouped for efficacy study when the average tumor volume reached 130 mm³ on day 7 after tumor inoculation and 412 mm³ for PKPD study on day 14.

### Reagents Used in the Study

TABLE 39
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike Chemical Materials | 10059859 |
| HP-β-CD | Energy chemical | E080016 |
| CMC-Na | Sigma | C4888 |
| Tween 80 | Sigma | P4780 |

### Vehicle Preparation

Preparation of 25% SBE-β-CD in double distilled water (ddH₂O)

To prepare 25% sufobutylether-β-cyclodextrin (SBE-β-CD) in ddH₂O, 50 g of SBE-β-CD was accurately weighed and transferred into a 500 mL glass bottle, followed by addition of 150 mL of ddH₂O. The solution was stirred until a clear solution was obtained. To this clear solution, additional ddH₂O was added to achieve the final volume of 200 mL, and the solution was mixed well to obtain 25% SBE-β-CD in ddH₂O.

Preparation of 11.1% HP-β-CD in ddH₂O

To prepare 11.1% 2-hydroxypropyl-β-cyclodextrin (HP-β-CD) in ddH₂O, 22.2 g of HP-β-CD was accurately weighed and transferred into a 500 mL glass bottle, followed by addition of 150 mL of ddH₂O. The solution was stirred until a clear solution was obtained. To this clear solution, additional ddH₂O was added to achieve the final volume of 200 mL, and the solution was mixed well to obtain 11.1% HP-β-CD in ddH₂O.

Preparation of 0.5% CMC-Na and 0.5% Tween 80 in ddH₂O

To prepare 0.5% sodium carboxymethyl cellulose (CMC-Na) and 0.5% Tween 80 in ddH₂O, 1.0 g of CMC-Na was accurately weighed and transferred into a 500 mL glass bottle, followed by addition of 160 mL of ddH₂O, and 1 mL of Tween 80. The solution was stirred until a clear solution was obtained. To this clear solution, additional ddH₂O was added to achieve the final volume of 200 mL, and the solution was mixed well to obtain 0.5% CMC-Na and 0.5% Tween 80 in ddH₂O.

### Testing Article Formulation Preparation

TABLE 40
Formulation of Compound 1 Preparation

| | | | | | Volume of | |
|---|---|---|---|---|---|---|
| | | | | | 25% SBE-β- | |
| | | | | Volume of | CD in ddH₂O | |
| | | Conc. of | | PEG400 to | to be added | Dosing |
| Compound | Dose | solution | Compound | be added | (mL) | Solution |
| ID | (mg/kg) | (mg/mL) | amount (mg) | (mL) (20%) | (80%) | observations |
| Compound | 10 | 1 | 15.21 | 2.943 | 11.770 | Solution |
| 1 | | | | | | |

*The dosing solution was prepared once every three days.

TABLE 41
Formulation of Encorafenib Preparation

| | | | | Volume | |
|---|---|---|---|---|---|
| | | | | of 0.5% | |
| | | | | CMC-Na | |
| | | | | and 0.5% | |
| | | | Com- | Tween 80 in | |
| | | Conc. of | pound | ddH₂O to | Dosing |
| Compound | Dose | solution | amount | be added | Solution |
| ID | (mg/kg) | (mg/mL) | (mg) | (mL) | observations |
| Encorafenib | 35 | 3.5 | 25.60 | 7.245 | Suspension |

* The dosing solution was prepared once every three days.

TABLE 42
Formulation of Dabrafenib Preparation

| | | | | | Volume | |
|---|---|---|---|---|---|---|
| | | | | Volume | of 11.1% | |
| | | | | of | HP-B- | |
| | | | | PEG400 | CD in | |
| | | Conc. | Com- | to be | ddH₂O to | Dosing |
| Com- | | of | pound | added | be added | Solution |
| pound | Dose | solution | amount | (mL) | (mL) | obser- |
| ID | (mg/kg) | (mg/mL) | (mg) | (10%) | (90%) | vations |
| Dabrafenib | 100 | 10 | 71.66 | 0.716 | 6.443 | Sus- |
| | | | | | | pension |

* The dosing solution was prepared once every three days.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3,000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

Tumor growth inhibition (TGI) was calculated for each group using the formula: TGI (%) 20=[1−(Tᵢ−T₀)/(Cᵢ−C₀)]×100; Ti is the average tumor volume of a treatment group on a given day, TO is the average tumor volume of the treatment group on the day of treatment start, Ci is the average tumor volume of the vehicle control group on the same day with Ti, and C0 is the average tumor volume of the vehicle group on the day of treatment start.

### Experimental Endpoint and Sample Collection

### Plasma Sample Preparation

The plasma samples were collected at predicted time for PK analysis (as shown in Table 36 and Table 37).

### Non-Termination Plasma Collection:

To collect non-termination plasma samples, blood samples were collected into the 1.5 mL Eppendorf tubes containing 3 μL of 0.5 M K2 ethylenediaminetetraacetic acid (EDTA-K2). Anticoagulant blood samples were centrifuged at 2500×g at 4° C. for fifteen minutes.

### Termination Plasma Collection:

To collect the termination plasma samples, mice were euthanized at predetermined time points after the treatment. Blood samples from the mice were collected into the 1.5 mL Eppendorf tubes containing 15 sL of 0.5 M K2 ethylenediaminetetraacetic acid (EDTA-K2). Anticoagulant blood samples were centrifuged at 2500×g at 4° C. for fifteen minutes.

The plasma samples were separated into 2 aliquots. The 1^{st} was used for PK and the 2^{nd} was used as a backup.

### Tumor Sample Preparation

Tumor samples were collected at predicted time for pharmacokinetic (PK), Western Blot (WB) and formalin-fixed paraffin-embedded (FFPE) sample analysis. The tumor was dissected out, and the tumor sample was split into four parts, from which the 1^{st} part was used for PK analysis, the 2^{nd} part was used for WB analysis, the third part was used for FFPE analysis, and the 4^{th} part was used as a backup for the study. PK and WB samples were transferred into pre-labeled tubes. Samples in tubes were snap-frozen in liquid nitrogen within two minutes after dissection. The samples were stored at −80° C. until analysis. Tumor samples for FFPE analysis were collected and fixed in formalin for twenty-four to forty-eight hours, after which FFPE blocks were prepared for analysis.

### Pharmacokinetics Assay

The harvested plasma and tumor samples were processed for pharmacokinetics analysis by LC-MS/MS with a concentration range of 0.200˜200 ng/mL for encorafenib, Dabrafenib and Compound 1 in tumor homogenate, 1.00˜1,000 ng/mL for Dabrafenib in plasma and 10.0˜10,000 ng/mL for Compound 1 and encorafenib in plasma.

### Western Blotting Assay

### Protein Extraction & Protein Quantification

Previously snap-frozen samples were retrieved from −80° C. ultralow freezer, and 30-100 mg of tumor tissue was cut up. The tissue samples were placed in a 2 mL microcentrifuge tube, and 450 μL of RIPA buffer containing 1% Protease inhibitor cocktail and 1% phosphate inhibitor cocktail 2 were added to the tube. Tumor samples were grinded with Tissuelyser at 50 Hz for five minutes, and the tissue lysates were kept on ice for thirty minutes. Tissue lysates were centrifuged at 15,000 rpm at 4° C. for ten minutes. The supernatants were gently transferred to a new microcentrifuge tube and kept of ice. Protein concentrations in the supernatants were measured using the Pierce™ BCA Protein Assay Kit. Based on the result of BCA protein quantification, the samples were diluted to the same final concentration of 4 μg/μL using RIPA buffer, 4×LDS sample buffer, and 10× sample reducing agent. Diluted samples were heated at 100° C. for ten minutes. Remaining denatured samples were used for western blot analysis or kept in the −80° C. ultralow freezer for long term storage.

### Western Blotting

Extracted protein samples (5 μL per slot) were loaded to NuPAGE® Novex 4-12% Bis-Tris gel. Electrophoresis was run with MES running buffer at 80 V for thirty minutes, and 120 V for ninety minutes. Protein samples were transferred to nitrocellulose (NC) membranes with iBlot® 2 Gel Transfer Device (P3, seven minutes). The membrane was cut and parts of interest were kept. The membrane was washed with 5 to 10 mL of 1×TBST once for five minutes. Non-specific protein was blocked by 5-10 mL of 1×TBST with 5% nonfat milk at room temperature for one hour. The membrane was washed with 5-10 mL 1×TBST three times for five minutes each. After washing, the membrane was incubated with 5 to 10 mL of diluted primary antibody (BRAF V600E Rabbit Monoclonal antibody diluted at a ratio of 1:1000, phospho-p44/42 MAPK antibody, p44/42 MAPK mouse mAb and GAPDH antibody diluted at a ratio of 1:2000) in 1× TBST with 5% bovine serum albumin (BSA) containing 0.1% Tween 20 at 4° C., with gentle agitation overnight. After overnight incubation, the membrane was washed with 5 to 10 mL of TBST three times for ten minutes each. The membrane was incubated with 5 to 10 mL of Goat Anti-Rabbit IgG H&L (HRP) and Goat Anti-Mouse IgG H&L (HRP) at a ratio of 1:3000 in 1× TBST with 5% nonfat milk and 0.1% Tween 20 with gentle agitation for one hour at room temperature. After incubation, the membrane was washed with 5 to 10 mL of TBST three times for ten minutes each. After washing, HRP substrate from West Femto Maximum Sensitivity kit was added to the NC membranes. Samples were imaged, and chemiluminescence was detected by Amersham Imager 680.

### Data Management and Statistical Analyses

A one-way ANOVA followed by Dunnett's multiple comparison test was performed to compare tumor volume among vehicle group and treatment groups. All data were analyzed using GraphPad Prism, * indicates p<0.01, **** indicates p<0.0001 and ns indicates p>0.05.

### Results

### Results for Efficacy Study

### Body Weight Change

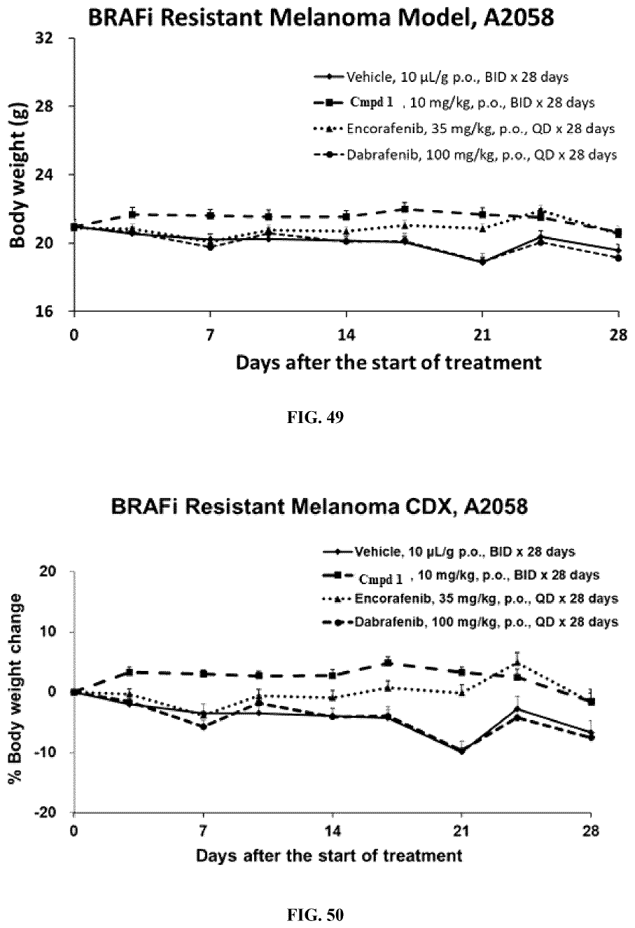

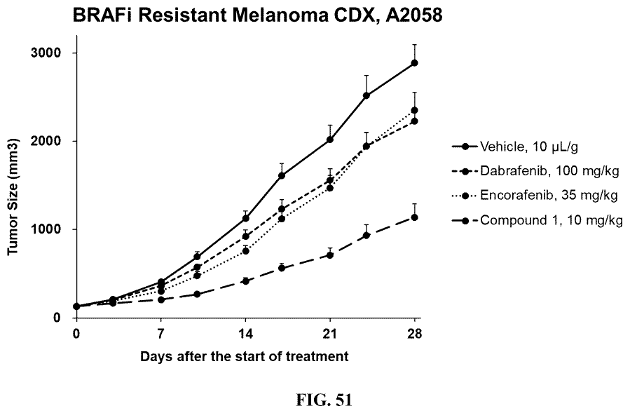

Animal body weight was monitored regularly as an indirect measurement of toxicity. Body weight changes after administration of Compound 1, encorafenib and dabrafenib are shown in FIG. 49 and FIG. 50.

### Tumor Growth Curve

Tumor growth curves are shown in FIG. 51.

### Tumor Volume Trace

Mean tumor size over time in female BALB/c nude mice bearing A2058 human melanoma xenografts is shown in Table 43.

TABLE 43
Tumor Volume Trace over Time (mm³)^{a}

| | Group# | | | |
|---|---|---|---|---|
| | Group 1, | Group 2, | Group 3, | Group 4, |
| | Vehicle, | Compound 1, | Encorafenib, | Dabrafenib, |
| | p.o., | 10 mg/kg, p.o., | 35 mg/kg, p.o., | 100 mg/kg, |
| | BID × 28 | BID × 28 | QD × 28 | p.o., |
| Days | days | days | days | QD × 28 days |
| 0 | 130 ± 3 | 130 ± 4 | 130 ± 4 | 130 ± 4 |
| 3 | 210 ± 8 | 167 ± 8 | 189 ± 9 | 203 ± 6 |
| 7 | 407 ± 24 | 205 ± 12 | 302 ± 23 | 361 ± 28 |
| 10 | 691 ± 55 | 267 ± 16 | 477 ± 45 | 572 ± 29 |
| 14 | 1124 ± 86 | 414 ± 38 | 756 ± 64 | 921 ± 74 |
| 17 | 1616 ± 136 | 562 ± 53 | 1122 ± 120 | 1232 ± 105 |
| 21 | 2019 ± 163 | 711 ± 79 | 1472 ± 142 | 1558 ± 132 |
| 24 | 2518 ± 229 | 933 ± 119 | 1944 ± 160 | 1948 ± 150 |
| 28 | 2888 ± 208 | 1139 ± 153 | 2354 ± 201 | 2227 ± 140 |

^{a}Data are shown as Mean + SEM.
b. Mouse#1-4 was euthanized on PG-D24 due to the tumor size over 3000 mm³.
Mouse#1-4 was found dead on PG-D26.

### Tumor Growth Inhibition Analysis

Tumor growth inhibition of Compound 1, encorafenib and dabrafenib in the treatment of female BALB/c nude mice bearing A2058 xenografts was calculated based on tumor volume measured on PG-D24 and PG-D28. Anti-tumor activity and tumor growth inhibition data is provided in Table 44 and Table 45.

TABLE 44
Tumor Growth Inhibition Analysis on PG-D24 (T/C and TGI)

| | Tumor Size | | | |
|---|---|---|---|---|
| | (mm³)ª | T/C^{b} | TGI^{c} | p |
| Treatment | on PG-D24 | (%) | (%) | value^{d} |
| Group 1, Vehicle, p.o., BID × 28 days | 2518 ± 229 | / | / | / |
| Group 2, Compound 1, 10 mg/kg, | 933 ± 119 | 37.06 | 66.37 | * |
| p.o., BID × 28 days | | | | |
| Group 3, Encorafenib, 35 mg/kg, | 1944 ± 160 | 77.22 | 24.04 | ns |
| p.o., QD × 28 days | | | | |
| Group 4, Dabrafenib, 100 mg/kg, | 1948 ± 150 | 77.37 | 23.87 | ns |
| p.o., QD × 28 days | | | | |

TABLE 45
Tumor Growth Inhibition Analysis on PG-D28 (T/C and TGI)

| | Tumor | | | |
|---|---|---|---|---|
| | Size | | | |
| | (mm³)ª | | | |
| | on PG- | T/C^{b} | TGI^{c} | p |
| Treatment | D28 | (%) | (%) | value^{d} |
| Group 1, Vehicle, p.o., BID × 28 days | 2888 ± 208 | / | / | / |
| Group 2, Compound 1, 10 mg/kg, | 1139 ± 153 | 39.45 | 63.41 | **** |
| p.o., BID × 28 days | | | | |
| Group 3, Encorafenib, 35 mg/kg, | 2354 ± 201 | 81.52 | 19.37 | ns |
| p.o., QD × 28 days | | | | |
| Group 4, Dabrafenib, 100 mg/kg, | 2227 ± 140 | 77.13 | 23.96 | * |
| p.o., QD × 28 days | | | | |

^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T0)/(C − C0))*100, where T = mean tumor size at the terminal end of treatment group; T0 = mean tumor size at day 0 of treatment group; C = mean tumor size at the terminal end of control group; and C0 = mean tumor size at day 0 of control group.
^{d}A one-way ANOVA followed by Dunnett's multiple comparison test was performed to compare tumor volume among vehicle group and treatment groups. All data were analyzed using GraphPad Prism, **** indicates p < 0.0001, * indicates p < 0.05 and ns indicates p > 0.05.
^{e}Mouse#1-4 was euthanized on PG-D24 due to the tumor size over 3000 mm³. Mouse#8-3 was found dead on PG-D26.

### Results for PKPD Study

### Pharmacokinetics Results

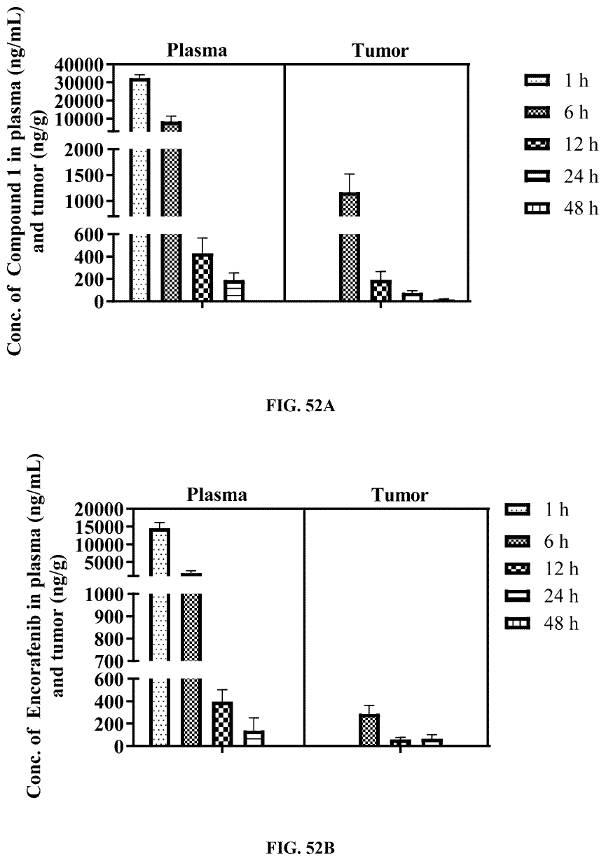

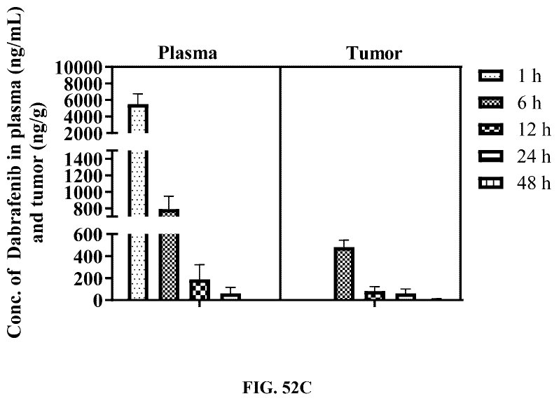

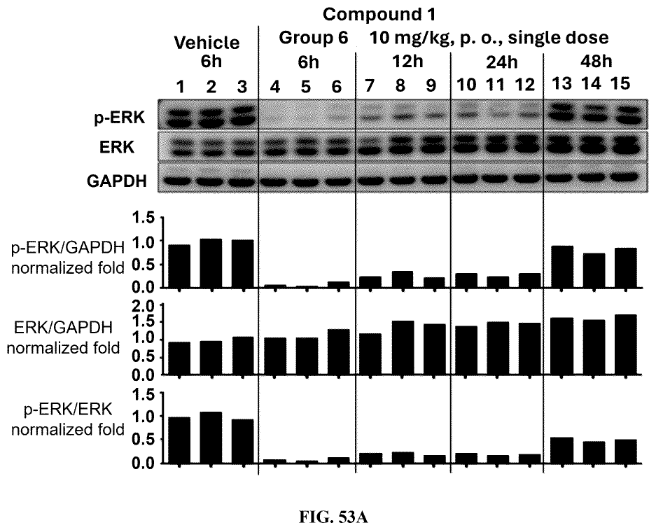

The concentrations of Compound 1, encorafenib and dabrafenib in the treatment of female BALB/c nude mice bearing A2058 xenografts are shown in Table 46 and FIG. 52.

TABLE 46
Mean Concentration of Test Articles in Plasma and Tumor

| | | Concentration in | Concentration in |
|---|---|---|---|
| | Sample | Plasma | Tumor (ng/g) ± |
| Group | time | (ng/mL) ± SEM | SEM |
| Group 6, | 1 h | 32467 ± 1683 | / |
| Compound 1, 10 mg/kg, | 6 h | 8497 ± 2968 | 1170 ± 352 |
| p.o., | 12 h | 429 ± 137 | 191 ± 74.4 |
| Single Dose | 24 h | 190 ± 63.2 | 76.0 ± 18.3 |
| | 48 h | / | 16.1 ± 6.73 |
| Group 7, | 1 h | 14500 ± 1626 | / |
| Encorafenib, 35 mg/kg, | 6 h | 1892 ± 654 | 289 ± 73.0 |
| p.o., | 12 h | 397 ± 106 | 58.8 ± 18.8 |
| Single Dose | 24 h | 138 ± 112 | 64.7 ± 36.4 |
| | 48 h | / | 3.06 |
| Group 8, | 1 h | 5497 ± 1247 | / |
| Dabrafenib, 100 mg/kg, | 6 h | 790 ± 157 | 480 ± 63.7 |
| p.o., | 12 h | 189 ± 133 | 81.9 ± 40.2 |
| Single Dose | 24 h | 61.3 ± 53.4 | 60.1 |
| | 48 h | / | 8.55 ± 4.03 |

### Western Blotting Analysis Results

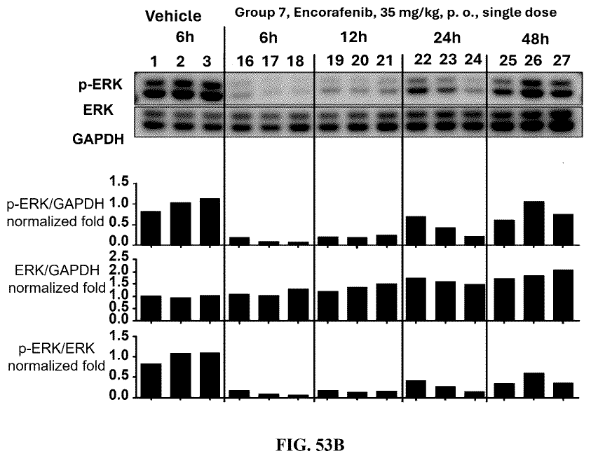

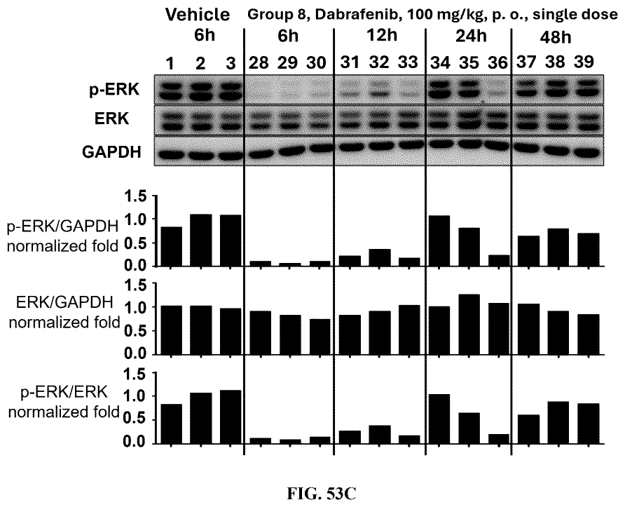

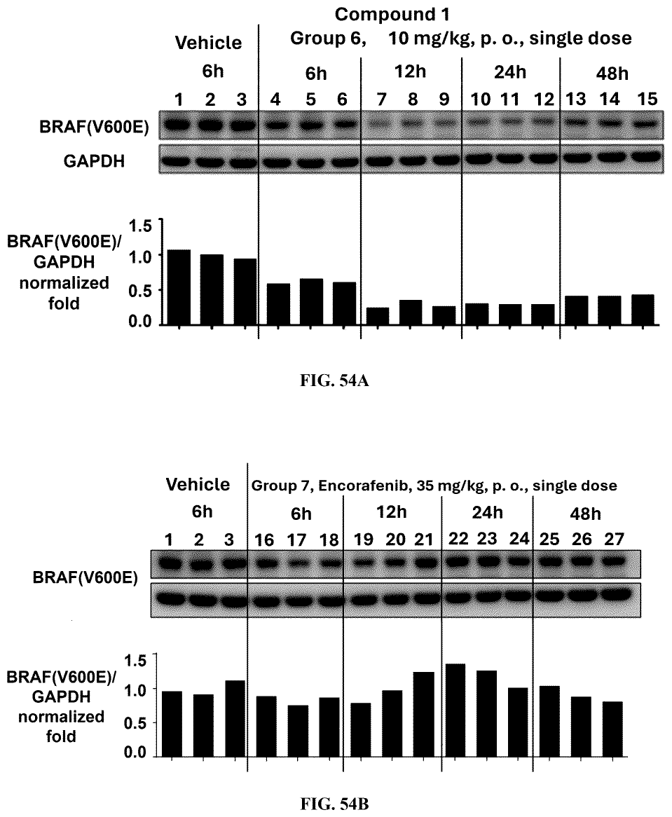

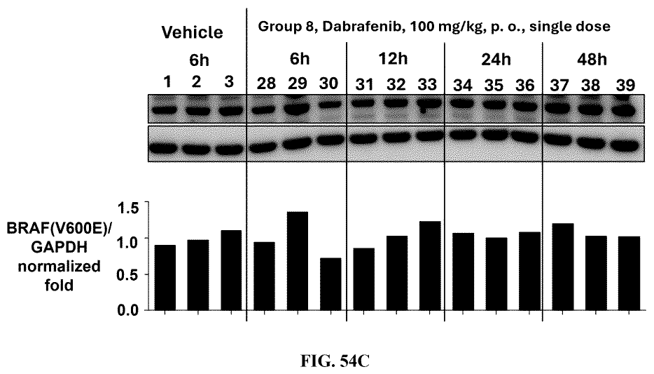

The levels of p-ERK, ERK and BRAF (V600E) expression in treated A2058 tumors are shown in FIG. 53 and FIG. 54.

### CONCLUSION

In this study, the therapeutic efficacy and the pharmacokinetic and pharmacodynamic effects of Compound 1, encorafenib and dabrafenib in the treatment of female BALB/c nude mice bearing A2058 xenografts was evaluated.

For the efficacy study, animal body weight was monitored regularly as an indirect measurement of toxicity. Body weight changes after administration of Compound 1, encorafenib and dabrafenib are shown in FIG. 39.

Mouse #1-3 from Group 1 (Vehicle) suffered over 10% body weight loss on PG-D7, PG-D21, and PG-D28. Mouse #1-1, #1-2, #1-4 and #2-2 from Group 1 (Vehicle) suffered over 10% body weight loss on PG-D21. Mouse #2-3 from Group 1 (Vehicle) suffered over 10% body weight loss on PG-D21 and PG-D28. Mouse #7-1, #7-3, #7-4, #8-2 from Group 4 (dabrafenib, 100 mg/kg) suffered over 10% body weight loss on PG-D21. Mouse #8-4 from Group 4 (dabrafenib, 100 mg/kg) suffered over 10% body weight loss on PG-D21 and PG-D28. The mice from Group 1, Group 3 and Group 4 were supplied with extra nutrients from PG-D7 to PG-D28. Other animals maintained their body weights well.

Tumor sizes of all treatment groups at various time points are provided in Table 43 and FIG. 51. The calculated T/C and TGI data are shown in Table 44 and Table 45 Table. Mean tumor size of vehicle treated mice reached 2518 mm³ on PG-D24. Compared with vehicle group, treatment with compound 1 at 10 mg/kg BID (TV=933 mm³, T/C=37.06%, TGI=66.37%, p<0.0001) exhibited significant antitumor activity. Encorafenib at 35 mg/kg QD (TV=1944 mm³, T/C=77.22%, TGI=24.04%, p>0.05) and dabrafenib at 100 mg/kg QD (TV=1948 mm³, T/C=77.37%, TGI=23.87%, p>0.05) had no significant antitumor activity.

For the pharmacokinetic study, the concentrations of compound 1, encorafenib and dabrafenib in plasma and tumor are shown in Table 46 and FIG. 52. The data showed that the concentration of compound 1, encorafenib and dabrafenib peaked at one hour in plasma and six hours in tumor.

The Western Blotting results of BRAF (V600E), p-ERK and ERK are shown in FIG. 53 and FIG. 54. The data showed that Compound 1, encorafenib and dabrafenib inhibited the expression of p-ERK from six hours to twenty-four hours post dose. Compound 1 inhibited the expression of BRAF from six hours to forty-eight hours post dose. Encorafenib and dabrafenib had no effect on the expression of BRAF.

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A crystalline compound of structure: 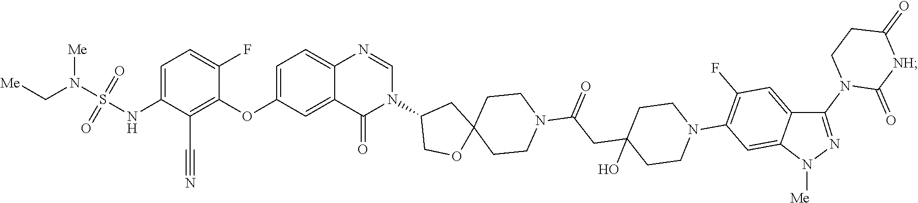 [SMILES: CCN(C)S(=O)(=O)Nc1ccc(F)c(Oc2ccc3ncn([C@H]4COC5(CCN(C(=O)CC6(O)CCN(c7cc8c(cc7F)c(N7CCC(=O)NC7=O)nn8C)CC6)CC5)C4)c(=O)c3c2)c1C#N]
or a pharmaceutically acceptable salt thereof.

2. The crystalline compound of claim 1, which is characterized by an X-ray powder diffraction (XRPD) pattern comprising at least five 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

3. The crystalline compound of claim 2, wherein the XRPD pattern comprises at least six 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

4. The crystalline compound of claim 2, wherein the XRPD pattern comprises at least seven 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

5. The crystalline compound of claim 2, wherein the XRPD pattern comprises at least eight 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

6. The crystalline compound of claim 2, wherein the XRPD pattern comprises at least nine 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

7. The crystalline compound of claim 2, wherein the XRPD pattern comprises at least ten 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

8. The crystalline compound of claim 2, wherein the XRPD pattern comprises at least eleven 2theta values selected from 7.5±0.2°, 8.8±0.2°, 10.0±0.2°, 10.5±0.2°, 12.6±0.2°, 14.7±0.2°, 15.4±0.2°, 16.4±0.2°, 16.7±0.2°, 18.6±0.2°, 22.7±0.2°, and 25.3±0.2°.

9. The crystalline compound of claim 2, wherein the XRPD pattern comprises at least the 2theta value of 16.4±0.2°.

10. The crystalline compound of claim 2, wherein the XRPD pattern comprises at least the 2theta value of 10.0±0.2°.

11. The crystalline compound of claim 2, wherein the XRPD pattern comprises at least the 2theta value of 15.4±0.2°.

12. The crystalline compound of claim 2, wherein the XRPD pattern comprises at least the 2theta value of 16.7±0.2°.

13. The crystalline compound of claim 2, wherein the XRPD pattern comprises at least the 2theta value of 8.8±0.2°.

14. The crystalline compound of claim 2, wherein the XRPD pattern comprises at least the 2theta value of 12.6±0.2°.

15. The crystalline compound of claim 2, wherein the XRPD pattern comprises at least the 2theta value of 14.7±0.2°.

16. The crystalline compound of claim 2, wherein the XRPD pattern comprises at least the 2theta value of 10.5±0.2°.

17. The crystalline compound of claim 2, wherein the XRPD pattern comprises at least the 2theta value of 25.3±0.2°.

18. The crystalline compound of claim 2, wherein the XRPD pattern comprises at least the 2theta value of 22.7±0.2°.

19. The crystalline compound of claim 1, characterized by an XRPD pattern having the characteristic 2theta values of FIG. 1.

20. The crystalline compound of claim 1, wherein the crystalline compound has differential scanning calorimetry (DSC) onset endotherm of about 194±20° C.

21. The crystalline compound of claim 1, wherein the crystalline compound has differential scanning calorimetry (DSC) onset endotherm of about 194±10° C.

22. A pharmaceutical composition comprising a crystalline compound of claim 1 and one or more pharmaceutically acceptable excipients.

23. A pharmaceutical composition prepared from a crystalline compound of claim 2 and one or more pharmaceutically acceptable excipients.

24. A method of treating a mutant BRAF mediated disorder comprising administering an effective amount of a crystalline compound of claim 1 to a human patient in need thereof.

25. The method of claim 24, wherein the mutant BRAF mediated disorder is a mutant BRAF mediated cancer.

26. The method of claim 25, wherein the cancer is melanoma.

27. The method of claim 25, wherein the cancer is lung cancer.

28. The method of claim 25, wherein the cancer is non-small cell lung cancer.

29. The method of claim 25, wherein the cancer is colorectal cancer.
